SEQUENCE LISTING (245 sequences)

SEQ ID NO: 1 - protein, 263 aa, Arabidopsis thaliana
MANSGNYGKRPFRGDESDEKKEADDDENIFPFFSARSQYDMRAMVSALTQVIGNQSSSHDNNQHQPVVYNQQDPNPPAPPTQDQGLLRKRHYRGVRQRPWGKWAAEIRDPQKAARVWLGTFETAEAAALAYDNAALKFKGSKAKLNFPERAQLASNTSTTTGPPNYYSSNNQIYYSNPQTNPQTIPYFNQYYYNQYLHQGGNSNDALSYSLAGGETGGSMYNHQTLSTTNSSSSGGSSRQQDDEQDYARYLRFGDSSPPNSGF

SEQ ID NO: 2 - protein, 422 aa, Zea mays
MERVKYCDCTVCSVQRSLCSTRRRRRRRRQIDRQLTKVDPRRRHGKRPLPAAEVEEEEEEEALPPGPPPAKHEQLEEPHHAAVSQLQGATFSGGGGSSSSSVIGGPSPPQAYAQYYYSARADNDASAVASALAHVIRASPDQLPPQQAPALYGAGVPGSLRLGDHPQASAHHYPGPGGHVAAAEEEQGRRRHYRGVRQRPWGKWAAEIRDPKKAARVWLGTFDTAEDAAIAYDEAALRFKGTKAKLNFPERVQGRTDLGFLVTRGIPDHRHPSAAVTLAAMPPPHHQHGHQTVVPYPDLMQYAQLLQGGRGGGGHAEAAVQQAHRQQQQQQLMTMMGGRPGVNLPSTFSPSSSASAPQILDFSTQQLIRPGPPSPSPPRAAAMPSSSAAAAPSTPSSTTTASSPSGGAWPYGGERHRNKKDA

SEQ ID NO: 3 - protein, 443 aa, Zea mays
MRISLRVLISSELGTSLCTAAPSLARASVRKSKSSALSLTHARIDRSSSTHRRRRQINGQLTKVDPRRRHGKRPLPADEEEEEEEELPPPPAKYEQLDQEEKHHVVVSQLQAGATFSGGRGSSSSSVAGPSPEAYAQYYYSARADHDASAVASALAHVIRASPDQLPPQQAACLYGAAGAPVLRQGEGDHPQPQAAAHHHPGGHVAAEEEQGLRRHYRGVRQRPWGKWAAEIRDPKKAARVWLGTFDTAEDAAIAYDEAALRFKGTKAKLNFPERVQGRTDLGFVVTRGIPDHHRHPRAAAVNLAAMPQAQAQPHLQHGRPTVMPYPYPYPDLMQYAQLLQGGRGGGDHAAAVQQQLMMMGGRGGNLPFSFSPPSSWSAPPQILDFSARQLITQPGPPSSPAAPGGAAPSTPSSTTTASSPSASASGSAWPYGGEHHRNKKDA

SEQ ID NO: 4 - protein, 357 aa, Zea mays
MCFELADQRGPQGGGGAGWPAKRRAGGVQDEGAAAAAGMAMAAAGPGEVMSEYYQAQELSTMVSALTHVVAGAPMGSAPAQRPMHGASGYYAHEMGSYRGAPSPELAGSELSSDTQSAGAAAMEEHQSAAALSSQEGPETPRRRYRGVRQRPWGKWAAEIRDPHKAARVWLGTFETAEAAARAYDEAALRFRGSRAKLNFPEDARLYPASTAGAAAPLAAAASTSPPVYSGGVQGSSDYLRYHQMLLQASTGSQGTLLPFYGGGMSNPYGGGAAMTGSYGGAGGGNTSGSLGSYYSFPASSVSVATVPSSTSSASGYYYSSPHDSQHSEASAAADWNWESALAWPDSSQYPPPPHTQ

SEQ ID NO: 5 - protein, 134 aa, Vitis vinifera
MSAMVSALTQVIGNTDKNPLHDLGNPSPISHHSATTPHDQPSQLLQDQGNQLRRRHYRGVRQRPWGKWAAEIRDPNKAARVWLGTFDTAEDAALAYDEAALRFKGNKAKLNFPERVQGRSELGYLTNPPSRWRW

SEQ ID NO: 6 - protein, 246 aa, Vitis vinifera
MSAMVSALTQVIGNTDKNPLHDLGNPSPISHHSATTPHDQPSQLLQDQGNQLRRRHYRGVRQRPWGKWAAEIRDPNKAARVWLGTFDTAEDAALAYDEAALRFKGNKAKLNFPERVQGRSELGYLTNRQDFLLPQQQQLPNPAVPPLPHPSLPRPSYPNLHHYAQLLPGGGGDLNHAMSSLYGREASTTQSLSTTSSSSSTTSHPQHHQRRRQREEEELQQPQLLQFSSLFGSSSSNDPHNNRRDD

SEQ ID NO: 7 - protein, 240 aa, Populus trichocarpa
MDVMVSALAQVIGSSHNSSAQVQENPLTSTQSSTENDQTQPAVQDQGNARRRHYRGVRQRPWGKWAAEIRDPKKAARVWLGTFETAEAAALAYDEAALRFKGSKAKLNFPERVPSGGTELGFFTRGQGLHTVTEPVSNHIMAPLARSQRSQEAYNPNNFQYPQFLGTTSGYGLSHVMPPAVPFGGETFLSPTSSSASSNSWPISSQQQQQQQEELLRLSMQFGSSYNSRYDPSKYKDEGL

SEQ ID NO: 8 - protein, 289 aa, Solanum lycopersicum
MDRIRRGKRRYESEEKEDRNYNHMYSSARSQHDMSTMVAVLSQVIGNKSTTNTNSSSSSSAHHKPLLTLNHQSNTTAAMQNQLPQLNQQQGNNEKRRRQYRGVRQRPWGKWAAEIRDPEKAARVWLGTFHTAEDAAIAYDEAALKFKGNKAKLNFPERVQSTTDQFGISYLITNTNHQQHQFQPTNFLPNSDQLQQHHYSNHNADDLKFGVSPSFYHPTGFNPKALDLVEPSKSSSMTYLVQQASSHQVQEEPRYINHQQEDENNLKFSSYFGTYSSSGPTLGEFEDQK

SEQ ID NO: 9 - protein, 244 aa, Solanum lycopersicum
MKRSSSNNDQRDEKDTSNIFPIYSSARSQHDMSAMVSALSQVIGNSSSSASGDSSSVHVNPLTLIQQHQSQSSTQDQERRRYRGVRQRPWGKWAAEIRDPKKAARVWLGTFETAEGAALAYDEAALRFKGNKAKLNFPERVQGQFFQCYDQPATSSNNTSEQNYPNVHHYADLLLRTDNNIDLNFDVSPNTFYHSFDISQSSMEVPVYHEEQQQVITTHEEEEEDFVKYRGSHFGNSTSSGGTK

SEQ ID NO: 10 - protein, 230 aa, Solanum tuberosum
MDRTRHGKRPYESEEKEDTNNNQMYSSARSQHDMSTMVSVLSQVIGNKSRTNTNSSSSSSAHHKPLLTLNHRSSTTAAMQNQLPQLNQQQGNNERRRRQYRGVRQRPWGKWAAEIRDPEKAARVWLGTFHTAEDAAIAYDEAALKFKGNKAKLNFPERVQSATDQFGISYLITTTNQFPANKFLPNSDQLQHHYAPAGGSNHNADDLNFGVSPSSYHPTGKSTVNDLSTQ

SEQ ID NO: 11 - protein, 269 aa, Glycine max
MEGRSISHSSEREEEYDLFPIYSERSQQDMSAMVSALTQVIGGSNSDSLQQHEGLLTSSHNNTSTQNNNEQSQAPQQEQGSVRRRHYRGVRQRPWGKWAAEIRDPKKAARVWLGTFETAEAAALAYDEAALRFKGSKAKLNFPERVQGTASEFGYHLTNQHSTSSHDQQASNPIITPHFATTQETYSPSHHFQYAQQQLMGGGSNSFNNNQDMLRFYGGHNMFVSSQQSASSSSSTALSQNQQDELLRFSMQFGASSHSDHSGNWRGGQ

SEQ ID NO: 12 - protein, 233 aa, Gossypium raimondii
MNAMVHALAQVIGNNNSNPLLQLHDDQHPNPTAQQNQSHQQPQPQDQGNARRRHYRGVRQRPWGKWAAEIRDPKKAARVWLGTFETAEAAALAYDDAALRFKGSKAKLNFPERVQGRLESSYLTTTRQELERTEAPPHPPPTYPNISQYAQLLSGGLPNTAFNYAMPSGAAYGSWPAFTTSSHSSSSSSSSTTLTSQQQGYMGGFSLHFGGSSPTSDHTNNMGDYDYYYSRDQ

SEQ ID NO: 13 - protein, 255 aa, Brassica rapa
MNNGKRPFRAGESEEKKEADDDENIFPFFSARSEYDTRAMVSALTQVIGNQSSTHDNNLHHPVEYDQQDPIQHVPPTQDHGNLRKIHYRGVRQRPWGKWAAEIRDPQKAARVWLGTFETAEAAALAYDEAALKFKGSKAKLNFPERAQLASNTSTITGLPNYYSSNNQTYYSNPQTNPQNIPYYNQYYYNQYLQQGGNSNDALSYSLAGGETGGSIYSHQTLSNTTSSPAGGSLRQQEDYTRFWHFGDSSPNSGV

SEQ ID NO: 14 - protein, 253 aa, Brassica rapa
MMPFGAGESDERKEADDEENIFPFLSARSQYDTRAMVSALTQVIGNQSSTHDSNQHHPVEYNQQDPIQHVPPTQDQGNLRKRHYRGVRQRPWGKWAAEIRDPQKAARVWLGTFETAEAAALAYDEAALKFKGSKAKLNFPERAQLASNASTITGLPNYHSSNNQMYYSNPQTNPQTMPYYNQYYYNQYLQQGGNSNDALSYSLAGGETGGSMYNHQSISNTTSSSSGGSSRPQQEQDYARFWHFGDSSPSSGF

SEQ ID NO: 15 - protein, 250 aa, Hordeum vulgare
MVSALSHVIRATPDQEPAYYPAGPAAVSREQQHQHAAAIAEEQGRKRHYRGVRQRPWGKWAAEIRDPKKAARVWLGTFDTAEDAAIAYDEAALRFKGTKAKLNFPERVQGRTDLGFVVTRGIPDRLQQQQHYPAAVGAPAMRPPLHQQQAVVPYPDLLRYAQLLQGAGSAGGAVNLPFGAMSPPSMSSSSSPHILDFSTQQLIRVSPASPAAAISGSATTGPSTSSSTTTASSPGAAWPYTGEQKNNKDS

SEQ ID NO: 16 - protein, 269 aa, Oryza sativa
  source 1..269: note = Subspecies Indica
MVTALAHVIRAAPDLHLPHHPSSSASAAAHPQQASSFYPTAAAAASSPSDQLAAAAAAAEEQGRRRHYRGVRQRPWGKWAAEIRDPKKAARVWLGTFDTAEDAAIAYDEAALRFKGTKAKLNFPERVQGRTDLGFLVTRGIPPAATHGGGYYPSSSPAAGACPPPRQQQTVVPYPDLMRYAQLLQGGVGGSYMPFGGAATMSSSTVSSSSAPQILDFSTQQLIRAGPPSPMPSSGSGSATAAASSTTSASSPGAWPYGGSERKKKDSSS

SEQ ID NO: 17 - protein, 269 aa, Oryza sativa; identical to SEQ ID NO: 16
  source 1..269: note = Subspecies Japonica

SEQ ID NO: 18 - protein, 393 aa, Setaria italica
MPGSIDPAPSADGRRRRRQIDRQLTKVDPRRHGKRPLPADKEEEDQPPPPPPAKHEQLEIEEHRYHVSQLQQGATFSAGGGGGGSSSSSAAGAAAGPSPEAYAQYYYSARADHDASAVASALAHVIRASPDQLPPHAFGGGGAPPGQGDYQQAAPPAAAAAAAEEEQAAGRRRHYRGVRQRPWGKWAAEIRDPKKAARVWLGTFDTAEDAAIAYDEAALRFKGTKAKLNFPERVQGRTDMGFLVTRGIPDRHHHQGGAAVTLAAMPPPHRQHHQTVVPYPDLMQYAQLLQGGGRGGGGAGDHHAEAAAQQAQARLMMMARGGVSLPFGAASFSSSSSSAPQILDFSTQQLIRPGPPSPAAAAPSTPSSTTTASSPGGSAWPYGGEHHRNKKDA

SEQ ID NO: 19 - protein, 294 aa, Sorghum bicolor
MTKKLISAMAGKQGFKEQQFNDQRRQQASIQGDDIAKSLVGFGGGGGRLISHEQEDAIIVAALRHVVSGYSTPPPEVVTVAGGEPCGVCGIDGCLGCDFFGAAPELTQQEAVNFGTGQMVATAAAAAAGGEHGQRTRRRRKKNMYRGVRQRPWGKWAAEIRDPRRAARVWLGTFDTAEEAARAYDCAAIEFRGARAKLNFPGHEALLPFQGHGHGGDACATAAANAETQTTPMLMTPSPCSADAAAAAPGDWQLGGGVDGGEGDEVWEGLLQDLMKQDEADLWFLPFSGAASSF

SEQ ID NO: 20 - protein, 236 aa, Theobroma cacao
MTAMVTALTHVMGTGGSDEQLSFTPSSVPLSQSAVKEEPDPPQPVQDQENTRRRHYRGVRQRPWGKWAAEIRDPKKAARVWLGTFDTAEDAALAYDRAALKFKGTKAKLNFPERVQGNTEVSYFTGHGDSSTVRPDQNPTPAATPPSWSQDSYPHLFQYAQLLSSSNDADISYYTSNLFNQEPLSPQFPSMAASPNISSQYHHQDQTRFSTKYESSSGSDYPEQYGKDSDPSNRSE

SEQ ID NO: 21 - protein, 232 aa, Elaeis guineensis
MVSALAHVISSSLSGVGVGVGRSESVVIQSELNPAMAGPESGSMERELSQPSEEQGNVRRRHYRGVRQRPWGKWAAEIRDPRKAARVWLGTYNTAEEAAIAYDEAALRFKGTKAKLNFPERVQGRTDLGFLVSRGIPERPPQPITPPPTASYPDLLQYAQLLQSRDEDLHSVASGLFVTDSFTSGSSQVSYHSTSGSSQEFLDFFSQMRSSSSSSSRQPRGDQKDKDSNQQQ

SEQ ID NO: 22 - protein, 234 aa, Elaeis guineensis
MVSALAHVISSSSPGVGVGVGVGGGETREIQPELSPAMAGTGSGSMEIRELSQPSQEQGNVRRRHYRGVRQRPWGKWAAEIRDPKKAARVWLGTFDTAEQAAIAYDEAALRFKGTKAKLNFPERVQGRTDLGFLLSRGIPERQPEPITPSAAATYPDLLQYAQLLQSRDEDFHNVASGLYIGGSFASGSSQMSPASMSGSSQEFLDFSSQFGTSSSSTSWPHGDQKDKDSSQHP

SEQ ID NO: 23 - protein, 244 aa, Malus domestica
MSAMVSALTQVIGTTDDHAAVQPNPTSISDSSLLVKQEPDRSQPVQDQEPVRRRHYRGVRQRPWGKWAAEIRDPKKAARVWLGTFETAEDAAIAYDNAALKFKGTKAKLNFPERVQGKTDLGILMGSSGSGAASTQRTQNLMTPAGHIVNPQPAPAPLMMSQQPETFPDLYQYARLLSGNDADFYNYSSYPFNQDPRFTSRFLPSSTHFSSSTASQDSQPPQQGQQDHEEDGGNEDRNWSNPRE

SEQ ID NO: 24 - protein, 268 aa, Malus domestica
MIFDPFCSSARSQHEMSAMVSALAQVLGSNNSQTPAVQEPVEPPLITPQSSAMELHDGQSPQQAQDQGNVRRRHYRGVRQRPWGKWAAEIRDPKKAARVWLGTFETAEAAAISYDEAALRFKGSKAKLNFPERVQGRITELGYLTTTSTQQNLPAEAQSITDHHQPLPDHQYQLQAFPNNVATPHDQYAPYFLSGNEGVNYDDLPTNLSERERFAFQTSETTSSSLLPFLPSHQQEDQQHPLNYSMPAFGSSSSSSSNPPPRNRNRRP

SEQ ID NO: 25 - protein, 272 aa, Malus domestica
  SITE 172: note = misc_feature - Xaa can be any naturally occurring amino acid
  SITE 218: note = misc_feature - Xaa can be any naturally occurring amino acid
MIFEPFCSSARSQHEMSAMVSALSQVLGSTNNQTPAVQVPMEPPMIAPQSSAMESHDDQSPQHARDQGTARRRHYRGVRQRPWGKWAAEIRDPKKAARVWLGTFETDEAAALAYDEAALRFKGSKAKLNFPERVQGRITELGSLTATSTQQNLPAGTSQGVITDHHHPLSDXQYQLQAFPNNVVTPPHDQYAQYFRSGHECISYDLPATLYEREIFAXQTSETTTSSSSLLPFLPSHQQEDQQHPLSYSMPGFGSSSSSSSNPPPRNRNRRP

SEQ ID NO: 26 - protein, 264 aa, Malus domestica
  SITE 115: note = misc_feature - Xaa can be any naturally occurring amino acid
MYDIATNSQFDVHKVDRKHGKRLLASVESEEKEEDQIFPVYSTRSQQDTSAMVSALAQVIGKNSDQINNPLDQVQGINPLITSQSSPTETQSQTVLQDQGNLRRQHYRGVRRRPXGKWAAEIRDPVKAARVWLGTFDTAEAAALAYDKAALKFKGSKAKLNFPERVQGISESGCLALITTDQQDSLNINPPQPNITRPTSSSVFDCTQYNNVTSSTSLLSSSSMPSQQAAELPSFSMQFGSSFSSSSSGPPHKYRKDFDSSHSR

SEQ ID NO: 27 - protein, 214 aa, Malus domestica
  SITE 110: note = misc_feature - Xaa can be any naturally occurring amino acid
MVSALAQVIGKNSDQINNPLGQVQGINPLTTSQSSPTETQSQPVLLQDQGNLRRQHYRGVRRRPSGKWAAEIRDPVKAARVWLGTFDTAEAAALAYDKAALKFKGSKAKXNFPERVQGSSESGCLTLITTDQQDSLNINPPQPNITRPTSSSVFDCTQHNNVIYCTSLLSSSSMPSQQAAELPSFSMQFGSSFSSSSSGPPHKYRKDFDSRHSR

SEQ ID NO: 28 - protein, 304 aa, Malus domestica
MFFRYTFLLGIRAVSSVVFRSSVVEAILVTTTALQYVQTTRTTISAATSTCSRVKLDKVDLKHGKRPLASVESEEKEEDQIFPVYSARSQQDTSAMVSALAQVIGNNSDQINNPLDQVQGISSLITSQSSPTETQSQPVLLQDQGNLRRQHYRGVRRRPWGKWAAEIRDPIKAARVWLGTFNTAEAAALAYDGAALRFKGSKAKLNFPERVVQGSSESGCLTITTQLQHSLNNIPPEANISRPTYSNVFDYARYNNVTSTSSSSMPSQQAAELRSFSMQFGSSSSSSSGPPYKYRKDFDRSHSR

SEQ ID NO: 29 - protein, 243 aa, Fragaria vesca
MSAMVSALTQVIGNPEEDNKQVQSNPASVKDEPDRSQPVQDQESTVRRRHYRGVRQRPWGKWAAEIRDPKKAARVWLGTFETAEDAAIAYDNAALKFKGTKAKLNFPERVQGNSSILIQGSSGTTSSGSSVSTERNRSRPALTVPHHDASYVAPSQPQQESSSFPDLYQYAQLLQSNDIDFSSNYQYPPNNPFNQDYHPQYSTPQFPPSTYYPSHQQQGQQDDHVQEDHQNENKNWNRRNPSE

SEQ ID NO: 30 - protein, 240 aa, Fragaria vesca
MSAMVSVLSRVISGDSSTDTDPNPALLQLPQQSSTATPELDQSHQQAAPDQAGSVRRRHYRGVRQRPWGKWAAEIRDPKKAARVWLGTFETAEAAALAYDEAALRFKGSKAKLNFPERVQGTSESGGYLTTQTVAHQPLISDYHQQQLVYPNNITTTDQYYPQFYGNLNYGQPDPRFYNQPATSSYPPFIPISQAQEEDRQQQPALSSPMPDFGSPSSSLHPEYPQYDTTRNFDNSHWRG

SEQ ID NO: 31 - protein, 224 aa, Fragaria vesca
MAKEQEHAIMVSALEQVIGGGIATRTSGTSQNHCQYATSAAEAGTNKKVVILVSDGDTCQVCKIDGCLGCEFFPPSNKHGKGKRVKKSKYRGVRQRPWGKWAAEIRDPRRAVRVWLGTFQTAEEAARAYDKAAVEFRGEKAKLNFPRISSEAGTSTAALTKQSMETDDEVHNQVNPSNEKSANQELGQGSDVKDDEIDRFIWKMLKDDDGDEDLSTMVNSNMLN

SEQ ID NO: 32 - protein, 255 aa, Fragaria vesca
MVSALTQVIGNTTHEQNPLQVIDHQVLGNPQVFSAENPVEQSQPAVVLQAQGNVRQHYRGVRRRPWGKWAAEIRDPHKAARVWLGTFETAEAAALAYDEAALRFKGSKAKLNFPERVQGISPSTSSGLYLAISTSTGHDHRLANSAPPAAPISRPTTYSINPNNVNIDYDALVSSSQNHGQERTMPQINVQTTLSSTSSASSMPSHHQQERVQQQEDQQQLLKFPIMPFGGSSSSSSDPPTKYRRDSSGSGDFRR

SEQ ID NO: 33 - protein, 239 aa, Eucalyptus grandis
MVSALSRVISTSDDAPSSADDPAAPVQEEHGDPPQQAPDQESVRKKHYRGVRQRPWGKWAAEIRDPKKAARVWLGTFETAEDAALAYDRAALKFKGTKAKLNFPERVQGKPEYAAYSNPSHQNSGVNVLPEQINPQPAPFVPYPHAAFPDLAQYAQLLSSNDAEFPYYVSNLYGQEPFGSQQSSSTSSSSISSSSYHYNQQQQQQEPQNEPSRTSFGWSPSNYDFQGYGDGFDPRNQGQ

SEQ ID NO: 34 - protein, 267 aa, Eucalyptus grandis
MEGRNWKRSKSQAGHVSEETEDGDRKRNNYAYPSSLMETARSQQDTSAIVSALAQVIANPAAAASHHASLSSSASLSQSSLHDHQAPDAQVGKNKKLEVSRNYRGVRQRPWGKYAAEIRDPKKAARVWLGTFDTAEGAALAYDEAALRFKGNKAKLNFPERVHSLPPPYGPACNASQPQSQLLPPAFSSCDNHVGAQLMDGCAMPPPRPSYVRGQGPTSSASASDYFQSLQPTPSLSSSSSMPSPFHQHDPQHSFDGFSSSWRSSHE

SEQ ID NO: 35 - protein, 267 aa, Eucalyptus grandis
MEGRNWKRSKSQAGHVSAEREDGDKKRNNYAYPSSLMETGRSQQDTSAIVSALAQVIANPAAAESHHASLSSSASLSQSSLHDHQAPDAQVGKNKKLEVSRNYRGVRQRPWGKYAAEIRDPKKAARVWLGTFDTAEGAALAYDEAALRFKGNKAKLNFPERVHSLPPPYGPASNASQPQSQQLPPAFSSCDNHVGAQLMDGCAMPPPCPSFVRGQGPTSSASASDYFQSLQPTPSSSSSSSTTSPFHQHDPQHSFDGFSSSWRSSQE

SEQ ID NO: 36 - protein, 282 aa, Eucalyptus grandis
MSGLKVADRGDKAPILYPGLDRERETSAMVLALARVVAGEVPGDAEESCPFPFPSGVLRLKRGHGDLSAEPSAEAQLRRAPGRESSVDDAARGIMEGPSMKTTNHATPTYEYSNSTAAMSMSNDEHQPRRKYRGVRQRPWGKWAAEIRDPVKAARVWLGTFETAEGAAQAYDVAALKYRGNKAKLNFPENVVARLSLAAPPATQMTVPDAARTHVTVPADTEHQLASGPDHGCGEWCSWLSPDPDLAHSNLLPSSSSPSSSSSSVSKAASFAFPTRSGSWPF

SEQ ID NO: 37 - protein, 387 aa, Eucalyptus grandis
MEEAVMPMYSPYCPPGETSAIVSALTHVVTGTRGGQHGGAYQSTLAPSFAYDSASAGSSSQLPWTYIGQKRERDEAGSSSQFLAEPPLSQRDYYGIYGGSFALRETSAISASLPGLQARTVAATSNVPPPPSGAGPPPYEGGERRRRYRGVRQRPWGKWAAEIRDPQKAARVWLGTFDTAEAAARAYDEAALRFRGNRAKLNFPENVRVIPPNVPTYGSPAAAAATLAAGSAPPAIAAGQLAPPYAGAVPLYPRGSEGFNLGDYWEYSQLLQGHHHHQPASLMEQMMYSSQMASSHSSLSLPSSSPPQPPPPPPSFPSSDLYGSSSSSGFGGASSFSSSVPLFFPQQPPLGYFRPPPPRPPHDQGSGGEDSEPPPSSDSSHYHSSTS

SEQ ID NO: 38 - protein, 348 aa, Eucalyptus grandis
MSIMVSALRRVVSGEVLSGDDQQQQFNLSGGYEPAALLGRSSSLDSVGVGQKRGRGDSTSSSEFLVSQDSVAFGGFPQAGSSSSSDARGHAGSTTVETAKADTADGTAPKYEYNYEAPTTMAASRLDPTVRRKYRGVRQRPWGKWAAEIRDPFKASRVWLGTFDTAEGAARAYDRAALQFRGSKAKLNFPENVRLRQQPAPVASAAFPSATHFTISRELPASGNFDATGYDGQAPMQQFPENDFREYHRDVAAHQERLQGRTMSLYEQMLFSNSSFGSQFQPAFSLSPSSSSSSSSVLARSPNSSLFMPSPPNVTSRSQESRRGSGDGAAVFSQHPWTDSSHYSSSSG

SEQ ID NO: 39 - protein, 256 aa, Eucalyptus grandis
MQQLHDLGMTRDDEHGIMVNALQHVISGSSSTRPEPIPAFRSSTPSHLSADGTAAEQRAPGLLSLPDVATCQVCRIDGCLGCNFFAPGRVAMAVQGGGDPAKAAQALGVGQSNKSAGRKRKGFYRGVRQRPWGKWAAEIRDPRRAARVWLGTFETAEQAARAYDRAALEFRGARAKLNFPLLPNDCTSTGAGKSRDMMEDGEAEETIKARAEGGESNDVREKAPSFERDDERGASEFWEKLEKEELEQWPVMHLPP

SEQ ID NO: 40 - protein, 246 aa, Citrus sinensis
MYGTAVVSALSQVIGNTQNSPTSLQLSQNPNFTTSSPNTSERDLSQRVEDQGNVRRRHYRGVRQRPWGKWAAEIRDPKKAARVWLGTFDTAEAAALAYDEAALRFKGSKAKLNFPERVQANLTTHRYQDHYYHAAAATTSQQVSNPPPPPPPRPLPLTQEVMYSNLFQYQQANYGIPSGFYGEYRYLPVTLPTTSSSSSSSSATSSQQPQQHELLRYGMQLESSSSSASDPHESTRRNSDTSHPGD

SEQ ID NO: 41 - protein, 233 aa, Citrus sinensis
MSAMVSALTQVIGTTTTDADADPSPAAVKEESSDNPLQQTQTQTQDQDQGTRRRHYRGVRQRPWGKWAAEIRDPKKAARVWLGTFETAEDAAMAYDKAALKFKGTKAKLNFPERVQGTTEFVYLDSSSSSSAFHHHHESVMPAPPPRPTSMHHGAYPDLLQYAQILSSDDATFNYYTSNLFNPQSSSSSSSTPSTFSSSTTSLEQQQEMTRFSSNYESLSGSDFQDHSNNPNG

SEQ ID NO: 42 - protein, 234 aa, Citrus sinensis
MSAMVSALTQVIGTTTTDADADPSPAAVKEESSDNPLQQTQTQTQDQDQEGTRRRHYRGVRQRPWGKWAAEIRDPKKAARVWLGTFETAEDAAMAYDKAALKFKGTKAKLNFPERVQGTTEFVYLDSSSSSSAFHHHHESVMPAPPPRPTSMHHGAYPDLLQYAQILSSDDATFNYYTSNLFNPQSSSSSSSTPSTFSSSTTSLEQQQEMTRFSSNYESLSGSDFQDHSNNPNG

SEQ ID NO: 43 - protein, 395 aa, Carica papaya
MCGGAIISDFIPTATTRSCKLTADYLWPDLNRNRKSKKSSKRSEVVDLDDDFEADFQGFKDDESDIDVDEDLDDIDAVFSDIKPFAFSATPLPRKTTASALSNGSKPVKAVEFNGLAEKSAKRKRKNQYRGIRQRPWGKWAAEIRDRRKGVRVWLGTFNTAEEAARAYDAEARRIRGKKAKVNFPDESPRASPKRAVNSMKPVAKAILNSAQPNLSQNVNYFNNLGQDYYNTMVFVDEKPQMNQFASMNSFPPRRNAGVKPFVPSDNTHMYFSSDPGSNSFGCSEFGWGDQATKTPEISSVLLDQPQFVEDCNPEKKLKCSSETMVPVQGNANKSLSEELLAFDNQMKYLQVPHLDSNWDSSLDAFLNGDAPQDAGNSMDLWAFDDLPSLVGGVF

SEQ ID NO: 44 - protein, 404 aa, Musa acuminata
  source 1..404: note = Subspecies malaccensis
MAACSSGTAKWKLREGKRKRARGWEKAKSKQECRAHQSRRPAQSSERLFLSQSRIIHSLSFLLLRLLLDRLPLSPLSAVNISVSLSRCCIYLRLSVVFLCRRRRCHHQRISPDPLSGRRKEEERSRNDKVDRRPGKRPLPPDELEKKEGDQQQAVSRFASSRADHDASAMVSALAHVISSSSSVVDTRGGEPASTQQGIKLEEAAGRGDTEAAQVSEEQGNVRRRHYRGVRQRPWGKWAAEIRDPRKAARVWLGTFDTAEDAAVAYDEAALRFKGTKAKLNFPERVQGRTDLGFLVSPGVPERQPPRVPLQLPATSYPDLLQYAQLLQSRDEDLQNVASGLYVGGTFTPVSSQTPTTSALGSSQHFLDFSSQSQYTNFSSSSSSSSSSSWVHGEQKDKDGSRPP

SEQ ID NO: 45 - protein, 227 aa, Musa acuminata
  source 1..227: note = Subspecies malaccensis
MVSALSRVISSSSSVIDASAGEPTVNQQGIKLEGADPGEKQAIQISEEQGTVRRHYRGVRQRPWGKWAAEIRDPKKAARVWLGTFDTAEDAAVAYDEAALRFKGSKAKLNFPQRVQGRAELSFLASPGIPRRQPQPPTRPPASSYPDLFRYAQLLQSGDDNLQSVASGLYVGSAFTSAPSQAPPSSTSGSLPQFLGFSSHSPYSSSSSSSGSWVYGDHKDKDSSRPP

SEQ ID NO: 46 - protein, 256 aa, Musa acuminata
  source 1..256: note = Subspecies malaccensis
MDRRHGKRPLPPDEAAPEEKAGELSCSPLARADQDASAIVSALAHVIGSCSPVAGVGGGEMRQDVSGSGTGSVENRTQPSEEQGNAGRRHYRGVRQRPWGKWAAEIRDPQKAARVWLGTFDTAVDAAIAYDEAALRFKGCKAKLNFPERVQGRSDLGFLTHRWQAQPPVQLPATSYPDLLRYARLLQSRDDDLHNRAVGLHPAGSSFMSTSSHTTPTSSLSGSSQELVGFSHHWQLRSSSSSSSWPQVDLQDEDED

SEQ ID NO: 47 - protein, 229 aa, Musa acuminata
  source 1..229: note = Subspecies malaccensis
MVSALSHVISSRSPPVGAGGGEPVMVQHDGKLSGSGSGSAEIRTQPSGEQGRRHYRGVRQRPWGKWAAEIRDPRKAARVWLGTFDTAEDAAIAYDEAALRFKGTKAKLNFPERVQGRTDLGFLVSGGGSERQPQPPTQRLPAANSYPNLLQYAQLLQSRDQDLHQAAFGLYAGSTFTSTSSQTSPTSMSAASSQEMLDFTCQSHFKSSSSSSSWPHGGHKHEDQQPPGM

SEQ ID NO: 48 - protein, 213 aa, Musa acuminata
  source 1..213: note = Subspecies malaccensis
MVSALTHVISSVSPVVGAGGDELVAEPDASCGSGPGSMEIGTQASEEQGRRHYRGVRQRPWGKWAAEIRDPKKAARVWLGTFDRAEDAAMAYDEAALRFKGTKAKLNFPERVQGRTDLGFLVTRAAPERQPQPPATSYPDLRQYAQLLQSGDADVHNAALGLYAGSTITSTSLSGSSQETQDLSSRSQFTSSSASSSWPQSGQKEKDQRPPTM

SEQ ID NO: 49 - protein, 368 aa, Musa acuminata
  source 1..368: note = Subspecies malaccensis
MCHNVANPHQLPDDSFAAEGSDGAPLSSYHRAQEISTIVSALAHVMASERRPRPVGMAVDSVSVVSSSSSSSSSSSSSLSCISSSYSSPSLGGQGGGARSQNRTRRVPSPPDLALRHHQGLGEFARYRGDASPDVAATEQYPQGGPLPILGYPVPAAAMEEPSPASSNPEEAERSEPRRKYRGVRQRPWGKWAAEIRDPHKAARVWLGTFETAEEAARAYDAAALRFRGRRAKLNFPENVRLQPSLSVPLATSNSPATTSDTITDYLAYTRLLQGGEEHPRIPPTSLLDQYMYSNYASPMCSTVNDGSSLPAPSIPTYSSVVSSSSTPYSPFYASSTTEQQTNWSGVSDIPETSWMGSSQFPPSSSGS

SEQ ID NO: 50 - protein, 387 aa, Musa acuminata
  source 1..387: note = Subspecies malaccensis
MCLKVANPHQSSDGSFAAAGSDEMEEDAAAAAGMMYSSVTAQAALLSGHRRSRETSTMVSALTRVMAGEQRPRPVPMAVDSMSAVSSSSSFSYIFSPPPSYSSPSTGGQSGGASSQTRTRPELPSHLALRYYRCLGEFGSYYGGASPDVAAVEQYPQAFLPMLQSPAPAAAAVEEASPASSNQEERERAAPKRKYRGVRQRPWGKWAAEIRDPYKAARVWLGTFETAEEAARAYDEAALRFRGSRAKLNFPENVRLQPSHSVALAAQVPPSNSPATSSGAVSDYLAYSRLLQGASEYQRLPPASLLDPFVHSGVNDSSSLPASSFHANSVPSSTVISPSSSSSSSYPPSYASSTPTERQMIWGGASGFPETSWTHSSQFPPSSSGDS

SEQ ID NO: 51 - DNA, 792 bp, Arabidopsis thaliana
  source 1..792: mol_type = other DNA
atggcgaattcaggaaattatggaaagaggccctttcgaggcgatgaatcggatgaaaagaaagaagccgatgatgatgagaacatattccctttcttctctgcccgatcccaatatgacatgcgtgccatggtctcagccttgactcaagtcattggaaaccaaagcagctctcatgataataaccaacatcaacctgttgtgtataatcaacaagatcctaacccaccggctcctccaactcaagatcaagggctattgaggaagaggcactatagaggggtaagacaacgaccatggggaaagtgggcagctgaaattcgggatccgcaaaaggcagcacgggtgtggctcgggacatttgagactgctgaagctgcggctttagcttatgataacgcagctcttaagttcaaaggaagcaaagccaaactcaatttccctgagagagctcaactagcaagtaacactagtacaactaccggtccaccaaactattattcttctaataatcaaatttactactcaaatccgcagactaatccgcaaaccataccttattttaaccaatactactataaccaatatcttcatcaaggggggaatagtaacgatgcattaagttatagcttggccggtggagaaaccggaggctcaatgtataatcatcagacgttatctactacaaattcttcatcttctggtggatcttcaaggcaacaagatgatgaacaagattacgccagatatttgcgttttggggattcttcacctcctaattctggtttttga

SEQ ID NO: 52 - DNA, 1269 bp, Zea mays
  source 1..1269: mol_type = other DNA
atggagcgcgtgaagtattgtgattgtactgtgtgcagtgtgcagcggtcattgtgttctacccgccggaggaggaggaggaggaggcagatcgaccggcagctgactaaggtggatccacggaggaggcatggcaagaggcccctacccgccgccgaggtggaagaggaggaggaggaggaggcgctgcccccgggcccgccgcccgcaaagcacgagcagctggaggagcctcaccacgccgccgtctcgcagctgcaaggagccacctttagcggcggcggagggtcgtcgtcgtcttccgtgatcggtggcccgtcgccgccgcaggcgtacgcgcagtactactactcggcgcgcgccgacaacgacgcctccgccgtggcctccgcgcttgcccacgtcatccgggcctcgcctgaccagcttccgccgcagcaggcgccggccttgtacggcgccggcgtcccgggcagccttcggctgggagaccacccgcaagcgtctgcgcaccactatcccggtcccggcggccacgtcgccgccgccgaggaggagcaaggtcggaggcggcactaccggggggtgaggcagcggccgtggggcaagtgggcggcagagatccgggaccccaagaaggcggcgcgggtgtggctcggcaccttcgacacggcggaggacgccgccatcgcctacgacgaggcggcgctgcggttcaagggcaccaaggccaagctcaacttcccggagcgcgtgcagggccgaaccgacctcggcttcctcgtcacccgcggcatcccggaccaccggcacccgtcggcggcggtgaccctggcagcaatgccgccgccgcaccaccagcacggccaccagaccgtcgtgccgtaccccgacctcatgcagtacgcgcagctgctgcagggcggccggggcggcggcggccacgccgaggcggcggtccagcaggcgcaccgtcagcagcagcagcagcagctcatgacgatgatgggcggtcggccgggagtcaacctgccctccacgttctcgccgtcgtcgtccgcatcggcgccgcagatactggacttctccacgcagcagctcatccggcccggcccgccgtcgccgtcgccgccgcgggccgcggcaatgccgtcctcgtcggccgctgcggcgccgtccacgccgtcgtccacgaccacagcgtcgtcgcccagcggcggtgcatggccgtacggcggggagcgccacaggaataaaaaagacgcgtga

SEQ ID NO: 53 - DNA, 1332 bp, Zea mays
  source 1..1332: mol_type = other DNA
atgcgcatctctctccgcgtacttatcagtagcgagctcggcacgtcattgtgcactgctgcaccgagcctagcgcgagcctccgtccgaaagtccaaatcttcagctctctccttgacgcatgcccggatcgatcggtccagctctacccaccggaggaggaggcagatcaacgggcagctgactaaggtggatccacggaggaggcatggcaagaggcccctacccgccgacgaggaggaggaagaggaggaggagctgcccccgccgccggcaaagtacgagcagctggatcaggaggagaagcatcacgttgtcgtctcgcagctgcaagcaggagctacctttagcggtggccgagggtcttcgtcgtcttccgtggccggcccttcgccggaggcgtacgcgcagtactactactcggcgcgcgccgaccacgacgcctccgccgtggcctccgcgctagcccatgtcatccgcgcctcgcccgaccaactcccgccgcagcaggccgcctgcttgtacggcgccgccggcgcgccggtcctgcggcagggagagggagaccatccgcaaccgcaagcggctgcgcaccaccatcccggtggccacgtcgccgccgaggaggagcaaggtctgaggcggcactaccgaggggtgaggcagcggccgtggggcaagtgggcggcggagatccgggaccccaagaaggcggcgcgggtgtggctcggcaccttcgacacggcggaggacgccgccatcgcctacgacgaggcggcgctgcggttcaagggcaccaaggccaagctcaacttcccggagcgcgtgcagggccgaaccgacctcggcttcgtcgtcacccgcggcatcccggaccaccaccggcacccgcgggcggcggcggtgaacctggcagcaatgccgcaggcgcaggcgcagccgcacttgcagcacggccgcccgaccgtcatgccgtacccgtacccgtaccctgacctcatgcagtacgcgcagctgctgcagggcggccggggcggcggcgaccacgcggcggcggtccagcagcagctcatgatgatgggcgggcggggcggcaacctgcccttctcgttctcgccgccgtcgtcctggagtgcgccgccgcagatactggacttctcggcgcggcagctcatcacccagcccggcccgccgtcgtctccggccgcccccggcggcgcggcgccgtccacgccgtcgtccacgaccacggcgtcgtcgcccagcgccagcgccagcggcagtgcatggccgtacggtggggagcaccacaggaataaaaaggacgcgtga

SEQ ID NO: 54 - DNA, 1074 bp, Zea mays
  source 1..1074: mol_type = other DNA
atgtgcttcgagctggcggaccagcgcggaccgcagggcggcggcggagcggggtggccggcgaagcggcgcgcgggaggcgtgcaggacgagggcgcggccgcggccgcggggatggcaatggcggccgccggccccggggaggtgatgtccgagtactaccaggcgcaggagctgtcgacgatggtgtcggcgctgacccacgtcgtggccggcgcgccgatggggagcgcccctgcgcagcggccaatgcacggcgcctccggctactacgcgcacgagatggggagctaccgcggcgcgccatcgccggagctcgcagggtcggagctgagttcggacacccagagcgcgggcgcggcggccatggaggagcaccagtcggcagctgcgctgtccagccaggagggtcccgagacgccgcggaggcgctaccgcggggtgcgccagcggccgtggggcaagtgggccgccgagatccgggacccgcacaaggcggcgcgcgtctggctcggcacgttcgagaccgccgaggccgcggcgcgcgcctacgacgaggccgcgctccgcttccgcggcagccgcgccaagctcaacttccccgaggacgcgcgcctctacccggcgtccacggcgggcgcggcggcgcctctcgccgcggcggcctcgacctccccacctgtctactccggtggcgtccagggctcgtcggactacctgaggtaccaccagatgcttctgcaagcgtccacgggcagccagggcaccctgctcccgttctatggcggcggcatgagcaatccctacggcggcggtgccgccatgaccggttcctacggtggagccggcggcggcaacacgagtggttccttgggctcctactactcgttcccggcctcctctgtttctgtcgccaccgtgccgtcctctacttcctctgcttcggggtactactactcgtcgccgcacgactcgcaacacagcgaggcgtccgcggcggcggactggaactgggagagcgcgctggcctggcccgactcgagccagtacccgccgccacctcacactcagtag

SEQ ID NO: 55 - DNA, 405 bp, Vitis vinifera
  source 1..405: mol_type = other DNA
atgtccgctatggtttcagctctcactcaagtcattggaaacactgacaaaaacccacttcatgatcttggaaacccatcaccaatctcccaccactccgccacaaccccacatgatcaaccttctcagcttcttcaagatcaagggaaccagctgaggagaagacactataggggagtaaggcaaagaccttgggggaagtgggcagctgagatacgtgatccaaataaggcagctcgagtatggctgggcactttcgacactgctgaggatgctgcacttgcctatgatgaagctgctcttaggttcaaaggaaacaaagccaagcttaacttccctgaaagagttcaaggccgaagcgaattaggttacctcacaaatcctccttccaggtggaggtggtga

SEQ ID NO: 56 - DNA, 944 bp, Vitis vinifera
  source 1..944: mol_type = other DNA
atgtccgctatggtttcagctctcactcaagtcattggaaacactgacaaaaacccacttcatgatcttggaaacccatcaccaatctcccaccactccgccacaaccccacatgatcaaccttctcagcttcttcaagatcaagggaaccagctgaggagaagacactataggggagtaaggcaaagaccttgggggaagtgggcagctgagatacgtgatccaaataaggcagctcgagtatggctgggcactttcgacactgctgaggatgctgcacttgcctatgatgaagctgctcttaggttcaaaggaaacaaagccaagcttaacttccctgaaagagttcaaggccgaagcgaattaggttacctcacaaatcgtcaagacttccttcttcctcagcaacaacagcttcccaaccctgctgttcctcctcttcctcatccatccctccctcgaccgtcatatcccaatcttcatcactatgctcagctccttccaggtggaggtggtgatttaaatcatgctatgtccagtctttatggtagagaagcttctactacgcagtctttgtcaactacatcttcatcttcttctacaacctctcatccacaacaccatcagcgaagacgacaacgagaagaagaagaattacaacaaccacaacttctacaattttcatcactgtttggaagttcttctagcaacgaccctcacaataacaggagagatgactgaaaaggtcagtaaagagggtacttagtattcccaataatccctttaagaagggttgcagtaaaagatgtgaatttcggttatgttgtcttatttttattttttattttttatttttaagcttaaacccttgcctattgatctttgtcttgagtttaagagtgaaaaagagaacccacaatgcatggaagtgattctactctata

SEQ ID NO: 57 - DNA, 723 bp, Populus trichocarpa
  source 1..723: mol_type = other DNA
atggatgtgatggtttcagctctagctcaagtaattggaagcagccacaatagctcagctcaagtgcaggaaaatccattgacctcaacacaatccagcacagaaaatgatcaaacccaaccagctgttcaagaccaagggaatgcaaggagacgacattatagaggagttaggcaaaggccttgggggaaatgggctgctgagatccgagatcccaagaaggcagctcgagtgtggctaggcactttcgaaacggctgaggctgccgcccttgcttacgatgaagcagctcttaggttcaaaggaagcaaggctaagctcaactttcctgaaagggttccatctggcgggaccgaattaggtttctttactagaggccaaggtttgcatacggttactgaacctgtctctaatcacattatggctcctctagctcgttcccaacgatcacaggaagcatataatccaaataattttcaatatccacaatttcttggaaccacatcaggctatggcttaagccatgtcatgccgcctgcagttccatttggtggagaaacatttctttcaccgacttcctccagtgcatcttctaattcgtggccgatatcttctcaacagcagcagcagcaacaagaggaacttttaagattatcaatgcagtttggaagttcttacaactctcgttatgatccttctaaatacaaagacgaggggctttga

SEQ ID NO: 58 - DNA, 870 bp, Solanum lycopersicum
  source 1..870: mol_type = other DNA
atggaccgaattagacgtggaaagaggcgctatgaatccgaggaaaaagaagatagaaattacaatcacatgtattcatcagctcgatctcaacatgatatgtctactatggttgcagtcctctctcaagttattggcaacaaaagtactactaacacgaattcttcttcttcttcctccgcacatcataagccattattaaccctaaatcatcagtctaatactactgcagctatgcaaaaccaattacctcagctcaatcaacaacaagggaataatgagaagagaagaagacagtacagaggtgtgaggcaaagaccatggggtaaatgggcggccgaaatccgtgacccggaaaaagctgctcgtgtatggttgggtacttttcacactgcggaagatgctgcaattgcatatgatgaagctgcacttaaatttaaaggaaacaaagctaaactcaactttccagaacgggttcaatccacaacggatcaatttgggatatcatacctaattactaatacgaatcatcaacaacatcaatttcagcccactaattttctcccaaattctgatcaattacaacaacatcactacagtaatcataatgctgatgatttgaaatttggcgtctcgccgagtttttatcaccctacagggttcaatcctaaagcactcgatttagtggaaccttcgaaatcatcatcgatgacatatttggtacaacaagcatcgtctcatcaggtacaagaagaaccaaggtatattaatcaccagcaagaagatgaaaacaatctgaaattttcatcttattttggcacttattcaagctcaggaccaactttgggggagtttgaagatcaaaaataa

SEQ ID NO: 59 - DNA, 735 bp, Solanum lycopersicum
  source 1..735: mol_type = other DNA
atgaaaagatcaagctctaataatgatcagagggatgaaaaagacactagtaacatattcccaatatactcatcagctcgatctcaacatgacatgtctgctatggtttctgctctctctcaagtcattggtaactcttcgtcttcagctagtggtgattcctcctcagtgcatgttaatccactaaccctaattcagcaacatcaatctcaatcgtctactcaagatcaagaaagaaggcgatatagaggagttcgtcaaaggccatggggtaaatgggcagcggaaattcgggacccgaaaaaggcagctagggtttggttaggtacttttgaaacagcggaaggtgcagcacttgcttatgatgaagctgctctaagattcaaaggaaacaaagctaaactcaatttccctgaaagagttcaaggacagtttttccaatgctatgatcaacctgccacgtcatcaaacaacacatctgaacaaaattaccctaatgttcatcactatgctgatttattacttcgtactgacaataatatcgatctaaatttcgatgtttcaccaaatactttttatcactcttttgatatttcacaatcatcaatggaagttccagtttatcatgaggagcagcaacaagttataactacgcacgaagaagaagaagaagattttgtgaaatatcgtggatcacattttggaaattctacttcaagtggtggaactaaatag

SEQ ID NO: 60 - DNA, 693 bp, Solanum tuberosum
  source 1..693: mol_type = other DNA
atggatcgaactagacatgggaagaggccctatgaatcggaggagaaagaagatacgaataataatcaaatgtattcatcagctcgatctcaacatgatatgtctactatggtttctgtcctctctcaagttattggcaacaaaagtaggactaacacaaattcttcttcctcctcctccgcacatcataagccattattaaccctaaatcatcggtctagtactactgcagctatgcaaaaccaattacctcagctcaatcagcaacaagggaataatgaaaggagaagaagacagtacagaggtgtgaggcaaagaccttggggtaaatgggcggccgaaatccgtgacccggaaaaagctgctcgagtatggttgggtacttttcacactgcggaagatgctgcaattgcatatgatgaagctgcacttaaatttaaaggaaacaaagctaaactcaactttccagaacgggttcaatccgcaactgatcaatttgggatatcatacctaattactactactaatcaatttccagccaataaatttctcccaaattctgatcaattacaacatcactacgctcccgcaggtggaagtaatcataatgctgatgatttgaactttggcgtctcgccgagttcttatcaccctacaggtaagagtactgtaaatgatttgagtacgcagtag

SEQ ID NO: 61 - DNA, 810 bp, Glycine max
  source 1..810: mol_type = other DNA
atggaaggaagatcaatatcacattcttctgaaagagaagaggaatatgatttgttcccaatatattctgaaaggtctcagcaagacatgtcagccatggtttctgctctcacacaagtcattggtggctccaatagtgattctcttcagcagcatgaaggccttttaacttcttcacataacaacacctcaacccagaataataatgaacaatcccaagcaccacaacaagaacaagggagtgtaagaagaagacactacagaggagtgaggcagcgtccatggggaaagtgggctgcagagattcgcgatccgaagaaagcagcgagggtatggcttggaacctttgaaacagctgaagctgcagctcttgcttatgatgaagctgctcttagattcaaaggaagcaaagcaaaactcaatttccctgaaagggttcaaggcacagctagcgaatttggttaccacctcacaaatcagcacagcactagtagtcatgatcagcaagcttctaaccctattattactccacactttgctactactcaagaaacttattcaccaagtcatcactttcaatatgcacaacaacaactcatgggtggtgggagtaacagcttcaataacaaccaagatatgcttcgtttctatgggggacataacatgtttgtttcttctcagcaatctgcatcatcgtcttcttccactgcactgtctcagaatcagcaggatgagttgctgagattttcgatgcaatttggagcttcttctcattctgaccattcagggaattggaggggtggacaatga

SEQ ID NO: 62 - DNA, 702 bp, Gossypium raimondii
  source 1..702: mol_type = other DNA
atgaacgctatggttcatgccctagctcaagttattggaaacaataacagtaaccctcttcttcaacttcatgatgatcaacatccaaaccccactgctcaacaaaaccaatctcatcaacagcctcaacctcaagatcaggggaacgccaggagaagacactatagaggagtaaggcaaagaccatgggggaaatgggcggccgaaattcgtgacccgaaaaaggcagctcgggtttggcttggaacgtttgaaacagctgaagcagcggccttagcttatgatgatgcagcacttaggtttaaaggaagcaaagcaaagcttaacttccctgaaagagttcaaggaagattagaatccagttatctcacaacaactcgtcaagaactggaacggacggaggcgccacctcatccgccaccaacctatcctaatatttctcagtacgcgcagctcctcagcggtggcttacccaatactgctttcaactacgccatgccaagtggtgctgcctatggatcctggccagcttttactactagttctcactcttcatcttcatcttcatcttccacaacgttaacttctcaacaacaagggtatatgggtggattttcattgcactttggtggttcatctccaacctcagatcataccaacaacatgggagattatgattattactattcaagagatcaatag

SEQ ID NO: 63 - DNA, 768 bp, Brassica rapa
  source 1..768: mol_type = other DNA
atgaataatgggaagaggccctttcgagctggtgaatctgaggagaagaaagaagccgatgacgacgagaacatatttccgttcttctcggctcgatctgaatacgacacgcgtgccatggtctcagccttgactcaagtcattggaaaccaaagcagcacgcatgataataatctacatcaccctgttgagtatgatcagcaagatcccatccaacatgttcctcctactcaagatcatggaaacttgaggaaaatacattatagaggggtaaggcaacgaccatggggaaaatgggctgccgaaattagggatccacaaaaggccgcacgcgtgtggcttgggacatttgagaccgctgaagctgcggccttagcttatgatgaagcagctcttaagttcaaaggaagcaaagccaaacttaatttccctgagagagctcaactagcaagtaacactagtaccattaccggtctaccaaactattactcttctaataatcaaacttactactcaaatccgcagactaatccacaaaacataccatattataaccaatactactataaccaatatcttcagcaagggggaaatagtaacgatgcattaagctatagtctggcaggtggggaaaccggaggctcaatatatagtcatcagacattatctaataccacttcttcaccagctggtggatctttaaggcaacaagaagattacacaagattttggcattttggggattcttctcctaattctggggtttga

SEQ ID NO: 64 - DNA, 762 bp, Brassica rapa
  source 1..762: mol_type = other DNA
atgatgccctttggagcaggtgaatcggatgaaaggaaagaagccgatgatgaggagaacatattccctttcttatcagcccgatcccaatacgacacacgtgccatggtctcagccttgactcaagtcattggaaaccaaagcagcactcatgatagtaatcaacatcaccctgtagagtataatcaacaagatcctatccaacatgttcctcctactcaagatcaaggaaacttgaggaagaggcattatagaggggtaaggcaacggccatggggaaaatgggctgccgaaattcgggatccacaaaaggcagctcgagtgtggctcgggacatttgagaccgctgaagctgcggccttagcttatgatgaagcagctcttaagttcaaaggaagcaaagccaaactcaatttccctgagagagctcaactagcaagtaacgctagtaccattaccggtctaccaaactatcactcttctaataatcaaatgtattactcaaatccgcagaccaatccacaaaccatgccatattataaccaatactactataaccaatatcttcagcaaggaggaaacagtaacgatgcattaagctatagtttggccggtggggaaactggaggttcaatgtataatcatcagtcaatatctaatacaacttcttcatcttctggtggatcttcaaggccacaacaagaacaagattacgccagattttggcattttggggattcttctcccagttcaggtttttga

SEQ ID NO: 65 - DNA, 653 bp, Hordeum vulgare
  source 1..653: mol_type = other DNA
atggtgtccgctctgtcccacgtcatccgcgccacacccgaccaggaaccagcctactaccccgccggacccgccgctgtctctagagaacagcagcatcagcacgctgcggccatcgcagaggaacaagggaggaagcgtcactacagaggggtgaggcagcggccatggggaaagtgggcggcggagatccgggaccccaagaaagcggctcgtgtgtggctcggcaccttcgacacggctgaggacgccgccatcgcctacgacgaagcggcgctgcgcttcaagggcaccaaggccaagctaaacttccccgagcgcgtccagggacgcaccgacctcggcttcgtcgtcacgcgcggcatacctgacagactgcagcaacaacagcactaccccgccgccgtgggggcgccggcaatgcggccgccgctgcaccagcagcaggccgtcgtgccgtaccctgacctcctgcggtacgcgcagctgttgcagggcgctggcagtgccgggggcgctgtcaacctgccgtttggggccatgtcgcccccgtcgatgtcgtcctcgtcgtcgccccacatactcgacttctcgacacagcagctcatccgagtgagcccggcgtcccccgccgcggcaatatcaggttcagc

SEQ ID NO: 66 - DNA, 1321 bp, Oryza sativa
  source 1..1321: mol_type = other DNA; sub_species = Indica
atggtcaccgcgctagcccacgtcatccgcgccgcccccgacctgcacctcccccaccacccatcctcctccgcctccgccgccgcccacccgcagcaggcttcttccttctacccgaccgccgccgccgccgcctcctcgccgtccgaccagctcgccgccgccgccgccgctgccgaagagcaaggtaccagctgatccattcaccgcacaagtaaattcctctctctcaccgcgccgcgtgccacgccccgattgcgaaagcatggcacgcgcgagcgcgccataatttggaagcttcgcatccgccaacgccaacgcccacgccgccgtcgtcctcgtcctacgttaatccccatggctctagctagctaattaccgggttaagtagcttcgaagcttgcaatgcatgcttaattagcggtaattgatccataattaattaattagttgaaccaaacgagggttaataagttaattaactcccaaatcgttgcctatttcgtgccggaagatacgctaaattaatagcgccatgtgagtattttatgcacacaccgtattacgttatccctgacgatcgaagggggagtccgaattagtcacggattaattagcaagttgcagtattattattattattattatgggattaattagtgggaattaattagcttagtgtgttttggtgggtgcagggaggaggcggcactacagaggtgttcgacagcggccatgggggaagtgggcggcggagataagggacccgaagaaggcggcgagggtgtggctggggacgttcgacacggcggaggacgccgccatcgcctacgacgaggcggcgctccggttcaaggggaccaaggccaagctcaacttccccgagcgcgtccagggccgcaccgacctcggcttcctcgtcacccgtggcatcccccccgccgccacccacggtggcggctactacccctcgtcgtcgccggcggcgggggcatgccccccgccgcggcagcagcagacggtcgtgccgtacccggacctcatgcggtacgcgcagctgctgcagggcggcgtcggcggcagttacatgccgttcggcggcgccgcgacgatgtcgtcgtcgacggtgtcgtcctcgtcggcgccgcagatactcgacttctcgacgcagcagctcatccgggccggcccgccgtcgccaatgccatcgtcgggctccggctcggcgaccgcggcggcgtcgtccacgacgtcggcgtcgtcgcccggtgcatggccgtacggcggctcggagcgcaagaagaaggattcgtcgtcgtga

SEQ ID NO: 67 - DNA, 807 bp, Oryza sativa
  source 1..807: mol_type = other DNA; sub_species = Japonica
atggtcaccgcgctagcccacgtcatccgcgccgcccccgacctgcacctcccccaccacccatcctcctccgcctccgccgccgcccacccgcagcaggcttcttccttctacccgaccgccgccgccgccgcctcctcgccgtccgaccagctcgccgccgccgccgctgccgaagagcaagggaggaggcggcactacagaggtgttcgacagcggccatgggggaagtgggcggcggagataagggacccgaagaaggcggcgagggtgtggctggggacgttcgacacggcggaggacgccgccatcgcctacgacgaggcggcgctccggttcaaggggaccaaggccaagctcaacttccccgagcgcgtccagggccgcaccgacctcggcttcctcgtcacccgtggcatcccccccgccgccacccacggtggcggctactacccctcgtcgtcgccggcggcgggggcatgccccccgccgcggcagcagcagacggtcgtgccgtacccggacctcatgcggtacgcgcagctgctgcagggcggcgtcggcggcagttacatgccgttcggcggcgccgcgacgatgtcgtcgtcgacggtgtcgtcctcgtcggcgccgcagatactcgacttctcgacgcagcagctcatccgggccggcccgccgtcaccaatgccatcgtcgggctccggctcggcgaccgcggcggcgtcgtccacgacgtcggcgtcgtcgcccggtgcatggccgtacggcggctcggagcgcaagaagaaggattcgtcgtcgtga

SEQ ID NO: 68 - DNA, 1182 bp, Setaria italica
  source 1..1182: mol_type = other DNA
atgcccggatcgatcgatccagctccatcggccgacgggaggaggaggaggaggcagatcgaccggcagctgactaaggtggatccccggaggcatggcaagaggcccctacccgccgacaaggaggaagaggatcagccgccgcccccgccgccggcgaagcacgagcagctggagatcgaggagcatcggtaccacgtctcgcagctgcagcaaggtgctacctttagcgccggcggcggcggcggagggtcgtcgtcgtcgtctgccgccggcgccgcggctggtccgtcgccggaggcgtacgcgcagtactactactcggcgcgcgccgaccacgacgcctccgccgtcgcctccgcgcttgcccacgtcatccgcgcctcccccgaccagctcccgccgcacgccttcggcggcggcggcgctccgccgggccagggagactaccagcaagcagcgccaccggccgccgccgccgccgccgccgaggaggagcaagcagcaggtcggaggcggcactaccgaggggtgaggcagcggccgtgggggaaatgggcggcggagatccgggaccccaagaaggcggcgcgggtgtggctcggcaccttcgacacggcggaggacgccgccatcgcctacgacgaggcggcgctccggttcaagggcaccaaggccaagctcaacttcccggagcgcgtccagggccgcaccgacatgggattcctcgtcacccgcggcatccccgaccggcaccaccaccaaggaggggcggcggtgacgctggcggcaatgccgcccccgcaccggcaacaccaccagaccgtcgtgccgtacccggacctcatgcagtacgcgcagctgctgcagggcggcggccggggcggcggcggcgccggagaccaccacgccgaggcggcggcccagcaggcgcaggcgcggctcatgatgatggcccgcggcggcgtcagcctgccgttcggcgccgcgtcgttctcgtcgtcctcctcgtcggcgccgcagatactggacttctccacgcagcagctcatccggcccggcccgccgtcgccggcagccgcggcgccatccacgccgtcatccacgaccacggcgtcgtcgcccggcggcagtgcatggccgtacggtggggagcaccacaggaataaaaaagacgcgtga

SEQ ID NO: 69 - DNA, 885 bp, Sorghum bicolor
  source 1..885: mol_type = other DNA
atgaccaagaagctcatctccgccatggccgggaagcaaggtttcaaggagcagcagttcaatgatcagaggagacagcaggcttcgattcaaggagacgacatcgccaagagcctggtggggttcggcggcggcggcggcaggctgatctcccatgagcaggaggacgccatcatcgtggcggcgctgcggcacgtggtgtccgggtacagcacgccgccgccggaggtcgtcacggtggcgggcggcgagccgtgcggggtctgcggcatcgacggatgcctcggctgcgacttcttcggggcggcgccggagctgacgcaacaggaagcagtgaacttcggcacagggcagatggtagcgacagctgcggcagcggcggccggaggggagcacgggcagaggacgcggcggcgtcggaagaagaacatgtaccgcggcgtgcggcagcggccgtgggggaagtgggcggcggagatccgcgacccgcggcgcgcggcgcgcgtgtggctgggcacgttcgacaccgcggaggaggccgccagggcctacgactgcgccgccatcgagttccgcggcgcgcgcgccaagctcaatttcccgggccacgaggcgttgctgccgttccagggccatggccatggcggcgacgcttgcgccaccgcggcggcgaacgccgagacgcagacgacaccgatgctgatgacgccgtcgccgtgcagtgcagacgccgcggcggcggcgccgggagactggcagctgggcggcggcgtggacggcggagagggagacgaggtgtgggaaggtctgctacaggacctgatgaagcaggacgaggcggacctctggttcttgccattttccggcgctgcatctagtttttga

SEQ ID NO: 70 - DNA, 711 bp, Theobroma cacao
  source 1..711: mol_type = other DNA
atgacagccatggttacagctcttactcatgtgatgggaaccggcgggagcgatgagcagcttagtttcacaccatcgtcggtgcccttatcacaatctgcggtgaaagaagagcctgaccctcctcaacctgttcaagatcaagaaaatacaaggagaagacattacagaggagtgaggcaaaggccatggggcaaatgggcagctgaaatacgcgaccccaagaaagcagcccgagtctggcttggcacttttgacacagctgaggatgcagcccttgcatatgatagagctgctcttaagttcaaaggaaccaaggcaaaactaaattttcctgaacgagttcaagggaatacggaggtaagctatttcacgggtcatggagattcaagtactgtccgtcctgaccaaaatcctacgccagcagctactcctccatcatggtcgcaggattcatatccccacttgtttcaatacgcacaactactttcaagtagtaatgatgctgatatctcttattacacttcaaatctcttcaatcaagaacctctatctccacagtttccgtcaatggcggcgtcacccaacatctcatcacaataccaccatcaagaccagacaagattttcaaccaagtatgagagctcttctggttctgattatccggagcaatatgggaaggactctgatccaagtaaccgaagcgagtag

SEQ ID NO: 71 - DNA, 699 bp, Elaeis guineensis
  source 1..699: mol_type = other DNA
atggtgtcggctctcgcgcatgtgatcagctcgagcttgtcgggggtgggagtgggagtgggaaggagcgagtctgtggtgattcagtccgagttgaacccagccatggccggaccggagtcgggaagcatggagagagagctcagccagccttcagaagagcaagggaatgtgaggaggagacactacaggggagtgaggcaaaggccatgggggaaatgggccgcagagataagggaccccaggaaggcagcgcgagtatggctagggacctacaacacggccgaagaagccgccatagcctacgatgaggcagccctgaggttcaaaggcaccaaggccaagctcaattttcccgagagagtccaaggccggactgacctcggcttcttagtcagcagggggatcccagagcgaccgccccagcccatcaccccaccgccgacagcatcataccctgacctccttcagtatgctcagctcctccaaagtagagatgaggacctgcacagtgtggcatcggggctcttcgttacagactctttcacctcggggtcttctcaagtgtcatatcattccacgtcaggctcctcgcaagagtttttggatttcttttcgcagatgaggagctcttcttcctctagctcaaggcagcctcgtggagaccagaaggataaagacagcaaccagcagcagtga

SEQ ID NO: 72 - DNA, 705 bp, Elaeis guineensis
  source 1..705: mol_type = other DNA
atggtatcggcccttgctcatgtgatcagctcgagctcaccgggggtgggagtgggagtgggagtgggagggggcgagactagggagatccagccggagttgagcccggccatggccgggacgggctcgggaagcatggagatcagggagctcagccaaccttcacaagagcaagggaatgtgaggagaaggcactacaggggagtgaggcaaagaccatgggggaaatgggcagcagaaataagggaccccaagaaggcagcaagagtatggttagggacctttgacacggccgaacaagccgccatagcctacgatgaggcggccctgaggttcaaaggcaccaaggccaagctcaatttccctgagagagtccaaggccggactgaccttggcttcttacttagtaggggaatcccagagcgacaaccagagcccatcaccccatcggcggcagctacatacccggacctacttcagtatgcccagctcctccaaagtagagacgaagacttccacaatgtggcatcagggctctacatcggaggctccttcgcgtcgggatcttctcaaatgtcaccggcatctatgtcgggttcttcgcaagagtttttggatttctcttcgcagtttggtacctcttcttcctctacctcatggcctcatggagaccagaaggataaagacagcagccagcatccgtga

SEQ ID NO: 73 - DNA, 735 bp, Malus domestica
  source 1..735: mol_type = other DNA
atgtcagccatggtttctgctctcactcaagttattggaaccaccgatgatcatgctgccgtgcaaccaaaccctacatccatctcagactcatccctcctagtcaaacaagaacccgaccgctctcaaccggttcaagatcaagagcccgtaaggaggcgacactatagaggagtgagacaaagaccttggggaaaatgggcagctgaaatacgtgacccaaagaaagcagccagagtttggcttgggactttcgagaccgccgaagatgccgccattgcatatgataacgcagctctcaagttcaaaggcacaaaagccaagctcaacttccccgaacgagttcaaggcaagaccgatttgggcatcctaatgggcagttctggtagtggtgctgcctcgactcaacgaacacaaaatctcatgacaccagctggccatattgttaatcctcagccagctcctgctccattaatgatgtcgcagcagccagaaactttccctgatctttatcagtacgcgcggcttctttccggcaatgatgctgatttctacaattattcttcctatccatttaatcaagatccaagatttacttcacggtttttgccatcatcgacgcatttttcatcttccactgcatcacaagattcacaaccaccgcaacaaggacaacaagatcatgaggaagatggggggaatgaggaccggaattggagtaatccaagggagtag

SEQ ID NO: 74 - DNA, 807 bp, Malus domestica
  source 1..807: mol_type = other DNA
atgatcttcgatccgttttgctcatctgctcgatctcaacatgagatgtctgccatggtctcagctctagctcaagttcttggaagcaataacagccagaccccggctgttcaagagccggtggagccgccactgatcacgccccagtccagcgccatggaattgcatgatggtcaatctcctcaacaggctcaagatcaagggaatgtgaggagaaggcactatagaggagtgaggcagcgaccatggggaaagtgggcagctgagattcgtgaccccaagaaggcagctcgagtgtggctcggcacctttgaaaccgcagaggccgcagcgatttcttatgatgaagcagctctcaggtttaaaggaagcaaagctaagctcaactttcccgaaagagttcagggaagaattactgaattaggttacctcactacaactagtactcaacaaaacttgccagctgaggctcaaagtattacggaccaccaccagcctcttcctgatcatcaatatcaactgcaggcattccctaataatgttgctactcctcatgatcaatatgcaccatattttctgagtggtaacgagggtgtaaattatgatgatttaccaactaatcttagtgaaagagaaagatttgctttccagacttcagaaactacttcatcatctttgttacctttcttaccatctcatcaacaagaagatcaacaacatcccttaaactattctatgccggcattcggttcttcttcttcttcgagttcaaatcctccccctaggaataggaaccgtcgaccttga

SEQ ID NO: 75 - DNA, 819 bp, Malus domestica
  source 1..819: mol_type = other DNA
atgatcttcgagccgttttgctcatcagctcgatctcaacatgagatgtctgccatggtctcagctctatctcaagttcttggaagcactaacaaccagaccccagctgttcaagtgccgatggagccaccaatgattgctccccagtccagcgccatggagtcgcatgatgatcaatcycctcaacacgctcgagatcaagggactgcgaggagaaggcactatagaggagtgaggcagagaccatgggggaagtgggcggctgagattcgtgacccgaagaaggcagctcgggtgtggctcggcacctttgaaaccgacgaggcagcggcacttgcttatgatgaagcagctctcaggtttaaaggaagcaaagctaagctcaactttcccgaaagagttcaaggaagaattactgaattaggttccctcactgccactagtactcaacaaaacttgccagctgggacatctcaaggagtaattactgaccaccatcaccctctttctgatyatcaatatcaactgcaggcattccctaataatgttgttactcctcctcatgatcaatatgcacaatattttcggagtggtcacgagtgcataagttatgatttaccagctactctttatgaaagagagatatttgctamccagacatcagaaactactacatcgtcatcatctttattacctttcttaccatctcatcaacaagaagatcaacaacatcccttaagctattctatgccggggttcgggtcttcttcttcttcgagttcaaatcctcctcctaggaataggaaccgtcgaccttga

SEQ ID NO: 76 - DNA, 795 bp, Malus domestica
  source 1..795: mol_type = other DNA
atgtatgacattgcaacgaattcgcaatttgatgttcataaagtggatcgaaaacatgggaagaggctgctggcttcggttgaatcggaggagaaagaggaggatcaaatctttcctgtttactcgactcgatctcagcaagacacttctgccatggtttcagctctagctcaggtgattggaaaaaatagtgaccaaattaataacccacttgatcaagtgcaaggaataaatccattaatcacctcacagtccagccctacagagactcaatctcaaacagtacttcaagatcaaggaaatttgaggagacaacattacagaggagtgaggcggagaccatsgggaaagtgggctgctgagattcgtgacccggtgaaggcagcccgagtgtggctgggcacttttgacacagctgaggctgcagcactagcttatgataaagcagcactcaagttcaarggaagcaaagctaagctaaacttcccygagagagttcaagggatctctgagtcaggttgcctcgcattaataactactgatcaacaagacagcttgaacattaatcctcctcagcctaatattactcgaccaacttcttccagtgttttcgattgtacacaatataataatgtcacatcttctacatccttgttgtcgtcgtcctccatgccatctcaacaagcggcagagctcccaagcttttcgatgcagttcggttcttctttttcgagttcaagttcaggtcctcctcataagtataggaaagactttgacagcagtcactcaagatga

SEQ ID NO: 77 - DNA, 645 bp, Malus domestica
  source 1..645: mol_type = other DNA
atggtttcagctctagctcaggtgatcggaaaaaatagtgaccaaattaataacccacttggtcaagtgcaaggaataaatccattaaccacctcacagtccagccctacggagactcaatctcaaccggtgctacttcaagatcaaggaaatttgaggagacaacattacagaggagtgaggcggagaccatcgggaaagtgggctgctgagattcgtgacccggtgaaggcagcccgagtgtggctgggcacttttgacacagctgaggctgcagcactagcttatgataaagcagcactcaagttcaaaggaagcaaagctaagttwaacttccccgagagagttcaagggagctctgagtcaggttgcctcacattaataactactgatcaacaagacagcttgaacattaatcctcctcagcctaatattactcgaccaacttcttccagtgttttcgattgtacacaacataataatgtcatatattgtacatccttgttgtcgtcgtcctccatgccatctcaacaagcggcagagctcccaagcttttcgatgcagttcggttcttctttttcgagttcaagttcgggccctcctcataagtataggaaagactttgacagccgtcactcaagatga

SEQ ID NO: 78 - DNA, 915 bp, Malus domestica
  source 1..915: mol_type = other DNA
atgtttttccggtatacttttctacttggtatcagagcagtttcatccgtcgtcttccgctcatccgtagtcgaagccattcttgtcaccaccaccgcactacaatatgtccagacaacccgcaccaccatctccgctgctacatccacctgttcacgggttaagctagacaaagtggatctaaaacatgggaagaggccactggcttcggttgaatcggaggagaaagaggaagatcaaatatttcctgtttactcagctcgatctcagcaagacacttctgccatggtttcagctctagctcaggtgatcggaaataatagtgaccaaattaataacccacttgatcaagtgcaaggaataagttcgttaatcacctcacagtctagccctaccgagactcaatctcaaccagtactacttcaagatcaagggaatttgaggagacaacactatagaggagtgaggcggagaccatggggaaagtgggcggcggagattcgtgatccgattaaggcagcccgagtgtggctcggcactttcaacacagccgaggctgcagctctagcttatgatggagctgctctcaggttcaaaggaagcaaagctaagctcaacttccccgaaagagtagttcaagggagctctgagtcaggttgtctcacaataactactcaattacaacacagcttgaacaatattcctcctgaggctaatatttctcgaccaacatattccaatgttttcgattatgcacgatataataatgtcacatctacatcgtcgtcctccatgccatctcaacaagccgcagagctccgaagcttttcgatgcagtttggttcttcttcaagttcgagttcgggtcctccttataagtataggaaagactttgacagaagtcactcaagatga

SEQ ID NO: 79 - DNA, 732 bp, Fragaria vesca
  source 1..732: mol_type = other DNA
atgtcagccatggtttctgctctcactcaagttatcggaaaccccgaagaagataacaagcaggtgcaatcaaaccctgcttcagtgaaagatgaaccggaccgctcacaaccggttcaagatcaagagagtactgtgagaagaaggcactatagaggagtgaggcaaaggccttggggcaaatgggcagccgaaatccgagaccctaagaaagcagctcgagtttggctaggaacttttgagacagctgaggatgcagccattgcatatgataatgcagctctcaaatttaaaggcacaaaagccaagctcaactttcctgaacgagttcaaggtaactccagcatccttattcagggttcatccggcacgacaagtagcggcagcagtgtctccactgaacgaaatcggagccgaccagccttaaccgttcctcatcatgatgcttcctatgttgcaccgtcccagccgcagcaggaaagtagtagcttccctgatctatatcaatatgcgcagcttcttcaaagcaatgatattgatttcagctcgaattatcagtaccctccaaataatccattcaatcaagattatcatccgcagtattctactccgcagtttccaccatcaacatattatccttcgcatcagcaacaaggacagcaagacgatcatgttcaggaagatcatcagaatgagaacaagaactggaataggcgtaatccgagcgagtag

SEQ ID NO: 80 - DNA, 723 bp, Fragaria vesca
  source 1..723: mol_type = other DNA
atgtctgccatggtctcagttctatctcgagtgattagtggagacagtagcactgatactgacccaaaccctgcactgcttcaacttccccaacagtccagcaccgccacaccggaacttgatcaatcccatcaacaagcggcgccagatcaagcaggaagcgtgaggaggagacattatagaggagtgaggcagagaccatgggggaaatgggcagccgagattcgtgatccaaagaaggcagcccgagtgtggctcggaacttttgaaacggcggaggctgctgcacttgcttatgatgaagcagctctcagattcaaaggaagcaaagctaagcttaacttcccggaaagagttcaagggacaagtgaatcaggcggttacctcacaactcaaacagttgctcatcagcctctcatttctgattatcatcagcaacaactggtatacccgaataatatcactactactgatcaatattatccacaattttatgggaacttaaactatggtcagccagatccgagattttataaccagcctgctacatcttcatatccacctttcataccaatatctcaagcacaagaagaagatcgacaacaacagccggccttaagctctcctatgccggatttcggttcaccgtcttcaagtttgcatcctgaatatccacagtatgatactacaagaaattttgacaatagtcattggagaggatag

SEQ ID NO: 81 - DNA, 675 bp, Fragaria vesca
  source 1..675: mol_type = other DNA
atggccaaggagcaggagcacgccatcatggtctccgccctcgaacaggtcatcggcggcggcattgccaccagaaccagtggaacctctcagaaccactgccagtatgccacgtcagcggctgaagcgggaacaaataagaaggttgtgatattagtttcggatggcgacacctgtcaggtatgcaagatcgatggctgcctcggctgcgagttcttcccgccaagcaataagcatggcaaagggaagagggtcaagaagagcaagtacaggggagtgaggcagaggccgtgggggaaatgggcggcggagattcgtgacccgcggcgggcggtgagggtgtggctcgggacgttccagacggcggaggaagcggcgagggcttacgacaaggcggcggtcgagttccgaggagagaaagccaagctcaacttcccacgaataagcagtgaagctggtaccagtactgcagcattgaccaagcaaagcatggagactgatgatgaggtccataatcaagtcaatccaagtaatgagaagtcagcgaatcaggaattgggacagggcagtgatgttaaagacgacgagattgatcgtttcatctggaaaatgcttaaagatgacgacggcgatgaagatttaagcacaatggtcaactcaaatatgttgaattga

SEQ ID NO: 82 - DNA, 768 bp, Fragaria vesca
  source 1..768: mol_type = other DNA
atggtatcggctctaactcaggtgattggaaacactactcatgaacaaaatccgcttcaagttattgatcatcaagttcttggaaacccccaagtcttttctgcagaaaaccctgttgagcaatctcaacctgcagtagttcttcaagctcaaggaaatgtgagacaacactataggggagtgaggcggaggccatgggggaagtgggcagccgagattcgtgatccacataaagcagccagagtgtggctcgggacttttgaaaccgcggaggctgctgcccttgcttatgatgaagcagctctcagatttaaaggaagcaaggctaagcttaacttccccgaaagagttcaagggatcagccctagtacttctagtggtctctaccttgcaataagtactagtaccggtcatgatcatcgactagccaattcagctcctccagctgctccaatttctcgaccaacaacatatagtattaatccaaataatgttaatattgattatgatgcacttgtatcatcgtctcaaaaccacggacaagaaagaactatgccgcagattaatgtacaaacgacgttgtcttctacatcctcggcctcttccatgccatctcatcatcaacaagaacgagtacagcagcaagaagatcagcagcagcttttaaagtttcccattatgccatttggcggttcgtcttcgagttcatcagatcctcctactaagtacaggagagactcctctgggagcggtgactttagaagatga

SEQ ID NO: 83 - DNA, 720 bp, Eucalyptus grandis
  source 1..720: mol_type = other DNA
atggtctcggctctgagccgggtcatctcgaccagcgacgacgcgccgagctcggccgacgacccggcagcgccggtgcaagaggagcatggggaccctccgcagcaggctccagatcaagagagtgttagaaaaaaacactatagaggagtaagacaaaggccatggggaaaatgggcggcggaaatacgcgaccccaagaaagcagcccgtgtttggcttggcactttcgagactgccgaggatgcggccttggcatatgatagggcagctctcaagttcaaaggcaccaaggctaagctcaatttccctgaacgagttcaaggcaaacccgagtatgccgcatactccaatccaagtcaccaaaactcaggtgtcaatgttttgcctgaacaaatcaatcctcagccggcaccttttgttccatatccacacgcggcttttccagacctggctcagtatgcacagcttctatcaagtaatgatgctgaatttccttattatgtttcgaatctctatggtcaggaaccttttggttcgcaacaatcttcatcaacgtcgtcctcatcaattagctcatcgtcctatcactacaaccagcaacaacagcaacaggagccgcaaaatgagccatcaagaacttcttttggatggtccccatctaattatgattttcaaggatatggagatggatttgatccaagaaatcaagggcaatag

SEQ ID NO: 84 - DNA, 804 bp, Eucalyptus grandis
  source 1..804: mol_type = other DNA
atggaggggagaaattggaagagatccaagtcccaggcaggccatgtatcggaggagacagaggatggtgatcggaagagaaacaattatgcatatccctcaagcttgatggaaacggcccgatctcaacaagacacatccgccatcgtatccgccctcgctcaagtcattgcaaaccctgctgccgccgcatctcatcatgcatccctcagttcatccgcatctttatctcaaagttcgcttcatgatcatcaggctccagatgcacaagtagggaagaacaagaagctggaggtgagtaggaactatagaggggtgaggcaaaggccgtggggtaagtatgcagctgagattcgggatccgaagaaggcagctcgagtgtggctcggaacattcgacactgcggagggagccgcgctggcttacgatgaggccgccctcaggttcaaaggcaacaaagccaagctcaacttccctgaaagagttcactctttgcctccaccctatggtcccgcttgcaatgcttctcagcctcaatctcagctgctccctccagctttcagctcatgtgacaatcatgttggggctcaactgatggatggttgtgccatgccacctccacgtcccagctatgtccgcggacaaggccctacttcttcagcttccgccagtgattattttcagtcactccagcctactccttcgttgtcgtcgtcttcttccatgccgtcccctttccatcaacacgatccccaacatagcttcgatggcttttcttcttcttggagatcctcccatgaatag

SEQ ID NO: 85 - DNA, 804 bp, Eucalyptus grandis
  source 1..804: mol_type = other DNA
atggaggggagaaattggaagagatccaagtcccaggcaggccatgtatcggcggagagagaggatggtgataagaagagaaacaattatgcatatccctcaagcttgatggaaacgggccgatctcaacaagacacatccgccatcgtatccgccctcgctcaagtcattgcaaaccctgctgccgccgaatctcatcatgcatccctcagttcatccgcatctttatctcaaagttcgcttcatgatcatcaggctccagatgcacaagtagggaagaacaagaagctggaggtgagtaggaactatagaggggtgaggcaaaggccatggggtaagtatgcagctgagattcgggatccgaagaaggcagctcgagtgtggctcggaacattcgacactgcggagggagccgcgctggcttacgatgaggccgccctcaggttcaaaggcaacaaagccaagctcaacttccctgaaagagttcactctttgcctccaccctatggtcccgctagcaatgcttctcagcctcaatctcagcagctccctccagctttcagctcatgtgacaatcatgttggggctcaactgatggatggttgtgccatgccacctccatgtcccagctttgtccgcggacaaggccctacttcttcagcttccgccagtgattattttcagtctctccagcctactccttcatcgtcgtcgtcttcttccacgacatcccctttccatcaacacgatccccaacatagcttcgatggcttttcttcttcttggagatcctcccaagaatag

SEQ ID NO: 86 - DNA, 849 bp, Eucalyptus grandis
  source 1..849: mol_type = other DNA
atgtcggggctgaaggtggctgatcggggtgacaaagcgccgatcctctatcccgggctggacagggaaagagagacgtcagccatggtgctggctctggcgcgagtggtggccggggaagtccccggcgatgccgaggagtcgtgtccttttccttttccctccggagtcctgaggctgaagagaggccatggagatctgtcggccgagccatcggcggaggcgcagcttcggagagcccctggaagagagtcgtccgttgatgatgctgcgagaggcatcatggaaggtcccagcatgaagacaacaaaccatgcaactccaacttatgagtacagcaacagtacagctgcgatgtcgatgtccaacgacgaacaccaaccgagaagaaaatacagaggagtgagacagaggccgtgggggaagtgggcagccgagatccgagacccagtcaaggccgcgagggtttggctaggcacgtttgagacggctgaaggggcggcccaggcgtatgacgtcgcagccttgaaataccgaggcaacaaggccaagctcaacttcccggagaacgtcgtggcccgcctgtcgctggcggctcctcccgcgacccagatgacggttccagacgccgcgaggacccatgtgaccgttccagcagacacggagcatcagttggcctcgggtccggatcatggctgcggtgaatggtgctcttggttgtctcctgatcctgacctcgcgcattctaatctgttgccatcatcttcttcgccgtcttcttcttcatcttcagtttcaaaagcagcatcttttgctttccccactcgatctggttcctggccattttag

SEQ ID NO: 87 - DNA, 1164 bp, Eucalyptus grandis
  source 1..1164: mol_type = other DNA
atggaagaggcggtcatgccgatgtactcgccatactgtccgcccggagagacgtcggcgatagtgtcggctctaacgcacgttgtcacggggaccagaggcggtcagcatggaggagcgtaccaatcgacattggctccttcattcgcttacgattcggcgtcggccggcagctcatcacagcttccatggacttatattgggcaaaagagagagcgtgacgaagcagggagttcatcacaatttttggccgagcctcctttaagtcagagggattactatgggatctatggtggcagttttgcattaagagaaacctctgctatttccgcttccttacctggactccaggcaaggactgtcgccgccacctccaacgtcccgccgccaccttctggagcgggcccgccgccctatgagggcggagagaggaggaggagatacaggggagtgaggcagaggccgtgggggaaatgggcggcggagatccgcgacccccaaaaagccgcaagggtctggctaggaaccttcgacacggcggaggccgccgcccgggcgtacgacgaggccgccctccggttccgtggcaaccgtgccaagctcaacttccctgaaaacgtcagagtcatcccgcccaacgtcccgacttacggttcccctgccgccgccgccgcgaccctcgccgccggctccgctcctccggcgatagcggcgggtcagctagctcctccgtatgcgggggcggtcccgctatatcctagaggatctgaaggcttcaatttgggggactactgggagtactctcagcttcttcaagggcatcatcatcatcaacctgcgagcttgatggagcaaatgatgtattcttcgcagatggcttcttcacactcgtcgttatcattgccttcttcatctccgccgcagccgccgccgccgcctccttcttttccttcatcagatttatacgggtcttcgtcatcatctggttttggcggcgcttcttcgttttcttcttcagttcctctgtttttccctcagcagcccccgttaggatacttccggcctccgccgccgcggcccccgcacgaccagggaagcggcggggaagactctgagccgccgccgtcatcggattcaagtcattaccattcatcaaccagttga

SEQ ID NO: 88 - DNA, 1047 bp, Eucalyptus grandis
  source 1..1047: mol_type = other DNA
atgtccataatggtttctgctctacgccgcgtcgtgtctggagaagtgttgtcgggcgatgatcagcaacagcagttcaatttgagcggagggtacgagccggcagcgttacttggccggtccagctctttggattccgtcggtgtcggacaaaagagaggaaggggagattcgacctcgtcatctgagttcttggtatctcaggattcggtagcttttggcggttttccgcaggcaggctcatcgtcatcttctgatgccagaggtcatgcaggctcaacaactgttgagacagcaaaagcagatacagcagatggtactgctccaaaatatgaatacaactatgaggccccgacgacgatggcagcatcaaggttggacccgactgtgaggaggaaatacagaggagtgagacagaggccgtgggggaagtgggcggccgagatccgagaccctttcaaggcctcgagggtgtggctcggcacgtttgacacggctgagggggcggctcgtgcctacgaccgggctgccctccagttcagaggcagcaaggccaaactcaacttccccgagaatgtccggctccggcaacaacctgctcctgtggcctctgcagcctttccgtcggcaacccacttcaccatttcccgagagttgccggcttctggcaatttcgatgccactggctacgacgggcaggctcctatgcagcagtttcctgaaaatgattttcgcgaatatcaccgcgatgttgctgctcatcaggaacgactgcagggacgaaccatgagtttgtacgagcagatgctgttttccaactcttcgttcgggagtcagtttcagccagctttctcgctctctccttcttcttcatcatcatcgagttcagtgctcgcgcggtctccaaactcctctcttttcatgccttctccgccgaacgtgacctcgaggtcccaagagagtcggagaggcagcggcgacggggcggccgtcttttctcagcatccctggacagattccagccattacagttcttcttcaggatag

SEQ ID NO: 89 - DNA, 771 bp, Eucalyptus grandis
  source 1..771: mol_type = other DNA
atgcagcaattgcacgacctcggcatgacccgagacgacgaacacggcatcatggttaacgctctccagcacgtgatatccgggagcagctccacgcggcccgagccgatcccggccttccggtcgtccacgccgtcgcacctctccgccgacgggaccgccgcggagcagcgggccccgggcctcctctcgcttcccgacgtggccacttgccaggtgtgcaggatcgacgggtgcctcgggtgcaacttcttcgcaccgggcagagtcgccatggcggtgcagggcggcggggaccccgccaaggctgcccaggcgctcggtgtgggccagagcaacaagagcgccggcaggaagaggaagggcttctacaggggagtgcggcagaggccgtgggggaagtgggcggcggagatccgggacccgcggagggcggcgcgggtgtggctcgggacgttcgagacggcggagcaggcggcccgcgcctacgacagggcggcgctcgagttccgcggtgcgcgggccaagctgaacttcccactgctgccgaatgactgcacgagcaccggtgccggcaagagcagggacatgatggaggacggcgaggctgaagaaacgatcaaagcgagggcggaagggggagagagtaacgatgtccgtgaaaaagctccgtctttcgagcgtgatgacgagagaggagcgagcgagttctgggagaagcttgagaaggaggagctggagcagtggccggtcatgcacttgcctccatga

SEQ ID NO: 90 - DNA, 741 bp, Citrus sinensis
  source 1..741: mol_type = other DNA
atgtacggtacagcagtggtttcagccctttctcaagttattgggaatacccaaaatagcccaactagtcttcaactctctcaaaaccctaatttcaccacttcttctcccaacacatctgaacgagacctatctcaacgcgtcgaagatcaagggaatgtgaggagaagacactacagaggagtgaggcaaaggccatggggcaaatgggcagctgagattcgtgatcctaagaaggcagcacgggtatggctaggaacttttgacacagctgaagcagcagcacttgcttacgatgaagcagctcttaggttcaaaggaagcaaagccaagctgaacttcccagaaagagttcaagctaatctcacgactcaccgttatcaagatcattattatcatgctgcggccgctacaacttcacagcaagtttctaatcctcctcctcctcctcctcctcgtcctctaccactaactcaagaggtaatgtattcgaatctttttcagtatcagcaagcaaactatggcatcccgtccggtttctacggcgagtataggtatctaccggtgactttaccgaccacttcttcatcttcatcttcgtcttctgcgacgtcgtctcagcaaccgcagcagcatgagttattaagatatggcatgcagcttgaaagctcttcatcatcagcttctgatcctcatgagagtacaaggagaaattctgatacaagtcacccaggtgattag

SEQ ID NO: 91 - DNA, 702 bp, Citrus sinensis
  source 1..702: mol_type = other DNA
atgtctgccatggtttccgctcttacacaagtaattggcaccactaccaccgacgccgacgccgacccctcgccggcagccgttaaagaagaatcctccgataacccacttcaacaaactcaaactcaaactcaagatcaagatcaaggtacaaggaggagacattacagaggagtaagacaaagaccttggggcaaatgggcagccgaaattagagaccctaaaaaggcagcacgagtatggctaggtacatttgagactgctgaagatgcagccatggcttatgataaagctgccctcaaattcaaaggcaccaaagctaagcttaatttccctgaaagagttcagggtactactgagtttgtgtatttagattcatcatcatcaagctctgcctttcatcatcatcatgagtcggtgatgcctgctcctcctcctaggcctacatcaatgcaccatggtgcatacccggacttgcttcaatatgctcaaattctatcttctgatgatgctacttttaattactatacgtccaatctctttaatccacaatcatcatcttcttcttcttctactccttcgacgttttcatcatctaccacctcattggaacaacaacaggagatgacgagattttcgtcaaattatgaaagcttgtctggttctgattttcaagatcatagtaacaacccaaatgggtag

SEQ ID NO: 92 - DNA, 705 bp, Citrus sinensis
  source 1..705: mol_type = other DNA
atgtctgccatggtttccgctcttacacaagtaattggcaccactaccaccgacgccgacgccgacccctcgccggcagccgttaaagaagaatcctccgataacccacttcaacaaactcaaactcaaactcaagatcaagatcaagaaggtacaaggaggagacattacagaggagtaagacaaagaccttggggcaaatgggcagccgaaattagagaccctaaaaaggcagcacgagtatggctaggtacatttgagactgctgaagatgcagccatggcttatgataaagctgccctcaaattcaaaggcaccaaagctaagcttaatttccctgaaagagttcagggtactactgagtttgtgtatttagattcatcatcatcaagctctgcctttcatcatcatcatgagtcggtgatgcctgctcctcctcctaggcctacatcaatgcaccatggtgcatacccggacttgcttcaatatgctcaaattctatcttctgatgatgctacttttaattactatacgtccaatctctttaatccacaatcatcatcttcttcttcttctactccttcgacgttttcatcatctaccacctcattggaacaacaacaggagatgacgagattttcgtcaaattatgaaagcttgtctggttctgattttcaagatcatagtaacaacccaaatgggtag

SEQ ID NO: 93 - DNA, 1188 bp, Carica papaya
  source 1..1188: mol_type = other DNA
atgtgtggaggtgctataatctccgatttcatacccaccgctactactcggtcgtgcaagttgaccgccgactacttatggcccgatctgaacaggaataggaagtctaagaagagctccaagaggtctgaggttgtcgacttggacgatgacttcgaggctgatttccagggcttcaaggacgacgaatccgatattgacgtcgacgaagatctcgacgatattgatgctgtcttctctgatattaagccctttgccttctctgcaactcctcttccccgcaaaaccacagcctctgctctctccaatggatcaaagcctgtgaaagctgtggaattcaatgggctggcagagaaatctgcaaaaagaaagaggaagaaccaatacaggggaatcaggcaacgcccttggggcaaatgggctgctgaaatccgtgaccgaaggaagggggtcagggtctggttaggtacttttaacactgctgaagaagctgcaagagcttatgatgctgaggcacggagaattcgtggcaagaaagccaaggtgaacttcccagatgaatctccacgtgcgtctccaaagcgtgccgtgaactcaatgaaaccagttgccaaggcaatcctgaattcagcacagccaaatctgagtcagaatgttaattacttcaacaaccttggtcaggattactacaatacaatggtttttgttgatgagaaaccacaaatgaatcagtttgcatcaatgaattcatttcctccacgtcgaaatgctggagttaaaccctttgtccctagtgacaacacccatatgtacttcagttctgacccagggagtaactcgtttgggtgttctgaatttggctggggagatcaggccacaaagactcctgaaatctcctctgttcttttggatcaacctcagtttgtggaggattgtaacccagagaagaagttgaagtgtagttctgaaactatggtgcctgttcaaggcaatgccaacaagtctctgtctgaggagctgctagcttttgacaatcaaatgaagtacttacaagtgcctcatcttgactcgaactgggattcatctcttgatgctttccttaacggcgatgcgcctcaggatgctgggaactcaatggacctttgggcctttgatgaccttccttctctggttgggggagtcttttga

SEQ ID NO: 94 - DNA, 1215 bp, Musa acuminata
  source 1..1215: mol_type = other DNA; sub_species = malaccensis
atggcggcctgtagctcgggaactgcgaaatggaagctccgagaaggaaagaggaagagggcgagggggtgggaaaaggccaaatcaaagcaagaatgtagagctcatcaaagccgtcgcccagcccaatcatcagagcgtctgttcttatcacaatcccgaatcatccactctttatctttccttctgctgcgactgctcctcgatcggctgcctctctctcctctctctgctgtaaatatctccgtctctctctctcgctgctgtatatatctccgtctatctgtagtctttctttgtcgtcgacgtcgctgtcaccaccaacgtatatcaccggatcctttgtcggggcgacggaaggaagaggaaaggtcaagaaacgataaggtggatcgaaggcctgggaagaggccccttcctcctgatgagctggagaagaaggaaggagaccagcagcaggcggtctcacgcttcgcctcgtcacgcgccgaccacgacgcatcggccatggtgtccgctctcgcgcacgtcatcagctcgagctcatcggttgtcgacacccgaggaggggagccagcgtcgacccaacagggcataaaactggaggaggctgcaggtcgcggcgacacggaagccgcacaggtctcggaagagcaagggaatgtgaggagaagacactacaggggagtgaggcaaaggccgtggggcaaatgggcagcggagataagggaccccaggaaggcggctcgtgtgtggctcgggaccttcgacacggccgaggacgcggccgtcgcctacgacgaggcagcgttgcggttcaaaggcaccaaagccaagctcaactttcccgagagagtccaaggccggaccgacctcggcttcctcgtcagcccaggggtccccgagaggcagccaccccgagtaccgctacagctgcctgcgacctcgtaccccgacctgctgcagtacgctcaactcctccagagtagggatgaagacttgcagaatgtagcttcggggctctacgtcggcggcaccttcacgccggtgtcctctcagacgccaacaacttctgctttagggtcttcacagcacttcttggacttctcatctcagtctcagtatacgaacttttcttcgtcttcttcttcttcgtcttctagctcatgggttcatggagaacagaaggacaaagacggcagtcgacctccatga

SEQ ID NO: 95 - DNA, 684 bp, Musa acuminata
  source 1..684: mol_type = other DNA; sub_species = malaccensis
atggtctctgctctctctcgcgtgatcagctcgagctcgtcggtcatcgacgcctcagccggggagccgacggtgaaccaacaagggattaaactggagggcgctgatcctggggagaaacaagccatccagatctcagaagagcaagggactgtgagaagacactacaggggggtgaggcaaaggccatggggcaaatgggcagcagaaataagggatcccaagaaggctgcacgtgtgtggctggggaccttcgacaccgccgaggacgcggccgtcgcttacgatgaggcagcattgaggttcaaaggcagcaaggccaagctcaacttcccccagcgagtccaaggccgggcggagctcagcttcctcgccagcccggggatccccagaaggcaaccacagccaccgacgcggccgccggcgtcttcatatccagacttgtttcgatacgctcagctcctccagagtggagacgacaacctgcagagcgtagcctcgggcctctacgtcggaagcgccttcacgtcggcgccctctcaggccccgccatcttctacgtcgggatctctaccgcagttcttgggcttctcgtctcactcgccgtacagttcttcttcttcttcttctggttcatgggtttatggagatcacaaggataaagacagcagtcgacctccctga

SEQ ID NO: 96 - DNA, 771 bp, Musa acuminata
  source 1..771: mol_type = other DNA; sub_species = malaccensis
atggatcgaagacatggaaagaggcctcttcctcctgacgaggcggcaccggaggagaaagcgggagagctctcgtgctcgccactggcccgggccgatcaggatgcgtcggccatcgtgtcggccctcgcccacgtgatcggctcttgctcgccggtggccggcgtcggagggggtgagatgcgacaagatgttagtggatcaggaacaggctcggtggagaacaggactcagccctccgaagagcaaggaaatgcgggacgaaggcattataggggagtgaggcaaaggccatgggggaaatgggcagcggagataagggatccccagaaggctgctcgtgtatggcttgggaccttcgacacggcggtggatgcggccatcgcctacgacgaggccgccctcaggttcaaaggctgcaaagccaagctcaacttccccgagagagtccaaggccgctctgacctcggcttcttgacacatcgatggcaagctcagccgccggtgcaactcccggcgacttcgtacccggacttgcttcggtacgctcgactcctccagagtagggacgacgacctgcacaaccgggctgtcgggctccaccccgcgggaagctccttcatgtcaacctcctcgcacacaacgccgacttcttctttgtctgggtcttcgcaggagctggtgggtttctcacaccattggcagttgaggagctcttcttcttcgagctcgtggcctcaggttgacctgcaggacgaggacgaagactag

SEQ ID NO: 97 - DNA, 690 bp, Musa acuminata
  source 1..690: mol_type = other DNA; sub_species = malaccensis
atggtctcggccctctctcacgtgatcagctcccgctcgccgcccgtcggcgccggaggaggcgagccagtcatggtccagcatgatggaaaactcagcggatctgggtctggctcggcggagatcaggacgcagccctccggtgagcaaggcagaagacattacaggggagtgaggcagaggccatgggggaaatgggcagcggagataagggatcccaggaaagcagctcgtgtatggctggggacgttcgacacggcggaggacgccgccattgcctacgatgaagccgccctgcggttcaaaggcaccaaagccaagctcaacttccccgagcgagtacagggccgcaccgacctcggcttcttggtgagcggcggaggctcggaacggcaaccccagccaccgacgcagcggctgccggcggctaattcgtacccgaacctgctccagtacgcgcaactcctccagagcagggaccaagacctgcaccaggcggccttcggcctctacgctgggagcaccttcacgtccacgtcctcccagacatcgccgacttccatgtccgcggcgtcttcgcaagagatgctggacttcacctgccaatcgcatttcaagagctcctcttcttcgagttcgtggcctcatggcggccacaagcacgaagaccagcagccaccaggcatgtaa

SEQ ID NO: 98 - DNA, 642 bp, Musa acuminata
  source 1..642: mol_type = other DNA; sub_species = malaccensis
atggtgtctgccctcacccacgtgatcagctctgtctccccggtcgtcggtgccggaggagatgagctggtggcggaaccggatgctagttgtggatcagggccaggttccatggagatcggaacacaggcttccgaagagcaaggtaggaggcactacaggggagtgaggcaaaggccgtgggggaaatgggcagcagagatcagagacccgaagaaggccgctcgtgtatggctggggaccttcgacagggccgaggacgccgccatggcctacgacgaggccgccctaaggttcaagggcactaaagccaagctcaacttccccgagagagtccaaggccgcaccgacctcggcttcctggtgactcgagcagcacccgaacggcaaccccaaccgccggcgacttcatatccggacctgcgtcagtacgctcagctcctccagagcggggacgcagacgtgcacaacgcggccctcggcctctacgccggaagcaccatcacgtcgacgtccttgtctggctcttcgcaagagacgcaggatttatcttctcgatcgcagttcacgagctcttccgcttccagctcgtggcctcagagtggccagaaggagaaagatcagcggccaccaaccatgtaa

SEQ ID NO: 99 - DNA, 1107 bp, Musa acuminata
  source 1..1107: mol_type = other DNA; sub_species = malaccensis
atgtgtcacaacgtggcgaacccacaccagctgcccgacgacagcttcgccgccgaggggagtgatggcgcaccgctctcgagttaccatcgggcgcaggagatatccaccatagtgtcagcgctggcgcatgtcatggcttccgagcggaggccgcggccggttgggatggcagtggactccgtgtcggtcgtctcttcctcgtcttcctcctcctcctcctcatcatcttcgctgtcctgcatctcttcttcgtactcgtccccgtctctgggcggtcaaggtggcggcgcaagaagtcagaacaggacgagacgggtgccgtcgccgcctgacttggcgttgaggcaccatcaaggccttggcgagttcgcacgctaccgaggcgacgcatcaccagacgttgcagcaacagagcagtatccacaaggaggccctctgccgatactaggataccctgttcctgcagcagccatggaagagccctcccctgcttcctccaacccggaagaggcggagagaagcgagccgagaaggaagtaccgcggcgtccgccagcggccgtggggaaaatgggcggcggagatccgggatccccacaaggcggcgcgggtgtggctgggcacgttcgagactgccgaggaggcagcgcgggcgtacgacgcggccgccctccgcttccgtggcagaagagccaaactcaacttcccggagaacgtccgactgcagccgtcgctctccgtcccgcttgcgacgtccaattcccctgccaccacgtcggataccataaccgattacttggcgtacacgaggctgttgcaggggggcgaggagcacccgcggatccctccgacgagtctcttggatcagtacatgtactccaattacgcttcgcccatgtgttcgactgtgaacgacggcagttctttacctgctccctccatccctacctactcctccgttgtatcttcatcatcaactccgtactctccattctatgcttcgagcacgacggagcagcagacgaactggagtggagtgtcggatattccggagacatcatggatgggttccagccagtttcctccatcttcttcgggcagctga

SEQ ID NO: 100 - DNA, 1164 bp, Musa acuminata
  source 1..1164: mol_type = other DNA; sub_species = malaccensis
atgtgtctaaaagtggcgaacccacaccagtcgtcggacggcagcttcgctgcggcggggagcgatgagatggaagaggacgcggcggcggcggcggggatgatgtattcttcggtgacggcccaggcggcgttgctttcgggtcatcgccgctctcgggagacatccaccatggtctcggccctgacacgcgtcatggccggcgagcagaggccgaggccagttccgatggctgtggactccatgtcggccgtctcttcttcctcgtcgttctcttacatcttttctcctcctccgtcgtattcctcgccatcgaccggtggtcaaagtggcggcgcaagtagtcagacgagaacgagaccggagctgccgtctcacctggcactgaggtactaccgatgtcttggcgagttcgggagctactacggcggagcatcacccgatgttgcagcagtggagcagtatccacaagcatttctgccgatgctgcaatcccctgctcctgcagcagcagccgtggaggaggcctcccctgcttcctcgaaccaagaggaaagggaaagggcggcgccgaaaaggaagtatcgcggcgtccgccagcggccgtggggcaaatgggcggccgagatccgcgacccctacaaggctgctcgggtatggctggggaccttcgagacggccgaggaggcggcgcgggcgtacgacgaggccgccctccgcttccgtggcagcagagccaagctcaacttccccgagaacgtccgccttcagccgtcccactccgtcgctctggcggcgcaggtgccgccgtccaactcccccgctacctcttccggcgccgtgagcgactacttggcgtactcgaggctcctgcagggcgcctcggagtaccagaggctccctccggcgagcctgttggacccgttcgtgcactctggcgtgaacgacagcagttctctgcctgcctcgtcctttcacgccaactccgttccatcttcgactgttatctctccttcttcctcctcctcctcctcttatcctccatcctacgcttcgagcacaccaacggagcggcagatgatctggggcggggcgtcggggtttcctgagacatcatggactcattcaagccagtttcctccgtcatcttctggggacagctga

SEQ ID NO: 101 - protein, 490 aa, Arabidopsis thaliana
MYKQPRQELEAYYFEPNSVEKLRYLPVNNSRKRFCTLEPFPDSPPYNALSTATYDDTCGSCVTDELNDFKHKIREIETVMMGPDSLDLLVDCTDSFDSTASQEINGWRSTLEAISRRDLRADLVSCAKAMSENDLMMAHSMMEKLRQMVSVSGEPIQRLGAYLLEGLVAQLASSGSSIYKALNRCPEPASTELLSYMHILYEVCPYFKFGYMSANGAIAEAMKEENRVHIIDFQIGQGSQWVTLIQAFAARPGGPPRIRITGIDDMTSAYARGGGLSIVGNRLAKLAKQFNVPFEFNSVSVSVSEVKPKNLGVRPGEALAVNFAFVLHHMPDESVSTENHRDRLLRMVKSLSPKVVTLVEQESNTNTAAFFPRFMETMNYYAAMFESIDVTLPRDHKQRINVEQHCLARDVVNIIACEGADRVERHELLGKWRSRFGMAGFTPYPLSPLVNSTIKSLLRNYSDKYRLEERDGALYLGWMHRDLVASCAWK

SEQ ID NO: 102 - protein, 542 aa, Zea mays
MSARDSNHSYKCSDDSQMPYYNNSAPVGENGRIHMAENSLGHHYSSSDIGSQRINNSNPQVFEAQYCTLESSSANGVYPAQSSTSSHSISPLSGSPLSQHDSHSDHTYSSPPSASCLTEVADLLIKQKELENSIVGPGLDISSDCSRSLLQAHVPVRPDNWRQLLGINGGDLMQVVIACGKAVAENDVFATELLISELGHLVSVSGDPMQRLGAYMLEGIVARLSSSGSMLYKSLKCKEPTSSELMSYMHLLYEICPFYKFGYMSANGAIAEAIKGENFVHIIDFQIAQGSQWVTLLQALAARPGGPPYIRITGIDDSNSAYARGGGLDIVGRTLCDVANSCGLPFEFNAVPAASHEVELQHLAIRHGEIIAVNFAYQLHHVPDESVSTENHRDRIIRMIKSINPRVVTLVEQESNTNTAPFFPRYMETLNYYTAMFESIDVALPRDDRRRMSAEQHCVARDIVNLIACEGAERVERHELFGKWKSRFAMAGFRPYPLSSVVNNTINTLLHTYNSYYRLEERDGVLYLGWKNRVLVVSSAWC

SEQ ID NO: 103 - protein, 554 aa, Zea mays
MADTPTSRMVHPFGDAPRQTPKQFLYSGNPQHLCHPYQSAPDTHVVLQRRYTVRSQSHSPNNAGSEDHETHKQYTLESSAASGCSRHGSPSSQSVHAGSGSPVSHDDSHSGSTNGHGSPVSASCVTGEDPTDLKQKLKDLEAVMLGTSETDPEIVNSLEISAANQLSLEPEEWEHMVSMPRGNLKELLIACARAVERNNSYAIDLMITELRKMVSVSGEPLERLGAYMVEGLVARLAASGSSIYKALKCKEPRSSDLLSYMHFLYEACPYFKFGYMSANGAIAEAIKGEDRIHIIDFHIAQGAQWVSLLQALAARPGGPPFVRVTGIDDSVSAYARGGGLELVGRRLTHIAGLYKVPFQFDAVAISGSEVEEEHLGVVPGEAVAVNFTLELHHIPDETVSTANHRDRILRLVKGLSPKVLTLVEQESNTNTAPFAQRFAETLDYYTAIFESIDLALPRDDRERINIEQHCLAREIVNLVACEGEERVERHEVFGKWKARLMMAGFSPSPLSALVNATIKTLLQSYSPDYKLAERDGVLYLGWKNRPLIVSSAWH

SEQ ID NO: 104 - protein, 558 aa, Zea mays
MADPPTSRMVQHPFGDIPSQTPKQFLYSGSSQHLCHPYQSASDAHVAPQRHYTVRSQSQSQSPDAGSEDFETHSRQYTLDSSSASGCSGHGSPSCQSVHAGSRSPVSHSHDDSHSGSTNGNGSPASASCVTEDPTDLKQKLKDLEAVMLGTDTDPETVDSLEIAIADRLSVEPEEWKNNMVSVPRGDLKELLIACARAVEQNNGYSIDLMVPELRKMVSVSGEPLERLGAYMVEGLVARLAASGSSIYKALRCKEPRSSDLLSYMHFLYEACPYFKFGYMSANGAIAEAVKGEDRIHIIDFHIAQGAQWVSLLQALAARPGGPPFVRVTGIDDPVSAYARGGGLELVGKRLSHIAGLYKVPFQFDAVAISGSEVEEGHLGVVPGEAVAVNFTLELHHIPDETVSTANHRDRVLRLVKGLSPRVLTLVEQESNTNTAPFAQRFAETLDYYAAIFESIDLALPRGDRERINIEQHCLAREIVNLVACEGEERVERHEVFGKWKARLMMAGFRPSPLSALVNATIKTLLQSYSPDYKLAEREGVLYLGWKNRPLIVSSAWH

SEQ ID NO: 105 - protein, 484 aa, Vitis vinifera
MQASQQHRSSGMSNRLYYLPPQEAEAHCLPQFQSFDHQLCYNDGSQGTNFSFQGSSERYCTLESSPNSQQDSQSYPSDPHHSPDNTYGSPMSASCITDDVSDLKHKLRELETVMLGPDSDIINSYDNNDLLMAQWLMDELRQMVSVSGEPIQRLGAYMLEGLVARLASSGSSIYKALRCKEPASADLLSYMHILYEVCPYFKFGYMSANGAIAEAMKDENRVHIIDFQIGQGSQWITLIQAFSARPGGPPHIRITGIDDSTSAYARGGGLNIVGQRLSRLAESVKVPFEFHAADMSGCEVQLENLGARPGEALAVNFAFMLHHMPDESVSTQNHRDRLLRLVKSLSPKVVTLVEQESNTNTAAFFPRFLETLNYYTAMFESIDVTLPREHKKRISVEQHCLARDVVNIIACEGVERVERHELLGKWRLRFAMAGFTPYPLSSLVNATIKRLLENYSDKYRLEEREGALYLGWMDRDLVASCAWK

SEQ ID NO: 106 - protein, 533 aa, Populus trichocarpa
MQTSQKKTISDGSRRYGDQPMQYQESYCWPPIQNIDAGSQGTHLSPMTSDQYCTLESSSETSAYPVQNSPSTASFSPNESVVSQPNSRSYPSDLQDSSENACGSPTSESYVTHKLRELETAMLGPDSDNLDMHSMTAMPGPNQIVSEAEKWKFLVEMMSRGDLKEALCTCALAIANGDMFTVEWLMSELRQMVSVTGEPIQRLGAYMLEGLVARLASSGSSIYNALRCKEPAGADLLSYMLLLYEACPYFKFGYMSANGAIADAMKDEISVHIIDFQIAQGSQWVTLIQALAARPGGPPRIRITGIDDSTSAYARGGGLDIVGKRLLKLAESYKVPFEFHTAGVSASEIQIENLGIQPGEAVAVNFALTLHHLPDESVGTQNHRDRLLRLVKSLSPKVVTLVEHESNTNTVPFFARFVETLNYYLAIFESIDVTLPRENKKRISVEQHCLAREVVNIVACEGAERVERHEPLGKWRSRFEMAGFTPYPLSSFVNSTIKILLENYSEKYTLEERDGALFLGWMNRPLVASCAWR

SEQ ID NO: 107 - protein, 553 aa, Solanum lycopersicum
MQASQGPQRSCSVHKLYNKPMQQVQQNYTPCRASDNSNYNDGSNSQAQVSLTTENEKFFTVDTFPATDCAIYDGDPSVSVSSNRSPFSSQCSQSNMFEQRRSYEKTAGSPVSLCSGVDDSNGKKHELRELNNKLLRPESDIDDSCSCSLNGVVSKHFSLTRRNQVLDVASRLDLKELLVACAEAVDEADTSTAEVLMDALEKRVSVYGEPMQRLSAYMLEGLRARLLSSGSNIYKKLKCNEPTSSELLSYMQVLYHITPYFKFAYMSANVVISEAMKNENRIHIIDFQIAQGSQWVFLIHYLARRPGGPPFLRITGIDDSQSAHARGGGLQLVGERLASIAKSCGVPFEFHTAALSGCMVKLENLRVRHGESLAVNFPYMLHHMPDESVSTMNHRDRLLRLVKSLSPKIVALVEQEMNTNTAPFLPRFRETLDYHKAIFESVDVTRPRNDMQRIRSEEHCIARDVVNLIACEGADRVERHEVFGKWRSRLLMAGFTPCPLSPSVAEAIKVMLKEYSSNYKLAESQGALYIGWNNRALATSSAWQLPHSLPLGS

SEQ ID NO: 108 - protein, 544 aa, Solanum tuberosum
MQASQGPQRPCSVHKLCNQPMQQVQQYYTPCHASDNSSYNDGSNSGTEVSLKTQNEKFFTMDSFPATDCAIYDADPSISVSSNRSPFSPQCSQSNMFEKRRSSENTSGSPVSLCSGVDDSNGKKHELWELNNKLLRPESDIDDSCSCSLNGVVSKHFSLTRRNQVLDVASRLDLKELLVACAEAVDEADTSTAEVLMDALEKRVSVSGEPMQRLSAYMLEGLRARLLSSGSNIYKKLKCNEPTSSELLSYMQVLYNITPYFKFAYMSANVVISEAMKNENRIHIIDFQIAQGSQWMFLIHYLARRPGGPPFLRITGVDDSQSAHARGGGLQLVGERLASIAKSCGVPFEFHNAALSGCMVKLENLRVRHGESLAVNFPYMLHHMPDESVSTMNHRDRLLRLVKSLSPKIVALVEQEMNTNTAPFLPRFRETLDYHKAMFESVDVTRPRNDMQRIRSEEHCIARDVVNLIACEGADRVERHEVFGKWRSRLLMAGFTPCPLSPSVAETIKVMLKEYSSNYKLAESQGALYIGWNNRALATSSAWQ

SEQ ID NO: 109 - protein, 545 aa, Glycine max
MQMSQKHKMSYDSSRFTSEPVQNLGSCCFLQSGNLDYYSSSDNSSHATYPSVCTFEQYCTLESSTNNNLPSLNSSSTVSFSPNNSPVSKLQSKSNVLSSQNSLELVNDSLENESCLTLNNDELRHKIRELESALLGHDTYILDTYDTIIPEESDSFMLEAERWKRMMEMISRGDLKEMLCTCAKTVAVNDMETTEWLMSELRKMVSVSGDPIQRLGAYMLEALVARLASSGSTIYKVLKCKEPTGSELLSHMHLLYEICPYLKFGYMSANGAIAEAMKEESEVHIIDFQINQGIQWVSLIQALAGRPGGPPKIRITGFDDSTSAYAREGGLEIVGARLSTLAQSYNVPFEFHAIRASPTEVELKDLALQPGEAIAVNFAMMLHHVPDESVDSGNHRDRLVRLAKCLSPKIVTLVEQESHTNNLPFFPRFVETMNYYLAIFESIDVALPREHKERINVEQHCLAREVVNLIACEGEERVERHELLKKWRSRFTMAGFAPYPLNSFITCSIKNLQRSYRGHYTLEERDGALCLGWMNQVLITSCAWR

SEQ ID NO: 110 - protein, 584 aa, Gossypium raimondii
MDSHQLFGYGVTGAGLSYSTFPSIPNRLFSSLKSDIGNSPNSPFFTPFDCDTNTTLSDSQEQHSSTENLSGLSPSCNSSLESNTCFHRLSPSLGCRSESLLFSSGGTSYTQDANSGHKVIYTLQELETALMAPDENEDLTTPSVSHGESSRPQTAGQRSRAWSQERQGSLALLPQTSFVSKHRQSTEVSHVEKRQKSIEDFSWLGIPPGNLKQLLIACAKVLSENNMDEFDKLIAKARGAVSISGEPVQRLGAYMVEGLVARKEASGSSIYRALHCREPEGKDLLSYMQVLYEICPYLKFGHMAANGAIAEACRTEDRIHIIDFQIAQGTQWMTLLQALAARPGGAPHVRITGIDDPISKYARGGGLEAVKRRLEALSEKFNIPVEFQGMPVFAPDITRDMLDVRPGEALAVNFPLQLHHTPDESVDVNNPRDGLLRMVKSLSPKVTTLVEQESNTNTAPFLPRFIETLEYYLAMFESIDETLPRDRKERVNVEEHCLARDIVNIIACEGKERVERHELFGKWKSRLTMAGFRQYPLSSYVNSVIRGLLRCYSKHYKLVEKDGAMLLGWKDRNLISASAWHCDS

SEQ ID NO: 111 - protein, 535 aa, Brassica rapa
MQTSQKHHSAAGLHMLYPQVHCSPQFQVIDKKNKKSAFSDTTPSKESFFTLESSTASGPLPSYESPSVSITSGRSPFSPQASCISDLHPSPENIYESPLSGASSHVYDEAHVKNKIRELEVSLLSVDPKVEEYSGFSPAAGKSWNWDELLALTPQLDLKEVLVEAAQAVAEGDFAAACGFIDVLEQMVSVSGTPIQRLGTYMAEGLRARLQGTGGNIYRALKCNEPTGRELMSYMGVLYEICPYWKFAYNAANAAILEAVAGEKRVHIIDFQIAQGTQYMFLINELAKLPGGPPLLRVTGVDDSQSRFARGGGLNLVGEKLANKAQSCGVPFEFHDAIMSGCKVHREHLGVEPGFAVVVNFPYVLHHMPDESVSVENHRDRLLRLIKSLGPKLVTLVEQESNTNTSPFLSRFVETLDYYTAMFESIDAARPRDDKQRISAEQHCAARDIVNMIACEERERVERHEVLGKWRVRMMMAGFMGWPVSSSAAFAASEMLKGYDKNYKLGENEGALYLFWKRRPMATCSAWKPNPNQIV

SEQ ID NO: 112 - protein, 557 aa, Hordeum vulgare
MADTPTSRMIHPFSNMQRQNPKQFQFQYPDNPQHPCHPYQPSPDTHVVPQHHYSLKSHSSDASYENHVAQMKHTLVDSSAAAGCMRHDSPSSHSFTPPSIRSGSGSPSSHDDSHSDSTDGSPVSASCVTVTTEDPNDLKQKLKDLEAEMLGPDAAEIVNSLESSVAKQLSLEPEKWAQMMDFPRGNLKELLLACARAVEEKNMYAVDVMVPELRKMVSVSGTPLERLGAYMVEGLVARLASSGHSIYKALRCKEPKSSDLLSYMHFLYEACPYFKFGYMSANGAIAEAVKGEDRIHIIDFHIAQGAQWISLLQALAARPGGPPTVRITGIDDSVSAYARGGGLDLVGRRLSHIAGLCKVPFEFRSVAMAGEEVEEGHLGVVPGEALAVNFTLELHHIPDETVSTANHRDRILRLVKGLRPKVLTLVEQESNTNTAPFPQRFAETLDYYTAIFESIDLTLPRDDRERVNMEQHCLAREVVNLIACEGAERVERHEVFGKWKARLTMAGFRPSPLSSLVNATISKLLQSYSDNYKLAERDGALYLGWKKRPLVVSSAWH

SEQ ID NO: 113 - protein, 524 aa, Oryza sativa
  source 1..524: note = Subspecies Indica
MPYYNNSVPSGGNGRFYITQNHQDAHYASSDDGSQKIGSSPQAFEAPYCTLESSSANGAHPAHSSASSHSISPISGSPLSHHDSHSDHTYNSPPSASCVTEITDLQIKLRELENAILGPELDIAYDSPESALQPNIMATPENWRQLLGINTGDLKQVIIACGKAVAENDVRLTELLISELGQMVSVSGDPLQRLGAYMLEGLVARLSSSGSKIYKSLKCKEPTSSELMSYMHLLYEICPFFKFGYMSANGAIAEAIKGENFVHIIDFQIAQGSQWMTLIQALAARPGGPPFLRITGIDDSNSAYARGGGLDVVGMRLYKVAQSFGLPFEFNAVPAASHEVYLEHLDIRVGEVIVVNFAYQLHHTPDESVSTENHRDRILRMVKSLSPRLVTLVEQESNTNTRPFFPRYLETLDYYTAMFESIDVALPRDDKRRMSAEQHCVARDIVNLIACEGAERVERHEVFGKWKARLTMAGFRPYPLSSVVNSTIKTLLHTYNSFYRLEERDGVLYLGWKNRVLVVSSAWC

SEQ ID NO: 114 - protein, 541 aa, Oryza sativa
  source 1..541: note = Subspecies Japonica
MSARASNHAYICSDDSQMPYYNNSVPSGGNGRFYITQNHQDAHYASSDDGSQKIGSSPQAFEAPYCTLESSSANGAHPAHSSASSHSISPISGSPLSHHDSHSDHTYNSPPSASCVTEITDLQIKLRELENAILGPELDIAYDSPESALQPNIMATPENWRQLLGINTGDLKQVIIACGKAVAENDVRLTELLISELGQMVSVSGDPLQRLGAYMLEGLVARLSSSGSKIYKSLKCKEPTSSELMSYMHLLYEICPFFKFGYMSANGAIAEAIKGENFVHIIDFQIAQGSQWMTLIQALAARPGGPPFLRITGIDDSNSAYARGGGLDIVGMRLYKVAQSFGLPFEFNAVPAASHEVYLEHLDIRVGEVIVVNFAYQLHHTPDESVSTENHRDRILRMVKSLSPRLVTLVEQESNTNTRPFFPRYLETLDYYTAMFESIDVALPRDDKRRMSAEQHCVARDIVNLIACEGAERVERHEVFGKWKARLTMAGFRPYPLSSVVNSTIKTLLHTYNSFYRLEERDGVLYLGWKNRVLVVSSAWC

SEQ ID NO: 115 - protein, 542 aa, Setaria italica
MSARASNHPYKISNDSQMPYYSDSAPVGENGRFHAMQNNLDHHYSSPDDGSQRINSSNTQVFEAQYCTLESSSANGIYPAQSSTSSHSISPLSGSPLSQHDGHSDHTYSSPPSASCLTEVADLQIKLKELENVILGPELDITSDSPESFLQANVQLRPDNWRQLLGIDAGDLKQVIIACGKAVAENDVFATELLISELGQLVSVSGDPMQRLGAYMLEGIVARLSSSGSMLYKSLKCKEPTGSELMSYMHLLYEICPFYKFGYMSANGAIAEAIKGENFVHIIDFQIAQGSQWITLIQALAARPGGPPYIRITGIDDSNSAYARGGGLDIVGRRLHSVAQSCGLPFEFNAVPAASHEVQLEHLAVRPGEIIVVNFAYQLHHVPDESVSIENHRDRIIRMIKSINPRVVTLVEQESNTNTAPFFPRYMETLNYYTAMFESIDVALPRDDRRRMSAEQHCVARDIVNLIACEGPERVERHELFGKWKARFAMAGFRPYPLSSVVNNTINTLLRSYNSCYNLEERDGVLYLGWKNRVLVVSSAWC

SEQ ID NO: 116 - protein, 547 aa, Sorghum bicolor
MADTPTSRMVHPFGDVPRQTPKQFLYSGNSQHLCYPYQSASDTHVVPQRHCTMRSHSPDAGSEDHDNHKQYTLDSSATSGCSRHDSPSSQSVHAGSGSPVSLEDSHSGSTNGNGSPVSASCVTEDPTDLKQKLKDLEAAMLGTDPEIVNSLEISIADQLSLEPEEWKHMMSMPGGNLKELLIACARAVEYNNSYAIDLMIPELRKKVSVSGEPLERLGAYMVEGLVARLAASGSSIYKALKCKEPRSSDLLSYMHFLYEACPYFKFGYMSANGAIAEAVKGEDRIHIIDFHIAQGAQWISLLQALAARPGGPPFVRITGIDDSVSAYARGGGLELVGRRLSHIAGLYKVPFQFDAVAISSSEVEEGHLGIVPGEAVAVNFTLELHHIPDETVSTANHRDRILRLVKGLSPKVLTLVEQESNTNTAPFAQRFAETLDYYTAIFESIDLALPRDDRERINIEQHCLAREIVNLVACEGEERVERHEVFGKWKARLMMAGFRPSPLSALVNATIKTLLQSYSPDYKLAERDGVLYLGWKNRPLIVSSAWH

SEQ ID NO: 117 - protein, 596 aa, Theobroma cacao
MDAHQLFSYGVTGASLSYSTSYSTVPSIPNRLFSSLKSDIGNSPNSPFSSQFDSPFSTQFDCDTNTTLSDSQEQHSSTENLSGLSPSCNSSFESNTYCHKLSPSLDCKREILPLCSGGTSYIQDANSSHKIIYTLQELETALMAPDADEEVTTPNVSCGESSRPQTTGQRSRAWSQEHQGSLVLQPQTSFVSRHRQSTEVSHAEKRQKAIGDLSLQDIPPGNLKQLLIVCAQALSENNMDDFDKLIAKARNAVSICGEPIQRLGAYMVEALVARKEASGSNIYRARRCREPEGKDLLSYMQILYEICPYLKFGYMAANGAIAEACRTEDRIHIIDFHIGQGTQWVTLLQALAARPGGAPHVRITGIDDPLSKYARGGGLEAVGRRLAALSEKFNIPVEFHGVPVFAPDITRGMLDVRPGEALAVNFPLQLHHTPDESVDVNNPRDGLLRMVKSLSPKVTTLVEQESNTNTAPFLPRFIETLDYYLAMFESIDETLPRDRKERINVEQHCLARDIVNVIACEGKERVERHELFGKWKSRLTMAGFRQYPLSSYVNSVIRSLLRCYSKHYKLVEKDGAMLLGWKDRNLISASAWHCDS

SEQ ID NO: 118 - protein, 538 aa, Elaeis guineensis
MQASERDKHSNKSQKPYHPHNSMPVREAESHFLSQNHQSLNYVSSDDGSSQRNVHPHTSGQFCTLESSLATPGYATHNSPSSLAFSPNSGSPLSQQECQSDNTYGSPISVSCITEDPNDLKLKLRELETAMLGPDPDIVESFETTYPSNTSMDPEKWRQVMGIPRGDLKQMLIACARAVAENDILVVEWLISELRQMVSVSGEPIQRLGAYMLEGLVAKLSSSGSAIYKALRCKEPTSSELLSYMHILYEVCPYFKFGYMSANGAIAEAVKGENMVHIIDFQIAQGSQWMTLIQALAARPGGPPCLRITGIDDSVSAYARGGGLHLVGQRLSRLAQACNVPFEFHAAAISGCDVELEHLGVRPGEALAVNFAFQLHHMPDESVSTRNHRDRLLRMVKSLSPKVVTLVEQEANTNTAPFFPRFLETLDYYSAMFESIDVTLPRENKERISVEQHCLARDIVNIIACEGAERVERHELFGKWRSRFTMAGFKPHPLSPLVNATIKTLLENYCEHYRLEERDGVLYLGWKNRALVVSCAWR

SEQ ID NO: 119 - protein, 539 aa, Elaeis guineensis
MQASNKCGSSDESPELYLQHNSMPRREEEFRLRPQNYQSFDQLFSDNGSLSQSCPLQASHVQYCTRDSALTTACYALRDSPSTLSFSPSFGSPVSQQDYHSDNINGSPLNVSCLTEDPADLKHKLRELEVAMLGPDFDTIDSSESSFRSYLMSKPEMWKQMMGIPEGDLKETLIACARAVADNDAQVMDWLIPELRQMVSVSGEPIQRVGAYMLEGLIARLSSSGSSIYKALKCKEPTSSDLLSYMHILYDVCPYFKFAYLSANGAIAEALKNEDKVHIIDFQIAQGSQWMTLLQALAARPGGPPHVRITGIDDSMSAYARGGGLHIVGQRLFRFAESCSVPFEFHAADVSCCDLEVKDLGVHPGEALAVNFPFQLHHVPDESVSTENHRDRILRMVKSLSPQVVTLVEQESNTNTAPLFQRFVETLNYYTAIFESIDVTLPRDSKERINVEQHCLARDIVNIIACEGAERVERHELFGKWKSRFTMAGFGPYPLSPLVNATIKSLLAKYCENYTLEERDGVLYLGWKNRRLSVSSAWR

SEQ ID NO: 120 - protein, 553 aa, Malus domestica
  SITE 143: note = misc_feature - Xaa can be any naturally occurring amino acid
  SITE 149: note = misc_feature - Xaa can be any naturally occurring amino acid
MQASKEQRISGMSNQLYYQPIQEVEAYCLPRFQTLDPRLHYNESSQSTHLPLQSFHEHYCTLESSWSNGSYTVYNTPSNVSFSPSGSPMSQQDSHSYLSDQYHSPDQTYSSPMSGSCITDDATDLKYKLKQLETVMLGPDSNXLDNYCXTFPNGASNSLPDEDSWGQIMESISKKDLKQVLIFCAKAVADNDLLMAQWMMDELRQMVSVSGEPIQRLGAYLLEGLVARKASSGSNIYKALRCKEPASCELLSYMHILYEVCPYFKFGYMSANGAIAEAMKDEDSVHIIDFQIGQGSQWVTLIQAFASRPGGPPHIRITGIDDSMSAYARGGGLNIVGKRLSKLAELVKVPFEFHATSMSGCDVQLEHLCVRPGEALAINFAFMLHHMPDESVSTQNHRDRLLRLVKSLSPKVVTLVEQESNTNTAAFYPRFVETLNYYTAMFESIDVTLPRDHKERINVEQHCLAREVVNIIACEGVERVERHELLGKWRLRFAMAGFTPYPLSSLVNATIKTLLDNYSDKYRLEERDGALYLGWKNRDLVASCAWRCKPSTN

SEQ ID NO: 121 - protein, 551 aa, Fragaria vesca
  source 1..551: note = Subspecies Vesca
MQASKQHRSSGMSKRLHYQPKQEVEAYCLPPFRILDHQPAYNESSQSTHSTAQSFHERYCTLESSSTNGSYNTLYTSSSTVSFSPSGSPMSQHEQDSHYQHHSPDHMYGSPISGSCITDDATDFKYKLKELETAMLGDSNIFDNYCSSLQNGARNTRPEVDSWGQIMDSISKKDLNQVLIFCAKAVADNDLLMAQWMMDELRQMVSVSGEPIQRLGAYMLEGLVARRASSGSSICKALRCKEPASSELLSYMHILYEVCPYFKFGYMSANGAIAEAMKDEDKVHIIDFQIGQGSQWLTLIQAFAARPGGPPHIRITGIDDSTSAYARGGGLNIVGKRLSKLAEMFKVPFEFHAAAISGCDVQLENLGVRPGEALAMNFAFMLHHMPDESVSTQNHRDRLLRLVKSLSPKVVTLVEQESNTNTAAFFPRFVETLNYYTAMFESIDVTLPRDHKERINVEQHCLAKEVVNIIACEGIERVERHELLGKWRSRFAMAGFTPYPLSSLVNATIKTLLKNYSDKYRLQERDGALYLGWKNRDLVASCAWKCKPGTN

SEQ ID NO: 122 - protein, 466 aa, Beta vulgaris
  source 1..466: note = Subspecies vulgaris
MQNPRNTQGFSCTFDSSLLIGADNFCGSPSTESISGNGSTVSLIDPEYSSSDSHDIDNLRNQLRELENVMMGTTSGSFETENWQRLVEMIKRGELKEVLIACAKAVSDDDLLTAEWLISELQHMVSVSGEPIQRLGAYMLEGLIARFSSSGISIYKTLNCNEPTSGELLSHMQLLYEVCPYFKFGYMSANGAIADALKNEDRIHIIDFQIAQGSQWVSLIQALAARPGGPPQVRITGIDDSTSEYARGGGLNIVGERLSKLAKACNLPFEFQAAALRCGTEVKLENFVLRPEEALAVNFPFRLHHIPDESVGPENHRDTLLRLVKSWSPKVVTLVEYELNTNTAPFFPRFLETLDYYSAIFESINVTLPTDHKERINVEQHCLAKEVVNIIACEGAERIERHELHGKWKSRFLMAGFNPYPLSPLVNGTIRTLLQKYSRSYGLEERNGALYLGWMNRVLVTACAWQ

SEQ ID NO: 123 - protein, 525 aa, Eucalyptus grandis
MQTSQKKSHLSMSNKICYQSALEIGTYCLPQFHTLDCYPSYCTLESSSASGGYAIYNPPSTISFSSDGSPVSLQDSVSYPPDALHSPDNTYASSISGSCVTDDANDFLHKLRELENVMLGPDSDISINNDRSFQKGIHVASPGSKLKDAIERKDLKRVLIASAKAVSENDLWTANWLMDELRGMVSVSGEPIQRLGAYMLEGLVARLASSGSSIYKALRCKEPESSELLSYMHILYEVCPYFKFGYMSANGAIADAMKNENRVHIIDFQIGQGSQWITLIQAFAARPGGPPHIRITGFDDSTSAYARGGGLEIVGKRLTKLAQLYKVPFEFHAALIPGSDLLLHHLDVRRGEALAVNFAFMLHHMADESVSIQNHRDRLLRLVKSLSPKVVTLVEQESNTNTAAFLPRFIEAMDYYAAMFESIDVTLHREHKERINVEQHCLARDVVNIIACEGAERVERHELLGKWRLRFAMAGFTPFPLSSLVNGTIKTLLENYSHRYRLEERDGALYLGWMNRDLVASCAWK

SEQ ID NO: 124 - protein, 545 aa, Citrus sinensis
MQFSRMHKMSDGTQKFYDQPVQKLASKNWPPHQNIDHQPSSDDSNERARLSVDTFEQYCTLESSSGTGSHGAPNSPSTASFSPEKTQVSWPNQQSYSSELYQSPDRTCGSPVSGSCITQNENDLRHKLRELETVMLGPDLDTPAMYNVTSPKEDQISSESERWKCLVEIISRGDLKELLCACAKAIENNDMYAAESLMAESRQMVSVSGDPIQRLGAYMLEGLIARLASSGSSIYKALRCKEPASAELLSYMHLLYEICPYFKFGYMSANGAIAEAMKDENKIHIIDFLIAQGSQWIILIMALASRPGGPPHIRITGIDDSTAAYARGGGLEIVGQRLSKLADLYKVPFEFNAAAISGSEVQLENLEVRPGEALAVNFSMMLHHMPDESVSIQNHRDRLLRLVKGLSPKVVTLVEQEANTNTAPFFHRFLETMNHYGAIFDSIDVALPRDSKDRINVEQHCLAREIVNLIACEGAERVERHEPFGKWRSRFIMAGFTPYPLSPFVNATIKTLLENYNDNYTLEERDGALFLGWKNQAIIVSSAWR

SEQ ID NO: 125 - protein, 536 aa, Musa acuminata
  source 1..536: note = Subspecies Malaccensis
MQASTNYKVLSKQHQSYNQNLGRDPESRFMPQNYLTSHCDSSADGFQQGNSHHQTIHDQFYTLESSSEAANFTTHNSPSSPSYSPISRSPASHQDSQSDNNYDSPISYSCITEDSNDLKHKLREIEAAMLGPDSDSIDSFENAYSSYISLEQEKWQQVMGTPRGDLKQILIACARAVENNDILVVEWLIPKLRQMVSVSGEPIQRLGAYLLEGLVAKLASSGSSIYKALKCKEPTSSDLLSYMHILYDVCPYFKFGYMSANGAIAEALKGENMVHIIDFQIAQGSQWVTLIQALAARPGGPPRVRITGIDDSVSAYARGGGLHIVGQRLSRLAKSCNVPFEFHGAALSGCDLELEHLDIRPGEALAVNFAFQLHHMPDESVSTRNYRDRLLQMIKSLSPTVVTLVEQESNTNTAPFFPRFLETVDYYAAIFESIDVTLPRENKERINVEQHCLARDIVNIIACEGDERVERHELFGKWRSRFMMAGFRPYPLSPLVNATIKMLLENYCENYRLEERDGVLYLGWKNRALVVSCAWK

SEQ ID NO: 126 - DNA, 1473 bp, Arabidopsis thaliana
  source 1..1473: mol_type = other DNA
atgtacaagcagcctagacaagagcttgaggcttattattttgagcctaactctgttgagaagcttaggtacttaccggttaacaactctcgtaaacggttttgtacgctcgagccatttcctgactctcctccttataatgctctatctactgctacatatgatgatacatgtggctcttgtgtaacggatgagttgaatgacttcaaacacaagattagggaaatcgaaacggtgatgatggggccggactcgttggacttacttgtcgattgcacggactcatttgattccacggcgagtcaagagattaatggttggagatcaactctagaggctatctcgaggcgggatttaagagctgatcttgtttcatgtgccaaagctatgtcggaaaatgatcttatgatggctcattcaatgatggagaagttgcggcagatggtttcggtttctggtgagcctattcaacggttgggagcttacttattggaaggtctagtggcgcagctagcttcgtcgggcagttctatatacaaagcacttaataggtgtcctgaaccggctagcacagagcttctctcttacatgcacattctctatgaggtttgtccttacttcaagtttggatacatgtcagcaaatggtgctattgctgaggcaatgaaggaagaaaacagagttcacattattgatttccaaatcggtcaagggagtcaatgggtcactcttatccaggcttttgcagctaggcctggtgggcctccgcggattcggataacgggtatcgatgatatgacttcagcgtatgctcgtggaggtggcctaagcattgttggaaatagactcgctaagcttgctaagcagttcaacgttccattcgagtttaactcggtatcggtgtcagtttccgaggttaaacctaaaaacctcggggttcgacctggggaagctctagccgtaaactttgcctttgtgcttcatcatatgccagacgaaagcgtgagcaccgagaatcaccgcgaccggttattgagaatggtgaagagcttatctcccaaggtggtgactcttgtggaacaagagtcaaacacaaacacagccgctttcttcccgaggttcatggagacaatgaactactatgccgcgatgtttgagtcaattgatgtgactctaccaagagatcacaaacagaggattaatgtggagcaacattgtctagcaagagatgtcgtgaacatcatcgcatgtgaaggagctgatcgggtggagcgacacgagctcctaggaaaatggaggtcacggtttgggatggcgggtttcactccttacccgttgagtcccttggtgaattcaaccattaaaagtctgcttaggaactactcagacaagtacaggctagaagaaagagatggagctttgtatcttggttggatgcatcgagatttggttgcctcgtgtgcttggaaatga

SEQ ID NO: 127 - DNA, 1629 bp, Zea mays
  source 1..1629: mol_type = other DNA
atgtcagctagggattctaatcattcatataaatgttcggatgattctcaaatgccgtactacaataactcagcacctgtaggagaaaatggtaggatccatatggcagaaaacagtctaggacatcattactcatcctccgatattggatcacaaaggatcaacaattccaaccctcaagtatttgaagcacagtactgcactctggaatcatcctcagcaaatggtgtttatcctgcacaaagctccacatcttctcatagcatctcccctttaagtgggagccccctgtctcagcatgacagccactcagaccacacatatagttctcctccaagtgcttcctgtctaactgaggttgcagatctcctgattaaacaaaaagaattagagaattccattgttggacctggactggacattagttctgactgctctcggagcttgttgcaagcccatgttccagtgagaccagacaactggagacaacttctgggaattaatggaggagacttgatgcaagtagtcatagcatgtggtaaggctgttgcagagaatgatgtctttgcaacagaactgttaatatctgagcttggtcacctggtatctgtatctggagatccaatgcaacgacttggagcctatatgcttgaaggaattgttgctagactttcttcctccggcagtatgctatataaatctttgaaatgtaaagaacctacaagctctgagctcatgtcatatatgcatctcctttacgagatctgcccattctacaagtttggttacatgtcagcaaatggtgccattgccgaggctattaagggcgagaactttgttcacataattgatttccaaattgctcaggggagccagtgggttactctgctacaggcccttgctgcaagacctggggggccaccatacattagaatcactggtatagatgactcaaattctgcttatgccagaggaggcgggcttgatatagttgggaggacgttgtgtgatgttgctaattcatgtggtcttccttttgagttcaatgccgttccagctgctagccatgaggttgagcttcagcatcttgctataagacatggggagattattgctgtgaattttgcctatcagctacatcatgttcctgacgaaagtgtaagtacagaaaatcatcgggataggattataagaatgatcaagagcatcaatcctagggttgttactctcgttgaacaggagtcaaatacaaacacagctccattcttcccaaggtacatggaaactctcaactactatacagccatgtttgagtcgatagatgttgctctcccaagggatgataggaggcggatgagcgcagaacaacactgtgttgcaagggatattgttaatttaatcgcatgcgagggtgctgaaagggttgagaggcacgagctgtttggaaaatggaagtcaagatttgcaatggctggatttagaccgtacccactgagttcagttgtgaacaacactatcaacacattgttgcatacctacaacagctactacaggcttgaggagagagatggtgtcctttaccttggatggaaaaacagagtattggttgtatcttcggcatggtgttga

SEQ ID NO: 128 - DNA, 1665 bp, Zea mays
  source 1..1665: mol_type = other DNA
atggctgacactccaacgtcccgcatggtgcacccctttggcgacgccccaaggcagactcccaagcaattcctgtattctggtaacccgcagcacctctgccatccgtaccagtcagctccggacacccatgtcgtgctccagcgccgttacaccgtgaggtctcagtctcactcaccaaataatgctggttctgaagaccatgagactcacaagcagtacacgctggagtcctcagcggcttctggttgttcaaggcatggttctccctccagccaaagcgtccatgccgggagtggcagccctgtgtctcacgacgacagccactccggctccacgaatggccatggatcacctgtgagcgcgtcgtgtgtcaccggcgaggaccctactgatctcaagcagaaactgaaggatcttgaggctgtaatgctggggacgtctgagactgaccccgagatagtcaacagtctcgagatctccgcggcaaaccaactctcgttggagcccgaggagtgggagcacatggtgagcatgcccagagggaacctgaaggagctgctgattgcctgcgctagggcagtggaacgtaacaacagctacgccattgatctgatgatcacagagctgaggaagatggtttcggtgtccggcgagccgcttgagaggctgggagcctacatggtggaaggtcttgttgccaggctcgcggcctccggcagctcaatctacaaggctttgaagtgtaaggagcccaggagctctgatctcctctcctacatgcacttcctgtatgaggcctgcccctacttcaagttcggctacatgtcagccaacggcgcgatcgcggaggccatcaagggagaggacaggatccatatcatcgacttccacatcgcgcaaggggctcagtgggtctctctcctccaagcccttgcagccaggcctggcgggccaccgttcgtgagggtcaccggcattgatgattcagtctcagcttacgctcgaggcggcggtctggagttggttggcaggaggttgacacatattgctggcctctacaaggtgccctttcagttcgacgcagtcgctatctcaggcagtgaggtggaggaggagcatctgggcgtcgtcccaggcgaagccgtcgccgtcaacttcacccttgagctgcaccacatccccgacgagaccgtgagcacggcgaaccaccgggaccggatcctgaggctggtgaagggcctgtcgcccaaggtgctcacgctggtggagcaggagtccaacaccaacacggcccccttcgcgcagcggttcgcagagacgctggactactacacggccatcttcgagtccatcgacctggcgctgccgagggacgacagggagcggatcaacatcgagcagcactgcctggcccgggagatcgtgaacctggtggcctgcgagggcgaggagcgggtggagcggcacgaggtgttcggcaagtggaaggcgcggctgatgatggccgggttcagcccgtccccgctcagcgcgctggtgaacgccaccatcaagacgctgctgcagagctactcgccggactacaagctcgccgagagggacggcgtgctctacctcggctggaagaacaggcccctgatcgtctcatcggcatggcactag

SEQ ID NO: 129 - DNA, 1677 bp, Zea mays
  source 1..1677: mol_type = other DNA
atggccgaccctccaacttcccgcatggtgcagcaccccttcggcgacatcccaagccagactcccaagcaattcctatactctggtagctcacagcacctctgccatccataccagtcagcttcggacgcccacgtcgcgccgcagcgtcactacaccgtgaggtctcagtctcagtctcagtcaccggatgctgggtctgaagactttgagacgcacagcaggcagtacacgctggactcctcttcggcttccggttgctcagggcacggttctccctcctgtcagagtgtccatgccgggagtcgcagccctgtgtctcactctcatgacgacagccactccggctccaccaatggcaacggatcgcctgcgagcgcgtcgtgtgtcaccgaggaccctactgacctcaagcagaaactgaaggatcttgaggctgtgatgctggggacggacacggacccagagaccgtggacagtctcgagatcgccatagcggaccggctttcggtggagcccgaggagtggaagaacaacatggtgagcgtgcctagaggggacctgaaggagctgctgatcgcctgcgccagggcagtggagcaaaacaacggctactccatcgacctgatggtcccagagctgaggaagatggtgtcggtgtccggcgagccgctcgagaggctgggagcctacatggtggaaggtctcgtcgccaggctcgccgcctctggcagctccatctacaaggctctgaggtgtaaggagcccaggagctccgacctcctctcctacatgcacttcctgtacgaggcctgcccctacttcaagttcggctacatgtcggccaacggcgcgatcgcggaggccgtcaagggagaagacaggatccatatcatcgacttccacatcgctcagggggcgcagtgggtctctctcctccaagcccttgcggcccggcctggcgggccaccgttcgtgagggtcaccggcattgatgatcccgtttcagcttacgcccgaggcggcggcttagagttggtcggcaagaggctgtcacacatcgctggcctctacaaggtgccctttcagttcgacgcggtcgccatctccggcagcgaggtggaggaggggcatctgggcgtcgtccctggcgaagccgtcgccgtcaactttactctagagctgcaccatatccccgacgagaccgtgagcacggcgaaccacagggaccgggtcctgaggctggtcaagggcctgtcgcccagggtgctcacgctggtggagcaggagtccaacaccaacacggcccccttcgcgcagcggttcgcggagacgctggactactacgcggccatcttcgagtccatcgacctggcgctgcccaggggcgacagggagcggatcaacatcgagcagcactgcctggctcgggagatcgtcaacctggtggcctgcgagggcgaggagcgggtggagcggcacgaggtgttcggcaagtggaaggcgcgcctcatgatggccgggttcaggccgtccccgctcagcgcgctggtcaacgccaccatcaagacgctgctgcagagctactcgccggactacaagctcgccgagagggaaggcgtgctctacctcggctggaagaacaggcccctcattgtttcatcggcatggcactag

SEQ ID NO: 130 - DNA, 1455 bp, Vitis vinifera
  source 1..1455: mol_type = other DNA
atgcaagcatctcagcagcatagaagttcaggcatgtctaacaggttgtattacctacctccgcaagaggctgaggcccattgcttgcctcagttccagagttttgaccaccagctgtgctacaacgatggcagccaaggaaccaacttctcctttcagggttccagtgagcgttactgtactctggagtcatctcccaactcccagcaagattctcagtcgtatccgtctgatcctcaccattcccccgacaacacctacggctctcctatgagtgcatcctgtattactgatgatgtaagcgacctgaagcacaagctgagagaattggagactgtaatgctggggcctgattctgatatcatcaatagctatgacaataatgatctgctgatggcccaatggctgatggatgagttgcggcagatggtgtcggtctctggtgaaccgatccaaaggctgggagcttacatgttggaagggcttgtagcacggctggcctcctccgggagttccatctacaaagctctgagatgcaaagaaccggccagcgctgaccttctctcgtatatgcacattctgtatgaggtctgcccctacttcaagttcggatacatgtctgcgaatggtgccattgcagaagccatgaaggatgaaaacagggttcatattatcgacttccagattggtcaggggagccaatggattactctaatccaggccttttcagctcggcctggggggccaccccacatccgcataacaggtatcgacgattcaacatctgcttatgcccgtggcgggggactcaacattgtgggacaaaggctatccaggcttgctgagtcagtcaaagttccattcgagttccatgcagcagacatgtccggctgtgaagtccagctggaaaatctcggggctcgaccaggtgaagcgttggcggtgaattttgccttcatgctgcatcacatgccggatgagagtgtcagcacccagaatcaccgggacaggctactgaggctggtcaagagcttatctcccaaggtggtcaccctcgttgagcaagaatcaaacacaaataccgctgcattcttccccaggttccttgagacattgaattattatacagccatgttcgaatcaattgacgtgactcttccaagggagcataagaagcggataagcgtcgagcaacactgcctggcaagggatgttgttaacataatcgcatgcgagggggttgagagggtggagcgccatgaacttctcggaaaatggaggttgaggtttgcaatggcggggtttactccatacccattaagctccctggtgaacgccaccattaaaagacttctagaaaactacagtgacaagtataggctcgaggagagggaaggagcgctctatcttggctggatggacagagatttggttgcgtcgtgtgcgtggaagtag

SEQ ID NO: 131 - DNA, 1602 bp, Populus trichocarpa
  source 1..1602: mol_type = other DNA
atgcaaacatcccagaagaaaacaatttcagatgggtctcgtaggtatggtgaccaacctatgcaatatcaggagtcctattgttggccgcccatccagaatattgatgcaggaagtcaagggacgcacctttcacctatgacttctgatcaatactgtaccctagaatcatcctctgaaactagtgcttacccagttcagaattctccatccacagctagtttctcgccaaatgaaagtgtggtttcacagccaaattctcggtcatatccttcagatctgcaggattcctctgagaatgcttgtggttcaccaacaagtgagtcttatgtcacccacaagttaagagaactggaaactgctatgctggggcctgattcagataatcttgacatgcacagcatgactgcaatgcctggacccaaccagattgtatctgaggcagagaaatggaagtttttggttgagatgatgtccagaggagacttgaaagaagcactttgcacttgtgctctagccattgcaaatggtgatatgttcacggttgaatggttaatgtcagagttacgccagatggtgtcagttactggtgagcctatccaacgtttgggagcttacatgctggaaggccttgttgcaaggttggcatcatcaggaagttctatctacaatgccctgaggtgtaaggagcctgctggtgctgatctcctttcctacatgcttttactctatgaagcctgcccatacttcaagtttgggtatatgtcagcaaatggagcaattgctgatgcaatgaaggatgaaattagtgtccatataattgattttcaaattgcgcaaggcagccagtgggtcaccttaatccaggctcttgcagctcggcctggaggacccccacgtattcggattacaggaattgatgattccacatcagcttatgcccggggaggaggacttgatatagtggggaagagactgttgaagctcgctgagtcatacaaggtaccatttgagttccatactgctggagtttctgcttctgaaattcaaattgaaaatcttggaattcaacccggggaagctgtggcagttaattttgcattaacgctccaccacttgccagatgaaagtgtgggcactcaaaatcatcgggacaggttattgaggttggttaaatccttgtctcccaaggtggtaactcttgttgaacacgaatctaatactaatacagtccctttctttgcccgttttgttgagacactaaactattatctagctatctttgaatcaattgatgttacattaccaagagaaaacaagaaacgaatcagtgttgaacagcactgtcttgctcgggaggttgttaatattgtagcatgtgagggtgctgagagagttgaacgccatgaacctcttggcaagtggagatctcggtttgagatggctggatttacaccttatccactaagctcttttgtcaattctactatcaagattctgctggagaactactctgaaaagtatactcttgaagagagagatggagctctctttcttggctggatgaaccgacctttggttgcctcttgtgcatggagatga

SEQ ID NO: 132 - DNA, 1662 bp, Solanum lycopersicum
  source 1..1662: mol_type = other DNA
atgcaagcatctcagggacctcaaaggtcgtgtagtgtacataaattgtacaataagccaatgcagcaagttcagcaaaactacaccccttgccgtgcttctgacaacagcaattataatgatggtagcaactcccaggcacaggtttctttaacgacagagaatgagaagttcttcactgtggacacatttccggctactgactgtgccatctacgatggagatccttccgtaagcgtctcttccaacaggagtcctttctcttctcaatgttctcagtcgaacatgtttgagcaacgtcgttcttacgagaaaactgctggttcacctgtaagtttgtgttcaggagttgatgacagcaacgggaagaagcatgagctgcgggaactgaataataagttgctaaggcctgaatccgatattgatgacagctgcagttgctcattaaatggtgtagtctcaaaacatttttccttgacaaggcggaatcaagtattggacgtagcctcaagattggacttgaaagagttgcttgttgcctgtgctgaagcagtagatgaagctgatacctcgactgcagaagttctgatggatgctttggagaaaagggtatcggtttatggggaacctatgcaacgactgagtgcatacatgttggaaggtctaagagcacgattattgtcctccgggagtaacatatacaaaaaactgaagtgcaatgaaccaactagctcagaattgttgtcctacatgcaagtcctgtatcacatcaccccttacttcaagttcgcttatatgtccgccaatgttgtcataagcgaagccatgaagaatgagaatagaatccatatcattgattttcaaattgcacagggaagtcaatgggtgttcctcatccactatctggctcgtcggcctggtggtccgccatttcttcgcatcacaggcattgatgattcccaatcagctcatgcacgcggtggtggacttcagctagttggcgaaaggttagcgagcattgccaagtcctgcggagtgcctttcgaattccatactgctgcattgtcaggctgtatggtcaaactagagaacctaagagttagacatggagaaagcctggcagttaacttcccttacatgttgcaccacatgccagacgagagcgtaagcactatgaaccatcgggaccgcctattaagactagtcaagagcttgtctcccaaaatcgtggccttagttgaacaagaaatgaacaccaataccgcccctttccttccaaggttccgtgaaactctagattaccacaaagcaatattcgaatcagtcgatgtaactcgcccgaggaatgacatgcaacggatcagatcagaggagcattgtattgcacgggatgttgtcaatctcatagcatgtgaaggggctgatagagtggaaaggcatgaagtttttggcaagtggaggtcaagacttttgatggctggatttactccatgcccgctgagtccatcggttgctgaggccattaaggtcatgttgaaggagtatagctcaaattataagcttgctgaaagccagggggcgctttatattggatggaacaacagagctttagcaacttcttctgcttggcaattacctcattcgctgcctttgggatcttga

SEQ ID NO: 133 - DNA, 1635 bp, Solanum tuberosum
  source 1..1635: mol_type = other DNA
atgcaagcatctcagggacctcaaaggccatgtagtgtacataaattgtgcaatcagccaatgcagcaagttcagcaatactacaccccttgccatgcttctgataacagcagttataatgatggtagcaactccggaacagaggtttctttaaagacacagaatgaaaagttcttcactatggactcatttccggctactgactgtgccatctacgatgcagatccttccataagcgtctcttccaacaggagtcctttctctcctcaatgttctcagtcaaacatgtttgagaaacgtcgttcctctgagaacacttctggttcacctgtaagtttgtgttcaggagttgatgacagcaacggaaagaagcatgagctgtgggaactgaataataagttgctaaggcctgaatccgatattgatgacagctgcagttgctcgttaaatggtgtagtctcaaaacatttttccttgacaaggcggaatcaagtattggacgtagcctcaagattggacttgaaagagttgctcgttgcctgtgctgaagcagtagatgaagctgatacctcgactgcagaagttctgatggatgctttggagaaaagggtatcggtttctggggaacctatgcaacgactgagtgcatacatgttggaaggtctaagagcacgattattgtcctccgggagtaacatatacaaaaaactgaagtgcaacgaaccaactagctcagaattgttgtcctacatgcaagtcctctataacatcaccccttacttcaagttcgcttatatgtccgccaatgttgtcataagcgaagccatgaagaacgagaatagaatccatatcattgattttcaaattgcacagggaagtcaatggatgttcctcatccactatctggctcgtcggcctggtggtccgccatttctccgcatcacaggcgttgatgattcccaatcagctcatgcacgcggtggtggacttcagctagtcggcgaaaggttagcgagcattgccaagtcctgcggagtgcctttcgaattccataatgctgcattgtcaggctgtatggtcaaactagagaacctacgagttagacatggagaaagcctggcagttaacttcccttacatgttgcaccacatgccagacgagagcgtaagcactatgaaccatcgagaccgcctattaagactagtcaagagcttgtctcccaaaatcgtggccctagttgaacaagaaatgaacaccaatactgcccctttccttccaaggttccgtgaaactctagattaccacaaagcaatgttcgaatcagtcgatgtaactcgcccgaggaatgacatgcagcggatcagatcagaggagcattgtattgcacgggatgttgtcaatctcatagcatgtgaaggggctgatagagtggaaaggcatgaagtttttggcaagtggaggtcaagacttttgatggctggatttacaccatgcccactgagtccatcggttgctgagaccatcaaggtcatgctgaaggagtatagctcaaattataagcttgctgaaagccagggggcgctttatattggatggaacaacagagctttagcaacttcttctgcttggcaataa

SEQ ID NO: 134 - DNA, 1638 bp, Glycine max
  source 1..1638: mol_type = other DNA
atgcaaatgtctcagaaacataaaatgtcctatgactctagcaggttcaccagtgagcctgtgcaaaatctggggtcatgttgcttcctgcaaagtgggaacctagactactactcatcctctgataacagtagccatgcaacctacccttctgtttgtactttcgaacaatattgcaccctggaatcttctacaaacaacaatttaccttccctaaattcttcatctactgtcagtttctcacctaacaatagcccagtctcaaagctgcaatcaaaatcaaacgtgttgagttcgcagaattctcttgaacttgttaatgattcgctagaaaacgaatcttgtttgacacttaacaatgacgagctgaggcacaagataagagagcttgaaagtgctttgttgggacatgatacatatattctggatacttatgataccataattccagaagaatctgattcatttatgttagaggcagaaagatggaagagaatgatggaaatgatatctagaggggatttgaaagagatgctctgtacatgtgcaaaaacagtggcagtaaatgatatggagacaactgaatggttgatgtcggagttgcgtaaaatggtctccgtgtctggtgatccaatccaacgactgggagcatacatgttggaggctcttgttgcaaggctggcctcttcaggaagcacaatctacaaagtcttgaaatgtaaagagcctactggtagtgaactcctttcacacatgcatctactttatgaaatctgtccatatctcaaatttgggtacatgtccgcaaatggagctattgctgaagccatgaaggaggaaagtgaagtccacattattgattttcagattaaccagggaattcagtgggtaagcctaatccaagctcttgctggcagacctggaggaccccccaagattagaataacaggttttgatgactccacttcagcttatgccagggaagggggccttgaaattgttggagcgaggttatcaacgcttgcacaatcatataatgtaccctttgagttccatgctatacgagcttctcccactgaggttgaactcaaagaccttgcacttcaacctggtgaagctattgctgtgaactttgccatgatgttgcaccatgttccagatgaaagtgtggatagtgggaaccatcgtgaccggttggtgagattggcaaagtgcttgtctcctaagattgtgacactagttgagcaagaatcacataccaacaacctcccattcttcccccgtttcgttgagacgatgaactactacttggctatttttgaatcaattgatgttgctctgccgagggagcacaaagaaaggattaatgtggagcagcattgtttggctcgagaagttgtcaacttaatagcatgtgaaggggaggaaagagtggaacgtcacgagcttctgaagaagtggagatcgcgttttactatggctggattcgcaccatatccactgaactcgtttattacttgttcaataaaaaatcttcagcgaagctaccgaggacattacactctagaggagagagatggggctctgtgtcttggttggatgaatcaagttcttattacctcttgtgcttggaggtga

SEQ ID NO: 135 - DNA, 1755 bp, Gossypium raimondii
  source 1..1755: mol_type = other DNA
atggactcacaccagcttttcggatacggtgtgactggtgcaggcctatcctactcaacttttccttcaatacctaataggctgtttagttctttgaaatctgatataggaaactcacccaactcacctttttttactccatttgattgtgataccaacactacgttgagtgacagtcaggagcagcatagctctactgagaatctctcaggacttagcccttcttgcaactcttcgcttgaatctaacacttgtttccatcggttgagtccctctctgggctgcaggagtgaaagcttattgttcagttctggtggaacttcttatacgcaagatgcaaattctggccacaaagtaatatacactttgcaggaattggagaccgcacttatggcacctgatgagaatgaagacttaaccacccctagtgtttcccacggagaaagtagtagaccacagactgcaggccagcgatctagggcatggagtcaggagcgccaaggatctttggcacttctgcctcagacatcctttgtctctaagcataggcaatcgactgaagtttctcatgttgaaaaacgccagaaatcaatagaggatttctcttggctgggtattccacccggcaatctgaagcagttgctgattgcttgtgctaaagttctctctgaaaacaatatggatgaatttgataagttgattgcgaaggccaggggtgctgtctctatcagtggggaaccggtccagcgtcttggtgcttacatggtggaagggttggtggcaaggaaggaagcatcagggtctagcatatatcgtgcccttcattgcagagagcctgaaggcaaggacttgctgtcctacatgcaagtcctgtatgaaatctgcccttacttaaagttcgggcacatggcagccaatggagccattgctgaagcatgccgaaccgaagatcgcattcacattattgatttccagattgctcagggaacacaatggatgactctcttgcaggcacttgcagctagaccaggtggagctcctcatgtccggattacaggaattgatgatcctatttctaaatatgctcgtggtggtggattggaagcagttaaaagacgtttggaagcactatctgagaagttcaatattccagttgagtttcagggaatgccagtttttgcaccagatatcacaagggacatgcttgatgtcaggcctggggaggctctggctgtcaacttccctctgcagctccatcacacccctgacgagagcgttgatgtgaacaatcctagggatgggcttctacgtatggtaaagtcactgtctcccaaggttaccacattggtagagcaagaatcaaacacgaacacggcccctttcctccccaggttcatagagactctggagtactacttggcaatgtttgagtccatagatgaaacactgccaagggacagaaaggaacgagttaacgtggaagaacattgcctggccagggatattgtgaatatcattgcttgtgagggaaaggagagggttgagcgtcatgaactctttggtaagtggaagtctagattaacaatggcaggatttcgacaataccctttaagctcttatgtcaattccgtgatacgaggcttgctgaggtgttactctaaacattataagctggtagagaaggatggcgctatgttgttggggtggaaggataggaaccttatatcagcttctgcttggcactgtgacagctga

SEQ ID NO: 136 - DNA, 1608 bp, Brassica rapa
  source 1..1608: mol_type = other DNA
atgcaaacgtctcagaaacatcacagtgcggctggactgcacatgctgtaccctcaagtccactgttcacctcagttccaagtcatagacaagaagaataagaagagcgccttctctgataccaccccatctaaagaaagcttcttcaccctcgaatcctccactgcttctggtcctcttccttcctacgagtccccttccgtgagcatcacatctggccgcagcccgttttctcctcaggcctcatgcatctccgacctccatccttcccctgaaaacatctacgaatctcccttgagtggtgcgtcttctcacgtttatgatgaagctcacgtcaagaacaagatccgagagcttgaggtctcgctgctgagtgttgaccccaaagttgaagagtactcaggcttcagccccgctgctgggaagtcatggaactgggatgagctcttggcgttgactccacagctggacttgaaagaggtgctggttgaggcagcgcaggcggttgccgaaggggacttcgctgcagcgtgtgggttcatcgatgtcttggaacagatggtgtcggtctcgggcactccgatccaaaggctaggtacttacatggcggaagggcttagggcgaggcttcaaggtacaggtggcaatatctacagagcgttgaagtgcaatgaaccaacggggagagagctcatgtcttacatgggagttctctatgagatctgcccttactggaaattcgcgtacaatgctgcaaacgctgctatcttggaagcagtggctggggagaagagagtccacatcatcgatttccagattgctcagggaacacagtacatgtttctcatcaacgagcttgctaaactccccggtgggccaccgttgcttcgtgttactggtgtggatgattcccagtccaggtttgcccgtgggggagggcttaacttggtaggtgagaagctcgcgaacaaggcgcagtcgtgcggtgtaccgtttgagttccacgatgcgatcatgtccgggtgcaaggtccaccgggagcatctcggtgtggagcctggctttgccgtcgttgtgaacttcccttacgtgctgcaccacatgcctgatgagagcgtgagcgttgagaatcacagagacaggctgctccgtctgatcaagagcctcggaccgaaactggtgacgctagtggagcaagagtccaacaccaacacttctccctttttgtcaaggtttgtggagacgcttgattactacacagcgatgttcgagtcaatagatgcggctcggccacgggatgataagcagaggatcagcgcggagcagcactgcgcggcgagggatatagtgaacatgatagcgtgcgaggagagggagagagttgagagacacgaggtgcttgggaagtggagggtgaggatgatgatggccggcttcatgggctggcctgtcagctcatccgcagcgtttgcagcgagtgagatgcttaaaggttatgacaaaaactacaagctaggagaaaatgaaggagcgctctatctcttctggaagaggagacccatggctacatgttccgcttggaaaccaaacccaaaccagattgtgtga

SEQ ID NO: 137 - DNA, 1674 bp, Hordeum vulgare
  source 1..1674: mol_type = other DNA
atggccgatactccgacttcccggatgatccaccccttcagcaacatgcagaggcagaacccgaagcagttccagttccagtacccggacaacccacagcatccctgccacccttaccagccatctccagacacccacgtcgtgccacagcatcactacagcctcaagtctcactcgtcagatgctagctacgagaaccatgttgctcagatgaagcacactctggtggactcctcggccgcggccggctgcatgaggcacgactcgccctccagccatagcttcactcctccgtccatcaggagcggcagtggcagcccttcgtctcacgacgacagccactccgactccacggacgggtctcctgtcagtgcttcatgcgtcactgtgacgaccgaggatcctaacgatctgaagcagaagctcaaggacctcgaggctgagatgcttgggccagacgccgctgagatagttaacagcctcgagagctcggtggcgaagcagctctcgctggagccggagaagtgggcgcagatgatggactttcccaggggcaacctcaaggagctgctgcttgcttgtgccagagctgtggaagagaagaacatgtacgcggtcgacgtgatggtgccggagctgaggaagatggtttcggtctccggtacgccgctcgagaggctgggagcctacatggtggaagggctcgtcgccaggctcgcctcctccggccactccatctacaaggccttgaggtgcaaggagcccaagagctccgacctgctgtcctacatgcatttcctgtacgaggcctgcccctacttcaagttcggctacatgtcggccaacggcgccatcgcggaggccgtcaagggggaggacaggatccacataatcgacttccatatcgctcaaggagctcagtggatctccctcctccaggcccttgcggccaggcccggcggaccgccgaccgtgaggatcaccggcattgatgactcggtgtcagcctacgcgcgaggcggcgggctggacctcgttgggaggagactgtcgcacatcgccggcctctgcaaggttccctttgagttccgctcggtcgccatggccggcgaggaggtggaggaggggcacctcggggtggtccccggggaggcactggcggtgaacttcaccctggagctgcaccacatcccggacgagacggtgagcacggcgaaccaccgggaccggatcctgcggctggtgaaggggctgaggcccaaggtgctgaccctggtggagcaggagtccaacaccaacacggccccgttcccgcagaggttcgccgagacgctggactactacacggccatcttcgagtccatcgacctcacgctgcccagggacgacagggagcgggtcaacatggagcagcactgcctggcgagggaggtggtgaacctgatcgcgtgcgagggcgcggagcgggtggagcgacacgaggtgttcggcaagtggaaggcgcgcctcaccatggccggcttcaggccctcgccgctcagctcgctggtcaacgccaccatcagcaagctgctgcagagctactccgacaactacaagctcgccgagagggacggggcgctctacctcggctggaagaagaggcccctcgtggtctcgtccgcctggcactag

SEQ ID NO: 138 - DNA, 1575 bp, Oryza sativa
  source 1..1575: mol_type = other DNA; sub_species = Indica
atgccgtactataataactcagtgccttcagggggaaatggtaggttctatataacacaaaatcatcaggatgctcactacgcttcttctgatgatgggtcacagaagattggttcgagccctcaagcatttgaagcgccgtactgcactctagaatcatcctcagcaaatggtgctcatcctgcccatagctctgcatcttctcacagcatctctcctattagcgggagccccctgtctcatcatgatagccactcagaccacacgtacaattcccctccaagtgcatcctgtgtcacagagattacagatctccagattaaactgagggagctggagaatgcaattcttgggcctgagctagacatagcttatgacagccctgagagcgccttgcaaccaaacataatggcaacaccagaaaattggagacagcttctgggaattaatacaggagatttgaagcaagtaatcatagcatgtggcaaggctgttgccgagaatgatgttcggctgacagaactactgatatctgagttaggtcagatggtgtctgtctctggtgatccattgcaacgattgggggcttatatgttggaaggccttgttgccagactttcttcctctggcagtaagatatataaatccttgaagtgtaaggagcctacaagctctgagctcatgtcatacatgcatcttctttacgagatatgcccattcttcaaatttggttatatgtcagctaatggcgccatcgctgaagctattaagggagagaactttgttcacataattgacttccaaattgcacaagggagtcaatggatgacattaatacaggcccttgcagcgaggcctggaggaccaccatttctacgaatcactggtatagatgactcgaattctgcttacgccagaggtggtggactggatgtagttggtatgagattgtataaagttgctcagtcttttggtctgccctttgagttcaatgctgttccagctgctagccatgaggtttatcttgagcatcttgatataagagttggtgaagttattgttgtgaattttgcctatcagctgcatcacactccagatgaaagtgtaagcacagaaaaccatcgggataggattttaagaatggtcaagagcctctcccctaggttggtgactcttgttgagcaggagtcaaatacaaacacacggccattcttcccaagatacttggagactctcgactactacacagccatgtttgagtcgatagatgtcgctctcccaagggatgataagagacggatgagcgcagagcaacactgtgtggcgcgggacattgtcaatttaatcgcatgtgagggtgctgagagggtagagaggcatgaggtctttggaaaatggaaggcaagattaacgatggcagggtttcggccatacccgctgagttcagtggtgaacagtaccatcaaaacgctgttgcatacttacaatagcttctacaggcttgaggagcgagatggtgtcctttaccttggatggaagaacagagtattggttgtatcttctgcatggtgttga

SEQ ID NO: 139 - DNA, 1626 bp, Oryza sativa
  source 1..1626: mol_type = other DNA; sub_species = Japonica
atgtcagctagggcttcaaaccatgcatatatatgttcagatgattctcaaatgccgtactataataactcagtgccttcagggggaaatggtaggttctatataacacaaaatcatcaggatgctcactacgcttcttctgatgatgggtcacagaagattggttcgagccctcaagcatttgaagcgccgtactgcactctagaatcatcctcagcaaatggtgctcatcctgcccatagctctgcatcttctcacagcatctctcctattagcgggagccccctgtctcatcatgatagccactcagaccacacgtacaattcccctccaagtgcatcctgtgtcacagagattacagatctccagattaaactgagggagctggagaatgcaattcttgggcctgagctagacatagcttatgacagccctgagagcgccttgcaaccaaacataatggcaacaccagaaaattggagacagcttctgggaattaatacaggagatttgaagcaagtaatcatagcatgtggcaaggctgttgccgagaatgatgttcggctgacagaactactgatatctgagttaggtcagatggtgtctgtctctggtgatccattgcaacgattgggggcttatatgttggaaggccttgttgccagactttcttcctctggcagtaagatatataaatccttgaagtgtaaggagcctacaagctctgagctcatgtcatacatgcatcttctttacgagatatgcccattcttcaaatttggttatatgtcagctaatggcgccatcgctgaagctattaagggagagaactttgttcacataattgacttccaaattgcacaagggagtcaatggatgacattaatacaggcccttgcagcgaggcctggaggaccaccatttctacgaatcactggtatagatgactcgaattctgcttacgccagaggtggtggactggatatagttggtatgagattgtataaagttgctcagtcttttggtctgccctttgagttcaatgctgttccagctgctagccatgaggtttatcttgagcatcttgatataagagttggtgaagttattgttgtgaattttgcctatcagctgcatcacactccagatgaaagtgtaagcacagaaaaccatcgggataggattttaagaatggtcaagagcctctcccctaggttggtgactcttgttgagcaggagtcaaatacaaacacacggccattcttcccaagatacttggagactcttgactactacacagccatgtttgagtcgatagatgtcgctctcccaagggatgataagagacggatgagcgcagagcaacactgtgtggcgcgggacattgtcaatttaatcgcatgtgagggtgctgagagggtagagaggcatgaggtctttggaaaatggaaggcaagattaacgatggcagggtttcggccatacccgctgagttcagtggtgaacagtaccatcaaaacgctgttgcatacttacaatagcttctacaggcttgaggagcgagatggtgttctttaccttggatggaagaacagagtattggttgtatcttctgcatggtgttga

SEQ ID NO: 140 - DNA, 1629 bp, Setaria italica
  source 1..1629: mol_type = other DNA
atgtcagctagggcttctaaccatccgtacaaaatttcaaatgattctcaaatgccatactacagtgactcagcacctgtaggagaaaatggtaggtttcatgcgatgcaaaataatctagatcatcactactcttctcctgatgatggatcacagaggattaatagttctaacacccaagtatttgaagcacagtactgcactctggaatcgtcgtcagcaaatggtatttatcctgcacaaagctccacatcttctcacagcatttcccctttaagtgggagccccctgtctcagcatgatggccactcagatcacacatatagttctcccccaagtgcttcctgtctaactgaagttgcagatctccagattaaattaaaagagttagagaatgttattcttggacctgaattggacattacttctgacagccctgagagcttcttacaagccaatgttcaattgagaccagacaactggagacaacttctgggaattgatgcaggagacttgaagcaagttatcatagcatgtggtaaggctgttgcggagaatgatgtctttgcaacagaactgctgatatctgagctaggtcagctcgtatctgtatccggagatccaatgcaaagactcggagcatatatgttggaaggaattgttgcaagactttcttcctctggcagtatgctatataaatctttgaaatgtaaagaacctacaggctctgagctcatgtcatacatgcatctcctttacgagatctgcccattctacaagtttggttatatgtcagcaaatggtgctattgccgaggctattaagggtgagaactttgttcacatcattgatttccaaattgctcaggggagccaatggattactctgatacaggcccttgctgcaaggcctgggggtccaccatacattagaatcactggtattgatgactcaaattctgcttatgccagagggggtgggcttgatatagttgggaggagattgcatagtgttgctcagtcatgtggtcttccctttgagttcaatgccgttccagctgctagccatgaggttcagcttgagcatcttgctgtaagacccggggagattattgttgtgaattttgcctatcagctgcatcatgttcctgatgaaagcgtaagcattgaaaatcatcgggataggattataagaatgatcaagagcatcaatcctagggttgttactcttgttgagcaggagtcaaatacaaacacagctccattcttcccgagatacatggaaactctcaactactatacagccatgtttgagtcgatagatgttgctctcccaagggatgacaggaggcggatgagcgcagaacaacactgtgttgcaagggatattgttaatttaatcgcatgtgagggtcctgaaagagttgagaggcatgagctgtttggaaaatggaaggcaagatttgcaatggctggatttaggccttacccactgagttcagtggtgaacaacaccatcaacactctgttgcgtagttacaacagctgttacaatcttgaggaaagagatggtgtcctttaccttggatggaagaacagagtattagtcgtatcttcagcatggtgttga

SEQ ID NO: 141 - DNA, 1644 bp, Sorghum bicolor
  source 1..1644: mol_type = other DNA
atggctgacactccaacttcccgcatggtgcacccctttggcgacgtcccaaggcagactcccaagcagttcctgtattctggtaactcacagcacctctgttatccataccagtcagcttcggacacccatgtcgtgccgcagcgtcattgcaccatgaggtctcattcaccagatgctggctctgaagaccatgataatcacaagcagtacacactggactcctcagcaacttctggttgttcaaggcatgattctccctccagtcaaagtgtccatgctgggagtggcagccctgtatctcttgaagacagccactctggctctacaaatggcaatggatcacctgtgagtgcatcgtgtgtcactgaggaccctactgatctcaagcagaaactgaaggatcttgaggctgcaatgctgggtacagacccagagatcgtcaacagtctcgagatctccatagcagaccaactttcgttggagcccgaggagtggaagcacatgatgagcatgcccggagggaacctgaaggagctgctgattgcctgcgctagagcggtggaatataacaacagctacgccattgatctgatgatcccagagctgaggaagaaggtttcagtgtctggtgaaccacttgagaggctgggagcctacatggtggaaggtcttgttgccaggctcgccgcctctggcagctcaatctacaaagctttgaagtgtaaggagcccaggagttctgatctcctctcctacatgcacttcctgtatgaggcctgcccctacttcaagttcggctacatgtcagcgaacggcgcaatcgcagaggctgtcaagggagaagacaggatccatatcattgacttccacatcgctcaaggggctcagtggatctctctccttcaagcccttgcagccaggcctggcgggccaccgttcgtgaggatcaccggcattgatgattcagtctcagcttacgcccgaggcggtggtctggagttggttggcaggaggctgtcacacattgctggcctctacaaggtgccatttcagttcgacgcagtcgctatctccagcagcgaagtggaggaggggcatctcggcatcgtccctggcgaagccgtcgctgtcaacttcaccctggagctgcaccacatccccgacgagaccgtgagcacggcgaaccaccgagatcggatcctgaggctggtgaagggcctgtcgcccaaggtgctcacgctggtggagcaggagtccaacaccaacacggcccccttcgcgcagcggttcgcggagacgctggactactatactgccatcttcgagtccatcgacctggcgctgccgagggacgacagggagcggatcaacatcgagcagcactgcttggctcgggagatcgtgaacctggtggcctgcgagggcgaggaacgggtggaacggcacgaggtgttcggcaagtggaaggcgcggctgatgatggccgggttcaggccgtccccgctcagcgcgctggtcaacgccaccatcaagacgctgctgcagagctactcgccggactacaagctcgccgagagggacggcgtgctctacctcgggtggaagaacaggcccctgattgtctcgtcggcatggcactag

SEQ ID NO: 142 - DNA, 1791 bp, Theobroma cacao
  source 1..1791: mol_type = other DNA
atggacgcacaccagcttttcagttacggtgtgactggtgcgagcctatcctactcaacctcttattcaactgttccttctatacctaatcggctgtttagttctttgaaatctgacataggaaactcacccaactcgcccttttcgtctcaatttgattcgcccttttcgactcaatttgattgtgataccaacaccacgttgagtgacagccaggagcagcatagctccacggagaatctctcgggacttagcccttcttgtaactcttcatttgaatctaacacttattgccataaattgagtccctctctggactgcaagcgtgaaatcctaccactctgttctggtggaacttcttatatacaggatgcaaattctagccacaaaataatatacactttgcaggaattagagactgcactaatggcacctgatgcggatgaagaagtaaccaccccgaatgtttcctgtggagaaagtagtagaccacagacaacaggccagcgatctagggcatggagtcaggagcaccaaggatctttggtgcttcagcctcagacatcctttgtttctaggcataggcagtcaactgaagtttctcatgctgagaaacgtcagaaagcaataggggatttgtctttgcaggatattccacctggcaatcttaagcaattattgattgtgtgtgctcaagctctctctgaaaacaatatggatgattttgataagttgattgcgaaggccaggaatgctgtgtctatctgtggagaaccaatccagcgccttggtgcttacatggtggaagcgttggtggcaaggaaggaggcatcagggtcgaacatatatcgtgcccgtcgttgcagagagcctgaaggcaaggacttactttcctacatgcagatcctgtatgaaatttgcccctatttgaagtttggctacatggcagccaatggggccattgctgaagcatgcagaactgaagatcgcattcacattatcgatttccacattggtcagggaacacaatgggtgaccctcttgcaggcgcttgcagccagacctggtggagctcctcatgtccgcattacaggaattgatgatcctctttctaaatatgctcgtggtggtggattggaagctgttggcagacgtttggctgcactatctgaaaagttcaatattccagttgagtttcatggagtgccagtttttgcaccggatatcacgagggggatgcttgatgtcaggcctggggaggctctggctgtcaacttccctctgcagctccatcacacccctgatgagagcgttgatgtgaacaaccctagggatgggcttctgcgaatggtaaaatcactttctcccaaagttaccacattggtagagcaagaatcaaacacaaacacagcccctttcctccccaggttcatagagactctggattactacttggcaatgtttgagtccatcgatgagacactgccaagggacagaaaggagcgaattaatgtggagcagcactgcctggccagagatattgtgaatgtcattgcttgtgagggaaaggagagggtggagcgccatgagctctttggtaagtggaagtctaggctaacaatggcaggattccggcaataccctttaagctcttatgtgaattctgtgataagaagcctgctgaggtgttactcaaagcattataaactggtggagaaggatggcgcaatgttgctgggctggaaggataggaaccttatatcggcttctgcttggcactgtgacagctga

SEQ ID NO: 143 - DNA, 1617 bp, Elaeis guineensis
  source 1..1617: mol_type = other DNA
atgcaagcttccgagagggacaaacattcaaataagtctcagaaaccataccatcctcacaattccatgcctgttagagaagcagaatctcattttctttctcagaaccaccaatccttaaactatgtatcatctgatgatgggtccagtcagaggaatgttcaccctcatacaagtggacaattttgcaccctagaatcctccttggcaactcctgggtatgctactcacaactctccatcttctcttgctttctcccctaacagtgggagcccgctatcacagcaagaatgccagtcagacaacacctatgggtccccaataagtgtttcatgtattactgaagacccaaacgatctcaaactcaagttgagggaattagagactgctatgctggggcctgacccagatatagttgaaagctttgagacaacctacccaagtaacacctcaatggatccggaaaaatggagacaagtgatgggaattcccagaggagacttgaaacagatgctcatagcttgtgctagagctgtggctgagaatgatattcttgtggtggaatggctaatttcagagttaaggcagatggtttcggtttctggggagccaattcaacggctgggtgcctacatgttggaaggccttgttgctaagctgtcatcctcagggagtgctatctataaagctttgaggtgtaaagaacctacaagctctgaactcctgtcctacatgcacatcctctatgaggtctgtccatattttaagtttgggtacatgtccgcaaatggtgccattgctgaagctgttaagggggaaaatatggttcatattattgatttccagatagcacagggaagtcaatggatgactctaattcaggcccttgcagcgaggcctggtgggccaccatgcttaagaattactggcattgatgactctgtttcagcctatgcgagaggtggtggacttcatcttgtggggcagagattatctcggcttgctcaggcatgcaatgtgccctttgaattccatgcagcagccatatcaggctgtgatgtagaacttgagcatcttggtgttcgacctggggaagccttggctgtgaactttgctttccagttgcatcacatgccggatgaaagcgtgagcacaaggaatcaccgtgacaggctcttgagaatggttaagagcctgtctccaaaggtggtgactctcgtggagcaagaagccaacaccaacactgctcctttctttccaagattcttggagactctggattactattctgccatgtttgaatcgattgatgtgactcttccgagggagaacaaggaacgtatcagtgtggaacagcactgcttggcaagggacatagttaatatcattgcttgtgaaggagctgaaagggtggaaagacatgagctttttgggaaatggaggtcaaggttcacaatggctggatttaagccacacccattgagtccattggtgaatgccactattaagacactgttagagaactactgtgaacactataggcttgaggagagagatggtgtactataccttggttggaagaacagagcattggtcgtctcttgcgcatggagatga

SEQ ID NO: 144 - DNA, 1620 bp, Elaeis guineensis
  source 1..1620: mol_type = other DNA
atgcaagcttctaataaatgtggaagttccgatgagtctccagagttgtaccttcagcacaattcgatgcctagaagagaagaggaatttcggcttaggccacagaactaccagtcttttgaccaacttttctctgataatggatccctatcccaaagttgcccccttcaagcatctcacgtgcaatattgcacgcgtgactctgccttgacaacagcttgttatgcacttcgtgactctccatcaactcttagcttctccccttcttttgggagccctgtctcacagcaagattatcactcagacaacatcaatggatctccattaaatgtttcatgtttaactgaagatcccgctgatttgaaacacaagctaagggaattggaggttgcaatgcttggtcctgacttcgataccattgacagctcggaaagtagcttcaggagctacttaatgtccaaaccagagatgtggaaacaaatgatgggcatccctgaaggagacctgaaggaaacactcattgcttgtgcgcgagctgtggccgacaatgatgctcaggtaatggattggctaataccggagttaaggcagatggtttctgtttctggagaacccatccaacgggtaggagcttacatgctggaaggccttattgctaggctgtcttcttcaggcagttctatctacaaagctttgaagtgtaaagaacctacgagctctgacctcctctcttacatgcacattctctatgatgtgtgcccttacttcaaatttgcatacttgtctgcaaatggtgccattgctgaagctttgaagaatgaagataaggttcacattattgatttccagattgcacagggaagccaatggatgactctgctgcaggcccttgctgcaaggcctggtgggccaccacatgtgagaatcactggcatcgatgactccatgtctgcatatgcccgaggcgggggactacacattgtggggcagcggttgtttcgcttcgctgaatcatgtagtgtgccgtttgaattccatgctgctgatgtgtcttgttgtgacttagaggtcaaggatctaggcgttcatcctggcgaagccttggctgtaaactttcccttccaactgcatcatgtgccagatgagagtgtgagcaccgaaaatcaccgtgaccgaattttgagaatggtcaagagcttgtccccccaggtggttactcttgtggagcaagaatctaatacaaatactgctcctttgttccaacggtttgtggagactctcaattactatactgcaatatttgaatcaatagatgtgactcttcctagggacagcaaagagaggatcaatgtggaacagcactgcttggcaagagacatagtgaacataattgcctgtgagggtgcagaaagggtggagcggcatgagctttttgggaaatggaagtcgaggtttacaatggctggatttggtccttacccattgagtccattggtaaatgccactataaagtcacttctggcaaagtactgcgagaattatacactcgaagagagagatggtgtcctttatcttggatggaagaataggcgtttgtctgtctccagtgcatggagatga

SEQ ID NO: 145 - DNA, 1662 bp, Malus domestica
  source 1..1662: mol_type = other DNA
atgcaagcatcaaaggagcagagaatttcagggatgtcaaaccagttatattatcagccaatccaagaagtagaggcctactgcttgcctcgtttccaaactttggacccgcggctacattacaatgagagcagccaaagtacccacctcccattgcagagtttccatgaacactactgcactttggagtcttcctggtctaatggtagctacaccgtttataacaccccatcaaatgtcagtttctcacctagtggaagcccgatgtcacagcaagactctcattcgtatctgtctgatcagtaccattcccctgaccagacctacagctctccgatgagtggatcctgcataaccgatgatgcaactgacttaaagtacaagctgaaacaattagaaactgtaatgctgggtcctgattccaatrttcttgataactattgcaytactttcccgaatggggcgagcaattccctgccagacgaggacagctggggacagattatggaatcaatctctaaaaaggatttgaagcaagtcctcatcttctgtgcaaaagcagtagcagataatgatctgttaatggcacaatggatgatggatgagttacgccagatggtttcagtttcgggtgaaccaattcagaggttgggtgcatacttgctggaaggactagttgcgcggaaggcatcttcggggagtaacatctacaaagcactgagatgcaaagaaccagcgagttgcgaactcctttcttacatgcatattctctatgaggtttgcccttacttcaaattcgggtacatgtctgcaaatggagccattgcagaagccatgaaggatgaggacagtgttcacatcattgatttccaaatmggtcaggggagtcagtgggtcactctaatccaagcttttgcatccaggcctggtggaccaccccacatccgtataacaggtattgacgattccatgtcagcttatgcccgtggaggaggactaaacattgtgggaaagaggttgtctaagcttgcagagttggtcaaggttccattcgagttccatgccacttccatgtctggttgtgacgtacagctggagcatctttgtgttcgacctggggaggctttggctatcaactttgcgttcatgctgcaccacatgcccgatgagagcgtcagcactcagaatcaccgggataggctgttgaggttggttaagagcttgtctccraaagtrgtgaccctcgttgagcaagaatctaacacaaacactgctgcattctatccacgctttgtagaaacactaaattactacacagctatgttcgagtcaattgatgtgactcttccgagagatcacaaggagcggatcaatgtggagcaacactgcttggctagggaggttgttaacataatagcatgcgagggcgttgagagggtggaaagacacgagcttcttggaaagtggaggttgcggtttgcaatggctggatttacaccgtaccctttaagctccttggtaaatgccaccatcaagacgctgctggataactactctgacaaatatcggcttgaagagcgagatggggcactctatcttggctggaagaacagagacttggttgcttcttgtgcatggaggtgcaaaccgagcacgaattga

SEQ ID NO: 146 - DNA, 1656 bp, Fragaria vesca
  source 1..1656: mol_type = other DNA; sub_species = Vesca
atgcaagcatcaaagcagcacagaagttcaggtatgtcgaaacggttgcactatcaaccgaagcaagaagtagaagcctattgcttgcctccattccggatcttggaccaccaaccagcatacaatgagagcagccaaagtacccactccacggctcagagtttccatgagaggtactgcacattggagtcatcctcaacaaatggcagctacaacactctttatacctcctcatcaactgtcagtttctcacccagcggaagcccgatgtcacagcatgagcaagattctcattaccagcaccattctcctgatcatatgtacggctctccaataagtggctcctgcattactgatgatgcaaccgacttcaagtacaagctaaaagaactagaaactgcaatgcttggcgattccaatatctttgacaactattgcagttcccttcagaatggggcaagaaataccaggccagaagtggacagctggggacagattatggactcaatatctaagaaggatttaaatcaagtcctgatcttctgtgcaaaagcagtagcggataatgatctgttgatggcacaatggatgatggatgaattacgccagatggtttcggtttctggggaaccaattcaaaggttgggagcatacatgttggaaggactagttgcgcgtcgagcatcctcagggagcagcatctgtaaagcattgagatgcaaagaaccagcaagttctgaactcctgtcttacatgcatattctttacgaggtctgtccctacttcaagtttgggtatatgtctgcaaatggagccattgcggaagccatgaaggatgaagataaagtccacatcattgattttcaaattggtcaggggagtcagtggttgactttaatccaggcttttgcagcaagacctggaggaccaccccatattcgaataaccgggattgatgattcgacgtcagcttatgcacgaggtggaggactcaatattgtgggaaagaggctatctaagcttgcagagatgtttaaggtgccatttgagttccatgctgctgctatctctggttgtgatgttcagctggagaatcttggggttagaccaggggaggctttggctatgaactttgcattcatgctgcatcacatgccagatgagagcgtcagcactcagaatcaccgtgatcggctactgaggttggttaagagcttgtctccaaaagttgtgaccctggttgagcaagaatcaaacacaaacactgctgcattttttccgaggtttgtagaaacactaaattactacacggctatgtttgagtcaatagatgtaactcttccgagggatcacaaggagaggatcaatgttgagcaacattgcttggccaaggaagttgtgaacataatagcatgcgagggaattgagagggtggaaagacatgagcttctcgggaagtggaggtcgcggtttgcaatggcaggatttactccataccctttaagttccttggtaaatgcaaccatcaagacactgctcaagaactactcagacaagtataggcttcaagagagagatggtgcgctctatctcggctggaagaacagagatttggttgcttcttgtgcatggaagtgcaaacctggcaccaattag

SEQ ID NO: 147 - DNA, 1401 bp, Beta vulgaris
  source 1..1401: mol_type = other DNA; sub_species = vulgaris
atgcagaatccaagaaatactcaagggttttcttgcacttttgattcttcgttgttaattggtgccgataacttttgtggatctccatcgacagaaagcatctctggtaacgggagcacagtttcacttattgatcccgaatattcttcaagtgactcacatgatattgataatctaaggaatcagctcagagagctcgagaacgtcatgatgggaaccacttcaggtagctttgaaaccgagaattggcaacgtttggtggaaatgataaagagaggggaacttaaggaggtgctaattgcgtgcgcaaaagcagtatctgatgatgacttgctgacagctgagtggttgatttctgagctacaacatatggtttcagtttctggtgaaccaattcaaaggttgggagcctacatgttagaagggttaattgcaagattttcttcttcaggaatttccatttacaaaaccctgaattgtaacgaacctacaagtggtgaactcctctcacatatgcagttgctctatgaagtctgtccgtatttcaagtttgggtatatgtcagctaacggggcaattgccgatgccctgaaaaacgaggaccgcattcacataattgattttcagattgctcaagggagtcaatgggttagtcttatccaagctttggctgcccggccgggtgggcccccacaagttcgtataacaggcatcgacgattccacatctgagtatgctcgtggaggaggacttaacattgtaggggaaagactgtctaagcttgcaaaggcttgcaacctaccattcgagttccaagctgcagccctcaggtgtgggactgaggttaagcttgagaactttgtactgcgacccgaagaagcattagccgtaaattttcctttcagattgcaccatataccagatgagagtgtgggacctgagaatcaccgagacacgttattgaggctcgttaagagctggtcacccaaggtggtaaccctcgttgagtatgaattgaacacaaatacagctccattttttcctcgttttctcgagacactggattattattctgccattttcgagtcaatcaatgttacactccctacagatcacaaggagaggatcaatgtggagcagcattgtttagctaaggaggttgttaacattatagcatgtgaaggcgctgagagaatagaacggcatgagctgcacgggaagtggaaatcccggtttctaatggctggttttaacccatatcctctgagcccactcgtaaatggtaccatcaggactttattgcagaagtacagtaggagttatggacttgaagagagaaatggagctctttacctcggatggatgaaccgagttttggttacagcttgtgcatggcagtaa

SEQ ID NO: 148 - DNA, 1578 bp, Eucalyptus grandis
  source 1..1578: mol_type = other DNA
atgcaaacatcccagaagaagagtcatttaagcatgtcgaacaaaatttgttatcagtctgctcttgagatcggcacctattgcttgcctcagtttcatacattagattgttacccttcctactgcacccttgagtcatcctccgctagtggaggttatgctatatacaacccaccatcaaccatcagcttctcatccgatgggagtcccgtatccttgcaagattctgtgtcttatccacctgatgcgctacactccccagataatacctacgcctcctccataagtggatcttgtgtcactgacgatgcaaatgattttttgcacaagctgagagaattggagaacgtaatgcttggacctgattcggatatttccattaacaatgacaggagtttccagaaggggatccatgtagcttcaccagggagcaaactaaaagatgcaatagaacggaaggatttgaaacgagttctcattgcctctgcgaaggcagtttcagaaaatgatctatggacagcgaactggttgatggatgagttgcgtggaatggtctcagtttctggggaaccgatccagcggctgggagcatatatgttggaaggtctcgttgcacgtttggcctcctcaggaagctccatctataaagctctgcgatgcaaagaacctgaaagctcagagctcctctcctacatgcacattttatatgaggtctgtccatacttcaaatttggatacatgtcagcaaatggggcgatagccgacgcaatgaagaatgagaatagagtccacatcattgacttccaaattggtcagggcagccagtggatcactttaatccaggcatttgcagcaaggcctggcgggccaccccacataagaataactggttttgatgattccacgtcagcttatgcccgtggaggaggattggaaattgtggggaagaggcttactaagcttgctcagttgtacaaagtcccattcgagttccatgctgcattgataccaggttcggatttactgcttcaccatcttgatgttaggcgcggggaggctttggccgtaaatttcgcgtttatgcttcaccacatggctgatgagagtgtgagcattcagaaccatcgggaccggctcctgaggctggtcaagagcctgtcaccgaaggtggtgaccctagtggagcaagaatctaacacgaatactgctgcctttttgccacggttcattgaggcgatggactattatgcagccatgtttgagtcgattgatgtgactcttcacagagagcacaaggagaggattaatgtagagcagcactgcctggcaagagatgttgtcaatataatcgcatgcgagggggctgagagagtcgaaaggcacgagcttctggggaagtggaggttgcgttttgcgatggcaggatttacaccattccctttgagttccctagtgaacggcaccatcaagacgctgttggagaattactctcacagatataggcttgaggagagagatggagctctttatttgggatggatgaaccgggacttggtagcgtcctgtgcatggaagtga

SEQ ID NO: 149 - DNA, 1638 bp, Citrus sinensis
  source 1..1638: mol_type = other DNA
atgcagttttcgcggatgcataagatgtcagatggaactcagaagttctatgaccagcctgtgcaaaagctggcctccaaaaattggcctccccatcagaacatagaccaccagccatcctctgatgacagcaacgaacgagcacgcctatcagtagatacttttgaacaatactgcacccttgagtcatcctctggtacaggcagtcacggtgctcctaattctccatccactgccagtttctcacctgaaaaaacccaagtttcttggccaaaccagcagtcatattcctcagaactgtatcagtcgcctgatcgtacctgtggctcacctgtaagtgggtcttgtataacacaaaatgaaaatgatctgaggcacaaactgcgagaattggaaaccgttatgctgggacctgatttggatacccctgccatgtacaatgttaccagcccaaaggaagaccaaatctcatcagaatcagagaggtggaagtgtttggtagagataatctccagaggggatctgaaagagttactttgcgcttgtgcaaaggctatagagaataatgatatgtacgcagctgaaagtttgatggcagaatcacgacagatggtgtcagtttctggtgatcctatccaacgtttaggagcctacatgttggaagggctcattgcaaggttggcctcgtcaggaagttctatctacaaagccctgagatgcaaagagcctgccagtgctgagctcctatcttacatgcatttactgtatgagatctgtccatacttcaagtttggatatatgtcagcaaatggggcaattgctgaagccatgaaggacgaaaataaaatccacataatcgattttctaattgctcaaggcagccagtggatcatacttattatggctctcgcttctcgtcctggagggcccccacacattcgaattacaggcattgatgattctacagcagcttatgctcgcgggggaggacttgaaattgtgggtcagagactatcaaagctcgcagacttatataaggtgccttttgagttcaatgctgctgcaatttctggttctgaggttcaacttgaaaaccttgaagttcgacctggtgaagcccttgccgtgaatttttccatgatgctccatcacatgccggatgaaagtgtgagcatccagaatcatcgagacaggctcctgaggctggtaaaaggcttgtctcctaaggtggtgacacttgttgagcaggaagccaacactaatactgccccattttttcaccgtttccttgaaacgatgaatcactatggcgctatctttgattcaattgatgttgctctgccaagagatagcaaggaccgaatcaatgtggagcagcactgcttggcacgtgagattgtcaaccttatagcatgcgaaggagctgagagagtggaacgccatgagccttttggcaagtggagatcgcgttttataatggccgggtttacaccctatccattgagcccttttgtgaatgcaaccatcaaaactctgctggaaaactacaatgacaactacacacttgaagagagggatggagcgctctttcttggctggaaaaatcaagctataattgtttcctctgcatggaggtga

SEQ ID NO: 150 - DNA, 1611 bp, Musa acuminata
  source 1..1611: mol_type = other DNA; sub_species = Malaccensis
atgcaagcttcaacaaactacaaagtattaagtaaacagcatcagtcatacaatcaaaaccttgggagagatccggaatcccgttttatgcctcaaaattacctaacttcacattgtgactcctctgctgatggattccaacaagggaactcccatcatcagacaattcatgaccaattctacactctggagtcctcctcggaagctgcgaattttaccactcataactctccatcttctcctagttactccccgatcagcaggagccccgcatctcatcaagattctcagtcagataacaactatgactctcccataagctattcatgtattaccgaagactcaaatgatcttaagcataagttacgggagatagaggctgcaatgcttgggcctgattcagatagcattgacagctttgagaatgcttactccagctatatctccttggagcaagagaagtggcaacaagtgatgggaacccctagaggagacttgaaacagatcctaatagcctgtgctcgagctgtagaaaacaatgatattcttgtggttgagtggctaattccaaagttaaggcagatggtatcagtctctggggaaccaattcaacgtctgggagcatacttgttggaaggcctcgtagctaagctggcttcctcgggaagttccatatacaaggctttgaagtgtaaagaacccactagttcagacctcctctcatacatgcacattctttatgatgtatgcccatacttcaagtttgggtatatgtctgcaaatggagcaatagctgaggctcttaagggtgagaacatggttcatattatcgatttccagattgcccagggaagtcaatgggtaactctcattcaagctcttgcagcacgacctggtgggcctccacgtgtaagaataacagggattgatgactctgtttctgcctacgcccgtggtggtgggctacatattgtggggcagaggttatctcggcttgccaagtcatgcaatgtgcccttcgaattccatggcgcagctctctcagggtgtgatttagaacttgagcatcttgacattcgacctggggaggcattggccgttaactttgctttccaactgcatcacatgccagatgagagtgtgagcacaaggaactaccgagatagacttctacagatgattaagagcttgtctccaacagttgttactcttgtggagcaggaatcaaacacaaacactgctccattctttccaagatttttagaaactgtagactactatgctgccatctttgagtcgatagatgtgactcttccaagggagaacaaggaaaggatcaacgtggagcagcattgcctagcacgagacatagttaacatcattgcatgtgagggagatgaaagggtggagcgccatgagcttttcgggaaatggaggtcgaggtttatgatggctggattcaggccatatcctctgagtcctttagtgaatgcaacaatcaagatgcttttagagaactactgtgagaactatcgtcttgaagagagagacggtgtgctttatcttggatggaagaatagagctttggttgtctcttgtgcatggaaataa

SEQ ID NO: 151 - protein, 243 aa, Arabidopsis thaliana
MYGQCNIESDYALLESITRHLLGGGGENELRLNESTPSSCFTESWGGLPLKENDSEDMLVYGLLKDAFHFDTSSSDLSCLFDFPAVKVEPTENFTAMEEKPKKAIPVTETAVKAKHYRGVRQRPWGKFAAEIRDPAKNGARVWLGTFETAEDAALAYDIAAFRMRGSRALLNFPLRVNSGEPDPVRITSKRSSSSSSSSSSSTSSSENGKLKRRRKAENLTSEVVQVKCEVGDETRVDELLVS

SEQ ID NO: 152 - protein, 282 aa, Arabidopsis thaliana
MATPNEVSALFLIKKYLLDELSPLPTTATTNRWMNDFTSFDQTGFEFSEFETKPEIIDLVTPKPEIFDFDVKSEIPSESNDSFTFQSNPPRVTVQSNRKPPLKIAPPNRTKWIQFATGNPKPELPVPVVAAEEKRHYRGVRMRPWGKFAAEIRDPTRRGTRVWLGTFETAIEAARAYDKEAFRLRGSKAILNFPLEVDKWNPRAEDGRGLYNKRKRDGEEEEVTVVEKVLKTEESYDVSGGENVESGLTAIDDWDLTEFLSMPLLSPLSPHPPFGYPQLTVV

SEQ ID NO: 153 - protein, 192 aa, Arabidopsis thaliana
MVSMLTNVVSGETEPSASATWTMGHKREREEFSLPPQPLITGSAVTKECESSMSLERPKKYRGVRQRPWGKWAAEIRDPHKATRVWLGTFETAEAAARAYDAAALRFRGSKAKLNFPENVGTQTIQRNSHFLQNSMQPSLTYIDQCPTLLSYSRCMEQQQPLVGMLQPTEEENHFFEKPWTEYDQYNYSSFG

SEQ ID NO: 154 - protein, 268 aa, Arabidopsis thaliana
MHYPNNRTEFVGAPAPTRYQKEQLSPEQELSVIVSALQHVISGENETAPCQGFSSDSTVISAGMPRLDSDTCQVCRIEGCLGCNYFFAPNQRIEKNHQQEEEITSSSNRRRESSPVAKKAEGGGKIRKRKNKKNGYRGVRQRPWGKFAAEIRDPKRATRVWLGTFETAEDAARAYDRAAIGFRGPRAKLNFPFVDYTSSVSSPVAADDIGAKASASASVSATDSVEAEQWNGGGGDCNMEEWMNMMMMMDFGNGDSSDSGNTIADMFQ

SEQ ID NO: 155 - protein, 220 aa, Arabidopsis thaliana
MSAMVSALTQVVSARSQTEAEGAHSSSSSAGHKRGWLGIDSAPIPSSFARVDSSHNPIEESMSKAFPEEAREKKRRYRGVRQRPWGKWAAEIRDPHRAARVWLGTFDTAEAAARAYDEAALRFRGNKAKLNFPEDVRILPPPPPLLRSPADTVANKAEEDLINYWSYTKLLQSSGQRSFLERGQEESSNIFEHSPMEQPLPPSSSGPSSSNFPAPSLPNT

SEQ ID NO: 156 - protein, 391 aa, Arabidopsis thaliana
MCVLKVANQEDNVGKKAESIRDDDHRTLSEIDQWLYLFAAEDDHHRHSFPTQQPPPSSSSSSLISGFSREMEMSAIVSALTHVVAGNVPQHQQGGGEGSGEGTSNSSSSSGQKRRREVEEGGAKAVKAANTLTVDQYFSGGSSTSKVREASSNMSGPGPTYEYTTTATASSETSSFSGDQPRRRYRGVRQRPWGKWAAEIRDPFKAARVWLGTFDNAESAARAYDEAALRFRGNKAKLNFPENVKLVRPASTEAQPVHQTAAQRPTQSRNSGSTTTLLPIRPASNQSVHSQPLMQSYNLSYSEMARQQQQFQQHHQQSLDLYDQMSFPLRFGHTGGSMMQSTSSSSSHSRPLFSPAAVQPPPESASETGYLQDIQWPSDKTSNNYNNSPSS

SEQ ID NO: 157 - protein, 231 aa, Arabidopsis thaliana
MHSGKRPLSPESMAGNREEKKELCCCSTLSESDVSDFVSELTGQPIPSSIDDQSSSLTLQEKSNSRQRNYRGVRQRPWGKWAAEIRDPNKAARVWLGTFDTAEEAALAYDKAAFEFRGHKAKLNFPEHIRVNPTQLYPSPATSHDRIIVTPPSPPPPIAPDILLDQYGHFQSRSSDSSANLSMNMLSSSSSSLNHQGLRPNLEDGENVMTTISTEDDRRRQQHASPDRPIK

SEQ ID NO: 158 - protein, 218 aa, Arabidopsis thaliana
MHSGKRPLSPESMAGNREEKKELCCCSTLSESDVSDFVSELTGQPIPSSIDDQSSSLTLQEKSNSRQRNYRGVRQRPWGKWAAEIRDPNKAARVWLGTFDTAEEAALAYDKAAFEFRGHKAKLNFPEHIRVNPTQLYPSPATSHDRIIVTPPSPPPPIAPDILLDQYGHFQSRSSDSSANLSMNMLSSSSSSLNHQGLRPNLEDGENVKNISIHKRRK

SEQ ID NO: 159 - protein, 212 aa, Arabidopsis thaliana
MVSALSRVIENPTDPPVKQELDKSDQHQPDQDQPRRRHYRGVRQRPWGKWAAEIRDPKKAARVWLGTFETAEEAALAYDRAALKFKGTKAKLNFPERVQGPTTTTTISHAPRGVSESMNSPPPRPGPPSTTTTSWPMTYNQDILQYAQLLTSNNEVDLSYYTSTLFSQPFSTPSSSSSSSQQTQQQQLQQQQQQREEEEKNYGYNYYNYPRE

SEQ ID NO: 160 - protein, 248 aa, Arabidopsis thaliana
MYGKRPFGGDESEEREEDENLFPVFSARSQHDMRVMVSALTQVIGNQQSKSHDNISSIDDNYPSVYNPQDPNQQVAPTHQDQGDLRRRHYRGVRQRPWGKWAAEIRDPKKAARVWLGTFETAESAALAYDEAALKFKGSKAKLNFPERVQLGSNSTYYSSNQIPQMEPQSIPNYNQYYHDASSGDMLSFNLGGGYGSGTGYSMSHDNSTTTAATTSSSSGGSSRQQEEQDYARFWRFGDSSSSPHSGY

SEQ ID NO: 161 - protein, 413 aa, Arabidopsis thaliana
MDNVRGSIMLQPLPEIAESIDDAICHELSMWPDDAKDLLLIVEAISRGDLKLVLVACAKAVSENNLLMARWCMGELRGMVSISGEPIQRLGAYMLEGLVARLAASGSSIYKSLQSREPESYEFLSYVYVLHEVCPYFKFGYMSANGAIAEAMKDEERIHIIDFQIGQGSQWIALIQAFAARPGGAPNIRITGVGDGSVLVTVKKRLEKLAKKFDVPFRFNAVSRPSCEVEVENLDVRDGEALGVNFAYMLHHLPDESVSMENHRDRLLRMVKSLSPKVVTLVEQECNTNTSPFLPRFLETLSYYTAMFESIDVMLPRNHKERINIEQHCMARDVVNIIACEGAERIERHELLGKWKSRFSMAGFEPYPLSSIISATIRALLRDYSNGYAIEERDGALYLGWMDRILVSSCAWK

SEQ ID NO: 162 - protein, 593 aa, Arabidopsis thaliana
MVEQTVVREHIKARVMSLVRSAEPSSYRNPKLYTLNENGNNNGVSSAQIFDPDRSKNPCLTDDSYPSQSYEKYFLDSPTDEFVQHPIGSGASVSSFGSLDSFPYQSRPVLGCSMEFQLPLDSTSTSSTRLLGDYQAVSYSPSMDVVEEFDDEQMRSKIQELERALLGDEDDKMVGIDNLMEIDSEWSYQNESEQHQDSPKESSSADSNSHVSSKEVVSQATPKQILISCARALSEGKLEEALSMVNELRQIVSIQGDPSQRIAAYMVEGLAARMAASGKFIYRALKCKEPPSDERLAAMQVLFEVCPCFKFGFLAANGAILEAIKGEEEVHIIDFDINQGNQYMTLIRSIAELPGKRPRLRLTGIDDPESVQRSIGGLRIIGLRLEQLAEDNGVSFKFKAMPSKTSIVSPSTLGCKPGETLIVNFAFQLHHMPDESVTTVNQRDELLHMVKSLNPKLVTVVEQDVNTNTSPFFPRFIEAYEYYSAVFESLDMTLPRESQERMNVERQCLARDIVNIVACEGEERIERYEAAGKWRARMMMAGFNPKPMSAKVTNNIQNLIKQQYCNKYKLKEEMGELHFCWEEKSLIVASAWR

SEQ ID NO: 163 - protein, 529 aa, Arabidopsis thaliana
MQTSQKHHSAAGLHMLYPQVYCSPQFQAKDNKGFSDIPSKENFFTLESSTASGSLPSYDSPSVSITSGRSPFSPQGSQSCISDLHHSPDNVYGSPLSGVSSLAYDEAGVKSKIRELEVSLLSGDTKVEEFSGFSPAAGKSWNWDELLALTPQLDLKEVLVEAARAVADGDFATAYGFLDVLEQMVSVSGSPIQRLGTYMAEGLRARLEGSGSNIYKSLKCNEPTGRELMSYMSVLYEICPYWKFAYTTANVEILEAIAGETRVHIIDFQIAQGSQYMFLIQELAKRPGGPPLLRVTGVDDSQSTYARGGGLSLVGERLATLAQSCGVPFEFHDAIMSGCKVQREHLGLEPGFAVVVNFPYVLHHMPDESVSVENHRDRLLHLIKSLSPKLVTLVEQESNTNTSPFLSRFVETLDYYTAMFESIDAARPRDDKQRISAEQHCVARDIVNMIACEESERVERHEVLGIWRVRMMMAGFTGWPVSTSAAFAASEMLKAYDKNYKLGGHEGALYLFWKRRPMATCSVWKPNPN

SEQ ID NO: 164 - protein, 526 aa, Arabidopsis thaliana
MEATQKHMIQEGSSMFYHQPSSVKQMDLSVQTFDSYCTLESSSGTKSHPCLNNKNNSSSTTSFSSNESPISQANNNNLSRFNNHSPEENNNSPLSGSSATNTNETELSLMLKDLETAMMEPDVDNSYNNQGGFGQQHGVVSSAMYRSMEMISRGDLKGVLYECAKAVENYDLEMTDWLISQLQQMVSVSGEPVQRLGAYMLEGLVARLASSGSSIYKALRCKDPTGPELLTYMHILYEACPYFKFGYESANGAIAEAVKNESFVHIIDFQISQGGQWVSLIRALGARPGGPPNVRITGIDDPRSSFARQGGLELVGQRLGKLAEMCGVPFEFHGAALCCTEVEIEKLGVRNGEALAVNFPLVLHHMPDESVTVENHRDRLLRLVKHLSPNVVTLVEQEANTNTAPFLPRFVETMNHYLAVFESIDVKLARDHKERINVEQHCLAREVVNLIACEGVEREERHEPLGKWRSRFHMAGFKPYPLSSYVNATIKGLLESYSEKYTLEERDGALYLGWKNQPLITSCAWR

SEQ ID NO: 165 - DNA, 732 bp, Arabidopsis thaliana
  source 1..732: mol_type = other DNA
atgtacggacagtgcaatatagaatccgactacgctttgttggagtcgataacacgtcacttgctaggaggaggaggagagaacgagctgcgactcaatgagtcaacaccgagttcgtgtttcacagagagttggggaggtttgccattgaaagagaatgattcagaggacatgttggtgtacggactcctcaaagatgccttccattttgacacgtcatcatcggacttgagctgtctttttgattttccggcggttaaagtcgagccaactgagaactttacggcgatggaggagaaaccaaagaaagcgataccggttacggagacggcagtgaaggcgaagcattacagaggagtgaggcagagaccgtgggggaaattcgcggcggagatacgtgatccggcgaagaatggagctagggtttggttagggacgtttgagacggcggaagatgcggctttagcttacgatatagctgcttttaggatgcgtggttcccgcgctttattgaattttccgttgagggttaattccggtgaacctgacccggttcggatcacgtctaagagatcttcttcgtcgtcgtcgtcgtcgtcctcttctacgtcgtcgtctgaaaacgggaagttgaaacgaaggagaaaagcagagaatctgacgtcggaggtggtgcaggtgaagtgtgaggttggtgatgagacacgtgttgatgagttattggtttcataa

SEQ ID NO: 166 - DNA, 849 bp, Arabidopsis thaliana
  source 1..849: mol_type = other DNA
atggctacaccaaacgaagtatcagctcttttcctcatcaagaagtatctcctcgacgaattgtctccgttgcctactactgccaccaccaatcgatggatgaacgatttcacgtcatttgatcaaaccggtttcgagttttctgaatttgaaaccaaaccggaaataatcgatctcgtcactcccaaaccggagatttttgatttcgatgtgaaatctgaaattccatctgaatcgaacgattccttcacgttccaatcgaatcctcctcgcgttactgttcaatccaatcgaaaaccgccgttgaagatcgcaccaccgaaccgaaccaagtggattcaattcgcaaccggaaatcctaaaccggaacttcccgtaccggttgtagcagcagaggagaagaggcattacagaggagtgaggatgaggccgtgggggaaattcgcggcggagattcgagacccgactcgtcgtggaactcgtgtttggctcgggacgtttgagacggcgatcgaagcggctagagcttacgacaaagaagcgtttagactacgaggatcaaaggcgattctgaatttcccgcttgaagttgacaagtggaatccacgcgctgaagatggtcgtggcctgtacaacaaacggaagagagacggcgaggaggaggaagtgacggtggttgagaaagtgctaaagacggaggagagttacgacgttagcggcggcgagaatgttgagtcaggtttgacggcgatagatgactgggatttgacggagtttctgagcatgccgcttttatcgccgttatctccacacccaccgtttggttatccacaattgaccgttgtttga

SEQ ID NO: 167 - DNA, 579 bp, Arabidopsis thaliana
  source 1..579: mol_type = other DNA
atggtgtctatgctgactaatgttgtctctggtgagaccgaaccctcggcatctgcgacatggacgatgggtcataagagagaaagagaagagttttctttgcctcctcaaccattgattaccggttcagctgtgactaaagaatgtgaaagctcaatgtccttggagaggccaaaaaaatatagaggagtaaggcaacgaccatggggaaaatgggcggcggagattcgagacccacacaaggcgacacgtgtatggcttgggacattcgagacagccgaggccgccgcaagagcctatgatgcggcagcacttcgctttagaggaagcaaagcaaagcttaatttccccgaaaatgttggaactcagacgattcaacgaaattctcatttcttgcaaaactctatgcaaccttctctgacatacatcgatcaatgtccaactctattatcttactctcgatgtatggagcaacaacaaccattagtaggcatgttgcagccaacagaagaggaaaatcactttttcgaaaaaccatggaccgaatatgatcaatacaattactcctcttttggttaa

SEQ ID NO: 168 - DNA, 807 bp, Arabidopsis thaliana
  source 1..807: mol_type = other DNA
atgcattatcctaacaacagaaccgaattcgtcggagctccagccccaacccggtatcaaaaggagcagttgtcaccggagcaagagctttcagttattgtctctgctttgcaacacgtgatctcaggggaaaacgaaacggcgccgtgtcagggtttttccagtgacagcacagtgataagcgcgggaatgcctcggttggattcagacacttgtcaagtctgtaggatcgaaggatgtctcggctgtaactactttttcgcgccaaatcagagaattgaaaagaatcatcaacaagaagaagagattactagtagtagtaacagaagaagagagagctctcccgtggcgaagaaagcggaaggtggcgggaaaatcaggaagaggaagaacaagaagaatggttacagaggagttaggcaaagaccttggggaaaatttgcagctgagatcagagatcctaaaagagccacacgtgtttggcttggtactttcgaaaccgccgaagatgcggctcgagcttatgatcgagccgcgattggattccgtgggccaagggctaaactcaacttcccctttgtggattacacgtcttcagtttcatctcctgttgctgctgatgatataggagcaaaggcaagtgcaagcgccagtgtgagcgccacagattcagttgaagcagagcaatggaacggaggaggaggggattgcaatatggaggagtggatgaatatgatgatgatgatggattttgggaatggagattcttcagattcaggaaatacaattgctgatatgttccagtga

SEQ ID NO: 169 - DNA, 663 bp, Arabidopsis thaliana
  source 1..663: mol_type = other DNA
atgtctgccatggtctcggccttgacacaggtggtttctgctcgctctcagactgaggctgaaggtgctcactcttcttcctcttcggctggacataaaagaggatggcttggaatcgattctgctcctattccctcatcatttgctcgtgtagactcttcacataatccgatcgaagaatccatgagcaaggcatttccagaggaagcaagggagaaaaaaaggaggtacagaggagtaaggcagagaccatggggcaaatgggcagctgagatacgtgatccacatagagccgctagggtttggctcgggacgtttgatacagcggaggccgcggctagagcctacgacgaggctgcactccggttccgtggaaataaagcaaagctaaatttcccagaggatgtaaggattcttcctcctccccctcctcttcttcgttcaccagctgacacggtggcgaataaagcagaagaggatctgataaattattggagttatacaaagttgttgcaaagttcaggccaacggtcatttctcgagcgaggacaagaagagagtagtaacatatttgaacattcaccaatggaacaacctctgcctccttcaagttctggtccaagttcctctaattttcctgcaccttctctacctaatacatag

SEQ ID NO: 170 - DNA, 1176 bp, Arabidopsis thaliana
  source 1..1176: mol_type = other DNA
atgtgtgtcttaaaagtggcaaatcaggaagataacgttggcaaaaaagccgagtctattagagacgatgatcatcggacgttatctgaaatcgatcaatggctttacttattcgcagccgaagacgaccaccaccgtcatagcttccctacgcagcagccgcctccatcgtcgtcgtcctcatctcttatctcaggtttcagtagagagatggagatgtctgctattgtctctgctttgactcacgttgttgctggaaatgttcctcagcatcaacaaggaggcggtgaaggtagcggagaagggacttcgaattcgtcttcttcctcggggcagaaaaggaggagagaggtggaggaaggtggcgccaaagcggttaaggcagctaatactttgacggttgatcaatatttctccggtggtagctctacttctaaagtgagagaagcttcgagtaacatgtcaggtccgggcccaacatacgagtatacaactacggcaactgctagtagcgaaacgtcgtcgtttagtggggaccaacctcggcgaagatacagaggagttagacaaagaccatggggaaagtgggcggctgagattcgagatccatttaaagcagctagagtttggctcggtacgttcgacaatgctgaatcagcagcaagagcttacgacgaagctgcacttcggtttagaggcaacaaagccaaactcaacttccctgaaaacgtcaaactcgttagacctgcttcaaccgaagcacaacctgtgcaccaaaccgctgctcaaagaccgacccagtcaaggaactcgggttcaacgactacccttttgcccataagacctgcttcgaatcaaagcgttcattcgcagccgttgatgcaatcatacaacttgagttactctgaaatggctcgtcaacaacaacagtttcagcaacatcatcaacaatctttggatttatacgatcaaatgtcgtttccgttgcgtttcggtcacactggaggttcaatgatgcaatctacgtcgtcatcatcatctcattctcgtcctctgttttccccggctgctgttcagccgccaccagaatcagctagcgaaaccggttatctccaggatatacaatggccatcagacaagactagtaataactacaataatagtccatcctcctga

SEQ ID NO: 171 - DNA, 696 bp, Arabidopsis thaliana
  source 1..696: mol_type = other DNA
atgcatagcgggaagagacctctatcaccagaatcaatggccggaaatagagaagagaaaaaagagttgtgttgttgctcaactttgtcggaatctgatgtgtctgattttgtctctgaactcactggtcaacccatcccatcatccattgatgatcaatcttcgtcgcttactcttcaagaaaaaagtaactcgaggcaacgaaactacagaggcgtgaggcaaagaccgtggggaaaatgggcggctgagattcgtgacccgaacaaggcagctcgtgtgtggcttgggacgttcgacactgcagaagaagccgccttagcgtatgataaagctgcatttgagtttagaggtcacaaggccaagcttaacttccccgagcatattcgtgtcaaccctactcaactctatccatcgcccgctacttcccatgatcgcattatcgtgacaccacctagtccacctccaccaattgctcctgacatacttcttgatcaatatggccactttcaatctcgaagtagtgattccagtgccaacttgtccatgaatatgctgtcttcttcgtcttcatctttgaatcatcaagggctaagaccaaatttggaggatggtgaaaacgtcatgacgactatatccacagaagatgaccgacggagacaacaacatgcctcacctgatcgaccgatcaaatga

SEQ ID NO: 172 - DNA, 639 bp, Arabidopsis thaliana
  source 1..639: mol_type = other DNA
atggtctccgctctcagccgtgtcatagagaatccgacagacccgccggtcaaacaagagcttgataaatcggatcaacatcaaccagaccaagatcaaccaagaagaagacactatagaggcgtaaggcagagaccatggggtaaatgggcggcagaaatccgcgatccaaagaaagcagcccgtgtctggctcgggactttcgagacggcagaggaagctgctttagcctatgaccgagctgccctcaaattcaaaggcaccaaggctaaactgaacttccctgaacgggtccaaggccctactaccaccacaaccatttctcatgcaccaagaggagttagtgaatccatgaactcacctcctcctcgacctggtccaccttcaactactactacttcgtggccaatgacttataaccaggacatacttcaatacgctcagttgcttacgagtaacaatgaggttgatttatcatactacacgtcgactctcttcagtcaacctttttcaacgccttcttcatcttcttcttcctcccaacagacgcagcaacagcagctacaacaacaacaacagcagcgtgaagaagaagagaagaattatggttacaattattataactacccaagagaataa

SEQ ID NO: 173 - DNA, 747 bp, Arabidopsis thaliana
  source 1..747: mol_type = other DNA
atgtatgggaagaggccttttggaggtgatgaatctgaagaaagggaagaagatgagaacttgttcccggtcttctcggcccgatctcaacacgacatgcgtgttatggtctcggccttgactcaagtaatcggaaaccaacaaagcaaatctcatgataacatcagctctattgatgataactatccttctgtgtataatccacaagaccctaatcaacaagttgcgcctactcatcaagaccaaggggacttgaggaggagacattatagaggtgtaaggcaaaggccatggggaaagtgggcagctgaaatccgagacccaaaaaaggcggcacgtgtgtggctcgggacatttgaaaccgctgaatctgcggccttagcttatgatgaagcagccctaaagttcaaaggaagcaaagcaaaactcaatttcccggagagggttcagcttggaagtaactctacatattactcctccaaccaaattccacaaatggaaccacaaagtataccgaactataatcaatactatcatgatgcgagtagtggtgatatgctaagttttaatttgggcggtgggtatgggagtggtaccggatattcaatgtctcatgataatagtactacgactgctgctacaacttcttcgtcttctggtggctcttctaggcaacaagaagagcaagattatgccagattctggcgctttggggattcttcttcctctcctcattcgggatattaa

SEQ ID NO: 174 - DNA, 1242 bp, Arabidopsis thaliana
  source 1..1242: mol_type = other DNA
atggacaatgtaagaggttcaataatgttgcagccactgccagagatagctgagagtatcgatgatgctatctgccatgaactctccatgtggcctgatgatgctaaagatttgttattgatagtggaggcaatatcaaggggagacttgaagttggtacttgttgcttgtgcaaaagctgtttctgagaataatcttctaatggcacgatggtgtatgggtgagttgcgcggtatggtttcgatttctggtgagccaatccagagattgggagcttatatgttagaagggcttgttgctaggcttgctgcttctggtagttcgatatataagtctctccagtccagagaaccagagagttatgaatttttatcttatgtgtatgttctgcatgaggtttgtccatatttcaagtttggatacatgtcagcgaatggtgcgattgcagaagcaatgaaggatgaagagaggattcacattattgacttccaaattggacaagggagccagtggatagcacttatccaggcttttgcagctaggcctggtggggctccaaatattcgaattaccggagttggtgatggatctgtcttggttacagtcaagaagagactagagaaacttgcaaagaagtttgatgttccattcaggttcaatgcggtttcaaggccaagttgtgaagttgaagtggaaaatcttgatgtccgagatggcgaagcccttggagtgaactttgcttacatgctgcatcatttgccagatgagagtgtaagcatggaaaaccacagggaccggttgctgaggatggtgaagagtctatcacctaaagtagtcactcttgtggaacaagaatgcaacacgaacacttcccctttccttcctaggttccttgagacattaagttattacacggcaatgttcgaatctatcgatgttatgcttccgagaaatcacaaggaaaggatcaatatcgagcagcactgcatggcaagggatgtcgtcaacatcatagcttgtgaaggagccgagaggatcgaaagacacgagcttctcgggaaatggaagtcaaggttttccatggcgggttttgagccataccccttgagctcaatcatttcagccaccattagagccctcttgagagattacagcaacgggtatgcgattgaagaaagagatggtgctctgtaccttggttggatggaccgaatcttggtctcatcttgtgcatggaagtga

SEQ ID NO: 175 - DNA, 1782 bp, Arabidopsis thaliana
  source 1..1782: mol_type = other DNA
atggtggaacaaactgtggttagagaacatatcaaagcgagagtcatgtcattagtgaggtctgcagagccatcctcatataggaacccgaagctttacacgttgaatgagaatggtaataacaatggtgtcagttctgctcaaatctttgatccagataggtcaaagaatccttgtctgactgatgattcttacccaagccaaagttatgagaagtactttcttgattcgccaactgatgagtttgttcaacatcctattggctctggtgcctcggttagctcatttggctctttagactcatttccttatcagtcaagaccagttcttggatgttccatggaatttcagttaccgttggattcaacctctacttcatctacaaggcttttgggagattaccaagcggtttcttacagtccgagcatggatgtagtggaggagtttgatgatgagcaaatgagatcaaagattcaagagcttgaaagagcgcttctcggcgatgaagatgataaaatggttggaatagataacctcatggagattgacagcgaatggtcgtaccaaaacgaaagcgaacagcatcaagactcgcccaaagaatcatcgtctgcagattccaactctcatgtaagtagcaaagaagtggtgtctcaagccactccaaagcaaatcttgatatcttgtgctcgtgcgctatctgaaggaaaattagaagaagctttgtcaatggtaaatgagctgaggcagatagtttctatccaaggagacccttctcagagaatcgcagcttacatggtggaaggtctagctgcaagaatggccgcttcaggaaaattcatctacagagcattgaaatgcaaagagcctccttcggatgagaggcttgcagctatgcaagtcctgtttgaagtctgcccttgtttcaagttcgggtttttagcagctaatggtgcgatacttgaagcaatcaaaggtgaagaagaagttcacataatcgatttcgatataaaccaagggaaccaatacatgacactgatacgaagcattgctgagttgcctggtaaacgacctcgcctgaggttaacaggaattgatgaccctgaatcagtccaacgctccattggagggctaagaatcatcggtctaagactcgagcaactcgcagaggataatggagtatccttcaaattcaaagcaatgccttcaaagacttcgattgtctctccatcaacactcggttgcaaaccaggagaaaccttaatagtgaactttgcattccaacttcaccacatgcctgacgagagtgtcacaacagtaaaccagcgggacgagctacttcacatggtcaaaagcttaaacccaaagcttgtcacggtcgttgaacaagacgtgaacacaaacacttcaccgttctttcccagattcatagaggcttacgaatactactcagcagttttcgagtctctagacatgacacttccaagagaaagccaagagaggatgaatgtagaaagacagtgtctcgctagagacatagtcaacattgttgcttgcgaaggagaagaacggatagagagatacgaggctgcgggaaaatggagagcaaggatgatgatggctggattcaatccaaaaccaatgagtgctaaagtaaccaacaatatacaaaacctgataaagcaacaatattgcaataagtacaagcttaaagaagaaatgggtgagctccatttttgctgggaggagaaaagcttaatcgttgcttcagcttggaggtaa

SEQ ID NO: 176 - DNA, 2453 bp, Arabidopsis thaliana
  source 1..2453: mol_type = other DNA
cacttttattatatctcaatctccttcttcttcctcaagctgattcttgagtatgcaaggaaaacttctcttttgtccccttgtcaacaaaaatctcccattcaacaaaatttcttcagcttctctccttcaaaaggtttttattctccttggtttgaacaagttacactgtagagagtgatttggctaaaagaagattgattcaggagctgtgtagtgtattgctggggtttttccaatgcaaacgtctcagaaacatcacagtgcggctggactgcacatgctgtatcctcaagtctactgttcacctcagtttcaagcgaaagacaacaaaggcttctctgatatcccgtccaaagaaaacttttttaccctcgaatcctccactgcttctggtagtcttccttcgtatgactctccttctgtgagcattacatctggccggagcccgttttctcctcagggctctcagtcttgcatctcggatctccatcattcccctgacaacgtctatggatcacctttgagtggggtgtcatctcttgcctatgatgaagctggagtcaagagtaagattcgggaacttgaggtctcgctgctgagtggtgatacaaaagttgaagaattctcaggttttagccccgctgctgggaagtcgtggaactgggatgaacttttggctttgactccacagttggacttgaaagaggtgctggtggaggctgctcgggcagtcgctgatggggatttcgctacagcgtatggattccttgatgtcttggaacagatggtgtcagtctcaggcagtccgatccaacggctaggcacttacatggcagaagggcttagagcgaggcttgaggggtctgggagcaatatatacaaatccttgaaatgcaatgaaccaacgggaagggaactgatgtcttacatgagtgttctctatgaaatctgcccctactggaaattcgcgtacacaactgcgaatgttgagatcttggaagcaatagctggggaaaccagagtccacattatcgattttcagattgcacagggatcacaatacatgtttttgattcaggagcttgcgaaacgccctggtgggccgccgttgctgcgtgtgacgggtgtggatgattcacagtccacctatgctcgtgggggaggactcagcttggtaggtgagaggcttgcaactttggcgcagtcatgtggtgtcccgtttgagtttcacgatgccatcatgtctgggtgcaaggtgcagcgggaacatctcgggttggaacctggctttgctgttgttgtgaacttcccatatgtattacaccacatgccagacgagagcgtaagtgttgaaaatcacagagacaggctgctgcatctgatcaagagcctctccccaaaactggttactctagtagagcaagaatccaacacaaacacctcgccattcctgtcacggtttgtggaaacactggattactacacagcgatgtttgagtcgatagatgcagcacggccacgggatgataagcagagaatcagcgcagaacaacactgtgtagcaagagacatagtgaacatgatagcatgtgaggagtcagagagagtagagagacacgaggtactggggatatggagggtcagaatgatgatggctgggttcacgggttggccggtcagcacatctgcagcgtttgcagcgagtgagatgctgaaagcttatgacaaaaactacaaactgggaggccatgaaggagcgctctacctcttctggaagagacgacccatggctacatgttccgtgtggaagccaaacccaaactagattgggtaagttatagtgatgatggttacttgagtggataaagaagagcacaacaaaaacacatctgtcgctgtaaattttttaggatgtgcaatgatgttttaagttgtaacacaacctaagttatatatgtatacaaaccaaacctggtggttgtttttctcttgtaaattgtcatgtggttgtgggtgggttgctagtaatgaaatataaccaaaacattgattaggtcattttatgatcttcttctatatataccggtataagtcaactggcggctgaacaaaggtcgtgaggtaacaaaatatgagacaaatctacaggtcagattgggttctgaattctgataaggtcttaaaaaggagctcaccaacccacaaaaccatggattgaacaagtacaggtcattgccttcattttattctttacttttctaaggctcaagcttcctttattgcctttaataacaatatactaatgagtattttgcactcagtaacaaaattcaggagagtaattttttgccctaacatgttacttttatgtgttaagagtttagaattttggatctatgattttagtttttgttagggaatcatattcatataaataaaatattgccatt

SEQ ID NO: 177 - DNA, 1581 bp, Arabidopsis thaliana
  source 1..1581: mol_type = other DNA
atggaagctactcagaaacatatgattcaagaaggatcttcaatgttttaccatcaaccatcttcagtcaaacaaatggacctttctgttcagacatttgattcttactgcacacttgaatcctcttcaggcaccaagtctcatccttgtcttaacaacaaaaacaactcttcttcaaccacaagcttttcctctaatgaaagtcccatttctcaagccaacaacaacaacttatcaaggtttaacaatcattcccctgaagaaaacaataactctcccttgagcggttcttcagcgacaaacaccaacgagacagagcttagtctcatgcttaaagatttggagactgcaatgatggagcctgatgtagacaacagctataacaatcaaggcgggtttggacagcagcatggagttgtttcttcagctatgtatagatctatggagatgatctctaggggagatttaaaaggagtgctctatgaatgcgcaaaagctgttgaaaactatgatttggaaatgactgattggttgatctctcagttacagcaaatggtttctgtttctggtgagcctgttcagcgcttaggagcttatatgcttgaaggtctcgttgctaggttagcttcttcgggtagctccatctataaagcattgagatgcaaagacccaacgggtcctgagcttcttacttatatgcatatcttgtatgaagcctgcccttatttcaaattcggttatgaatctgctaatggagctatagctgaagctgtgaagaacgaaagttttgtgcacattatcgatttccagatttctcaaggtggtcaatgggtgagtttgatccgtgctcttggtgctagacctggtggacctccgaacgttaggataacgggaattgatgatccgagatcatcgtttgctcgtcaaggaggacttgagttagttggacaaagacttgggaagctagctgaaatgtgcggtgttccgtttgagttccatggagctgctttatgctgcacggaagtcgaaatcgagaagctaggagttagaaatggagaagcgctcgcggttaacttcccgcttgttcttcaccacatgcctgatgagagtgtaactgtggagaatcacagagatagattgttgagattggtcaaacacttgtcaccaaacgttgtgactctggttgagcaagaagcgaatacaaacactgcgccgtttcttccccggtttgtcgagacaatgaaccattacttggcagttttcgaatcaatagatgtgaaactcgctagagatcacaaggaaaggatcaatgttgagcagcattgtttggctagagaggttgtgaatcttatagcttgtgaaggtgttgaaagagaagagaggcacgagccactagggaaatggaggtctcggtttcacatggcgggatttaaaccgtatcctttgagctcgtatgtgaacgcaacaatcaaaggattgcttgagagttattcagagaagtatacacttgaagaaagagatggagcattgtatttaggatggaagaatcaacctcttatcacttcttgtgcttggaggtaa

SEQ ID NO: 178 - DNA, 1266 bp, Zea mays
  source 1..1266: mol_type = other DNA
atggaaagggtgaagtactgcgattgcaccgtgtgctctgttcagagaagcctgtgttctactagaagaagaaggcgcaggcggaggcagatcgataggcagcttactaaggttgacccaagacgcaggcatggcaagaggccacttccagctgctgaggttgaggaggaagaagaggaagaggctttgccaccaggtccaccaccagctaagcacgagcaacttgaggaaccacaccacgctgctgtgtctcaacttcagggcgctactttttctggcggcggtggatcatctagcagctctgttattggcggcccatctccacctcaagcctacgctcagtactactactctgccagggctgacaacgacgcttctgcagttgcttctgctctggctcatgtgatcagggcttctccagatcagcttccaccacaacaagctccagcactttacggcgctggtgttccaggttctcttaggcttggcgatcatccacaggctagcgctcatcattatccaggcccaggtggtcatgtggctgctgctgaagaggagcaaggtagaaggcggcattacaggggtgttaggcaaagaccatggggcaagtgggcagctgagatcagggatccaaagaaggctgctagggtttggctgggcaccttcgatacagctgaggatgctgctatcgcctacgatgaggctgctcttaggttcaagggcaccaaggccaagctgaacttcccagaaagagtgcagggcaggaccgatctgggcttccttgttactaggggcatcccagatcacaggcacccaagtgctgctgttactctggctgctatgccgccaccacatcatcagcatggccatcagactgtggtgccatacccagaccttatgcagtacgctcaactgcttcaaggcggtagaggtggtgggggtcatgctgaagcagctgttcaacaggctcatcggcagcagcaacagcaacaactgatgactatgatgggcggcaggccaggcgttaacctgccatctacattcagcccaagctctagcgcctctgctccacagatcctggatttctctacccagcagcttatcagacctggcccaccttctccatcaccaccaagagctgccgctatgccatcttcatctgcagccgctgctccaagcaccccatcttctacaactaccgctagctctccatctggtggcgcttggccatacggtggtgaaaggcataggaacaagaaggacgcc

SEQ ID NO: 179 - DNA, 1332 bp, Zea mays
  source 1..1332: mol_type = other DNA
atgcgcatctctctccgcgtacttatcagtagcgagctcggcacgtcattgtgcactgctgcaccgagcctagcgcgagcctccgtccgaaagtccaaatcttcagctctctccttgacgcatgcccggatcgatcggtccagctctacccaccggaggaggaggcagatcaacgggcagctaactaaggtggatccaaggaggaggcatggcaagaggcccctacccgccgacgaggaggaagaagaggaggaggaactgccccctccgcctgcaaagtacgagcagctggatcaggaggagaagcatcacgttgtcgtctcgcagctgcaagcaggagctacctttagcggtggccgagggtcttcgtcgtcttcagtggcaggtccttcgcctgaggcgtacgcgcaatactactactcggcacgtgctgaccacgacgcttccgccgtggcctccgcgttagctcatgtaatacgcgcttctcccgaccaacttccacctcagcaggccgcatgcttgtacggtgctgccggcgctccagtattaaggcagggagagggagatcatcctcaacctcaagcggctgcgcaccatcatccaggtggccacgtcgccgctgaggaagagcaaggtctaagaaggcactacagaggggtgaggcagaggccgtggggtaaatgggcggctgagatacgggaccctaaaaaggcggcgcgggtgtggctaggcacattcgacacggctgaggatgctgcaattgcctatgacgaggcggcgctgaggttcaagggcaccaaggccaagctcaacttccctgagcgcgttcagggccgaaccgacctcggcttcgtcgtcacacgaggaattccggaccatcatcgtcatccacgggcggcggcagttaacctggcagcaatgccgcaggctcaggctcagccgcacttgcaacacggacgaccaaccgtcatgccgtatccgtacccataccctgacctaatgcagtacgcgcagctgcttcagggcggccggggaggtggtgaccatgcggctgcagttcagcaacagctcatgatgatgggaggacgaggaggcaacctgccattctcgttctcgccgccatcgtcctggagtgcgcctccgcagatactggacttctcggcgaggcaactaattacccaacccggaccaccttcttctccggctgctccaggtggcgcggctccatccacaccgtcatctactactactgcgtcatcgccaagcgccagcgcaagcggaagtgcatggccatacggtggggagcaccacaggaataaaaaggacgcgtga

SEQ ID NO: 180 - DNA, 1074 bp, Zea mays
  source 1..1074: mol_type = other DNA
atgtgcttcgagctggctgatcagcgaggaccgcagggaggcggaggagcgggatggcctgctaaaaggagagctggaggcgttcaagacgaaggtgctgccgcggctgcaggaatggcaatggctgctgctggccccggggaggtgatgtccgagtactaccaggcgcaggagctgtcgacgatggtgtctgctctgactcacgtcgttgctggcgctccgatgggaagcgcccctgctcagaggccaatgcacggcgcatcaggctactacgcgcacgagatgggaagttaccgcggtgcaccatcgcctgagctcgcaggttcggagctgagttctgatacccagagtgcgggtgcggctgccatggaggagcaccaatcggcagctgcgctttccagtcaagaaggtcccgagactccgaggagacgctaccgtggggtgcgccaaaggccgtggggcaagtgggccgctgaaatccgtgatcctcacaaggctgcgagagtatggctcggcacgttcgagactgctgaggccgcggcgcgcgcatatgacgaggctgcgctacgcttccgaggcagtcgtgccaaacttaacttccccgaggacgcgcgcttatatccggcgtcaacggctggcgcggctgcacctctcgctgctgctgcttcgacttcaccacctgtctactccggtggagtccagggctcgtcggactacctgaggtaccaccagatgcttctgcaagcgtccacgggcagccagggtaccctgctcccattctatggcggcggcatgagtaatccatacggcggcggtgctgccatgaccggttcatacggtggagccggcggcggcaacacgagtggttccttgggctcctactactcgttcccggcctcctctgtttctgtcgccaccgtgccgtcctctacttcctctgcttcggggtactactactcgtctccgcacgactcgcaacatagcgaggcgtctgcggctgctgactggaattgggagagcgcgctggcatggcctgactcgagccagtacccgccaccacctcacactcagtag

SEQ ID NO: 181 - DNA, 1629 bp, Zea mays
  source 1..1629: mol_type = other DNA
atgtcagctagggattctaatcattcatataaatgttcggatgattctcaaatgccgtactacaataactcagcacctgtaggagaaaatggtaggatccatatggcagaaaacagtctaggacatcattactcatcctccgatattggatcacaaaggatcaacaattccaaccctcaagtatttgaagcacagtactgcactctggaatcatcctcagcaaatggtgtttatcctgcacaaagctccacatcttctcatagcatctcccctttaagtgggagccccctgtctcagcatgacagccactcagaccacacatatagttctcctccaagtgcttcctgtctaactgaggttgcagatctcctgattaaacaaaaagaattagagaattccattgttggacctggactggacattagttctgactgctctcggagcttgttgcaagcccatgttccagtcagaccagacaactggagacaacttctgggaattaatggaggagacttgatgcaagtagtcatagcatgtggtaaggctgttgcagagaatgatgtctttgcaacagaactgttaatatctgagcttggtcacctggtatctgtatctggagatccaatgcaacgacttggagcctatatgcttgaaggaattgttgctagactttcttcctccggcagtatgctatataaatctttgaaatgtaaagaacctacaagctctgagctcatgtcatatatgcatctcctttacgagatctgcccattctacaagtttggttacatgtcagcaaatggtgccattgccgaggctattaagggcgagaactttgttcacataattgatttccaaattgctcaggggagccagtgggttactctgctacaggcccttgctgcaagacctggggggccaccatacattagaatcactggtatagatgactcaaattctgcttatgccagaggaggcgggcttgatatagttgggaggacgttgtgtgatgttgctaattcatgtggtcttccttttgagttcaatgccgttccagctgctagccatgaggttgagcttcagcatcttgctataagacatggggagattattgctgtgaattttgcctatcagctacatcatgttcctgacgaaagtgtaagtacagaaaatcatcgggataggattataagaatgatcaagagcatcaatcctagggttgttactctcgttgaacaggagtcaaatacaaacacagctccattcttcccaaggtacatggaaactctcaactactatacagccatgtttgagtcgatagatgttgctctcccaagggatgataggaggcggatgagcgcagaacaacactgtgttgcaagggatattgttaatttaatcgcatgcgagggtgctgaaagggttgagaggcacgagctgtttggaaaatggaagtcaagatttgcaatggctggatttagaccgtacccactgagttcagttgtgaacaacactatcaacacattgttgcatacctacaacagctactacaggcttgaggagagagatggtgtcctttaccttggatggaaaaacagagtattggttgtatcttcggcatggtgttga

SEQ ID NO: 182 - DNA, 1665 bp, Zea mays
  source 1..1665: mol_type = other DNA
atggctgacactccaacgtcccgcatggtgcacccctttggcgacgccccaaggcagactcccaagcaattcctgtattctggtaacccgcagcacctctgccatccgtaccagtcagctccggacacccatgtcgtgctccagcgccgttacaccgtgaggtcacagtctcactcaccaaataatgctggttctgaagaccatgagactcacaagcagtacacgctggagtcctcagcggcttctggttgttcaaggcatggttctccctccagccaaagcgtccatgccgggagtggcagccctgtgtctcacgacgacagccactccggctccacgaatggccatggatcacctgtgagcgcgtcgtgtgtcaccggcgaggaccctactgatctcaagcagaaactgaaggatcttgaggctgtaatgctggggacgtctgagactgaccccgagatagtcaacagtctcgagatctccgcggcaaaccaactctcgttggagcccgaggagtgggagcacatggtgagcatgcccagagggaacctgaaggagctgctgattgcctgcgctagggcagtggaacgtaacaacagctacgccattgatctgatgatcacagagctgaggaagatggtttcggtgtccggcgagccgcttgagaggctgggagcctacatggtggaaggtcttgttgccaggctcgcggcctccggcagctcaatctacaaggctttgaagtgtaaggagcccaggagctctgatctcctctcctacatgcacttcctgtatgaggcctgcccctacttcaagttcggctacatgtcagccaacggcgcgatcgcggaggccatcaagggagaggacaggatccatatcatcgacttccacatcgcgcaaggggctcagtgggtgtctctcctccaagcccttgcagccaggcctggcgggccaccgttcgtgagggtcaccggcattgatgattcagtctcagcttacgctcgaggcggcggtctggagttggttggcaggaggttgacacatattgctggcctctacaaggtgccctttcagttcgacgcagtcgctatctcaggcagtgaggtggaggaggagcatctgggcgtcgtcccaggcgaagccgtcgccgtcaacttcacccttgagctgcaccacatccccgacgagacggtgagcacggcgaaccaccgggaccggatcctgaggctggtgaagggcctgtcgcccaaggtgctcacgctggtggagcaggagtccaacaccaacacggcccccttcgcgcagcggttcgcagagacgctggactactacacggccatcttcgagtccatcgacctggcgctgccgagggacgacagggagcggatcaacatcgagcagcactgcctggcccgggagatcgtgaacctggtggcctgcgagggcgaggagcgggtggagcggcacgaggtgttcggcaagtggaaggcgcggctgatgatggccgggttcagcccgtccccgctcagcgcgctggtgaacgccaccatcaagacgctgctgcagagctactcgccggactacaagctcgccgagagggacggcgtgctctacctcggctggaagaacaggcccctgatcgtctcatcggcatggcactag

SEQ ID NO: 183 - DNA, 1677 bp, Zea mays
  source 1..1677: mol_type = other DNA
atggccgaccctccaacttcccgcatggtgcagcaccccttcggcgacatcccaagccagactcccaagcaattcctatactctggtagctcacagcacctctgccatccataccagtcagcttcggacgcccacgtcgcgccgcagcgtcactacaccgtgaggtcccagtctcagtctcagtcaccggatgctgggtctgaagactttgagacgcacagcaggcagtacacgctggactcctcttcggcttccggttgctcagggcacggttctccctcctgtcagagtgtccatgccgggagtcgcagccctgtgtctcactctcatgacgacagccactccggctccaccaatggcaacggatcgcctgcgagcgcgtcgtgtgtcaccgaggaccctactgacctcaagcagaaactgaaggatcttgaggctgtgatgctggggacggacacggacccagaaaccgtggacagtctcgagatcgccatagcggaccggctttcggtggagcccgaggagtggaagaacaacatggtgagcgtgcctagaggggacctgaaggagctgctgatcgcctgcgccagggcagtggagcaaaacaacggctactccatcgacctgatggtcccagagctgaggaagatggtgtcggtgtccggcgagccgctcgagaggctgggagcctacatggtggaaggactcgtcgccaggctcgccgcctctggcagctccatctacaaggctctgaggtgtaaggagcccaggagctccgacctcctctcctacatgcacttcctgtacgaggcctgcccctacttcaagttcggctacatgtcggccaacggcgcgatcgcggaggccgtcaagggagaagacaggatccatatcatcgacttccacatcgctcagggggcgcagtgggtgtctctcctccaagcccttgcggcccggcctggcgggccaccgttcgtgagggtcaccggcattgatgatcccgtttcagcttacgcccgaggcggcggcttagagttggtcggcaagaggctgtcacacatcgctggcctctacaaggtgccctttcagttcgacgcggtcgccatctccggcagcgaggtggaggaggggcatctgggcgtcgtccctggcgaagccgtcgccgtcaactttactctagagctgcaccatatccccgacgaaaccgtgagcacggcgaaccacagggaccgggtcctgaggctggtcaagggcctgtcgcccagggtgctcacgctggtggagcaggagtccaacaccaacacggcccccttcgcgcagcggttcgcggagacgctggactactacgcggccatcttcgagtccatcgacctggcgctgcccaggggcgacagggagcggatcaacatcgagcagcactgcctggctcgggagatcgtcaacctggtggcctgcgagggcgaggagcgggtggagcggcacgaggtgttcggcaagtggaaggcgcgcctcatgatggccgggttcaggccgtccccgctcagcgcgctggtcaacgccaccatcaagacgctgctgcagagctactcgccggactacaagctcgccgagagggaaggcgtgctctacctcggctggaagaacaggcccctcattgtttcatcggcatggcactag

SEQ ID NO: 184 - DNA, 23 bp, synthetic construct
  source 1..23: mol_type = other DNA
atggcgaattcaggaaattatgg

SEQ ID NO: 185 - DNA, 21 bp, synthetic construct
  source 1..21: mol_type = other DNA
tcacttcctcaatagcccttg

SEQ ID NO: 186 - DNA, 23 bp, synthetic construct
  source 1..23: mol_type = other DNA
atgaggcactatagaggggtaag

SEQ ID NO: 187 - DNA, 22 bp, synthetic construct
  source 1..22: mol_type = other DNA
tcaagctctctcagggaaattg

SEQ ID NO: 188 - DNA, 23 bp, synthetic construct
  source 1..23: mol_type = other DNA
atgcaactagcaagtaacactag

SEQ ID NO: 189 - DNA, 23 bp, synthetic construct
  source 1..23: mol_type = other DNA
tcaaaaaccagaattaggaggtg

SEQ ID NO: 190 - DNA, 55 bp, synthetic construct
  source 1..55: mol_type = other DNA
atggtctcagccttgactcaagtcattggaaacatgtacggacagtgcaatatag

SEQ ID NO: 191 - DNA, 31 bp, synthetic construct
  source 1..31: mol_type = other DNA
ggggacaagtttgtacaaaaaagcaggcttg

SEQ ID NO: 192 - DNA, 56 bp, synthetic construct
  source 1..56: mol_type = other DNA
atggtctcagccttgactcaagtcattggaaacatggctacaccaaacgaagtatc

SEQ ID NO: 193 - DNA, 31 bp, synthetic construct; identical to SEQ ID NO: 191
  source 1..31: mol_type = other DNA

SEQ ID NO: 194 - DNA, 24 bp, synthetic construct
  source 1..24: mol_type = other DNA
tcttatatctagtgtattttcacc

SEQ ID NO: 195 - DNA, 25 bp, synthetic construct
  source 1..25: mol_type = other DNA
ctttgggaataggtttttttttttc

SEQ ID NO: 196 - DNA, 23 bp, synthetic construct
  source 1..23: mol_type = other DNA
gaccaatagatgatatacaagtg

SEQ ID NO: 197 - DNA, 23 bp, synthetic construct
  source 1..23: mol_type = other DNA
ttctagtcacttcaatgatctgc

SEQ ID NO: 198 - DNA, 25 bp, synthetic construct
  source 1..25: mol_type = other DNA
atgtacaagcagcctagacaagagc

SEQ ID NO: 199 - DNA, 25 bp, synthetic construct
  source 1..25: mol_type = other DNA
tcatttccaagcacacgaggcaacc

SEQ ID NO: 200 - DNA, 27 bp, synthetic construct
  source 1..27: mol_type = other DNA
tttccaagcacacgaggcaaccaaatc

SEQ ID NO: 201 - DNA, 20 bp, synthetic construct
  source 1..20: mol_type = other DNA
cctgagttgcagagtggaga

SEQ ID NO: 202 - DNA, 30 bp, synthetic construct
  source 1..30: mol_type = other DNA
tcttttatatgactacttatgatacaattc

SEQ ID NO: 203 - DNA, 24 bp, synthetic construct
  source 1..24: mol_type = other DNA
atggttaagttcacaactttcctc

SEQ ID NO: 204 - DNA, 22 bp, synthetic construct
  source 1..22: mol_type = other DNA
gggattgtggttttgagtgtag

SEQ ID NO: 205 - DNA, 18 bp, synthetic construct
  source 1..18: mol_type = other DNA
taggtcacgattcgtgcc

SEQ ID NO: 206 - DNA, 25 bp, synthetic construct
  source 1..25: mol_type = other DNA
cacaaataagcaagtaagaatcttt

SEQ ID NO: 207 - DNA, 25 bp, synthetic construct
  source 1..25: mol_type = other DNA
atggacaatgtaagaggttcaataa

SEQ ID NO: 208 - DNA, 22 bp, synthetic construct
  source 1..22: mol_type = other DNA
tcacttccatgcacaagatgag

SEQ ID NO: 209 - DNA, 21 bp, synthetic construct
  source 1..21: mol_type = other DNA
cttccatgcacaagatgagac

SEQ ID NO: 210 - DNA, 24 bp, synthetic construct
  source 1..24: mol_type = other DNA
agtagttagagccaccatacccaa

SEQ ID NO: 211 - DNA, 24 bp, synthetic construct
  source 1..24: mol_type = other DNA
gttcagatgtaaagtcctcaactg

SEQ ID NO: 212 - DNA, 21 bp, synthetic construct
  source 1..21: mol_type = other DNA
ggctcctcttaacccaaaggc

SEQ ID NO: 213 - DNA, 22 bp, synthetic construct
  source 1..22: mol_type = other DNA
cacaccatcaccagaatccagc

SEQ ID NO: 214 - DNA, 22 bp, synthetic construct
  source 1..22: mol_type = other DNA
accaccattaacgtgcgtcaac

SEQ ID NO: 215 - DNA, 24 bp, synthetic construct
  source 1..24: mol_type = other DNA
gatcttggcgagagaatcggtatc

SEQ ID NO: 216 - DNA, 23 bp, synthetic construct
  source 1..23: mol_type = other DNA
ctctcgttccagagctcgcaaaa

SEQ ID NO: 217 - DNA, 23 bp, synthetic construct
  source 1..23: mol_type = other DNA
aagaacacgcatcctacgcatcc

SEQ ID NO: 218 - DNA, 20 bp, synthetic construct
  source 1..20: mol_type = other DNA
tttgcctttgtgcttcatca

SEQ ID NO: 219 - DNA, 20 bp, synthetic construct
  source 1..20: mol_type = other DNA
catcgcggcatagtagttca

SEQ ID NO: 220 - DNA, 20 bp, synthetic construct
  source 1..20: mol_type = other DNA
acgaaaaccaatccaaccac

SEQ ID NO: 221 - DNA, 20 bp, synthetic construct
  source 1..20: mol_type = other DNA
attggacgctaggcatcaag

SEQ ID NO: 222 - DNA, 20 bp, synthetic construct
  source 1..20: mol_type = other DNA
gaggttccaaaagtggctga

SEQ ID NO: 223 - DNA, 20 bp, synthetic construct
  source 1..20: mol_type = other DNA
cgttggtttgatgaatgtcg

SEQ ID NO: 224 - DNA, 20 bp, synthetic construct
  source 1..20: mol_type = other DNA
ggacgggaagatatgaagca

SEQ ID NO: 225 - DNA, 20 bp, synthetic construct
  source 1..20: mol_type = other DNA
agggaaattggtggtagcag

SEQ ID NO: 226 - DNA, 20 bp, synthetic construct
  source 1..20: mol_type = other DNA
caggcacgaattggaagagt

SEQ ID NO: 227 - DNA, 20 bp, synthetic construct
  source 1..20: mol_type = other DNA
aggcattagtccacccacag

SEQ ID NO: 228 - DNA, 24 bp, synthetic construct
  source 1..24: mol_type = other DNA
ggatatgacaaggaagatagagca

SEQ ID NO: 229 - DNA, 22 bp, synthetic construct
  source 1..22: mol_type = other DNA
tgcaataaactcttgcttggtc

SEQ ID NO: 230 - DNA, 18 bp, synthetic construct
  source 1..18: mol_type = other DNA
gcatcagctcggctcaat

SEQ ID NO: 231 - DNA, 21 bp, synthetic construct
  source 1..21: mol_type = other DNA
gcattcttcttctccaacacc

SEQ ID NO: 232 - DNA, 20 bp, synthetic construct
  source 1..20: mol_type = other DNA
cccggcttaacttccctaac

SEQ ID NO: 233 - DNA, 20 bp, synthetic construct
  source 1..20: mol_type = other DNA
tagatctggcgacgaaacct

SEQ ID NO: 234 - DNA, 20 bp, synthetic construct
  source 1..20: mol_type = other DNA
ggctagggagaggcagaaac

SEQ ID NO: 235 - DNA, 20 bp, synthetic construct
  source 1..20: mol_type = other DNA
tccaccttggagttggagtc

SEQ ID NO: 236 - DNA, 22 bp, synthetic construct
  source 1..22: mol_type = other DNA
aaagacgcgtgatcgagagctg

SEQ ID NO: 237 - DNA, 24 bp, synthetic construct
  source 1..24: mol_type = other DNA
acgtgttgctcaatcttgtctcac

SEQ ID NO: 238 - DNA, 22 bp, synthetic construct
  source 1..22: mol_type = other DNA
aagtagctgtgcggaggaatgg

SEQ ID NO: 239 - DNA, 22 bp, synthetic construct
  source 1..22: mol_type = other DNA
gggtaagtggcagcgacgattt

SEQ ID NO: 240 - DNA, 23 bp, synthetic construct
  source 1..23: mol_type = other DNA
tgatgacacccgagtggtctttg

SEQ ID NO: 241 - DNA, 23 bp, synthetic construct
  source 1..23: mol_type = other DNA
acaccgatggagtcttggagaac

SEQ ID NO: 242 - DNA, 23 bp, synthetic construct
  source 1..23: mol_type = other DNA
accttaccagcccttgacatatg

SEQ ID NO: 243 - DNA, 23 bp, synthetic construct
  source 1..23: mol_type = other DNA
gacttgaccaaacatctcacgac

SEQ ID NO: 244 - DNA, 21 bp, synthetic construct
  source 1..21: mol_type = other DNA
agtccgttgagatgcaccatg

SEQ ID NO: 245 - DNA, 22 bp, synthetic construct
  source 1..22: mol_type = other DNA
cacatacccacgcttcagatcc

(19) United States
(12) Patent Application Publication
(10) Pub. No.: US 2024/0294931 A1
(43) Pub. Date: Sep. 5, 2024

(54) Means and Methods for Improved Regeneration

(71) Applicants: VIB VZW, Gent (BE); Universiteit Gent, Gent (BE)

(72) Inventors: Lieven De Veylder, Drongen (BE); Jefri Heyman, Roosdaal (BE)

(21) Appl. No.: 18/600,428

(22) Filed: Mar. 8, 2024

Related U.S. Application Data
(63) Continuation of application No. 16/340,949, filed on Apr. 10, 2019, now Pat. No. 11,939,586.

(30) Foreign Application Priority Data
Oct. 11, 2016 (EP) 16193308.0

(51) Int. Cl. C12N 15/82; C07K 14/415; C12N 9/00; C12N 15/113

(52) U.S. Cl. CPC C12N 15/8241; C07K 14/415; C12N 9/00; C12N 15/113; C12N 2800/24; C12N 2800/30; C12N 2820/65; C12N 2830/50; C12N 2830/65

(57) ABSTRACT

The present invention relates to the field of plant genetic engineering, particularly the modulation of gene expression in plant cells for improved regeneration competence. The invention discloses means and methods wherein the Ethylene Response Factor (ERF) and Phytochrome A Signal Transduction 1 (PAT1) transcription factor complex activity in plant cells grants increased regeneration.

### CROSS REFERENCE TO RELATED APPLICATIONS

This application is a continuation of U.S. patent application Ser. No. 16/340,949, filed Apr. 10, 2019, pending, which is a national phase entry under 35 U.S.C. § 371 of International Patent Application PCTEP2017075937, filed Oct. 11, 2017, designating the United States of America and published in English as International Patent Publication WO WO2018/069392 on Apr. 19, 2018, which claims the benefit under Article 8 of the Patent Cooperation Treaty to European Patent Application Serial No. 16193308.0, filed Oct. 11, 2016, the entireties of which are hereby incorporated by reference.

### INCORPORATION BY REFERENCE

The ST.26 XML Sequence listing “10369 US 2024-05-20-ST26 Replacement Sequence Listing”, created on May 20, 2024, and having a size of 357,383 bytes, is hereby incorporated herein by this reference in its entirety.

### FIELD OF THE INVENTION

The present invention relates to the field of plant genetic engineering, particularly the modulation of gene expression in plant cells for improved regeneration competence. The invention discloses means and methods wherein the Ethylene Response Factor (ERF) and Phytochrome A Signal Transduction 1 (PAT1) transcription factor complex activity in plant cells grants increased regeneration.

### BACKGROUND

Most plant cells are totipotent, which requires the acquisition of stem cell potential (dedifferentiation) in response to proper stimuli and expressing this potential during subsequent morphogenesis (regeneration).

During dedifferentiation, genome reprogramming occurs to establish a stem cell status, and subsequently, gene activity patterns change in a systematic manner while entering the regeneration phase^{36,37}. Regeneration of a tissue damaged by injury represents a physiological response for organ recovery¹⁻³. Although this regeneration process is conserved across multicellular taxa, plants appear to display extremely high regenerative capacities, a feature widely used in tissue culture for clonal propagation and grafting^{4,5}. Regenerated cells arise predominantly from pre-existing populations of division-competent cells^{6,7}, however, the mechanisms by which these cells are triggered to divide in response to injury remain largely elusive⁸. In Arabidopsis thaliana, dividing root cells are mostly confined to specialized regions called meristems, of which the centre contains stem cells capable of self-renewal and differentiation into specific cell types. The stem cells' undifferentiated status is controlled by the quiescent centre (QC), containing a group of mitotically inactive cells, which are characterized by the expression of WUSCHEL RELATED HOMEOBOX5 (WOX5)¹⁰⁻¹². Under DNA damage-inducing conditions, most stem cells are selectively killed¹³. Although this mechanism efficiently prevents deleterious mutations to be propagated through the organism, the dead stem cells need to be replaced to maintain a functional meristem and growth. Previously, the ETHYLENE RESPONSE FACTOR 115 (ERF115), a member of the ethylene response transcription factor family, has been found to be a rate-limiting factor for QC cell division and essential for the re-establishment of a functional stem cell niche (SCN) under DNA stress conditions^{14,15}.

Members of the APETALA2 (AP2) family of proteins play critical roles in a variety of important biological events including development, plant regeneration, cell division, etc. Accordingly, it is valuable to the field of agronomic development to identify and characterize novel AP2 family members and develop novel methods to modulate embryogenesis, transformation efficiencies, oil content, starch content and yield in a plant. The AP2/ERF transcription factor called EBE, or ERF114, is the closest homologue of ERF115, and is prominently expressed in proliferating cells and calli. Ectopic overexpression of ERF114 for cotyledons precultured on phytohormone-containing medium resulted in larger calli as a result from more pronounced cell proliferation leading to an increased cell number³⁸.

Furthermore, another member of the AP2 family that has been implicated in a variety of critical plant cellular functions is the Baby Boom protein (BBM). The BBM protein from Arabidopsis is preferentially expressed in seed and has been shown to play a central role in regulating embryo-specific pathways. Overexpression of BBM has been shown to induce spontaneous formation of somatic embryos and cotyledon-like structures on seedlings³⁹.

Current transformation technology provides an opportunity to engineer plants with desired traits. Major advances in plant transformation have occurred over the last few years. However, in many major crop plants, serious genotype limitations still exist. Transformation of some agronomically important crop plants continues to be both difficult and time consuming. Optimization of medium components and/or explant material and source has led to success in some genotypes, mostly model genotypes, whereas the process of introgressing transgenes into production inbreds is still laborious, expensive and time consuming. Accordingly, methods are needed in the art to increase transformation efficiencies of plants. Especially the regeneration step during plant transformation is often long and requires several hormone treatments to obtain transgenic plants.

It would be advantageous to find alternatives or generic solutions for improved regeneration capacities during transformation and regeneration of plants, in particular with reduced use of hormones. Further, it is often necessary to reduce the activity of a transgene because the transgene may negatively affect the growth or fertility of the plant. So in combination with generic regeneration capacity improvements, the further optimization of transient expression or recombination systems used to excise or limit the presence of the transgene are needed in the art.

### SUMMARY OF THE INVENTION

The invention relates to the finding that the AP2 transcription factor Ethylene Response Factor 115 (ERF115) in complex with the Phytochrome A Signal Transduction 1 (PAT1) transcription factor revealed a function in sustaining the root meristem by promoting cell renewal after stem cell loss. Unexpectedly, ectopic expression of the ERF115-PAT1 complex lead to spontaneous callus formation thereby granting regeneration competence, even in the absence of phytohormone induction media. Means and methods to improve regeneration of plants include the ectopic expression of said ERF115-PAT1 complex as a suitable alternative for increasing plant transformation efficiency.

The first aspect of the invention relates to a combination of a first and second chimeric gene construct comprising the operably linked DNA elements being a) a promoter suitable for plant expression, b) a DNA region encoding an ERF with the SCL/PAT1 interaction motif, and a PAT1 transcription factor, for the first and second construct respectively, and c) a 3′ end region to terminate transcription and provide polyadenylation signals to obtain functional expression of said chimeric constructs in plant tissue. Alternatively, an aspect of the invention relates to a chimeric gene construct comprising the operably linked DNA elements being a) a promoter suitable for plant expression, b) a multicistronic DNA region encoding an ERF transcription factor with the SCL/PAT1 interaction motif, coupled to a DNA region encoding PAT1 and c) a 3′ end region comprising transcription termination and polyadenylation signals functioning in cells of a plant.

In one embodiment, said chimeric gene construct of the present invention comprises a b) DNA region that encodes an ERF transcription factor with SCL/PAT1 interaction motif that is ERF115. A more specific embodiment relates to said chimeric gene constructs, wherein said b) DNA region is selected from the list of SEQ ID NO: 51-100 encoding an ERF115 transcription factor protein corresponding with a protein sequence selected from the list of SEQ ID NO: 1 to 50, which is a list consisting of a number of ERF115 orthologue sequences from different plant species; and wherein said b) DNA region is selected from the list of SEQ ID NO: 126-150 encoding a PAT1 transcription factor protein that is a protein with corresponding sequence selected from the list of SEQ ID NO: 101-125, a list consisting of a number of PAT1 orthologue sequences from different plant species.

Another embodiment describes said chimeric constructs, further comprising a chimeric gene construct comprising the operably linked DNA elements being a) a plant expressible promoter, b) a DNA region encoding a site-specific recombinase that is capable of recognizing and implementing recombination at said recombination sites with the goal to remove the chimeric genes from the insertion sites, and c) a 3′ end region comprising transcription termination and polyadenylation signals functioning in cells of a plant, and wherein said chimeric constructs begin and end by flanking recombination sites to allow excision of said chimeric genes.

Another embodiment relates to a set of at least one recombinant vector comprising said chimeric gene constructs. In further embodiments, a plant, plant cell or plant seed comprising said chimeric gene construct(s) or comprising said recombinant vector(s) are described. And in a particular embodiment, said plant, plant cell or plant seed co-express ERF and PAT1 transcription factors. In one embodiment, said plant is a member of the Gramineae.

In another aspect, the invention relates to the use of said chimeric gene construct(s) of the present invention, or said recombinant vector(s) or said plant, plant cell or plant seed, to obtain increased regeneration of plant tissue cells. One alternative embodiment further relates to the use of a chimeric gene construct comprising a) a plant expressible promoter, b) a DNA region encoding PAT1, and c) a 3′ end region comprising transcription termination and polyadenylation signals functioning in cells of a plant, for obtaining increased regeneration of plan tissue cells.

Another embodiment relates to the use of the chimeric gene construct(s) of the present invention or said recombinant vector(s) or said plant, plant cell or plant seed, to obtain increased callus formation of plant tissue cells are described. Alternatively, the embodiment relates to the use of a chimeric gene construct comprising a) a plant expressible promoter, b) a DNA region encoding Phytochrome A Signal Transduction 1 (PAT1) and c) a 3′ end region comprising transcription termination and polyadenylation signals functioning in cells of a plant, to obtain increased callus formation of plant tissue cells.

A further embodiment relates to the use of said chimeric gene construct(s) or of said recombinant vector(s) or of said plant, plant cell or plant seed, to obtain increases callus formation of plant tissue cells, wherein said recombinase is activated prior to regeneration from callus to remove said chimeric constructs from said regenerated plant tissue cells.

Another aspect of the invention relates to a method for producing a transgenic plant, whereby the method comprises introduction of a plant cell with said chimeric gene construct(s) or with said recombinant vector(s) of the present invention, and isolating a plant regenerated from said method. Alternatively, said method for producing a transgenic plant comprises transient expression of said chimeric gene construct(s) during callus formation to improve plant cell regeneration, and isolating a plant regenerated from said method. In another embodiment, the invention relates to a method for producing a plant, whereby the method comprises introducing a plant cell with said chimeric gene construct(s) or said recombinant vector according, and wherein said site-specific recombinase is expressed prior to regeneration from callus, thereby excising said chimeric constructs comprising said DNA regions encoding ERF and PAT1 transcription factors, and said recombinase.

### DESCRIPTION OF THE FIGURES

The patent or application file contains at least one drawing executed in color. Copies of this patent or patent application publication with color drawing(s) will be provided by the Office upon request and payment of the necessary fee.

The drawings described are only schematic and are non-limiting. In the drawings, the size of some of the elements may be exaggerated and not drawn on scale for illustrative purposes.

FIGS. 1A-1L. ERF115 interacts with SCL21 and PAT1. FIG. 1A, Protein interactions identified through TAP using ERF115 and SCL21 as bait. Arrowheads point towards co-purifying proteins. FIG. 1B, Yeast two-hybrid interaction of ERF115 full length and fragments with SCL21 and PAT1. Growth was verified on control medium (+HIS) or selective medium (−HIS) supplemented with 15 mM 3-Amino-1,2,4-triazole (3-AT), using β-glucuronidase (GUS) as a negative control. FIG. 1C, Yeast two-hybrid interaction of ERF2 and ERF6 without (−) or fused to the conserved ERF115 interaction motif (+) with SCL21. The assay was performed similar to (FIG. 1B). FIGS. 1C-1I, Root tips expressing ERF115 (dots) (FIGS. 1D and 1E), SCL21 (no expression visible) (FIGS. 1F and 1G), and PAT1 (dots) (FIGS. 1H And 1I) under control conditions (FIGS. 1D, 1F, and 1H) or treated with bleomycin (FIGS. 1E, 1G, and 1I). Arrowheads indicate the quiescent center (QC). Cell walls are counterstained with propidium iodide (PI). Bars=50 μm. FIGS. 1J-1L, Expression of PAT1 (green) and ERF115 (red) in root tips 10 h15 to 11 h30 after recovery from bleomycin treatment. Arrowheads indicate the dividing QC cells, inset shows detail of a dividing QC cell co-expressing ERF115 and PAT1. Cell walls are counterstained (white) by SCRI Renaissance 2200 dye. Bars=25 μm.

FIGS. 2A-2J: ERF115 and PAT1 co-expression precedes stress-induced cell division. FIGS. 2A and 2B, ERF115 expression (green) upon bleomycin treatment of wild-type (FIG. 2A) and atm mutant (FIG. 2B) root tips. FIGS. 2C, 2D, ERF115 expression (green) upon hydroxyurea treatment of wild-type (FIG. 2C) and atr mutant (FIG. 2D) root tips. FIGS. 2E, 2F, Single cell death by laser ablation (FIG. 2E) or spontaneously occurring (FIG. 2F) triggers ERF115 expression (green). FIGS. 2A-2F, Cell walls are counterstained with PI. FIG. 2G, PAT1 (green) and ERF115 (red) expression in root tips 13 h to 16 h after recovery from bleomycin treatment. Cell walls are counterstained by SCRI Renaissance 2200 dye. FIG. 2H, Cells contacting dead cells express ERF115 (red) after 24-h recovery from bleomycin. Cell walls are counterstained with PI and SCRI Renaissance 2200 dye overlay. FIGS. 21, 2J, Wild-type (FIG. 2I) and ERF115^{SRDX} mutant (FIG. 2J) roots recovering from bleomycin at the indicated time points, showing anticlinal and replenishing divisions indicated by expression of the WOX5 marker (white). Cell walls are counterstained in gray with PI. FIGS. 2G-2J, grey arrowheads: anticlinal divisions, white arrowheads: replenishing divisions. FIGS. 2A-2J, Bars=50 μm.

FIGS. 3A-3D: ERF115-PAT1 activity is required for root tip regeneration. FIGS. 3A, 3B, Root tip excision showing the expression of ERF115 (dots) (FIG. 3A) and PAT1 (dots) (FIG. 3B) during the root tip regeneration process for the indicated time points (hpc=hours post cut). Cell walls are counterstained with PI. Bars=50 μm. FIG. 3C, Root tip excision showing the expression of ERF115 (red) and PAT1 (green) during the early root tip regeneration process for the indicated time points (hpc=hours post cut). Cell walls are counterstained in blue with SCRI Renaissance 2200 dye. Bar=50 μm. FIG. 3D, Quantification of root tip regeneration of wild-type, erf115, pat1-2, erf115-pat1-2 and ERF115^{SRDX} seedlings 72 h after excision. Data indicate mean±s.e.m. for n=4 with >11 technical repeats each (* indicates p-value<0.01 compared with wild type, ** indicates p-value<0.01 compared with *, Fisher's exact test).

FIGS. 4A-4K: ERF115-PAT1 activity triggers uncontrolled cell division. FIG. 4A, Wild-type (Col-0), ERF115^{OE}, PAT1^{OE} and ERF115-PAT1^{OE} 3-week-old seedlings. Bar=1 cm. FIG. 4B, Three-week-old ERF115-PAT1^{OE} seedling. Bar=0.5 cm. FIGS. 4C, 4D, WOX5-positive cells (blue) are visualized by the β-glucuronidase (GUS) staining in wild-type (FIG. 4C) and ERF115-PAT1^{OE} (FIG. 4D) roots at six days after stratification. Arrowheads indicate ectopic WOX5 expression. Bars=50 μm. FIGS. 4E, 4F, Wild-type (FIG. 4E) and ERF115-PAT1^{OE} (FIG. 4F) cotelydon. Blue GUS staining indicates cells ectopically expressing WOX5. Bars=0.1 mm. FIG. 4G, Relative PLT expression level in wild-type, ERF115^{OE}, PAT1^{OE} and ERF115-PAT1^{OE} 1-week-old seedlings. Data indicate mean±s.e.m. for n=3 (* indicates p-value<0.05 compared with wild type, Student's t-test). FIG. 4H, Relative WIND1 expression level in wild-type, ERF115^{OE}, PAT1^{OE} and ERF115-PAT1^{OE} 1-week-old seedlings. Data indicate mean±s.e.m. for n=3 (* indicates p-value<0.01 compared with wild type, ** indicates p-value<0.01 compared with *, Student's t-test). FIG. 4I, Relative WIND1 expression level in wild-type and ERF115^{SRDX} root tips control treated or treated with 0.6 mg/L bleomycin for 24 h. Data indicate mean±s.e.m. for n=3 (* indicates p-value<0.01 compared, n.s. indicates not significant, Student's t-test). FIG. 4J, Quantification of root tip regeneration of wild-type and WIND 1^{SRDX} seedlings 72 h after excision. Data indicate mean±s.e.m. for n=4 with >35 technical repeats each (* indicates p-value<0.01 compared with wild type, Fisher's exact test). FIG. 4K, Callus generated from wild-type and ERF115^{SRDX} hypocotyl segments. Bars=5 mm.

FIGS. 5A-5J: ERF115, PAT1, and SCL21 protein localization in tobacco. FIGS. 5A, 5B, Bimolecular fluorescence complementation using ERF115-headGFP and SCL21-tailGFP reveals interaction in the nucleus in N. benthamiana leaf pavement cells by fluorescence and light microscope overlay (FIG. 5A) or solely fluorescence signal (FIG. 5B). FIGS. 5C, 5D, Localization of the ERF115-GFP fusion protein in N. benthamiana leaf pavement cells by fluorescence and light microscope overlay (FIG. 5C) or solely fluorescence signal (FIG. 5D). FIGS. 5E, 5F, Localization of the SCL21-GFP fusion protein in N. benthamiana leaf pavement cells by fluorescence and light microscope overlay (FIG. 5E) or solely fluorescence signal (FIG. 5F). FIGS. 5G, 5H, Bimolecular fluorescence complementation using ERF115-tailGFP and PAT1-headGFP reveals interaction in the nucleus in N. benthamiana leaf pavement cells by fluorescence and light microscope overlay (FIG. 5G) or solely fluorescence signal (FIG. 5H). FIGS. 5I, 5J, Localization of the PAT1-GFP fusion protein in N. benthamiana leaf pavement cells by fluorescence and light microscope overlay (FIG. 5I) or solely fluorescence signal (FIG. 5J). White arrowheads indicate GFP positive nuclei. Bars=50 μm.

FIGS. 6A-6D: Mapping of the ERF115 interaction domain. FIG. 6A, Schematic overview of the ERF115 full-length protein and A-, B- and C-fragments. The B-fragment corresponds to the conserved AP2 DNA-binding domain. FIG. 6B, Negative control of the ERF115 fragment Y2H interaction using the GUS gene. FIG. 6C, Sequence alignment of the N-termini of the B-4 ERF subfamily revealing a (partially) conserved motif (box) in ERF115 (amino acids 1-89 of SEQ ID NO: 1), ERF114 (amino acids 1-87 of SEQ ID NO: 160), ERF113 (amino acids 1-37 of SEQ ID NO: 159), ERF111 (amino acids 1-182 of SEQ ID NO: 156), ERF110 (amino acids 1-74 of SEQ ID NO: 155), ERF109 (amino acids 1-133 of SEQ ID NO: 154), and ERF108 (amino acids 1-58 of SEQ ID NO: 153) as well as the schematic depicting the location of the motif (MVSALTQVIGN (amino acids 44-54 of SEQ ID NO: 1)) in ERF115. FIG. 6D, Schematic overview of the chimeric ERF2 and ERF6 proteins harboring the ERF115-conserved domain (depicted in grey colored box) at their N-termini.

FIGS. 7A-7D: PAT1 and ERF115 expression upon bleomycin treatment FIG. 7A, Expression of PAT1 (dots) under control conditions or treated with 0.6 mg/M bleomycin for 24 h. Bars=50 μm. FIG. 7B, Relative PAT1 expression level in wild-type root tips control treated or treated with 0.6 mg/L bleomycin for 24 h. Data indicate mean±s.e.m. for n=3. FIGS. 7C, 7D, Detail of the meristematic region of the root presented in FIG. 2 g for the 14 h (FIG. 7C) and 16 h (FIG. 7D) time points showing the separate ERF115 (red) and PAT1 (green) expression profiles together with the merged image. White arrowheads indicate dividing cells. Bars=25 μm.

FIGS. 8A-8D: Bleomycin triggers replenishing cell divisions. FIG. 8A, Seedlings marked for endodermis (dots) were treated with 0.6 mg/L bleomycin and allowed to recover from the stress for 24 h. Cell walls are counterstained using PI. Arrowheads indicate replenishing cell divisions. Bar=50 μm. FIG. 8B, pCYCD6; 1:GFP roots under control conditions are treated for 24 h with 0.6 mg/L bleomycin. Cell walls are counterstained using PI. Arrowheads indicate replenishing cell divisions. Bar=50 μm. FIG. 8C, Quantification of the total number of WOX5-positive cell divisions in wild-type (Col-0), ERF115^{SRDX} and pat1-2 seedlings during the first 14 h of recovery after treatment with 0.6 mg/L bleomycin for 24 h. Data indicate mean±s.e.m. for n>3 (no significant differences were found, Student's t-test). FIG. 8D, Quantification of replenishing versus anticlinal divisions of WOX5-positive cells in wild-type (Col-0), ERF115^{SRDX} and pat1-2 seedlings during the first 14 h of recovery after treatment with 0.6 mg/L bleomycin for 24 h. Data indicate mean±s.e.m. for n>3 (* indicates p-value<0.05 compared with wild type, Student's t-test).

FIGS. 9A-9H: Phenotypic data of ERF115-PAT1^{OE} seedlings. FIG. 9A, Wild-type (Col-0), ERF115^{OE}, PAT1^{OE} and ERF115-PAT1^{OE} seedlings at 7 DAS. Bar=1 cm. FIGS. 9B, 9C Hypocotyl sections of 2 days after germination old wild-type (Col-0) (FIG. 9B) and ERF115-PAT1^{OE} (FIG. 9C) seedlings. Bars=0.1 mm. FIG. 9D, Root length of wild-type (Col-0), ERF115^{OE}, PAT1^{OE} and ERF115-PAT1^{OE} plants at the indicated time points. Data indicate mean±s.e.m. for n>14 (* indicates p-value<0.01 at 7 DAS compared with wild type, ** indicates p-value<0.01 compared with wild type and *, *** indicates p-value<0.01 compared with ** , Student's t-test). FIG. 9E, Relative PSK5 expression levels in wild-type (Col-0), ERF115^{OE}, PAT1^{OE} and ERF115-PAT1^{OE} 1-week-old seedlings. Data indicate mean±s.e.m. for n=3 (* indicates p-value<0.01 compared with wild type, ** indicates p-value<0.01 compared with *, Student's t-test). FIGS. 9F-9H, PSK5:PSK5-GFP roots under control conditions, treated for 24 h with 0.6 mg/L bleomycin and 24 h post cut. Cell walls are counterstained using PI. Arrowheads indicate replenishing cell divisions. Bar=50 μm.

FIGS. 10A-10G: Ectopic WOX5 expression in ERF115-PAT1^{OE} seedlings. FIG. 10A, Relative WOX5 expression levels in wild-type (Col-0), ERF115^{OE}, PAT1^{OE} and ERF115-PAT1^{OE} 1-week-old seedlings. Data indicate mean±s.e.m. for n=3. FIGS. 10B-10E, Confocal microscope images of 1-week-old root tips of wild-type (Col-0) (FIG. 10B), ERF1150E (FIG. 10C), PAT1^{OE} (FIG. 10D) and ERF115-PAT1^{OE} (FIG. 10E) seedlings. WOX5 expression is visualized by the green fluorescence (white dots). The white arrowhead indicates ectopic WOX5-expressing cells in ERF115-PAT1^{OE} roots (FIG. 10E), shown in the enlargement. Bars=50 μm. FIGS. 10F, 10G, Images of WOX5-positive cells in wild-type (FIG. 10F) and ERF115-PAT1^{OE} (FIG. 10G) seedlings visualized by GUS staining on the indicated time points. Black arrowheads indicate ectopic WOX5 expression. Bars=0.5 mm. The inset in the final image represents a magnification of the WOX5-positive cells in the hypocotyl. Bar=50 μm.

FIGS. 11A and 11B: Absence of ERF115 induction in regeneration incompetent tissue. FIG. 11A, Callus generated from wild-type (Col-0), erf115, pat1-2, erf115 pat1-2 and ERF115^{SRDX} hypocotyl segments shows reduction in callus formation in erf115 pat1-2 and ERF115^{SRDX} seedlings. Bar=5 mm. FIG. 11B, Leaf blade cut of the PERF115:GUS reporter reveals the absence of ERF115 activation near the cut site at 24 hours post cut. Bar=1 mm.

FIGS. 12A-12F: ERF115 and PAT1 are both essential for callus formation. FIGS. 12A, 12B, GUS stained 3-week old hypocotyls grown on CIM after cutting them from 1-week old etiolated seedlings of ERF115 (pERF115:GUS) and PAT1 (pPAT1:GUS) reporter lines.

FIGS. 12C, 12D, calli regenerated from ERF115 (ERF1150E) overexpressor lines were not larger than those obtained from control plants (Col-0) FIGS. 12E, 12F, co-requirement of ERF115 and PAT1 for bigger callus induction was demonstrated by the observation that erf115 pat1-2 double mutants development poorly into calli, compared to those of the control plants.

FIG. 13: Yeast two-hybrid interaction network of ERF A fragments with SCL and PAT1 proteins. Growth was verified on selective medium (−HIS) supplemented with 15 mM 3-Amino-1,2,4-triazole (3-AT). Arrows point to interaction from bait to prey.

FIGS. 14A-14D: PAT1 overexpressor lines induce callus in the presence of auxin. Wild type (FIGS. 14A, 14C) and PAT1 overexpressing (FIGS. 14B, 14D) plants grown for two weeks on 10 μM 2,4-Dichlorophenoxyacetic acid (2,4D) (FIGS. 14A, 14B) or six weeks on ½ MS media containing 1 μM 2,4D (FIGS. 14C, 14D).

FIGS. 15A-15E: Spontaneous callus formation upon co-expression of ERF115 and SCL21. (FIG. 15A) 12-days-old ERF115-SCL21-overexpressing seedling. (FIG. 15B) 3-weeks-old ERF115-SCL21-overexpressing seedling displaying severe spontaneous callus formation. (FIG. 15C) In vitro grown 3-week-old ERF115^{OE} (left) and ERF115-SCL21-overexpressing (right) seedlings. (FIG. 15D) Longitudinal section of a 12-days-old ERF115-SCL21-overexpressing seedling. SAM, shoot apical meristem; Co, cotelydon; Hyp, hypocotyl. (FIG. 15E) Relative PSK expression levels in one-week-old control (Col-0), ERF115^{OE}, SCL21^{OE} and ERF115-SCL21-overexpressing seedlings. Bars represent standard error (n=3).

FIGS. 16A and 16B: ERF115 and PAT1 maize orthologues react to wounding and interact. (FIG. 16A) 7d-old seedlings of the B73 maize ecotype had their root tip excised. After 0.5 and 24 hours, the lower 1 mm of the root was taken for RT-qPCR analysis to analyse ERF115 expression levels of 3 maize candidate orthologues ERF115_1 (Zm10g15700), ERF115_2 (Zm02g16850) and ERF115_3 (Zm03g25810). (FIG. 16B) Yeast two-hybrid shows interaction between maize candidate orthologues of ERF115 and PAT1. Growth was verified on control medium (+HIS) or selective medium (−HIS).

### DETAILED DESCRIPTION TO THE INVENTION

The present invention will be described with respect to particular embodiments and with reference to certain drawings but the invention is not limited thereto but only by the claims. Any reference signs in the claims shall not be construed as limiting the scope. Of course, it is to be understood that not necessarily all aspects or advantages may be achieved in accordance with any particular embodiment of the invention. Thus, for example those skilled in the art will recognize that the invention may be embodied or carried out in a manner that achieves or optimizes one advantage or group of advantages as taught herein without necessarily achieving other aspects or advantages as may be taught or suggested herein.

The invention, both as to organization and method of operation, together with features and advantages thereof, may best be understood by reference to the following detailed description when read in conjunction with the accompanying drawings. The aspects and advantages of the invention will be apparent from and elucidated with reference to the embodiment(s) described hereinafter. Reference throughout this specification to “one embodiment” or “an embodiment” means that a particular feature, structure or characteristic described in connection with the embodiment is included in at least one embodiment of the present invention. Thus, appearances of the phrases “in one embodiment” or “in an embodiment” in various places throughout this specification are not necessarily all referring to the same embodiment, but may. Similarly, it should be appreciated that in the description of exemplary embodiments of the invention, various features of the invention are sometimes grouped together in a single embodiment, figure, or description thereof for the purpose of streamlining the disclosure and aiding in the understanding of one or more of the various inventive aspects. This method of disclosure, however, is not to be interpreted as reflecting an intention that the claimed invention requires more features than are expressly recited in each claim. Rather, as the following claims reflect, inventive aspects lie in less than all features of a single foregoing disclosed embodiment.

Where an indefinite or definite article is used when referring to a singular noun e.g. “a” or “an”, “the”, this includes a plural of that noun unless something else is specifically stated. Where the term “comprising” is used in the present description and claims, it does not exclude other elements or steps. Furthermore, the terms first, second, third and the like in the description and in the claims, are used for distinguishing between similar elements and not necessarily for describing a sequential or chronological order. It is to be understood that the terms so used are interchangeable under appropriate circumstances and that the embodiments, of the invention described herein are capable of operation in other sequences than described or illustrated herein. The following terms or definitions are provided solely to aid in the understanding of the invention. Unless specifically defined herein, all terms used herein have the same meaning as they would to one skilled in the art of the present invention. Practitioners are particularly directed to Sambrook et al., Molecular Cloning: A Laboratory Manual, 4^{th} ed., Cold Spring Harbor Press, Plainsview, New York (2012); and Ausubel et al., Current Protocols in Molecular Biology (Supplement 114), John Wiley & Sons, New York (2016), for definitions and terms of the art. The definitions provided herein should not be construed to have a scope less than understood by a person of ordinary skill in the art.

“About” as used herein when referring to a measurable value such as an amount, a temporal duration, and the like, is meant to encompass variations of ±20% or ±10%, more preferably ±5%, even more preferably ±1%, and still more preferably ±0.1% from the specified value, as such variations are appropriate to perform the disclosed methods.

“Gene” as used here includes both the promoter region of the gene as well as the coding sequence. It refers both to the genomic sequence (including possible introns) as well as to the cDNA derived from the spliced messenger, operably linked to a promoter sequence.

“Coding sequence” is a nucleotide sequence, which is transcribed into mRNA and/or translated into a polypeptide when placed under the control of appropriate regulatory sequences. The boundaries of the coding sequence are determined by a translation start codon at the 5′-terminus and a translation stop codon at the 3′-terminus. A coding sequence can include, but is not limited to mRNA, cDNA, recombinant nucleotide sequences or genomic DNA, while introns may be present as well under certain circumstances.

The terms “protein”, “polypeptide”, “peptide” are interchangeably used further herein to refer to a polymer of amino acid residues and to variants and synthetic analogues of the same. Thus, these terms apply to amino acid polymers in which one or more amino acid residues is a synthetic non-naturally occurring amino acid, such as a chemical analogue of a corresponding naturally occurring amino acid, as well as to naturally-occurring amino acid polymers. This term also includes post-translational modifications of the polypeptide, such as glycosylation, phosphorylation and acetylation. By “recombinant polypeptide” is meant a polypeptide made using recombinant techniques, i.e., through the expression of a recombinant or synthetic polynucleotide. When the chimeric polypeptide or biologically active portion thereof is recombinantly produced, it is also preferably substantially free of culture medium, i.e., culture medium represents less than about 20%, more preferably less than about 10%, and most preferably less than about 5% of the volume of the protein preparation.

For the purposes of the invention, “transgenic”, “transgene” or “recombinant” means with regard to, for example, a nucleic acid sequence, an expression cassette, chimeric gene construct or a vector comprising the nucleic acid sequence or an organism transformed with the nucleic acid sequences, expression cassettes or vectors according to the invention. A “transgenic plant” for the purposes of the invention is thus understood as meaning that the nucleic acids used in the method or use of the invention are not present in, or originating from, the genome of said plant, or are present in the genome of said plant but not at their natural locus in the genome of said plant, it being possible for the nucleic acids to be expressed homologously or heterologously. However, as mentioned, transgenic also means that, while the nucleic acids according to the invention or used in the inventive method are at their natural position in the genome of a plant, the sequence has been modified with regard to the natural sequence, and/or that the regulatory sequences of the natural sequences have been modified. Transgenic is preferably understood as meaning the expression of the nucleic acids according to the invention at an unnatural locus in the genome, i.e. homologous or, heterologous expression of the nucleic acids takes place.

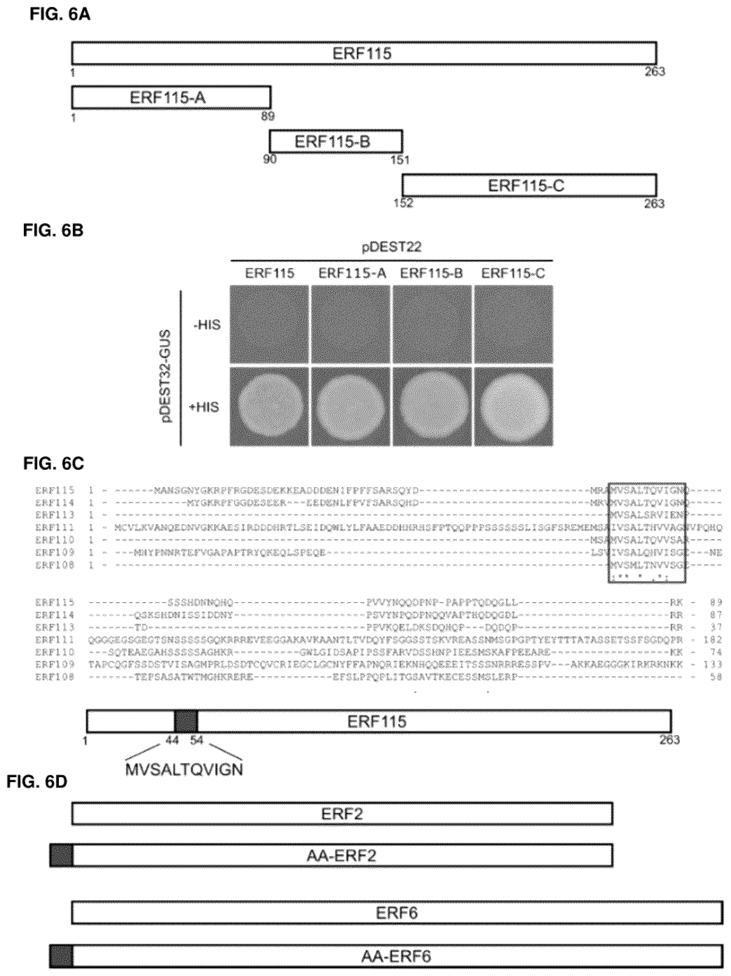

The current invention relates to the heterodimeric transcription factor complex ETHYLENE RESPONSE FACTOR115 (ERF115)-PHYTOCHROME A SIGNAL TRANSDUCTION1 (PAT1), which sustains meristem function by promoting cell renewal after stem cell loss, but moreover plays an important role in the high regenerative potential of plants, granting them the ability to improve their transformation efficiency by ectopic expression of the ERF115-PAT1 complex. In fact, the observed physical interaction between ERF115 and PAT1, combined with the co-expression of their genes and additive effects of the double mutants and overexpression lines on tissue regeneration, callus formation, and target gene expression (as exemplified below), strongly support the idea that ERF115 and PAT1 cooperate in determining the regeneration potential of cells. It is striking to note that the activation of the ERF115-PAT1 complex in response to neighbouring dead cells conceptually may be reminiscent to the regeneration process in decapitated Hydra, in which apoptosis is both necessary and sufficient to induce the regeneration-required Wnt3 production²⁴, indicating that plants and animals use the same general mechanism to repair injured tissues. Nevertheless, in view of their high regenerative power, plants appear to have evolved unique and more potent regeneration abilities that might be due to constant attack by herbivorous plants and insects¹⁻³. Together, the present invention relates to the finding that the ERF115-PAT1 complex plays a crucial role in granting plants their regenerative potential, in particular for the recovery of the root meristem, hence ensuring its indeterminate growth. Moreover, the interaction motif of ERF115 to allow complex formation with PAT1 was narrowed down to an 11-amino acid conserved region in its N-terminal part, and was named further herein as the “SCL/PAT1 interaction motif”. Said interaction motif has also been identified in other ERF proteins (see FIG. 6, as well as bold-labeled residues in ERF amino acid sequences of the examples), and therefore forms the essential feature to interact with GRAS TFs, and subsequently lead to spontaneous regeneration of plant cells with an active ERF-GRAS complex.

So, in a first aspect, the invention relates to a combination of a first and second chimeric gene construct, with both gene constructs comprising the following operably linked DNA elements: a) a plant expressible promoter, b) a DNA region encoding an Ethylene Response Factor (ERF) comprising the SCL/PAT1 interaction motif, or a DNA region encoding PAT1, for the first and second construct respectively, and c) a 3′ end region comprising transcription termination and polyadenylation signals functioning in cells of a plant. With “a combination” is meant that both, the first and the second gene construct, will be required to come to the invention. By combining said first and second chimeric gene constructs, one skilled in the art will obtain the desired outcome of an increased regeneration potential for plants in the present invention. With a “chimeric gene” or “chimeric construct” or “chimeric gene construct” is meant a recombinant nucleic acid sequence in which a promoter or regulatory nucleic acid sequence is operatively linked to, or associated with, a nucleic acid sequence that codes for an mRNA, such that the regulatory nucleic acid sequence is able to regulate transcription or expression of the associated nucleic acid coding sequence. The regulatory nucleic acid sequence of the chimeric gene is not operatively linked to the associated nucleic acid sequence as found in nature.

The term “operatively” or “operably” “linked” as used herein refers to a functional linkage between the promoter sequence and the gene of interest, such that the promoter sequence is able to initiate transcription of the gene of interest, and refers to a functional linkage between the gene of interest and the transcription terminating sequence to assure adequate termination of transcription in plant cells. In the present invention a “plant expressible promoter” comprises regulatory elements, which mediate the expression of a coding sequence segment in plant cells. For expression in plants, the nucleic acid molecule must be linked operably to or comprise a suitable promoter which expresses the gene at the right point in time and with the required spatial expression pattern. A “constitutive promoter” is used in some embodiments, and is a promoter that is transcriptionally active during most, but not necessarily all, phases of growth and development and under most environmental conditions, in at least one cell, tissue or organ. Examples of plant expressible promoters for constitutive expression are the Cauliflower mosaic virus (CaMV) 35S promoter. Specific expression patterns can be obtained by using tissue-preferred promoters to target enhanced expression of a sequence of interest within a particular plant tissue. Where low-level expression is desired, weak promoters will be used. Generally, by “weak promoter” is intended a promoter that drives expression of a coding sequence at a low level. By low level is intended at levels of about 1/1000 transcripts to about 1/100,000 transcripts to about 1/500,000 transcripts. Alternatively, it is recognized that weak promoters also encompasses promoters that are expressed in only a few cells and not in others to give a total low level of expression. Where a promoter is expressed at unacceptably high levels, portions of the promoter sequence can be deleted or modified to decrease expression levels. Such weak constitutive promoters include, for example, the core CaMV 35S promoter. Finally, in this application, also inducible promoter elements could be used in some embodiments. For instance, wound-inducible promoters include potato proteinase inhibitor (pin II) gene⁴⁰; and systemin⁴¹. Another inducible promoter is the maize In2-2 promoter⁴². Chemical-regulated promoters can be used to modulate the expression of a gene in a plant through the application of an exogenous chemical regulator. The promoter may be a chemical-inducible promoter, where application of the chemical induces gene expression, or a chemical-repressible promoter, where application of the chemical represses gene expression. Chemical-inducible promoters are known in the art and include, but are not limited to, the maize In2-2 promoter, which is activated by benzenesulfonamide herbicide safeners⁴², the maize GST promoter (GST-II-27, WO 93/01294), which is activated by hydrophobic electrophilic compounds that are used as pre-emergent herbicides, the PR-1 promoter⁴³, which is activated by BTH or benxo(1,2,3)thiaidazole-7-carbothioic acid s-methyl ester, the tobacco PR-1a promoter⁴⁴, which is activated by salicylic acid, the copper inducible ACE1 promoter⁴⁵, the ethanol-inducible promoter AlcA⁴⁶, an estradiol-inducible promoter⁴⁷, the XVE estradiol inducible promoter⁴⁸.

The term “terminator” encompasses a control sequence which is a DNA sequence at the end of a transcriptional unit which signals 3′ processing and polyadenylation of a primary transcript and termination of transcription. The terminator can be derived from the natural gene, from a variety of other plant genes, or from T-DNA. The terminator to be added may be derived from, for example, the nopaline synthase or octopine synthase genes, or alternatively from another plant gene, or less preferably from any other eukaryotic gene.

The ERF115 transcription factor has a function as a rate-limiting factor of quiescent center (QC) cell division, acting as a transcriptional activator of the phytosulfokine PSK5 peptide hormone. ERF115 marks QC cell division but is restrained through proteolysis, whereas QC proliferation is driven by brassinosteroid-dependent ERF115 expression, leading to two antagonistic mechanisms for regulation of ERF115 activity upon stem cells damage¹⁴. ERF115 is a member of the AP2/ERF family of proteins, which is a plant-specific class of putative transcription factors that regulate a wide variety of developmental processes and are characterized by the presence of an AP2 DNA binding domain that is predicted to form an amphipathic alpha helix that binds DNA (PFAM Accession PF00847). The AP2 domain was first identified in APETALA2, an Arabidopsis protein that regulates meristem identity, floral organ specification, seed coat development, and floral homeotic gene expression. The AP2/ERF proteins have been subdivided into distinct subfamilies based on the presence of conserved domains, one of these subfamilies concerning the ERF proteins⁴⁹. The ERF transcription factor comprising an SCL/PAT1 interaction motif of the present invention comprises a number of ERF transcription factors, for instance most of the ERFs that belong to the ERF115 subfamily contain said SCL/PAT1 interaction motif. The SCL/PAT1 interaction or binding motif is defined and used herein with reference to the 11 amino acid stretch present in the Arabidopsis thaliana ERF115 (see FIG. 6) starting at position 44 of SEQ ID NO:1. Said amino acid stretch has been demonstrated to be required for interaction with PAT1. In addition, said SCL/PAT1 interaction motif, as indicated (in bold) in all ERF115 protein sequences disclosed in the Example section, can be determined by a person skilled in the art by simply aligning the ERF sequences present in the public amino acid databases, with the At ERF115 as a reference (see Methods). As shown in FIG. 6, as a non-limiting example, said SCL/PAT1 interaction motif is also present in ERF108, ERF109, ERF110, ERF111, ERF113, and ERF114. In some embodiments, said DNA regions that encode the ERFs comprising such an SCL/PAT1 interaction motif comprise ERFs that are not ERF115, but that can be ERF114, ERF113, ERF111, ERF110, ERF109, or ERF108, but not ERF112, as the latter transcription factor does not contain an SCL/PAT1 interaction motif.

The DNA region (nucleotide sequence) encoding ERF is therefore meant to include a sequence encoding a member of the ERF protein family, preferably described, annotated as or homologous to the ERF115 protein, and at least comprising the SCL/PAT interaction motif. The Arabidopsis thaliana ERF115 DNA sequence comprising At ERF115 is provided in SEQ ID NO: 51, whereas the amino acid sequence annotated as A. thaliana ERF115 is provided in SEQ ID NO: 1. The Arabidopsis thaliana ERF108, ERF109, ERF110, ERF111, ERF112, ERF113, and ERF114 DNA sequences comprising At ERF108-At ERF 114 is provided in SEQ ID NO: 167-173, whereas the amino acid sequences annotated as A. thaliana ERF108-ERF114 are provided in SEQ ID NO: 153-160. Functional plant candidate orthologues for ERF115 are depicted in Example 6. The correspondence between the DNA sequence (nucleotide sequence) for ERF115 and the encoded ERF115 protein is as follows: SEQ ID NO:51 codes for the Arabidopsis thaliana ERF115 amino acid sequence presented in SEQ ID NO:1, SEQ ID NO: 52 encodes the protein in SEQ ID NO: 2, SEQ ID NO: 53 codes for SEQ ID NO:3, . . . until SEQ ID NO: 100 encodes for SEQ ID NO: 50.

The PAT1 protein encodes a cytoplasmic protein of 490 amino acids with sequence homologies to the plant-specific GRAS regulatory protein family. The GRAS protein family seems unique to plants and presently consists of >20 members. PAT1 shows the highest homology (45%-70% identity) to the protein sequences of the Scarecrow-like (SCL) Jan. 5, 2013 subgroup of the GRAS family and to SCL21 (SEQ ID NOs: 161-164). The protein functions in an early step of phyA signal transduction. Light signaling via the phytochrome A (phyA) photoreceptor controls basic plant developmental processes including de-etiolation and hypocotyl elongation⁵. The DNA region encoding PAT1 is therefore meant to include a sequence encoding a member of the PAT protein family, preferably described, annotated or homologous to the PAT1 protein or belonging to the PAT1 branch¹⁷. The Arabidopsis thaliana PAT1 DNA sequence including At PAT1 is provided in SEQ ID NO: 126, whereas the amino acid sequence annotated as A. thaliana PAT1 is provided in SEQ ID NO: 101. Functional plant candidate orthologues are depicted in Example 7. The correspondence between the DNA sequence (nucleotide sequence) for PAT1 and the encoded PAT1 protein is as follows: SEQ ID NO:101 codes for the Arabidopsis PAT1 amino acid sequence presented in SEQ ID NO:126, SEQ ID NO: 102 encodes the protein in SEQ ID NO: 127, SEQ ID NO: 103 codes for SEQ ID NO:128, . . . until SEQ ID NO: 125 encodes for SEQ ID NO: 150.

“Orthologues” and “paralogues” encompass evolutionary concepts used to describe the ancestral relationships of genes. Paralogues are genes within the same species that have originated through duplication of an ancestral gene; orthologues are genes from different organisms that have originated through speciation, and are also derived from a common ancestral gene. “Homologue”, “Homologues” of a protein encompass peptides, oligopeptides, polypeptides, proteins and enzymes having amino acid substitutions, deletions and/or insertions relative to the unmodified or wild-type protein in question and having similar biological and functional activity as the unmodified protein from which they are derived.

Functional orthologous ERF115 or PAT1 genes can be isolated from the (publically) available sequence databases. The “sequence identity” of two related nucleotide or amino acid sequences, expressed as a percentage, refers to the number of positions in the two optimally aligned sequences which have identical residues (×100) divided by the number of positions compared. A gap, i.e., a position in an alignment where a residue is present in one sequence but not in the other is regarded as a position with non-identical residues. The alignment of the two sequences is performed by the Needleman and Wunsch algorithm (Needleman and Wunsch (1970) J Mol Biol. 48: 443-453). The computer-assisted sequence alignment above, can be conveniently performed using standard software program such as GAP which is part of the Wisconsin Package Version 10.1 (Genetics Computer Group, Madision, Wisconsin, USA) using the default scoring matrix with a gap creation penalty of 50 and a gap extension penalty of 3. Sequences are indicated as “essentially similar” when such amino acid sequences have a sequence identity of at least about 75%, particularly at least about 80%, more particularly at least about 85%, quite particularly about 90%, especially about 95%, more especially about 100%, quite especially are identical. Alternatively, the skilled person can isolate orthologous plant ERF115 or PAT1 genes through methods of genetic hybridization. Such methods are well known to the skilled (plant) molecular biologist. Several examples of functional plant candidate orthologous ERF115 and PAT1 genes are represented in Examples 6 and 7, respectively.

The co-expression of multiple genes can be valuable in transgenic plants. The current invention relates to the co-expression of ERF and GRAS transcription factors, leading to the formation of a heterodimeric protein complex, in particular here for ERF115 and PAT1. It was demonstrated that this protein complex is active in Arabidopsis SCN cells nearby wounded cells to induce renewal of stem cells upon DNA damage. Moreover, the protein complex was shown to induce spontaneous callus formation and regeneration of plant cells. Hence, co-expression of ERF and PAT1 or PAT1-related GRAS transcription factors such as the SCL21 or SCL-TFs belonging to the PAT1 branch¹⁷, in particular co-expression of ERF115 and PAT1 is aimed for in order to gain regeneration competence in plant cells. To achieve co-expression within the plant cells, a multitude of techniques is known to the skilled person. These techniques include, but are not limited to co-transformation of multiple chimeric genes (here a first chimeric gene comprising ERF and a second chimeric gene comprising PAT1, either simultaneously or subsequently), crossing of transgenic plants (or ‘gene stacking’) wherein said transgenic plants each have one of the transgenes (one plant comprising a chimeric gene encoding ERF with another plant comprising a chimeric gene encoding PAT1), the use of multiple or bidirectional promoters to direct the expression of ERF115 and PAT1 on the same construct, the creation of a bicistronic or multicistronic construct wherein ERF115 and PAT1 are operably linked and under control of the same promoter. Multicistronic vectors or chimeric constructs can be made with IRES elements. However, these elements are quite large (500-600 bp). Alternative multicistronic vectors are made by using self-cleaving 2A peptides codes between the genes in the multicistronic vector or construct. Examples of commonly used 2A peptides used are T2A, P2A, E2A and F2A.

In one embodiment, a chimeric gene construct comprising the operably linked DNA elements: a) a plant expressible promoter, b) a multicistronic DNA region encoding an ERF comprising the SCL/PAT1 interaction motif, coupled to a DNA region encoding PAT1 and c) a 3′end region comprising transcription termination and polyadenylation signals functioning in cells of a plant is used. In said multicistronic DNA region, the above-mentioned elements can be used to obtain such a chimeric construct.

A particular embodiment relates to a chimeric gene construct of the present invention, wherein said DNA region encoding an ERF comprising the SCL/PAT1 interaction motif is ERF115.

In an alternative embodiment, said described chimeric gene constructs are flanked by recombination sites, and further comprise a chimeric gene construct wherein the DNA region encodes a site-specific recombinase protein which actively recognizes and exerts recombinase activity at those recombination sites within its reach to excise all DNA elements within the flanked sites. In an embodiment with said chimeric construct containing a DNA region encoding the site-specific recombinase, the DNA elements introduced by the chimeric gene constructs are all flanked by site-specific recombination sites that are directly repeated and are recognized by the site-specific recombinase whose expression is regulated by a presently disclosed promoter construct, such that expression of the site-specific recombinase results in the excision of the chimeric gene constructs. Examples of said site-specific recombinase are Cre or FLP recombinases.

The flanking recombination site sequences are defined to be the region of the gene on either side of the transcribed region, or alternatively in order to excise the full chimeric gene constructs the flanked recombination site sequences are present at the 5′ and 3′ end of the chimeric construct itself.

In one embodiment, the chimeric gene constructs are designed to contain DNA elements operably linked and flanked by recombination sites, as such that the chimeric construct with the site-specific recombinase DNA region, expressing the recombinase, specifically recognizes and implements recombination at the recombination sites flanking the chimeric constructs comprising the DNA region encoding ERF or PAT1, thereby excising the chimeric genes. The chimeric constructs can further comprise any of the operably linked sequences as those described herein. In an alternative embodiment, the chimeric gene construct comprising the DNA region encoding the site-specific recombinase as well contains flanking recombination site sequences. This allows to excise not only the chimeric gene constructs encoding the ERF and PAT1 proteins, but also to excise the chimeric gene construct enabling the recombination reaction by expression of the recombinase.

In some embodiments, the activity and/or amount of the ERF-PAT1 complex is reduced prior to regenerating a plant from a cell or tissue. In some of these embodiments, the polynucleotides encoding the ERF-PAT1 proteins are excised prior to the regeneration of a plant. In certain embodiments, the promoter and other regulatory elements that are operably linked to the heterologous polynucleotide encoding the ERF-PAT1 complex are excised along with the recombinase coding sequence. The polynucleotides encoding the ERF-PAT1 complex may be flanked by recombination sites and an appropriate site-specific recombinase is introduced into the mature embryo explant or callus grown therefrom to excise the polynucleotides encoding the ERF-PAT1 complex prior to regeneration of the mature embryo explant or callus into a plant. In some of those embodiments wherein an ERF, a PAT1, and a recombinase protein are provided to the plant cell upon expression of all three DNA regions, all three regions can be excised. The two, ERF and PAT1, can be present on the same or different chimeric gene constructs and, therefore, can be excised in one or two different excision reactions. In some of these embodiments, the DNA region encoding the site-specific recombinase for excising the ERF and PAT1 encoding chimeric genes can be located on the same chimeric gene as the ERF and PAT1 DNA region and all three polynucleotides can be excised through the activity of the site-specific recombinase. In order to control the excision of the ERF-PAT1 encoding chimeric genes, the expression of the site-specific recombinase that is responsible for the excision can be controlled by a late embryo promoter or an inducible promoter. The late embryo promoter may be GZ (Uead et al. (1994) Mol Cell Biol 14:4350-4359), ZM-LEG1 (U.S. Pat. No. 7,211,712). The inducible promoter that regulates the expression of the site-specific recombinase may also be a heat-shock, light-induced promoter, a drought-inducible promoter, including but not limited to Hva151, Dhn, and WSI18.

In a number of embodiments, in addition to the ERF-PAT1 protein complex, other proteins of interest (e.g., traits) may also be introduced along with or following the chimeric gene constructs comprising a presently disclosed design. In some embodiments, it might be of interest to include chimeric gene constructs comprising a DNA region encoding a protein of interest for stable introduction or transformation in the plant. In another embodiment, even such chimeric gene constructs comprising a DNA region encoding a protein of interest might be of interest to be flanked by recombination sites, and allow excision.

In the current invention, in some embodiments, the chimeric constructs comprising the DNA regions encoding PAT1, and/or an ERF comprising an SCL/PAT1 interaction motif, will be of interest for use in expression and activity during plant transformation and callus formation, to improve the regeneration and transformation efficiency. In other embodiments, the chimeric constructs comprising the DNA regions encoding a PAT1-related GRAS transcription factor part of the PAT1 branch (according to 17) and/or an ERF comprising an SCL/PAT1 interaction motif, will be of interest for use in expression and activity during plant transformation and callus formation, to improve the regeneration and transformation efficiency. In particular, said PAT1 branch transcription factors include SCL21, SCL13, SCL5 and SCL1. Depending on the desired outcome, the chimeric gene constructs for expression of ERF-PAT1 will be of use for transient utility, such as during callus formation, after which the expression is lowered or omitted.

In yet another embodiment the invention relates to a set of at least one recombinant vector comprising said chimeric gene or chimeric genes of the invention. The chimeric gene construct(s) can be part of a vector that comprises multiple chimeric gene constructs or multiple genes, such as a selectable marker gene. Selectable marker genes may be used to identify transformed cells or tissues. The chimeric gene or chimeric genes to be expressed are preferably cloned into a vector, or recombinant vector, which is suitable for transforming Agrobacterium tumefaciens, for example pBin19 (Bevan et al (1984) Nucl. Acids Res. 12-8711). In one embodiment, the first chimeric construct comprising the DNA region encoding ERF comprising an SCL/PAT1 interaction motif, and the second chimeric construct comprising the DNA region encoding PAT1 are both cloned in a different recombinant vector. In a specific embodiment, the first chimeric construct comprising the DNA region encoding ERF115, and the second chimeric construct comprising the DNA region encoding PAT1 are both cloned in a different recombinant vector. In said cases, co-transformation using both vectors is required to obtain co-expression and complex formation. In particular, when a third chimeric construct comprising a DNA region encoding a recombinase is added, or even a chimeric construct comprising a DNA region encoding a polynucleotide of interest, several recombinant vectors are necessary for introducing said genes. Alternatively, said first, second, and optionally third chimeric gene constructs can be cloned into the same recombinant vector, allowing the need of just one vector for expression of said chimeric genes. In another embodiment, the chimeric construct comprises the DNA region encoding both, an ERF comprising an SCL/PAT1 interaction motif and PAT1, whereby it will be sufficient to clone said chimeric construct into one recombinant vector to express the ERF-PAT1 complex from said chimeric construct.

In a specific embodiment, the chimeric construct comprises the DNA region encoding both, ERF115 and PAT1, whereby it will be sufficient to clone said chimeric construct into one recombinant vector to express the ERF115-PAT1 complex from said chimeric construct.

In yet another embodiment, a plant, plant cell or plant seed comprises said chimeric construct(s) or recombinant vector. Moreover, in one embodiment, a plant, plant cell, or plant seed is co-expressing ERF and PAT1. The term “plant” as used herein encompasses whole plants, ancestors and progeny of the plants and plant parts, including seeds, shoots, stems, leaves, roots (including tubers), flowers, and tissues and organs, wherein each of the aforementioned comprise the gene/nucleic acid of interest. The term “plant” also encompasses plant cells, suspension cultures, callus tissue, embryos, meristematic regions, gametophytes, sporophytes, pollen and microspores, again wherein each of the aforementioned comprises the gene/nucleic acid of interest.

Plants that are particularly useful in the invention include in particular monocotyledonous and dicotyledonous plants including fodder or forage legumes, ornamental plants, food crops, trees or shrubs selected from the list comprising Acer spp., Actinidia spp., Abelmoschus spp., Agave sisalana, Agropyron spp., Agrostis stolonifera, Allium spp., Amaranthus spp., Ammophila arenaria, Ananas comosus, Annona spp., Apium graveolens, Arachis spp, Artocarpus spp., Asparagus officinalis, Avena spp. (e.g. Avena sativa, Avena fatua, Avena byzantina, Avena fatua var. sativa, Avena hybrida), Averrhoa carambola, Bambusa sp., Benincasa hispida, Bertholletia excelsea, Beta vulgaris, Brassica spp. (e.g. Brassica napus, Brassica rapa ssp. [canola, oilseed rape, turnip rape]), Cadaba farinosa, Camellia sinensis, Canna indica, Cannabis sativa, Capsicum spp., Carex elata, Carica papaya, Carissa macrocarpa, Carya spp., Carthamus tinctorius, Castanea spp., Ceiba pentandra, Cichorium endivia, Cinnamomum spp., Citrullus lanatus, Citrus spp., Cocos spp., Coffea spp., Colocasia esculenta, Cola spp., Corchorus sp., Coriandrum sativum, Corylus spp., Crataegus spp., Crocus sativus, Cucurbita spp., Cucumis spp., Cynara spp., Daucus carota, Desmodium spp., Dimocarpus longan, Dioscorea spp., Diospyros spp., Echinochloa spp., Elaeis (e.g. Elaeis guineensis, Elaeis oleifera), Eleusine coracana, Eragrostis tef, Erianthus sp., Eriobotrya japonica, Eucalyptus sp., Eugenia uniflora, Fagopyrum spp., Fagus spp., Festuca arundinacea, Ficus carica, Fortunella spp., Fragaria spp., Ginkgo biloba, Glycine spp. (e.g. Glycine max, Soja hispida or Soja max), Gossypium hirsutum, Helianthus spp. (e.g. Helianthus annuus), Hemerocallis fulva, Hevea brasiliensis, Hibiscus spp., Hordeum spp. (e.g. Hordeum vulgare), Ipomoea batatas, Juglans spp., Lactuca sativa, Lathyrus spp., Lens culinaris, Linum usitatissimum, Litchi chinensis, Lotus spp., Luffa acutangula, Lupinus spp., Luzula sylvatica, Lycopersicon spp. (e.g. Lycopersicon esculentum, Lycopersicon lycopersicum, Lycopersicon pyriforme), Macrotyloma spp., Malus spp., Malpighia emarginata, Mammea americana, Mangifera indica, Manihot spp., Manilkara zapota, Medicago sativa, Melilotus spp., Mentha spp., Miscanthus sinensis, Momordica spp., Morus nigra, Musa spp., Nicotiana spp., Olea spp., Opuntia spp., Ornithopus spp., Oryza spp. (e.g. Oryza sativa, Oryza latifolia), Panicum miliaceum, Panicum virgatum, Passiflora edulis, Pastinaca sativa, Pennisetum sp., Persea spp., Petroselinum crispum, Phalaris arundinacea, Phaseolus spp., Phleum pratense, Phoenix spp., Phragmites australis, Physalis spp., Pinus spp., Pistacia vera, Pisum spp., Poa spp., Populus spp., Prosopis spp., Prunus spp., Psidium spp., Punica granatum, Pyrus communis, Quercus spp., Raphanus sativus, Rheum rhabarbarum, Ribes spp., Ricinus communis, Rubus spp., Saccharum spp., Salix sp., Sambucus spp., Secale cereale, Sesamum spp., Setaria sp., Sinapis sp., Solanum spp. (e.g. Solanum tuberosum, Solanum integrifolium or Solanum lycopersicum), Sorghum bicolor, Spinacia spp., Syzygium spp., Tagetes spp., Tamarindus indica, Theobroma cacao, Trifolium spp., Tripsacum dactyloides, Triticosecale rimpaui, Triticum spp. (e.g. Triticum aestivum, Triticum durum, Triticum turgidum, Triticum hybernum, Triticum macha, Triticum sativum, Triticum monococcum or Triticum vulgare), Tropaeolum minus, Tropaeolum majus, Vaccinium spp., Vicia spp., Vigna spp., Viola odorata, Vitis spp., Zea mays, Zizania palustris, Ziziphus spp., amongst others.

In a specific embodiment, said plant, plant cell or plant seed species for (co-) expression of said chimeric constructs or recombinant vectors is selected from the family of the Gramineae. This family, also called the Poaceae, are the large and nearly ubiquitous family of monocotyledonous flowering plants known as grasses. The Poaceae include the cereal grasses, bamboos and the grasses of natural grassland and cultivated lawns (turf) and pasture. Grasses have stems that are hollow except at the nodes and narrow alternate leaves borne in two ranks. The lower part of each leaf encloses the stem, forming a leaf-sheath. Grasses are unusual in that the meristem is located near the bottom of the plant; hence, they can quickly recover from cropping at the top. Many grasses demonstrate economic importance in food production, industry, and lawns. They have been grown as food for domesticated animals for up to 6,000 years and due to their grains, crops such as wheat, rice, maize (corn), oats, rye, sorghum, millet and barley have been the most important human food crops.

In yet another embodiment the invention provides the use of said chimeric genes, or recombinant vectors, or plant, plant cell, or plant seed to obtain increased regeneration of plant tissue cells. The terms “increased”, “improved” or “enhanced” are interchangeable and shall mean in the sense of the application at least a 3%, 4%, 5%, 6%, 7%, 8%, 9% or 10%, preferably at least 15% or 20%, more preferably 25%, 30%, 35%, 40%, 50%, 60%, 70%, 80%, 90%, 95% or more e.g. callus formation or transformed cells in comparison to control plants as defined herein. The choice of suitable control plants is a routine part of an experimental setup and may include corresponding wild type plants or corresponding plants without the chimeric constructs. The control plant is typically of the same plant species or even of the same variety as the plant to be assessed, following the same treatment. A “control plant” as used herein refers not only to whole plants, but also to plant parts, including embryos and tissue used for plant transformation. The term “regeneration” of plant tissue cells as used herein refers to a morphogenic response that results in the production of new tissues, organs, embryos, whole plants or parts of whole plants that are derived from a single cell or a group of cells. Regeneration may proceed indirectly via a callus phase or directly, without an intervening callus phase. “Regenerative capacity” refers to the ability of a plant cell to undergo regeneration.

In one embodiment, said use of said chimeric construct to obtain increased regeneration of plant tissue cells, relates to the chimeric construct(s) of the present invention comprising a DNA region encoding PAT1 and/or a DNA region encoding an ERF comprising an SCL/PAT1 interaction motif. In another embodiment, said use of said chimeric construct to obtain increased regeneration of plant tissue cells, relates to the chimeric construct(s) of the present invention comprising a DNA region encoding a GRAS transcription factor of the PAT1 branch¹⁷, and/or a DNA region encoding an ERF comprising an SCL/PAT1 interaction motif. In another embodiment, said use of said chimeric construct to obtain increased regeneration of plant tissue cells, relates to the chimeric construct(s) of the present invention comprising a DNA region encoding PAT1 and/or a DNA region encoding ERF115.

Alternatively, one embodiment provides the use of said chimeric genes to obtain increased callus formation of plant cells. Said increased callus formation will be the result of (temporary) expression and activity of the ERF-PAT1 complex, and will allow to improve the transformation efficiency, which is of massive importance in plant species that are currently experiencing difficulties in regeneration capacities or for species which currently require long and laborious hormone treatment to regenerate new plants from plant tissue cells. In one embodiment, said use of said chimeric construct to obtain increased callus formation of plant tissue cells, relates to the chimeric construct(s) of the present invention comprising a DNA region encoding PAT1 and/or a DNA region encoding an ERF comprising an SCL/PAT1 interaction motif. In another embodiment, said use of said chimeric construct to obtain increased callus formation of plant tissue cells, relates to the chimeric construct(s) of the present invention comprising a DNA region encoding a GRAS transcription factor of the PAT1 branch¹⁷, and/or a DNA region encoding an ERF comprising an SCL/PAT1 interaction motif. In another embodiment, said use of said chimeric construct to obtain increased callus formation of plant tissue cells, relates to the chimeric construct(s) of the present invention comprising a DNA region encoding PAT1 and/or a DNA region encoding ERF115.

The invention alternatively provides in the use of said chimeric genes and recombinant vector or plant, plant cell or plant seed to activate said recombinase just prior to regeneration of the formed callus tissue, leading to excision and removal of the chimeric gene constructs from the transformed calli, to allow further regeneration of said plant tissue into full plants or plant organs. Said use allows to obtain regenerated plants lacking any non-natural DNA, as all chimeric gene constructs that were at first introduced in order to increase regeneration potential, have been removed by the activity of said recombinase. Alternatively, said use will allow to obtain transgenic plants in which only additional chimeric constructs, lacking any flanking recombination sites, were introduced (meant for stable expression), and wherein the chimeric genes comprising DNA regions encoding ERF and PAT1 were only shortly introduced and expressed, just to allow higher regeneration competence, followed by excision via the flanking recombination sites.

In another aspect, the invention indeed also relates to a method for producing a transgenic plant, whereby the method comprises introducing a plant cell with said chimeric gene construct(s) or recombinant vector(s), and isolating a plant regenerated from said method. “Introducing” is intended to mean presenting to the organism, such as a plant, or the cell the chimeric gene or recombinant vector in such a manner that the sequence gains access to the interior of a cell of the organism or to the cell itself. Hence, the term “introduction” or “transformation” as referred to herein encompass the transfer of an exogenous polynucleotide or chimeric gene into a host cell, irrespective of the method used for transfer. Plant tissue capable of subsequent clonal propagation, whether by organogenesis or embryogenesis, may be transformed with a genetic construct of the present invention and a whole plant regenerated there from. The particular tissue chosen will vary depending on the clonal propagation systems available for, and best suited to, the particular species being transformed. Exemplary tissue targets include leaf disks, pollen, embryos, cotyledons, hypocotyls, megagametophytes, callus tissue, existing meristematic tissue (e.g., apical meristem, axillary buds, and root meristems), and induced meristem tissue (e.g., cotyledon meristem and hypocotyl meristem). The chimeric gene may be transiently or stably introduced into a host cell and may be maintained non-integrated, for example, as a plasmid. Alternatively, it may be integrated into the host genome. The resulting transformed plant cell may then be used to regenerate a transformed plant in a manner known to persons skilled in the art.

Transformation methods include, but are not limited to, stable transformation methods, transient transformation methods, virus-mediated methods, and sexual breeding. In one embodiment, the chimeric genes will be inserted by “Stable transformation”, which is intended to mean that the chimeric construct introduced into a plant integrates into a genome of the plant and is capable of being inherited by the progeny thereof. Protocols for introducing polypeptides or polynucleotide sequences into plants may vary depending on the type of plant or plant cell, i.e., monocot or dicot, targeted for transformation. Suitable methods of introducing polypeptides and polynucleotides into plant cells include microinjection (Crossway et al. (1986) Biotechniques 4:320-334), electroporation (Riggs et al. (1986) Proc. Natl. Acad. Sci. USA 83:5602-5606), Agrobacterium-mediated transformation (U.S. Pat. Nos. 5,563,055 and 5,981,840), direct gene transfer (Paszkowski et al. (1984) EMBO J. 3:2717-2722), and ballistic particle acceleration.

In another embodiment, the invention provides a method for producing a transgenic plant, whereby the method comprises transient expression of said chimeric gene construct(s) during callus formation to improve regeneration, and isolating a plant regenerated from said method. In such a method, one can make use of transient expression of the genes via inducible or temporal expression by selecting a suitable promoter, or one can also apply a “transient transformation” method, which is intended to mean that a chimeric gene is introduced into the plant and does not integrate into a genome of the plant or a polypeptide is introduced into a plant. The sequences can be provided to a plant using a variety of transient transformation methods. Such transient transformation methods include, but are not limited to, the introduction of the polypeptide of interest directly into the plant or the introduction of a polynucleotide encoding the polypeptide of interest into the plant. Such methods include, for example, microinjection or particle bombardment. Alternatively, the polynucleotide can be transiently transformed into the plant using techniques known in the art. Such techniques include viral vector system and the precipitation of the polynucleotide in a manner that precludes subsequent release of the DNA. Thus, the transcription from the particle-bound DNA can occur, but the frequency with which its released to become integrated into the genome is greatly reduced. Such methods include these particles coated with polyethylimine (PEI; Sigma #P3143). The chimeric gene may also be introduced into plants by contacting plants with a virus or viral nucleic acids. Generally, such methods involve incorporating a nucleotide construct within a viral DNA or RNA molecule.

The transfer of foreign genes into the genome of a plant is called transformation. Transformation of plant species is now a fairly routine technique. Advantageously, any of several transformation methods may be used to introduce the chimeric gene into a suitable ancestor cell. The methods described for the transformation and regeneration of plants from plant tissues or plant cells may be utilized for transient or for stable transformation. Transgenic plants, including transgenic crop plants, are preferably produced via Agrobacterium-mediated transformation. An advantageous transformation method is the transformation in planta. To this end, it is possible, for example, to allow the agrobacteria to act on plant seeds or to inoculate the plant meristem with agrobacteria. It has proved particularly expedient in accordance with the invention to allow a suspension of transformed agrobacteria to act on the intact plant or at least on the flower primordia. The plant is subsequently grown on until the seeds of the treated plant are obtained (Clough and Bent, Plant J. (1998) 16, 735-743). Methods for Agrobacterium-mediated transformation of rice include well known methods for rice transformation, (e.g. Aldemita and Hodges, Planta 199: 612-617, 1996; Chan et al. Plant Mol Biol 22 (3): 491-506, 1993). In the case of corn transformation, exemplified methods are described in either Ishida et al. (Nat. Biotechnol 14(6): 745-50, 1996) or Frame et al. (Plant Physiol 129(1): 13-22, 2002. Said methods are further described by way of example in B. Jenes et al., Techniques for Gene Transfer, in: Transgenic Plants, Vol. 1, Engineering and Utilization, eds. S. D. Kung and R. Wu, Academic Press (1993) 128-143 and in Potrykus Annu. Rev. Plant Physiol. Plant Molec. Biol. 42 (1991) 205-225). The nucleic acids or the construct to be expressed is preferably cloned into a vector, which is suitable for transforming Agrobacterium tumefaciens, for example pBin19 (Bevan et al (1984) Nucl. Acids Res. 12-8711). Agrobacteria transformed by such a vector can then be used in known manner for the transformation of plants, such as plants used as a model, like Arabidopsis or crop plants such as, by way of example, tobacco plants, for example by immersing bruised leaves or chopped leaves in an agrobacterial solution and then culturing them in suitable media. The transformation of plants by means of Agrobacterium tumefaciens is described, for example, by Hofgen and Willmitzer in Nucl. Acid Res. (1988) 16, 9877 or is known inter alia from F. F. White, Vectors for Gene Transfer in Higher Plants; in Transgenic Plants, Vol. 1, Engineering and Utilization, eds. S. D. Kung and R. Wu, Academic Press, 1993, pp. 15-38.

The genetically modified plant cells can be regenerated via all methods with which the skilled worker is familiar. Suitable methods can be found in the abovementioned publications by S. D. Kung and R. Wu, Potrykus or Hofgen and Willmitzer. Generally, after transformation, plant cells or cell groupings are selected for the presence of one or more markers which are encoded by plant-expressible genes co-transferred with the gene of interest, following which the transformed material is regenerated into a whole plant. To “isolate” or “select” transformed plants, the plant material obtained in the transformation is, as a rule, subjected to selective conditions so that transformed plants can be distinguished from untransformed plants. For example, the seeds obtained in the above-described manner can be planted and, after an initial growing period, subjected to a suitable selection by spraying. A further possibility consists in growing the seeds, if appropriate after sterilization, on agar plates using a suitable selection agent so that only the transformed seeds can grow into plants. Alternatively, the transformed plants are screened for the presence of a selectable marker such as the ones described above. Following DNA transfer and regeneration, putatively transformed plants may also be evaluated, and isolated, for instance using Southern analysis, for the presence of the gene of interest, copy number and/or genomic organisation. Alternatively or additionally, expression levels of the newly introduced DNA may be monitored using Northern and/or Western analysis, both techniques being well known to persons having ordinary skill in the art. The (re)generated transformed plants may be propagated by a variety of means, such as by clonal propagation or classical breeding techniques. For example, a first generation (or T1) transformed plant may be selfed and homozygous second-generation (or T2) transformants selected, and the T2 plants may then further be propagated through classical breeding techniques. The generated transformed organisms may take a variety of forms. For example, they may be chimeras of transformed cells and non-transformed cells; clonal transformants (e.g., all cells transformed to contain the expression cassette); grafts of transformed and untransformed tissues (e.g., in plants, a transformed rootstock grafted to an untransformed scion).

In the method provided in the present invention, introduction of said chimeric gene(s) will allow to improve plant regeneration during callus formation, due to the activity of the ERF-PAT1 complex, providing that introducing the chimeric genes will involve a callus-derived regeneration step. Hence said method is applicable to the above-mentioned transformation method comprising at least a step wherein dedifferentiated cells are present, and are regenerated into new plant tissue or organs, which can be isolated from said callus.

In an alternative embodiment of the invention, a method is provided for producing a plant, whereby the method comprises introducing a plant cell with said chimeric gene construct(s) or said recombinant vector according, and wherein said site-specific recombinase is expressed prior to regeneration from callus, thereby excising said chimeric constructs comprising said DNA regions encoding ERF and PAT1, and said recombinase. The chimeric gene constructs encoding the ERF-PAT1 complex may be flanked by recombination sites in some embodiments, as well as a chimeric gene construct comprising a DNA region encoding a site-specific recombinase which upon expression will recognize said flanking recombination sites and when introduced into the mature embryo explant or callus grown therefrom be activated to excise the chimeric constructs encoding the ERF-PAT1 complex prior to regeneration of the mature embryo explant or callus into a plant. In some of those embodiments wherein an ERF, a PAT1, and a recombinase protein are provided to the plant cell upon expression of all three DNA regions, all three regions can be excised. The two coding sequences, ERF and PAT1, can be present on the same or different chimeric gene constructs and, therefore, can be excised in one or two different excision reactions. In some of these embodiments, the DNA region encoding the site-specific recombinase for excising the ERF115 and PAT1 encoding chimeric genes can be located on the same chimeric gene as the ERF and PAT1 DNA region and all three polynucleotides can be excised through the activity of the site-specific recombinase. In said method, the produced regenerated plant will not be a transgenic plant when recombinase activity was adequately and fully functional, as the chimeric genes have been removed prior to regeneration of the transformed calli into new plant organs.

Said methods provided by the current invention are applicable to all plant species for which transformation/regeneration protocols involve a stage with callus formation or dedifferentiated transformed cells which are subsequently regenerative.

Such methods for improved regeneration capability involve the (transient) co-expression of ERF (comprising an SCL/PAT1 interaction motif) and PAT1 at a particular phase, wherein the ERF and PAT1 genes of the chimeric construct(s) can be of the same origin or homologous to the transformed or regenerated plant species, or in some embodiments, the ERF and PAT1 genes of the chimeric construct(s) can be of different origin or heterologous to the transformed or regenerated plant species.

If for instance in maize, the proposed functional candidate orthologue sequences for ERF115 and PAT1 are tested for their ability to improve the transformation protocol, the Arabidopsis ERF115 and PAT1 can be applied, or the functional corn orthologues can be applied for optimal improvement of regeneration during transformation of this crop. Based on phylogenetics, protein domain analysis and reciprocal BLAST analysis, ERF115 and PAT1 maize homologs were identified (see Example 6 and 7; ERF115 candidates encoded by SEQ ID NO: 52-54 with resulting amino acid sequences SEQ ID NO: 2-4; PAT1 candidates encoded by SEQ ID NO: 127-129 with resulting amino acid sequences SEQ ID NO: 102-104). Their coding sequence was optimized for synthesis and cloning, and the resulting nucleotide sequences (SEQ ID NO:178-183) were cloned in a transformation cassette driving ERF115 and PAT1 from the UBIL and EF1a promoter, respectively. Also, vectors expressing either ERF115 or PAT1 only were applied. Next, the chimeric constructs are transformed in maize (Zea mays L.) cv. B104 immature embryos by Agrobacterium tumefaciens transformation. Briefly, 12-14 days after fertilization in the greenhouse, ears of B104 plants are surface-sterilized (5% NaOCl with Tween²⁰, rinsed five times with sterile water); immature embryos are isolated and cocultivated with Agrobacteria for 3 days on cocultivation medium at pH 5.2 at 21° C. in the dark, with the scutellum side uppermost. After cocultivation and one week growth on non-selective resting medium, transformed embryogenic calli are selected on medium containing 1.5 mg/L phosphinothricin. After 3 weeks, transformation frequency is calculated by counting the number of calli generated per embryo, and regeneration potential is analysed through calli size and weight measurements. Such an experiment is applicable in many crops, and will allow to estimate the translatability and valorization potential for this unique ERF115-PAT1 complex regenerative function in planta.

An embodiment relates to the co-expression of the Arabidopsis thaliana ERF115 and Arabidopsis thaliana PAT1 in maize, leading to increased regeneration potential. Another embodiment relates to the induction of spontaneous callus formation in maize through the complex formation of maize ERF115_1 (SEQ ID NO:2) and PAT1_1 (SEQ ID NO:102). Another embodiment relates to the induction of spontaneous callus formation in maize through the complex formation of maize ERF115_1 (SEQ ID NO:2) and PAT1_2 (SEQ ID NO:103). Another embodiment relates to the induction of spontaneous callus formation in maize through the complex formation of maize ERF115_1 (SEQ ID NO:2) and PAT1_3 (SEQ ID NO:104). Another embodiment relates to the induction of spontaneous callus formation in maize through the complex formation of maize ERF115_2 (SEQ ID NO:3) and PAT1_1 (SEQ ID NO:102). Another embodiment relates to the induction of spontaneous callus formation in maize through the complex formation of maize ERF115_2 (SEQ ID NO:3) and PAT1_2 (SEQ ID NO:103). Another embodiment relates to the induction of spontaneous callus formation in maize through the complex formation of maize ERF115_2 (SEQ ID NO:3) and PAT1_3 (SEQ ID NO:104). Another embodiment relates to the induction of spontaneous callus formation in maize through the complex formation of maize ERF115_3 (SEQ ID NO:4) and PAT1_1 (SEQ ID NO:102). Another embodiment relates to the induction of spontaneous callus formation in maize through the complex formation of maize ERF115_3 (SEQ ID NO:4) and PAT1_2 (SEQ ID NO:103). Another embodiment relates to the induction of spontaneous callus formation in maize through the complex formation of maize ERF115_3 (SEQ ID NO:4) and PAT1_3 (SEQ ID NO:104). Another embodiment relates to the induction of callus formation in maize through the expression of PAT1_1 (SEQ ID NO:102). Another embodiment relates to the induction of callus formation in maize through the expression of PAT1_2 (SEQ ID NO:103). Another embodiment relates to the induction of callus formation in maize through the expression of PAT1_3 (SEQ ID NO:104). In a particular embodiment said induction of callus formation in maize through the expression of PAT1 alone occurs in the presence of low auxin concentrations.

It is to be understood that although particular embodiments, specific configurations as well as materials and/or molecules, have been discussed herein for compounds and methods according to the present invention, various changes or modifications in form and detail may be made without departing from the scope and spirit of this invention. The following examples are provided to better illustrate particular embodiments, and they should not be considered limiting the application. The application is limited only by the claims.

### EXAMPLES

1. ERF115 Interacts with SCL21 and PAT1

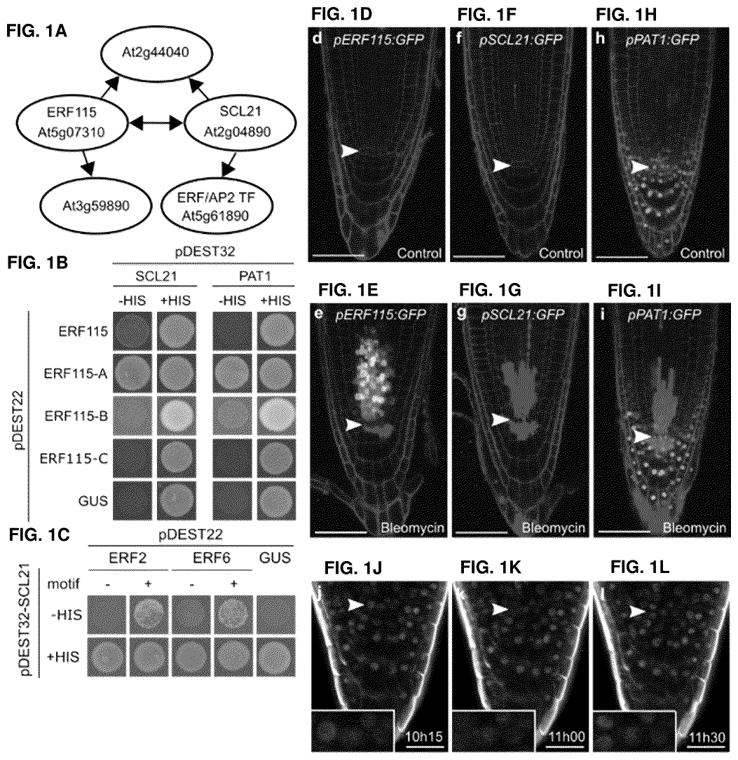

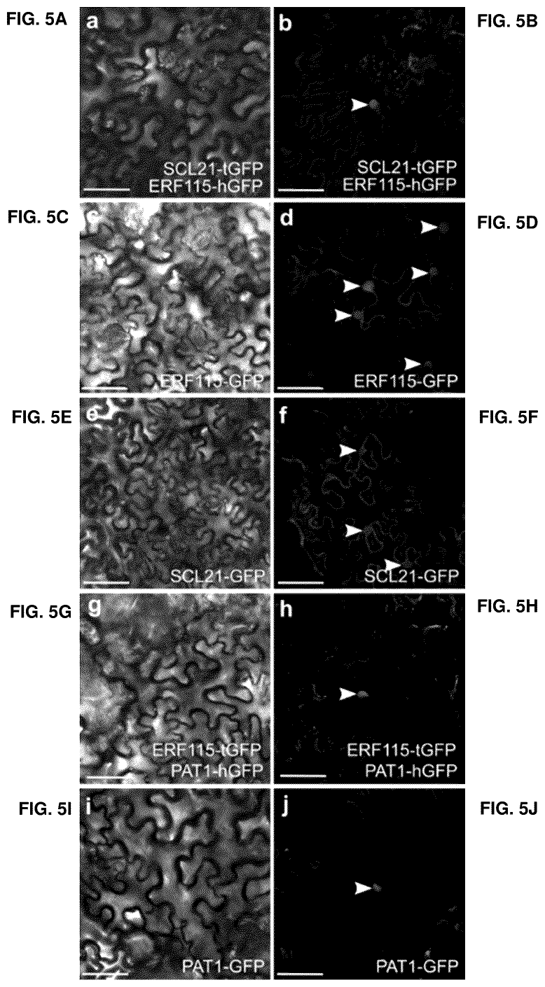

By means of tandem affinity purification (TAP), the SCARECROW-LIKE 21 (SCL21) protein was found to copurify with ERF115 (FIG. 1a). SCL21 is a member of the GIBBERELLIN-INSENSITIVE, REPRESSOR of ga1-3, SCARECROW (SCR) (GRAS) transcription factor family, including the known stem cell regulators SCARECROW (SCR) and SHORTROOT (SHR) that control the periclinal division of the endodermis/cortex initial daughter cell¹⁶. A reverse TAP experiment using SCL21 as bait verified copurification with ERF115, and also revealed interaction with ERF114, being the closest homolog of ERF115 (FIG. 1a). Bimolecular fluorescence complementation (BiFC) confirmed protein interaction between ERF115 and SCL21. Similarly, the SCL21 phylogenetically closest-related PHYTOCHROME A SIGNAL TRANSDUCTION1 (PAT1) protein¹⁷ interacted with ERF115 (FIG. 5g-j). Using the yeast two-hybrid system, the SCL21/PAT1 interaction domain of ERF115 was mapped to the N-terminal sequence that precedes the conserved APETALA2 domain (FIG. 1b FIG. 6a, b). Interaction with the full-length ERF115 was weak (with SCL21) or negative (with PAT1), suggesting intramolecular control of binding affinity. Sequence alignment of the ERF115 subclass B-4 of ERF transcription factors¹⁸ identified a conserved 11-amino-acid (AA) motif in the N-terminal region (FIG. 6c) that appeared sufficient for interaction, because chimeric protein fusions of this 11-AA domain with the distantly related ERF115 family members, ERF2 and ERF6, granted them the ability to interact with SCL21 (FIG. 1c, FIG. 6d).

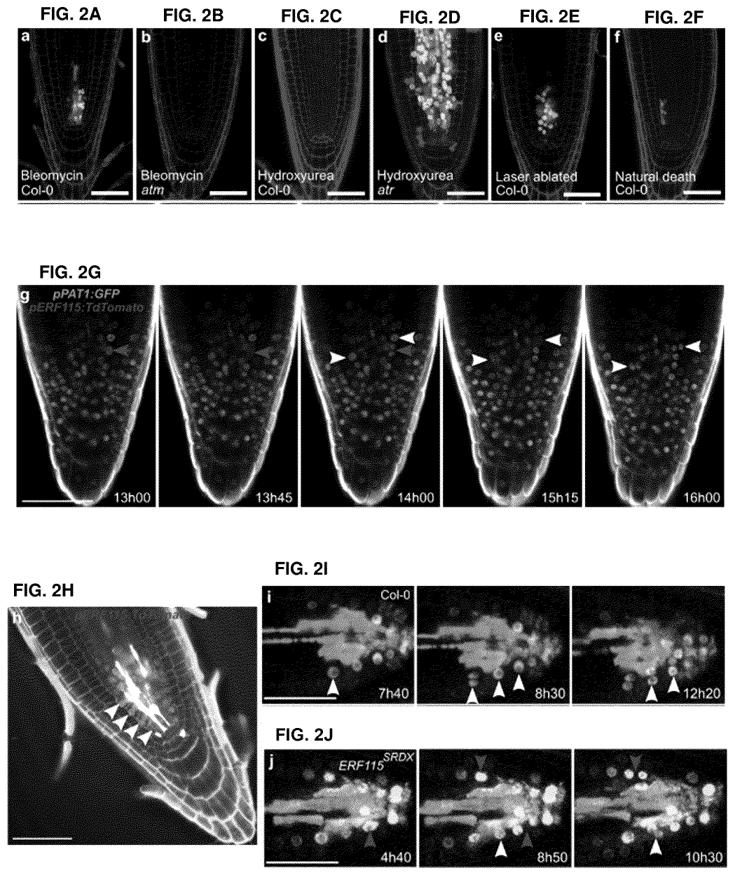

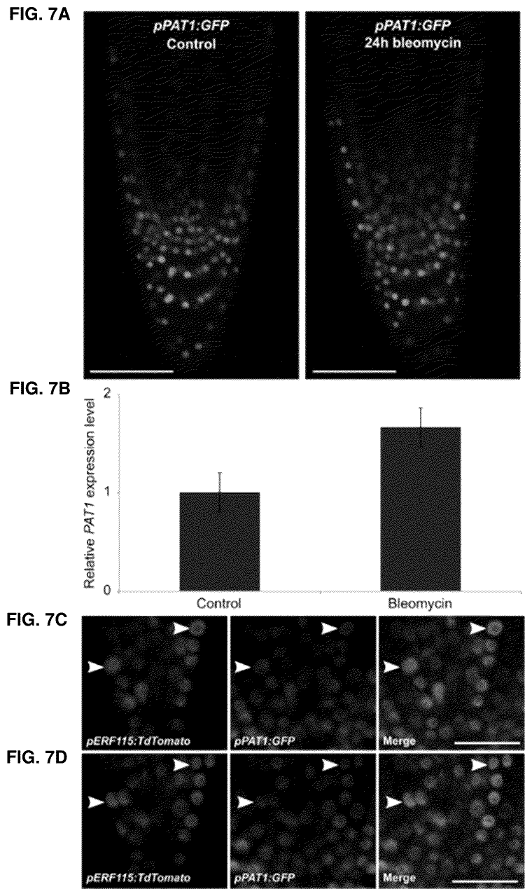

### 2. Endogenous Expression of ERF115 and PAT1 in the Root SCN

Whereas ERF115 transcripts are highly abundant in the stem cell niche (SCN) upon exposure to radiomimetic drugs such as bleomycin^{14,15} (FIG. 1d, e), transcriptional reporter lines failed to detect SCL21 expression in the root SCN under both control and DNA stress conditions (FIG. 1f, g), suggesting that SCL21 might not be a genuine interaction partner of ERF115 in the root meristem. On the contrary, PAT1 is transcribed in the SCN under both conditions (FIG. 1h, i) and showed a slight increase in expression in the meristem cells upon bleomycin treatment (FIG. 7a, b). Because ERF115 has been demonstrated to promote QC cell division after death of non-QC stem cells, ERF115 and PAT1 were expected to be co-expressed in cells engaged into a cell division cycle, a hypothesis confirmed through time-lapse imaging of dual reporter lines that allowed simultaneous visualization of ERF115 and PAT1 (FIG. 1j-l). Remarkably, ERF115 expression was not confined to the QC cells upon bleomycin treatment, but occurred as well in non-QC cells in direct contact with dead cells, which can be recognized through the uptake of propidium iodide (FIG. 2a). These data suggest an intimate relationship between cell death and ERF115 activity. Indeed, ERF115 was not induced in ataxia telangiectasia (atm) knockout plants that do not undergo meristematic cell death upon bleomycin treatment¹³ (FIG. 2b). Reciprocally, ERF115 hyperinduction could only be seen in ataxia telangiectasia and Rad3-related (atr) mutants displaying massive cell death upon treatment with the replication inhibitory drug hydroxyurea (FIG. 2d), in contrast to wild-type plants showing no cell death or ERF115 activation (FIG. 2c). Even a single cell death event, induced through cell-specific laser ablation (FIG. 2e) or naturally occurring (FIG. 2f), coincided with ERF115 expression in the cells in direct contact with the damaged cell. Time-lapse imaging demonstrated that the cell death events were tightly correlated with ERF115 induction, implying a yet to be identified communication from the dying cell to its neighbor.

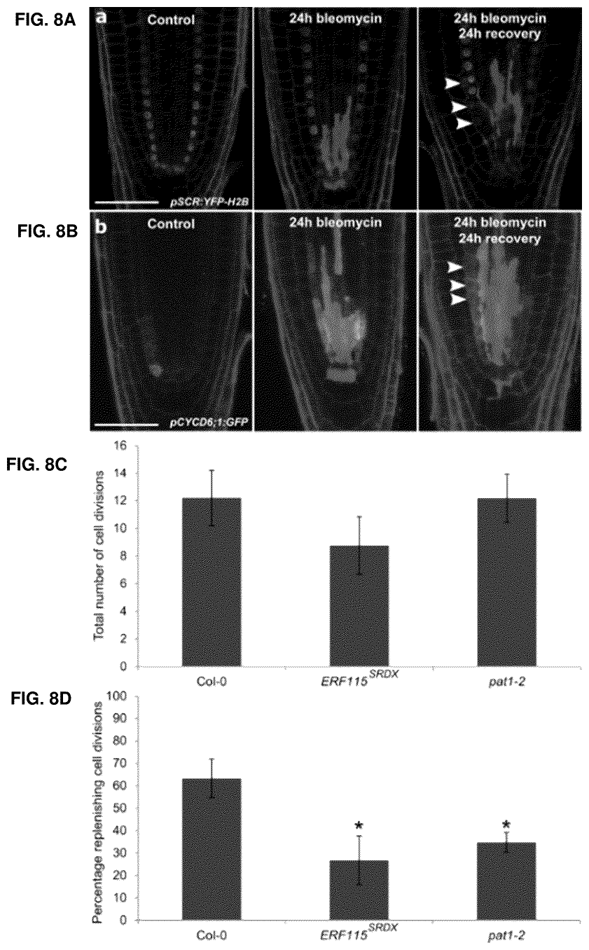

Spatial expression analysis suggested that ERF115 activity might not be restricted to the QC cells, such as observed under control conditions¹⁴, but might also be required in non-QC cells following meristematic cell death. By means of time tracking, these cells were captured to express PAT1 as well and to undergo cell divisions being perpendicular to the cell surface, being rarely observed in plants grown under control conditions (FIG. 2g, FIG. 7c, d). These cell divisions occurred predominantly in endodermal cells being in direct contact to the bleomycin killed cells (FIG. 2h and FIG. 8a) and were preceded by CYCD6; 1 expression, being a cyclin controlling periclinal endodermal cell divisions^{19,20} (FIG. 8b). These replenishing cell divisions depended on ERF115 and PAT1 activity, because they occurred less frequently after bleomycin treatment of seedlings overexpressing the dominant-negative ERF115^{SRDX} or being knockout for PAT1 (FIG. 2i, j, and FIG. 8c, d).

Bleomycin treatment mainly triggers vascular cell death and activates ERF115 expression in the neighboring non-dead vascular and endodermal cells (FIG. 2h). To study any tissue-specific response of ERF115 activation, we selectively killed a single cortex cell within the root meristem through laser ablation. Similar to the bleomycin treatment, ERF115 activation could be observed in the endodermal cells, but was absent in the epidermal cells being in direct contact with the ablated cortex cell, indicating that ERF115 induction following cell death required a tissue-specific input signal. PAT1 expression could also be detected in the cells surrounding ablated ones.

### 3. ERF115-PAT1 Function

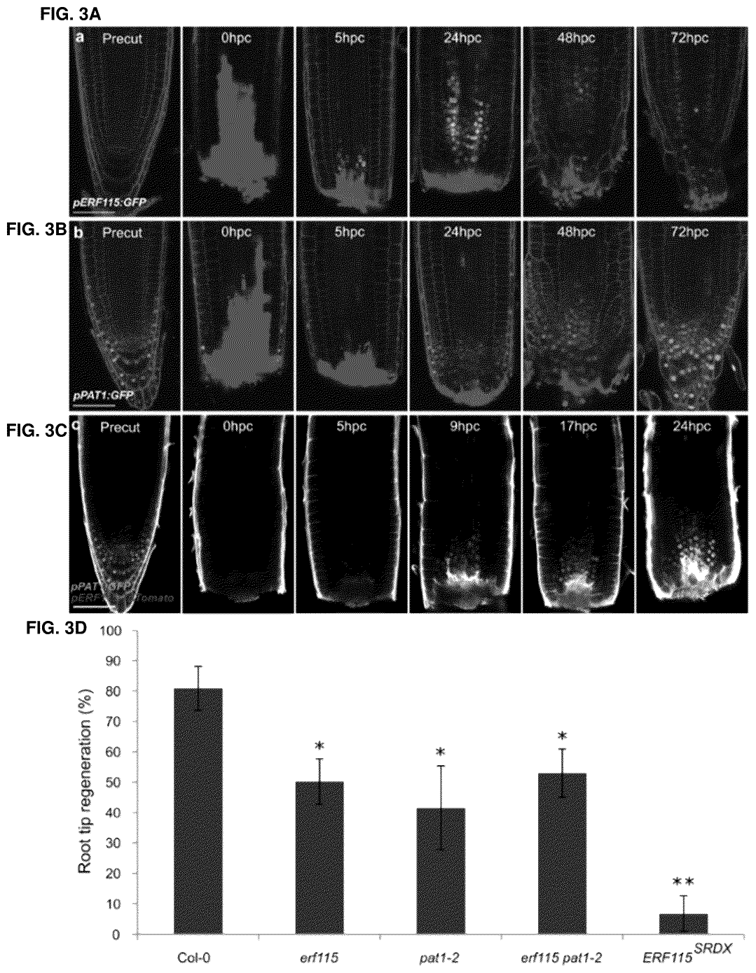

The induction of replenishing cell divisions following ERF115 expression raised the possibility that the ERF115-PAT1 transcription factor complex might play a generic role in tissue regeneration. Previously, it has been found that an excised root tip regenerates a fully functional SCN within less than three days²¹. Root tip excision triggered a rapid transcriptional induction of both ERF115 (FIG. 3a) and PAT1 (FIG. 3b) adjacent to the excision site. Co-expression of ERF115 and PAT1 is confined to the vascular and endodermal cell files (FIG. 3c), being the predominant tissues involved the regeneration of the excised root tissue²². De novo root meristem formation appeared to be dependent on the ERF115-PAT1 complex, because only 50% and 41% of erf115-1 and pat1-2 cut roots, respectively, were able to regenerate a novel SCN, compared with 81% for wild-type roots (FIG. 3d). The erf115-1 pat1-2 double mutant showed 53% root tip recovery, similar to that of the erf115-1 and pat1-2 single mutants, suggesting that the two proteins operate as an active complex in SCN regeneration, rather than in two independent pathways. The reason for the lack of a complete regeneration inhibition might be a functional gene redundancy, because ERF115^{SRDX} plants displayed a regeneration frequency of only 7% (FIG. 3d). Pre-cut roots displayed a growth phenotype being identical to that of control plants, excluding the possibility that the regeneration defects are a secondary consequence of impaired root growth.

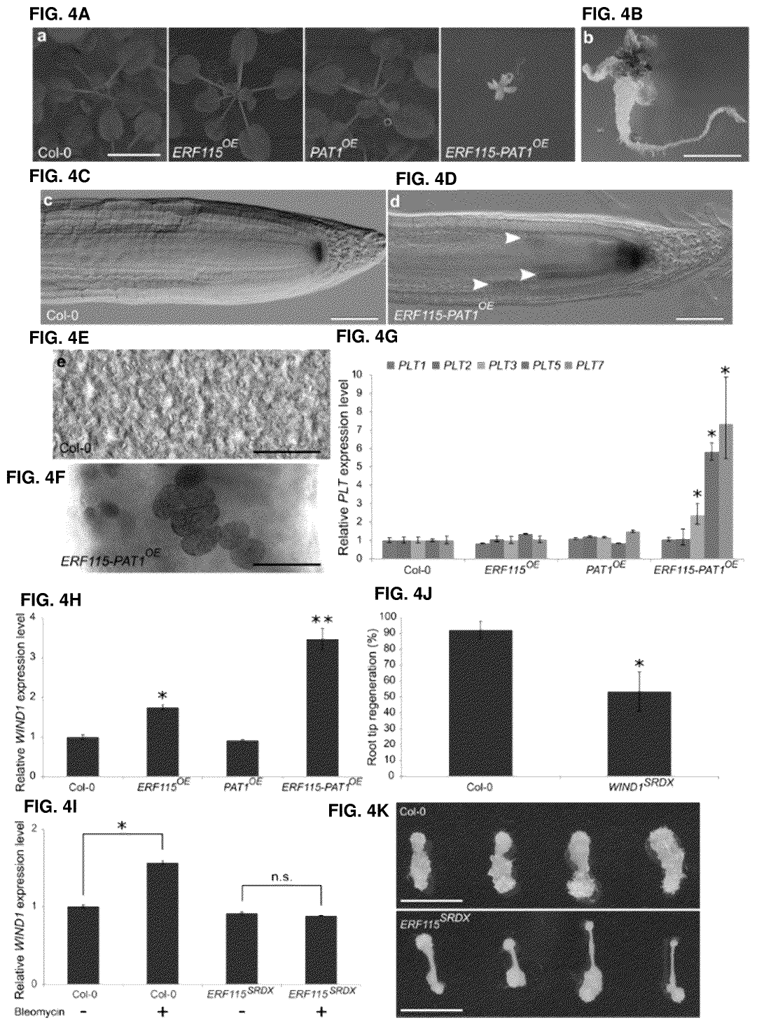

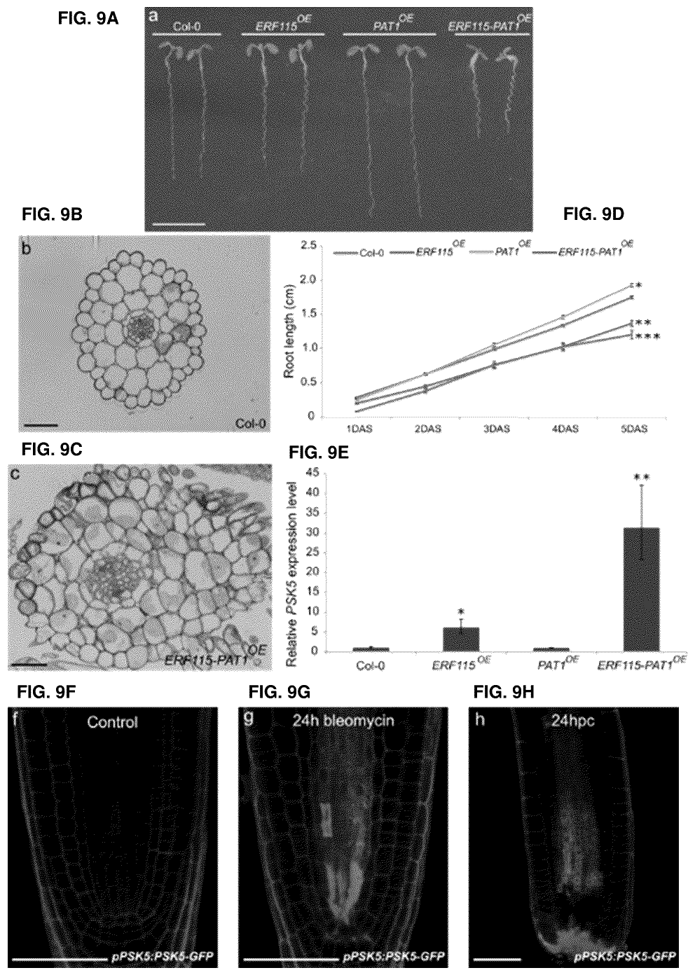

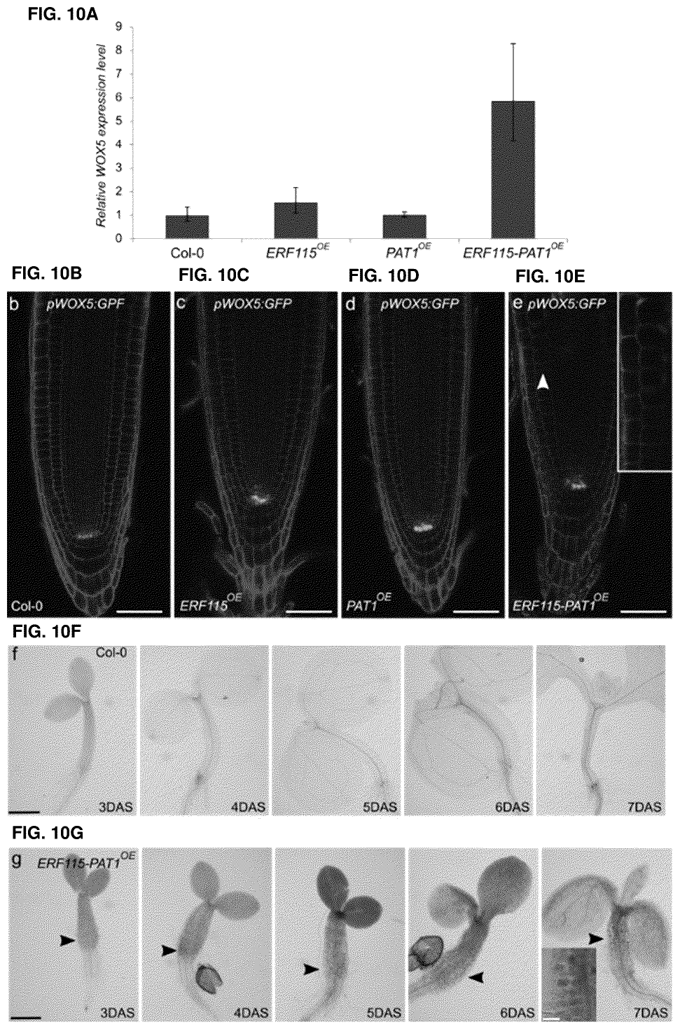

The root excision experiments suggest the ERF115-PAT1 complex to be not only responsible for the replacement of dead root (stem) cells in response to DNA damage, but for the regeneration process in general. The potent role of the ERF115-PAT1 complex in plant cell regeneration was confirmed through its ectopic overexpression. Whereas plants either overexpressing ERF115 or PAT1 displayed no outspoken growth phenotypes, seedlings expressing both (hereafter referred to as ERF115-PAT1^{OE}) displayed clear neoplastic growth, including hypocotyl thickening due to an increased number of cell layers, reduced root growth, and aberrant leaf formation (FIG. 4a-b, and FIG. 9a-d). Expression of the PHYTOSULFOKINE 5 (PSK5) peptide precursor gene, a known direct ERF115 target¹⁴, was hyperactivated in ERF115-PAT1^{OE} plants compared with control and single overexpression lines (FIG. 9e), indicative for a boosted activity of the ERF115-PAT1 complex. Correspondingly, PSK5 induction could as well be seen in the cells that display ERF115 and PAT1 expression following cell death, such as observed following bleomycin treatment or root tip excision (FIG. 9f-h) Strikingly, in ERF115-PAT1^{OE} seedlings, the QC-marker WOX5 was expressed ectopically (FIG. 4c-f, and FIG. 10a-e), preceding the development of callus-like structures, suggestive of the uncontrolled activation of regenerative divisions (FIG. 10f, g). Similarly, ERF115-PAT1^{OE} plants displayed a strong expression of the stem cell marker genes PLT3, PLT5 and PLT7 (FIG. 4g), known to play a crucial role in the initial plant regeneration stages²³.

### 4. WIND1 is Involved in ERF115-PAT1-Mediated Regeneration

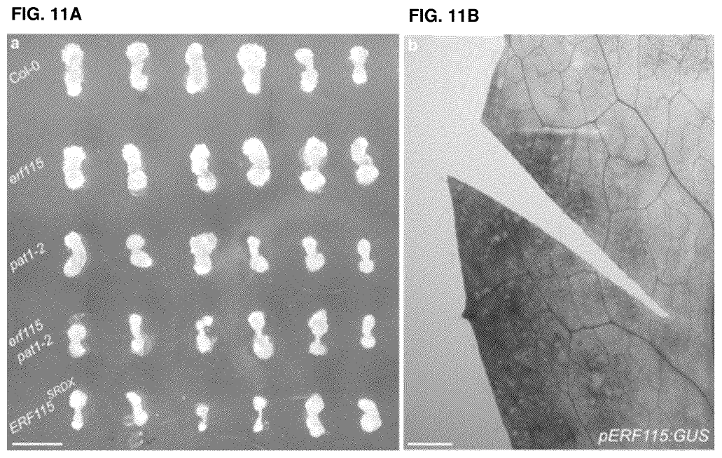

Next to PSK5, WOUND INDUCED DEDIFFERENTIATION 1 (WIND1) was identified as a putative ERF115 target gene through Tandem Chromatin Affinity Purification analysis¹⁴. WIND1 encodes a transcription factor involved in callus formation in response to wounding⁹. Correspondingly, WIND1 expression was found to be upregulated in ERF115^{OE} seedlings, which could be hyper-induced upon ERF115-PAT1 co-overexpression (FIG. 4h). Similarly, bleomycin-induced stem cell death also activates WIND1 (FIG. 4i), which appeared to be ERF115-dependent as this induction was abolished in ERF115^{SRDX} seedlings (FIG. 4i). In agreement with ERF115 and WIND1 operating in the same pathway, WIND 1^{SRDX} dominant negative seedlings display impaired regenerative capacities upon root tip excision (FIG. 4j), identical to erf115 and pat1-2 mutants. Conversely, whereas spontaneous callus is formed upon ERF115-PAT1^{OE}, ERF115^{SRDX} seedlings displayed impaired callus generation capacities when placed on callus inducing medium (FIG. 4k), similar to WIND 1^{SRDX} mutants⁹. A less pronounced effect on callus formation was observed for the pat1-2 single mutant, whereas the erf115 single mutant showed no callus phenotype. Contrary, the erf115 pat1-2 double mutant appeared to show impaired callus formation similar to ERF115^{SRDX} seedlings (FIG. 11a), again suggesting the co-involvement of ERF115 and PAT1 in callus formation. Combined, these observations suggest that WIND1 activity is at least in part involved in ERF115-PAT1-dependent tissue regeneration. However, in contrast to WIND1, ERF115 expression was not detected upon a mature leaf blade cut that does not trigger regeneration⁹ (FIG. 11b), suggesting that WIND1 activation can occur as well independent of ERF115 and suggesting that ERF115 activation upon injury is rather specific to tissues that are able to activate a regeneration program.

### 5. ERF115 and PAT1 are Required for Granting Regeneration Competence

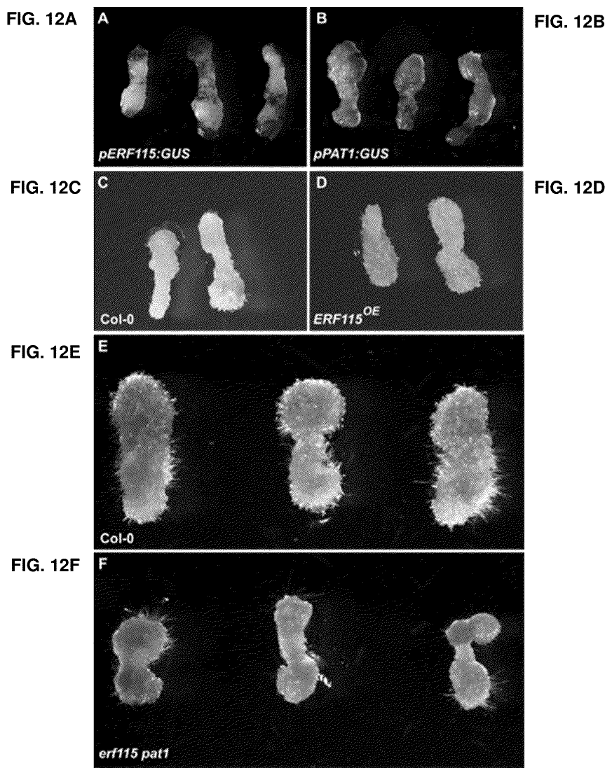

To test the need for co-presence of ERF115 and PAT1 to efficiently induce callus formation, it was initially tested whether both genes are expressed in developing calli. Seedlings of ERF115 (pERF115:GUS) and PAT1 (pPAT1:GUS) reporter lines were grown for callus induction (see methods). Histochemical GUS staining revealed both reporter lines to be strongly expressed in the calli (FIG. 12 a,b).

Subsequently, it was tested whether ectopic ERF115 or PAT1 expression do trigger enhanced callus formation. Similar as described above, calli were generated from ERF115 (35S:ERF115) and PAT1 (35S:PAT1) overexpression lines. In both cases, no calli bigger being than those obtained from control plants (Col-0) were observed (FIG. 12 c,d, data not shown), fitting with the observation that spontaneous callus formation was only observed in plants co-overexpression both ERF115 and PAT1. Co-requirement of ERF115 and PAT1 was finally demonstrated by the observation that called of erf115 pat1-2 double mutants development poorly into calli, compared to those of the control plants (FIG. 12 e,f).

### 6. ERF115 Ortholoque Sequences

SEQ ID NO: 1 depicts the amino acid sequence of the Arabidopsis thaliana Ethylene-responsive transcription factor ERF115 (263 aa).

Orthologue sequence search was supported by phylogenetic tree, orthologue searches in PLAZA (http://bioinformatics.psb.ugent.be/plaza/), and presence of putative SCL/PAT1-interaction domain (indicated in bold) (see also FIG. 6c).

The following species for identification of candidate ERF115 orthologue sequences were evaluated:
Zea mays (corn), Vitis vinifera (grape), Populus trichocarpa (poplar tree), Solanum lycopersicum (tomato), Solanum tuberosum (potato), Glycine max (soy bean), Gossypium raimondii (cotton), Brassica rapa (turnip), Hordeum vulgare (barley), Oryza sativa ssp. Indica,& Japonica (rice), Setaria italic (millet), Sorghum bicolor (sorghum), Theobroma cacao (cacao), Elaeis guineensis (oil palm), Malus domestica (apple), Fragaria vesca (strawberry), Eucalyptus grandis (eucalyptus), Citrus sinensis (orange tree), Carica papaya (papaya), Musa acuminate (banana)

> SEQ ID NO: 1: Arabidopsis thaliana ERF115-AT5G07310
MANSGNYGKRPFRGDESDEKKEADDDENIFPFFSARSQYDMRAMVSALTQVIGNQSSSHDNNQHQPVVY
NQQDPNPPAPPTQDQGLLRKRHYRGVRQRPWGKWAAEIRDPQKAARVWLGTFETAEAAALAYDNAALKF
KGSKAKLNFPERAQLASNTSTTTGPPNYYSSNNQIYYSNPQTNPQTIPYFNQYYYNQYLHQGGNSNDAL
SYSLAGGETGGSMYNHQTLSTTNSSSSGGSSRQQDDEQDYARYLRFGDSSPPNSGF*
> SEQ ID NO: 51: Arabidopsis thaliana ERF115-AT5G07310 coding sequence
> SEQ ID NO: 2: Zea mays ERF115 candidate orthologue-ZM10G15700
MERVKYCDCTVCSVQRSLCSTRRRRRRRRQIDRQLTKVDPRRRHGKRPLPAAEVEEEEEEEALPPGPPP
AKHEQLEEPHHAAVSQLQGATFSGGGGSSSSSVIGGPSPPQAYAQYYYSARADNDASAVASALAHVIRA
SPDQLPPQQAPALYGAGVPGSLRLGDHPQASAHHYPGPGGHVAAAEEEQGRRRHYRGVRQRPWGKWAAE
IRDPKKAARVWLGTFDTAEDAAIAYDEAALRFKGTKAKLNFPERVQGRTDLGFLVTRGIPDHRHPSAAV
TLAAMPPPHHQHGHQTVVPYPDLMQYAQLLQGGRGGGGHAEAAVQQAHRQQQQQQLMTMMGGRPGVNLP
STFSPSSSASAPQILDFSTQQLIRPGPPSPSPPRAAAMPSSSAAAAPSTPSSTTTASSPSGGAWPYGGE
RHRNKKDA*
> SEQ ID NO: 52: Zea mays ERF115 candidate orthologue-ZM10G15700 coding
sequence
> SEQ ID NO: 3: Zea mays ERF115 candidate orthologue-ZM02G16850
MRISLRVLISSELGTSLCTAAPSLARASVRKSKSSALSLTHARIDRSSSTHRRRRQINGQLTKVDPRRR
HGKRPLPADEEEEEEEELPPPPAKYEQLDQEEKHHVVVSQLQAGATFSGGRGSSSSSVAGPSPEAYAQY
YYSARADHDASAVASALAHVIRASPDQLPPQQAACLYGAAGAPVLRQGEGDHPQPQAAAHHHPGGHVAA
EEEQGLRRHYRGVRQRPWGKWAAEIRDPKKAARVWLGTFDTAEDAAIAYDEAALRFKGTKAKLNFPERV
QGRTDLGFVVTRGIPDHHRHPRAAAVNLAAMPQAQAQPHLQHGRPTVMPYPYPYPDLMQYAQLLQGGRG
GGDHAAAVQQQLMMMGGRGGNLPFSFSPPSSWSAPPQILDFSARQLITQPGPPSSPAAPGGAAPSTPSS
TTTASSPSASASGSAWPYGGEHHRNKKDA*
> SEQ ID NO: 53: Zea mays ERF115 candidate orthologue-ZM02G16850 coding
sequence
> SEQ ID NO: 4: Zea mays ERF115 candidate orthologue-ZM3G25810
MCFELADQRGPQGGGGAGWPAKRRAGGVQDEGAAAAAGMAMAAAGPGEVMSEYYQAQELSTMVSALTHV
VAGAPMGSAPAQRPMHGASGYYAHEMGSYRGAPSPELAGSELSSDTQSAGAAAMEEHQSAAALSSQEGP
ETPRRRYRGVRQRPWGKWAAEIRDPHKAARVWLGTFETAELAARAYDEAALRFRGSRAKLNFPEDARLY
PASTAGAAAPLAAAASTSPPVYSGGVQGSSDYLRYHQMLLQASTGSQGTLLPFYGGGMSNPYGGGAAMT
GSYGGAGGGNTSGSLGSYYSFPASSVSVATVPSSTSSASGYYYSSPHDSQHSEASAAADWNWESALAWP
DSSQYPPPPHTQ*
> SEQ ID NO: 54: Zea mays ERF115 candidate orthologue-ZM3G25810 coding
sequence
> SEQ ID NO: 5: Vitis vinifera ERF115 candidate orthologue-VV17G09050
MSAMVSALTQVIGNTDKNPLHDLGNPSPISHHSATTPHDQPSQLLQDQGNQLRRRHYRGVRQRPWGKWA
AEIRDPNKAARVWLGTFDTAEDAALAYDEAALRFKGNKAKLNFPERVQGRSELGYLTNPPSRWRW*
> SEQ ID NO: 55: Vitis vinifera ERF115 candidate orthologue-VV17G09050
coding sequence
> SEQ ID NO: 6: Vitis vinifera ERF115 candidate orthologue-VV17G09050
MSAMVSALTQVIGNIDKNPLHDLGNPSPISHHSATTPHDQPSQLLQDQGNQLRRRHYRGVRQRPWGKWA
AEIRDPNKAARVWLGTFDTAEDAALAYDEAALRFKGNKAKLNFPERVQGRSELGYLTNRQDFLLPQQQQ
LPNPAVPPLPHPSLPRPSYPNLHHYAQLLPGGGGDLNHAMSSLYGREASTIQSLSTISSSSSITSHPQH
HQRRRQREEEELQQPQLLQFSSLFGSSSSNDPHNNRRDD
> SEQ ID NO: 56: Vitis vinifera ERF115 candidate orthologue-VV17G09050
coding sequence
> SEQ ID NO: 7: Populus trichocarpa ERF115 candidate orthologue-
PT12G10850
MDVMVSALAQVIGSSHNSSAQVQENPLTSTQSSTENDQTQPAVQDQGNARRRHYRGVRQRPWGKWAAEI
RDPKKAARVWLGTFETAEAAALAYDEAALRFKGSKAKLNFPERVPSGGTELGFFTRGQGLHTVTEPVEN
HIMAPLARSQRSQEAINPNNFQYPQFLGTTSGYGLSHVMPPAVPFGGETFLSPTSSSASSNSWPISSQQ
QQQQQEELLRLSMQFGSSYNSRYDPSKYKDEGL*
> SEQ ID NO: 57: Populus trichocarpa ERF115 candidate orthologue-
PT12G10850 coding sequence
> SEQ ID NO: 8: Solanum lycopersicum ERF115 candidate orthologue-
SL06G068830
SDRIRRGKRRYESEEKEDRNYNHMYSSARSQHDMSTMVAVLSQVIGNKSTTNTNSSSSSSAHHKPLLTL
NHQSNTTAAMQNQLPQLNQQQGNNEKRRRQYRGVRQRPWGKWAAEIRDPEKAARVWLGTFHTAEDAAIA
YDEAALKFKGNKAKLNFPERVQSTTDQFGISYLITNTNHQQHQFQPTNFLPNSDQLQQHHYSNHNADDL
KFGVSPSFYHPTGFNPKALDLVEPSKSSSMTYLVQQASSHQVQEEPRYINHQQEDENNLKFSSYFGTYS
SSGPTLGEFEDQK*
> SEQ ID NO: 58: Solanum lycopersicum ERF115 candidate orthologue-
SL06G068830 coding sequence
> SEQ ID NO: 9: Solanum lycopersicum ERF115 candidate orthologue-
SL03G118190
MKRSSSNNDQRDEKDTSNIFPIYSSARSQHDMSAMVSALSQVIGNSSSSASGDSSSVHVNPLTLIQQHQ
SQSSTQDQERRRYRGVRQRPWGKWAAEIRDPKKAARVWLGTFETAEGAALAYDEAALRFKGNKAKLNFP
ERVQGQFFQCYDQPATSSNNTSEQNYPNVHHYADLLLRTDNNIDLNFDVSPNTFYHSFDISQSSMEVPV
YHEEQQQVITTHEEEEEDFVKYRGSHFGNSTSSGGTK*
> SEQ ID NO: 59: Solanum lycopersicum ERF115 candidate orthologue-
SL03G118190 coding sequence
> SEQ ID NO: 10: Solanum tuberosum ERF115 candidate orthologue-
ST06G025710
MDRTRHGKRPYESEEKEDTNNNQMYSSARSQHDMSTMVSVLSQVIGNKSRTNTNSSSSSSAHHKPLLTL
NHRSSTTAAMQNQLPQLNQQQGNNERRRRQYRGVRQRPWGKWAAEIRDPEKAARVWLGTFHTAEDAAIA
YDEAALKFKGNKAKLNFPERVQSATDQFGISYLITTTNQFPANNFLPNSDQLQHHYAPAGGSNHNADDL
NFGVSPSSYHPTGKSTVNDLSTQ*
> SEQ ID NO: 60: Solanum tuberosum ERF115 candidate orthologue-
ST06G025710 coding sequence
> SEQ ID NO: 11: Glycine max ERF115 candidate orthologue-GM06G17180
MEGRSISHSSEREEEYDLFPIYSERSQQDMSAMVSALTQVIGGSNSDSLQQHEGLLTSSHNNTSTQNNN
EQSQAPQQEQGSVRRRHYRGVRQRPWGKWAAEIRDPKKAARVWLGTFETAEAAALAYDEAALRFKGSKA
KLNFPERVQGTASEFGYHLTNQHSTSSHDQQASNPIITPHFATTQETYSPSHHFQYAQQQLMGGGSNSF
NNNQDMLRFYGGHNNFVSSQQSASSSSSTALSQNQQDELLRFSMQFGASSHSDHSGNWRGGQ*
> SEQ ID NO: 61: Glycine max ERF115 candidate orthologue-GM06G17180
coding sequence
> SEQ ID NO: 12: Gossypium raimondii ERF115 candidate orthologue-
GR08G21060
MNAMVHALAQVIGNNNSNPLLQLHDDQHPNPTAQQNQSHQQPQPQDQGNARRRHYRGVRQRPWGKWAAE
IRDPKKAARVWLGTFETAEAAALAYDDAALRFKGSKAKLNTPERVQGRLESSYLTTTRQELERTEAPPH
PPPTYPNISQYAQLLSGGLPNTAFNYAMPSGAAYGSWPAFTTSSHSSSSSSSSTTLTSQQQGYMGGFSL
HFGGSSPTSDHTNNMGDYDYYYSRDQ*
> SEQ ID NO: 62: Gossypium raimondii ERF115 candidate orthologue-
GR08G21060 coding sequence
> SEQ ID NO: 13: Brassica rapa ERF115 candidate orthologue-BR03G02730
MNNGKRPFRAGESEEKKEADDDENIFPFFSARSEYDTRAMVSALTQVIGNQSSTHDNNLHHPVEYDQQD
PIQHVPPTQDHGNLRKIHYRGVRQRPWGKWAAEIRDPQKAARVWLGTFETAEAAALAYDEAALKFKGSK
AKLNFPERAQLASNTSTITGLPNYYSSNNQTYYSNPQTNPQNIPYYNQYYYNQYLQQGGNSNDALSYSL
AGGETGGSIYSQTLSNTTSSPAGGSLRQQEDYTRFWHFGDSSPNSGV*
> SEQ ID NO: 63: Brassica rapa ERF115 candidate orthologue-BR03G02730
coding sequence
> SEQ ID NO: 14: Brassica rapa ERF115 candidate orthologue-BR10G25380
MMPFGAGESDERKEADDEENIFPFLSARSQYDTRAMVSALTQVIGNQSSTHDSNQHHPVEYNQQDPIQH
VPPTQDQGNLRKRHYRGVRQRPWGKWAAEIRDPQKAARVWLGTFETAEAAALAYDEAALKFKGSKAKLN
FPERAQLASNASTITGLPNYHSSNNQMYYSNPQTNPQTMPYYNQYYYNQYLQQGGNSNDALSYSLAGGE
TGGSMYNHQSISNTTSSSSGGSSRPQQEQDYARFWHFGDSSPSSGF*
> SEQ ID NO: 64: Brassica rapa ERF115 candidate orthologue-BR10G25380
coding sequence
> SEQ ID NO: 15: Hordeum vulgare ERF115 candidate orthologue-
HV53830G00010
MVSALSHVIRATPDQEPAYYPAGPAAVSREQQHQHAAAIAEEQGRKRHYRGVRQRPWGKWAAEIRDPKK
AARVWLGTFDTAEDAAIAYDEAALRFKGTKAKLNFPERVQGRTDLGFVVTRGIPDRLQQQQHYPAAVGA
PAMRPPLHQQQAVVPYPDLLRYAQLLQGAGSAGGAVNLPFGAMSPPSMSSSSSPHILDFSTQQLIRVSP
ASPAAAISGSATTGPSTSSSTTTASSPGAAWPYTGEQKNNKDS*
> SEQ ID NO: 65: Hordeum vulgare ERF115 candidate orthologue-
HV53830G00010 coding sequence
> SEQ ID NO: 16: Oryza sativa ssp. Indica ERF115 candidate orthologue-
OSINDICA_04G24720
MVTALAHVIRAAPDLHLPHHPSSSASAAAHPQQASSFYPTAAAAASSPSDQLAAAAAAAEEQGRRRHYR
GVRQRPWGKWAAEIRDPKKAARVWLGTFDTAEDAAIAYDEAALRFKGTKAKLNFPERVQGRTDLGFLVT
RGIPPAATHGGGYYPSSSPAAGACPPPRQQQTVVPYPDLMRYAQLLQGGVGGSYMPFGGAATMSSSTVS
SSSAPQILDFSTQQLIRAGPPSPMPSSGSGSATAAASSTTSASSPGAWPYGGSERKKKDSSS*
> SEQ ID NO: 66: Oryza sativa ssp. Indica ERF115 candidate orthologue-
OSINDICA_04G24720 coding sequence
> SEQ ID NO: 17: Oryza sativa ssp. Japonica ERF115 candidate orthologue-
OSINDICA_04G24720
MVTALAHVIRAAPDLHLPHHPSSSASAAAHPQQASSFYPTAAAAASSPSDQLAAAAAAAEEQGRRRHYR
GVRQRPWGRWAAEIRDPKKAARVWLGTFDTAEDAAIAYDEAALRFKGTKAKLNTPERVQGRTDLGFLVT
RGIPPALTHGGGYYPSSSPAAGACPPPRQQQTVVPYPDLMRYAQLLQGGVGGSYMPFGGAATMSSSTVS
SSSAPQILDFSTQQLIRAGPPSPMPSSGSGSATAAASSTTSASSPGAWPYGGSERKKEDSSS*
> SEQ ID NO: 67: Oryza sativa ssp. Japonica ERF115 candidate orthologue-
OSINDICA_04G24720 coding sequence
> SEQ ID NO: 18: Setaria italica ERF115 candidate orthologue-
SI007G08350
MPGSIDPAPSADGRRRRRQIDRQLTKVDPRRHGKRPLPADKEEEDQPPPPPPAKHEQLEIEEHRYHVSQ
LQQGATFSAGGGGGGSSSSSAAGAAAGPSPEAYAQYYYSARADHDASAVASALAHVIRASPDQLPPHAF
GGGGAPPGQGDYQQAAPPAAAAAAAEEEQAAGRRRHYRGVRQRPWGKWAAEIRDPKKAARVWLGTFDTA
EDAAIAYDEAALRFKGTKAKLNFPERVQGRTDMGFLVTRGIPDRHHHQGGAAVTLAAMPPPHRQHHQTV
VPYPDLMQYAQLLQGGGRGGGGAGDHHAEAAAQQAQARLMMMARGGVSLPFGAASFSSSSSSAPQILDF
STQQLIRPGPPSPAAAAPSTPSSTTTASSPGGSAWPYGGEHHRNKKDA*
> SEQ ID NO: 68: Setaria italica ERF115 candidate orthologue-
SI007G08350 coding sequence
> SEQ ID NO: 19: Sorghum bicolor ERF115 candidate orthologue-
SB02G026630
MTKKLISAMAGKQGFKEQQFNDQRRQQASIQGDDIAKSLVGFGGGGGRLISHEQEDAIIVAALRHVVSG
YSTPPPEVVTVAGGEPCGVCGIDGCLGCDFFGAAPELTQQEAVNFGTGQMVATAAAAAAGGEHGQRTRR
RRKKNMYRGVRQRPWGKWAAEIRDPRRAARVWLGTFDTAEEAARAYDCAAIEFRGARAKLNFPGHEALL
PFQGHGHGGDACATAAANAETQTTPMLMTPSPCSADAAAAAPGDWQLGGGVDGGEGDEVWEGLLQDLMK
QDEADLWFLPFSGAASSF*
> SEQ ID NO: 69: Sorghum bicolor ERF115 candidate orthologue-
SB02G026630 coding sequence
> SEQ ID NO: 20: Theobroma cacao ERF115 candidate orthologue-
LOC18608834
MTAMVTALTHVMGTGGSDEQLSFTPSSVPLSQSAVKEEPDPPQPVQDQENTRRRHYRGVRQRPWGKWAA
EIRDPKKAARVWLGTFDTAEDAALAYDRAALKFKGTKAKLNFPERVQGNTEVSYFTGHGDSSTVRPDQN
PTPAATPPSWSQDSYPHLFQYAQLLSSSNDADISYYTSNLFNQEPLSPQFPSMAASPNISSQYHHQDQT
RFSTKYESSSGSDYPEQYGKDSDPSNRSE*
> SEQ ID NO: 70: Theobroma cacao ERF115 candidate orthologue-
LOC18608834 coding sequence
> SEQ ID NO: 21: Elaeis guineensis ERF115 candidate orthologue-
LOC105060414
MVSALAHVISSSLSGVGVGVGRSESVVIQSELNPAMAGPESGSMERELSQPSEEQGNVRRRHYRGVRQR
PWGKWAAEIRDPRKAARVWLGTYNTAEEAAIAYDEAALRFKGTKAKLNFPERVQGRTDLGFLVSRGIPE
RPPQPITPPPTASYPDLLQYAQLLQSRDEDLHSVASGLFVTDSFTSGSSQVSYHSTSGSSQEFLDFFSQ
MRSSSSSSSRQPRGDQKDKDSNQQQ*
> SEQ ID NO: 71: Elaeis guineensis ERF115 candidate orthologue-
LOC105060414 coding sequence
> SEQ ID NO: 22: Elaeis guineensis ERF115 candidate orthologue-
LOC105051098
MVSALAHVISSSSPGVGVGVGVGGGETREIQPELSPAMAGTGSGSMEIRELSQPSQEQGNVRRRHYRGV
RQRPWGKWAAEIROPKKAARVWLGTFDTAEQAAIAYDEAALRFKGTKAKLNFPERVQGRTDLGFLLSRG
IPERQPEPITPSAAATYPDLLQYAQLLQSRDEDFHNVASGLYIGGSFASGSSQMSPASMSGSSQEFLDF
SSQFGTSSSSTSWPHGDQKDKDSSQHP*
> SEQ ID NO: 72: Elaeis guineensis ERF115 candidate orthologue-
LOC105051098 coding sequence
> SEQ ID NO: 23: Malus domestica ERF115 candidate orthologue-
LOC103433166
MSAMVSALTQVIGTTDDHAAVQPNPTSISDSSLLVKQEPDRSQPVQDQEPVRRRHYRGVRQRPWGKWAA
EIRDPKKAARVWLGTFETAEDAAIAYDNAALKFKGTKAKLNFPERVQGKTDLGILMGSSGSGAASTQRT
QNLMTPAGHIVNPQPAPAPLMMSQQPETFPDLYQYARLLSGNDADFYNYSSYPFNQDPRFTSRFLPSST
HFSSSTASQDSQPPQQGQQDHEEDGGNEDRNWSNPRE*
> SEQ ID NO: 73: Malus domestica ERF115 candidate orthologue-
LOC103433166 coding sequence
> SEQ ID NO: 24: Malus domestica ERF115 candidate orthologue-
LOC103420552
MIFDPFCSSARSQHEMSAMVSALAQVLGSNNSQTPAVQEPVEPPLITPQSSAMELHDGQSPQQAQDQGN
VRRRHYRGVRQRPWGKWAAEIRDPKKAARVWLGTFETAEAAAISYDEAALRFKGSKAKLNFPERVQGRI
TELGYLTTTSTQQNLPAEAQSITDHHQPLPDHQYQLQAFPNNVATPHDQYAPYFLSGNEGVNYDDLPTN
LSERERFAFQTSETTSSSLLPFLPSHQQEDQQHPLNYSMPAFGSSSSSSSNPPPRNRNRRP*
> SEQ ID NO: 74: Malus domestica ERF115 candidate orthologue-
LOC103420552 coding sequence
> SEQ ID NO: 25: Malus domestica ERF115 candidate orthologue-
LOC103454881
MIFEPFCSSARSQHEMSAMVSALSQVLGSTNNQTPAVQVPMEPPMIAPQSSAMESHDDQSPQHARDQGT
ARRRHYRGVRQRPWGKWAAEIRDPKKAARVWLGTFETDEAAALAYDEAALRFKGSKAKLNFPERVQGRI
TELGSLTATSTQQMLPAGTSQGVITDHHHPLSDXQYQLQAFPNNVVTPPHDQYAQYFRSGHECISYDLP
ATLYEREIFAXQTSETTTSSSSLLPFLPSHQQEDQQHPLSYSMPGFGSSSSSSSNPPPRNRNRRP*
> SEQ ID NO: 75: Malus domestica ERF115 candidate orthologue-
LOC103454881 coding sequence
> SEQ ID NO: 26: Malus domestica ERF115 candidate orthologue-
LOC103438752
MYDIATNSQFDVHKVDRKHGKRLLASVESEEKEEDQIFPVYSTRSQQDTSAMVSALAQVIGKNSDQINN
PLDQVQGINPLITSQSSPTETQSQTVLQDQGNLRRQHYRGVRRRPXGKWAAEIRDPVKAARVWLGTFDT
AEAAALAYDKAALKFKGSKAKLNFPERVQGISESGCLALITTDQQDSLNINPPQPNITRPTSSSVFDCT
QYNNVTSSTSLLSSSSMPSQQAAELPSFSMQFGSSFSSSSSGPPHKYRKDFDSSHSR*
> SEQ ID NO: 76: Malus domestica ERF115 candidate orthologue-
LOC103438752 coding sequence
> SEQ ID NO: 27: Malus domestica ERF115 candidate orthologue-
LOC103438754
MVSALAQVIGKNSDQINNPLGQVQGINPLTTSQSSPTETQSQPVLLQDQGNLRRQHYRGVRRRPSGKWA
AEIRDPVKAARVWLGTFDTAEAAALAYDKAALKFKGSKAKXNFPERVQGSSESGCLTLITTDQQDSLNI
NPPQPNITRPTSSSVFDCTQHNNVIYCTSLLSSSSMPSQQAAELPSFSMQFGSSFSSSSSGPPHKYRKD
FDSRHSR*
> SEQ ID NO: 77: Malus domestica ERF115 candidate orthologue-
LOC103438754 coding sequence
> SEQ ID NO: 28: Malus domestica ERF115 candidate orthologue-
LOC103415143
MFFRYTFLLGIRAVSSVVFRSSVVEAILVTTTALQYVQTTRTTISAATSTCSRVKLDKVDLKHGKRPLA
SVESEEKEEDQIFPVYSARSQQDTSAMVSALAQVIGNNSDQINNPLDQVQGISSLITSQSSPTETQSQP
VLLQDQGNLRRQHYRGVRRRPWGKWAAEIRDPIKAARVWLGTFNTAEAAALAYDGAALRFKGSKAKLNF
PERVVQGSSESGCLTITTQLQHSLNNIPPEANISRPTYSNVFDYARYNNVTSTSSSSMPSQQAAELRSF
SMQFGSSSSSSSGPPYKYRKDFDRSHSR*
> SEQ ID NO: 78: Malus domestica ERF115 candidate orthologue-
LOC103415143 coding sequence
> SEQ ID NO: 29: Fragaria vesca ERF115 candidate orthologue-
LOC101303581
MSAMVSALTQVIGNPEEDNKQVQSNPASVKDEPDRSQPVQDQESTVRRRHYRGVRQRPWGKWAAEIRDP
KKAARVWLGTFETAEDAAIAYDNAALKFKGTKAKLNFPERVQGNSSILIQGSSGTTSSGSSVSTERNRS
RPALTVPHHDASYVAPSQPQQESSSFPDLYQYAQLLQSNDIDFSSNYQYPPNNPFNQDYHPQYSTPQFP
PSTYYPSHQQQGQQDDHVQEDHQNENKNWNRRNPSE*
> SEQ ID NO: 79: Fragaria vesca ERF115 candidate orthologue-
LOC101303581 coding sequence
> SEQ ID NO: 30: Fragaria vesca ERF115 candidate orthologue LOC101309928
MSAMVSVLSRVISGDSSTDTDPNPALLQLPQQSSTATPELDQSHQQAAPDQAGSVRRRHYRGVRQRPWG
KWAAEIRDPKKAARVWLGTFETAEAAALAYDEAALRFKGSKAKLNFPERVQGTSESGGYLTTQTVAHQP
LISDYHQQQLVYPNNITTTDQYYPQFYGNLNYGQPDPRFYNQPATSSYPPFIPISQAQEEDRQQQPALS
SPMPDFGSPSSSLHPEYPQYDTTRNFDNSHWRG*
> SEQ ID NO: 80: Fragaria vesca ERF115 candidate orthologue LOC10130S928
coding sequence
> SEQ ID NO: 31: Fragaria vesca ERF115 candidate orthologue-LOC101292641
MAKEQEHAIMVSALEQVIGGGIATRTSGTSQNHCQYATSAAEAGTNKKVVILVSDGDTCQVCKIDGCLG
CEFFPPSNKHGKGKRVKKSKYRGVRQRPWGKWAAEIRDPRRAVRVWLGTFQTAEEAARAYDKAAVEFRG
EKAKLNFPRISSEAGTSTAALTKQSMETDDEVHNQVNPSNEKSANQELGQGSDVKDDEIDRFIWKMLKD
DDGDEDLSTMVNSNMLN*
> SEQ ID NO: 81: Fragaria vesca ERF115 candidate orthologue-LOC101292641
coding sequence
> SEQ ID NO: 32: Fragaria vesca ERF115 candidate orthologue-LOC101309634
MVSALTQVIGNTTHEQNPLQVIDHQVLGNPQVFSAENPVEQSQPAVVLQAQGNVRQHYRGVRRRPWGKW
AAEIRDPHKAARVWLGTFETAEAAALAYDEAALRFKGSKAKLNFPERVQGISPSTSSGLYLAISTSTGH
DHRLANSAPPAAPISRPTTYSINPNNVNIDYDALVSSSQNHGQERTMPQINVQTTLSSTSSASSMPSHH
QQERVQQQEDQQQLLKFPIMPFGGSSSSSSDPPTKYRRDSSGSGDFRR*
> SEQ ID NO: 82: Fragaria vesca ERF115 candidate orthologue-LOC101309634
coding sequence
> SEQ ID NO: 33: Eucalyptus grandis ERF115 candidate orthologue-
LOC104433731
MVSALSRVISTSDDAPSSADDPAAPVQEEHGDPPQQAPDQESVRKKHYRGVRQRPWGKWAAEIRDPKKA
ARVWLGTFETAEDAALAYDRAALKFKGTKAKLNFPERVQGKPEYAAYSNPSHQNSGVNVLPEQINPQPA
PFVPYPHAAFPDLAQYAQLLSSNDAEFPYYVSNLYGQEPFGSQQSSSTSSSSISSSSYHYNQQQQQQEP
QNEPSRTSFGWSPSNYDFQGYGDGFDPRNQGQ*
> SEQ ID NO: 83: Eucalyptus grandis ERF115 candidate orthologue-
LOC104433731 coding sequence
> SEQ ID NO: 34: Eucalyptus grandis ERF115 candidate orthologue-
LOC104426218
MEGRNWKRSKSQAGHVSEETEDGDRKRNNYAYPSSLMETARSQQDTSAIVSALAQVIANPAAAASHHAS
LSSSASLSQSSLHDHQAPDAQVGKNKKLEVSRNYRGVRQRPWGKYAAEIRDPKKAARVWLGTFDTAEGA
ALAYDEAALRFKGNKAKLNFPERVHSLPPPYGPACNASQPQSQLLPPAFSSCDNHVGAQLMDGCAMPPP
RPSYVRGQGPTSSASASDYFQSLQPTPSLSSSSSMPSPFHQHDPQHSFDGFSSSWRSSHE*
> SEQ ID NO: 84: Eucalyptus grandis ERF115 candidate orthologue-
LOC104426218 coding sequence
> SEQ ID NO: 35: Eucalyptus grandis ERF115 candidate orthologue-
LOC104425760
MEGRNWKRSKSQAGHVSAEREDGDKKRNNYAYPSSLMETGRSQQDTSAIVSALAQVIANPAAAESHHAS
LSSSASLSQSSLHDHQAPDAQVGKNKKLEVSRNYRGVRQRPWGKYAAEIRDPKKAARVWLGTFDTAEGA
ALAYDEAALRFKGNKAKLNFPERVHSLPPPYGPASNASQPQSQQLPPAFSSCDNHVGAQLMDGCAMPPP
CPSFVRGQGPTSSASASDYFQSLQPTPSSSSSSSTTSPFHQHDPQHSFDGFSSSWRSSQE*
> SEQ ID NO: 85: Eucalyptus grandis ERF115 candidate orthologue-
LOC104425760 coding sequence
> SEQ ID NO: 36: Eucalyptus grandis ERF115 candidate orthologue-
LOC104415849
MSGLKVADRGDKAPILYPGLDRERETSAMVLALARVVAGEVPGDAEESCPFPFPSGVLRLKRGHGDLSA
EPSAEAQLRRAPGRESSVDDAARGIMEGPSMKTTNHATPTYEYSNSTAAMSMSNDEHQPRRKYRGVRQR
PWGKWAAEIRDPVKAARVWLGTFETAEGAAQAYDVAALKYRGNKAKLNFPENvvARLSLAAPPATQMTV
PDAARTHVTVPADTEHQLASGPDHGCGEWCSWLSPDPDLAHSNLLPSSSSPSSSSSSVSKAASFAFPTR
SGSWPF*
> SEQ ID NO: 86: Eucalyptus grandis ERF115 candidate orthologue-
LOC104415849 coding sequence
> SEQ ID NO: 37: Eucalyptus grandis ERF115 candidate orthologue-
LOC104450834
MEEAVMPMYSPYCPPGETSAIVSALTHVVTGTRGGQHGGAYQSTLAPSFAYDSASAGSSSQLPWTYIGQ
KRERDEAGSSSQFLAEPPLSQRDYYGIYGGSFALRETSAISASLPGLQARTVAATSNVPPPPSGAGPPP
YEGGERRRRYRGVRQRPWGKWAAEIRDPQKAARVWLGTFDTAEAAARAYDEAALRFRGNRAKLNFPENV
RVIPPNVPTYGSPAAAAATLAAGSAPPAIAAGQLAPPYAGAVPLYPRGSEGFNLGDYWEYSQLLQGHHH
HQPASLMEQMMYSSQMASSHSSLSLPSSSPPQPPPPPPSFPSSDLYGSSSSSGFGGASSFSSSVPLFFP
QQPPLGYFRPPPPRPPHDQGSGGEDSEPPPSSDSSHYHSSTS*
> SEQ ID NO: 87: Eucalyptus grandis ERF115 candidate orthologue-
LOC104450834 coding sequence
> SEQ ID NO: 38: Eucalyptus grandis ERF115 candidate orthologue-
LOC104418178
MSIMVSALRRVVSGEVLSGDDQQQQFNLSGGYEPAALLGRSSSLDSVGVGQKRGRGDSTSSSEFLVSQD
SVAFGGFPQAGSSSSSDARGHAGSTTVETAKADTADGTAPKYEYNYEAPTTMAASRLDPTVRRKYRGVR
QRPWGKWAAEIRDPFKASRVWLGTFDTAEGAARAYDRAALQFRGSKAKLNFPENVRLRQQPAPVASAAF
PSATHFTISRELPASGNFDATGYDGQAPMQQFPENDFREYHRDVAAHQERLQGRTMSLYEQMLFSNSSF
GSQFQPAFSLSPSSSSSSSSVLARSPNSSLFMPSPPNVTSRSQESRRGSGDGAAVFSQHPWTDSSHYSS
SSG*
> SEQ ID NO: 88: Eucalyptus grandis ERF115 candidate orthologue-
LOC104418178 coding sequence
> SEQ ID NO: 39: Eucalyptus grandis ERF115 candidate orthologue-
LOC104416633
MQQLHDLGMTRDDEHGIMVNALQHVISGSSSTRPEPIPAFRSSTPSHLSADGTAAEQRAPGLLSLPDVA
TCQVCRIDGCLGCNFFAPGRVAMAVQGGGDPAKAAQALGVGQSNKSAGRKRKGFYRGVRQRPWGKWAAE
IRDPRRAARVWLGTFETAEQAARAYDRAALEFRGARAKLNFPLLPNDCTSTGAGKSRDMMEDGEAEETI
KARAEGGESNDVREKAPSFERDDERGASEFWEKLEKEELEQWPVMHLPP*
> SEQ ID NO: 89: Eucalyptus grandis ERF115 candidate orthologue-
LOC104416633 coding sequence
> SEQ ID NO: 40: Citrus sinensis ERF115 candidate orthologue-
LOC102629317
MYGTAVVSALSQVIGNTQNSPTSLQLSQNPNFTTSSPNTSERDLSQRVEDQGNVRRRHYRGVRQRPWGK
WAAEIRDPKKAARVWLGTFDTAEAAALAYDEAALRFKGSKAKLNFPERVQANLTTHRYQDHYYHAAAAT
TSQQVSNPPPPPPPRPLPLTQEVMYSNLFQYQQANYGIPSGFYGEYRYLPVTLPTTSSSSSSSSATSSQ
QPQQHELLRYGMQLESSSSSASDPHESTRRNSDTSHPGD*
> SEQ ID NO: 90: Citrus sinensis ERF115 candidate orthologue-
LOC102629317 coding sequence
> SEQ ID NO: 41: Citrus sinensis ERF115 candidate orthologue-
LOC102620304
MSAMVSALTQVIGTTTTDADADPSPAAVKEESSDNPLQQTQTQTQDQDQGTRRRHYRGVRQRPWGKWAA
EIRDPKKAARVWLGTFETAEDAAMAYDKAALKFKGTKAKLNFPERVQGTTEFVYLDSSSSSSAFHHHHE
SVMPAPPPRPTSMHHGAYPDLLQYAQILSSDDATFNYYTSNLFNPQSSSSSSSTPSTFSSSTTSLEQQQ
EMTRFSSNYESLSGSDFQDHSNNPNG*
> SEQ ID NO: 91: Citrus sinensis ERF115 candidate orthologue-
LOC102620304 coding sequence
> SEQ ID NO: 42: Citrus sinensis ERF115 candidate orthologue-
LOC102620304
MSAMVSALTQVIGTTTTDADADPSPAAVKEESSDNPLQQTQTQTQDQDQEGTRRRHYRGVRQRPWGKWA
AEIRDPKKAARVWLGTFETAEDAAMAYDKAALKFKGTKAKLNFPERVQGTTEFVYLDSSSSSSAFHHHH
ESVMPAPPPRPTSMHHGAYPDLLQYAQILSSDDATFNYYTSNLFNPQSSSSSSSTPSTFSSSTTSLEQQ
QEMTRFSSNYESLSGSDFQDHSNNPNG*
> SEQ ID NO: 92: Citrus sinensis ERF115 candidate orthologue-
LOC102620304 coding sequence
> SEQ ID NO: 43: Carica papaya ERF115 candidate orthologue-KM453703.1
MCGGAIISDFIPTATTRSCKLTADYLWPDLNRNRKSKKSSKRSEVVDLDDDFEADFQGFKDDESDIDVD
EDLDDIDAVFSDIKPFAFSATPLPRKTTASALSNGSKPVKAVEFNGLAEKSAKRKRKNQYRGIRQRPWG
KWAAEIRDRRKGVRVWLGTFNTAEEAARAYDAEARRIRGKKAKVNFPDESPRASPKRAVNSMKPVAKAI
LNSAQPNLSQNVNYFNNLGQDYYNTMVFVDEKPQMNQFASMNSFPPRRNAGVKPFVPSDNTHMYFSSDP
GSNSFGCSEFGWGDQATKTPEISSVLLDQPQFVEDCNPEKKLKCSSETMVPVQGNANKSLSEELLAFDN
QMKYLQVPHLDSNWDSSLDAFLNGDAPQDAGNSMDLWAFDDLPSLVGGVF*
> SEQ ID NO: 93: Carica papaya ERF115 candidate orthologue-KM453703.1
coding sequence
> SEQ ID NO: 44: Musa acuminata subsp. malaccensis ERF115 candidate
orthologue-LOC1039B8289
MAACSSGTAKWKLREGKRKRARGWEKAKSKQECRAHQSRRPAQSSERLFLSQSRIIHSLSFLLLRLLLD
RLPLSPLSAVNISVSLSRCCIYLRLSVVFLCRRRRCHHQRISPDPLSGRRKEEERSRNDKVDRRPGKRP
LPPDELEKKEGDQQQAVSRFASSRADHDASAMVSALAKVISSSSSVVDTRGGEPASTQQGIKLEEAAGR
GDTEAAQVSEEQGNVRRRHYRGVRQRPWGKWAAEIRDPRKAARVWLGTFDTAEDAAVAYDEAALRFKGT
KAKLNFPERVQGRTDLGFLVSPGVPERQPPRVPLQLPATSYPDLLQYAQLLQSRDEDLQNVASGLYVGG
TFTPVSSQTPTTSALGSSQHFLDFSSQSQYTNFSSSSSSSSSSSWVHGEQKDKDGSRPP*
> SEQ ID NO: 94: Musa acuminata subsp. malaccensis ERF115 candidate
orthologue-LOC103988289 coding sequence
> SEQ ID NO: 45: Musa acuminata subsp. malaccensis ERF115 candidate
orthologue-LOC104000654
MVSALSRVISSSSSVIDASAGEPTVNQQGIKLEGADPGEKQAIQISEEQGTVRRHYRGVRQRPWGKWAA
EIRDPKKAARVWLGTFDTAEDAAVAYDEAALRFKGSKAKLNFPQRVQGRAELSFLASPGIPRRQPQPPT
RPPASSYPDLFRYAQLLQSGDDNLQSVASGLYVGSAFTSAPSQAPPSSTSGSLPQFLGFSSHSPYSSSS
SSSGSWVYGDHKDKDSSRPP*
> SEQ ID NO: 95: Musa acuminata subsp. malaccensis ERF115 candidate
orthologue-LOC104000654 coding sequence
> SEQ ID NO: 46: Musa acuminata subsp. malaccensis ERF115 candidate
orthologue-LOC103970805
MDRRHGKRPLPPDEAAPEEKAGELSCSPLARADQDASAIVSALAHVIGSCSPVAGVGGGEMRQDVSGSG
TGSVENRTQPSEEQGNAGRRHYRGVRQRPWGKWAAEIRDPQKAARVWLGTFDTAVDAAIAYDEAALRFK
GCKAKLNFPERVQGRSDLGFLTHRWQAQPPVQLPATSYPDLLRYARLLQSRDDDLHNRAVGLHPAGSSF
MSTSSHTTPTSSLSGSSQELVGFSHHWQLRSSSSSSSWPQVDLQDEDED*
> SEQ ID NO: 96: Musa acuminata subsp. malaccensis ERF115 candidate
orthologue-LOC103970805 coding sequence
> SEQ ID NO: 47: Musa acuminata subsp. malaccensis ERF115 candidate
orthologue-LOC103977129
MVSALSHVISSRSPPVGAGGGEPVMVQHDGKLSGSGSGSAEIRTQPSGEQGRRHYRGVRQRPWGKWAAE
IRDPRKAARVWLGTFDTAEDAAIAYDEAALRFKGTKAKLNFPERVQGRTDLGFLVSGGGSERQPQPPTQ
RLPAANSYPNLLQYAQLLQSRDQDLHQAAFGLYAGSTFTSTSSQTSPTSMSAASSQEMLDFTCQSHFKS
SSSSSSWPHGGHKHEDQQPPGM*
> SEQ ID NO: 97: Musa acuminata subsp. malaccensis ERF115 candidate
orthologue-LOC103977129 coding sequence
> SEQ ID NO: 48: Musa acuminata subsp. malaccensis ERF115 candidate
orthologue-LOC103976772
MVSALTHVISSVSPVVGAGGDELVAEPDASCGSGPGSMEIGTQASEEQGRRHYRGVRQRPWGKWAAEIR
DPKKAARVWLGTFDRAEDAAMAYDEAALRFKGTKAKLNFPERVQGRTDLGFLVTRAAPERQPQPPATSY
PDLRQYAQLLQSGDADVHNAALGLYAGSTITSTSLSGSSQETQDLSSRSQFTSSSASSSWPQSGQKEKD
QRPPTM*
> SEQ ID NO: 98: Musa acuminata subsp. malaccensis ERF115 candidate
orthologue-LOC103976772 coding sequence
> SEQ ID NO: 49: Musa acuminata subsp. malaccensis ERF115 candidate
orthologue-LOC1O3979104
MCHNVANPHQLPDDSFAAEGSDGAPLSSYHRAQEISTIVSALAHVMASERRPRPVGMAVDSVSVVSSSS
SSSSSSSSSLSCISSSYSSPSLGGQGGGARSQNRTRRVPSPPDLALRHHQGLGEFARYRGDASPDVAAT
EQYPQGGPLPILGYPVPAAAMEEPSPASSNPEEAERSEPRRKYRGVRQRPWGKWAAEIRDPHKAARVWL
GTFETAEEAARAYDAAALRFRGRRAKLNFPENVRLQPSLSVPLATSNSPATTSDTITDYLAYTRLLQGG
EEHPRIPPTSLLDQYMYSNYASPMCSTVNDGSSLPAPSIPTYSSVVSSSSTPYSPFYASSTTEQQTNWS
GVSDIPETSWMGSSQFPPSSSGS*
> SEQ ID NO: 99: Musa acuminata subsp. malaccensis ERF115 candidate
orthologue-LOC103979104 coding sequence
> SEQ ID NO: 50: Musa acuminata subsp. malaccensis ERF115 candidate
orthologue-LOC103993551
MCLKVANPHQSSDGSFAAAGSDEMEEDAAAAAGMMYSSVTAQAALLSGHRRSRETSTMVSALTRVMAGE
QRPRPVPMAVDSMSAVSSSSSFSYIFSPPPSYSSPSTGGQSGGASSQTRTRPELPSHLALRYYRCLGEF
GSYYGGASPDVAAVEQYPQAFLPMLQSPAPAAAAVEEASPASSNQEERERAAPKRKYRGVRQRPWGKWA
AEIRDPYKAARVWLGTFETAEEAARAYDEAALRFRGSRAKLNFPENVRLQPSHSVALAAQVPPSNSPAT
SSGAVSDYLAYSRLLQGASEYQRLPPASLLDPFVHSGVNDSSSLPASSFHANSVPSSTVISPSSSSSSS
YPPSYASSTPTERQMIWGGASGFPETSWTHSSQFPPSSSGDS*
> SEQ ID NO: 100: Musa acuminata subsp. malaccensis ERF115 candidate
orthologue-LOC103993551 coding sequence

### 7. PAT1 Orthologue Sequences

SEQ ID NO: 101 depicts the amino acid sequence of the Arabidopsis thaliana Phytochrome A Signal Transduction 1 (PAT)1 (490 aa).

Orthologue sequence search was supported by phylogenetic tree, orthologue searches in PLAZA (http://bioinformatics.psb.ugent.be/plaza/).

The following species for identification of candidate PAT1 orthologue sequences were evaluated:
Zea mays (corn), Vitis vinifera (grape), Populus trichocarpa (poplar tree), Solanum lycopersicum (tomato), Solanum tuberosum (potato), Glycine max (soy bean), Gossypium raimondii (cotton), Brassica rapa (turnip), Hordeum vulgare (barley), Oryza sativa ssp. Indica, & Japonica (rice), Setaria italic (millet), Sorghum bicolor (sorghum), Theobroma cacao (cacao), Elaeis guineensis (oil palm), Malus domestica (apple), Fragaria vesca (strawberry), Beta vulgaris (beet), Eucalyptus grandis (eucalyptus), Citrus sinensis (orange tree), Carica papaya (papaya), Musa acuminate (banana)

> SEQ ID NO: 101: Arabidopsis thaliana PAT1-At5g48150
MYKQPRQELEAYYFEPNSVEKLRYLPVNNSRKRFCTLEPFPDSPPYNALSTATYDDTCGSCVTDELNDF
KHKIREIETVMMGPDSLDLLVDCTDSFDSTASQEINGWRSTLEAISRRIDLRADVSCAKAMSENDLMMA
HSMMEKLRQMVSVSGEPIQRLGAYLLEGLVAQLASSGSSIYNALNRCPEPASTELLSYMHILYEVCPYF
KFGYMSANGAIAEAMKEENRVHIIDFQIGQGSQWVTLIQAFAARPGGPPRIRITGIDDMTSAYARGGGL
SIVGNRLAKLAKQFNVPFEFNSVSVSVSEVKPKNLGVRPGEALAVNFAFVLHHMPDESVSTENHRDRLL
RMVKSLSPKVVTLVEQESNTNTAAFFPRFMETMNYYAAMFESIDVTLPRDHKQRINVEQHCLARDVVNI
IACEGADRVERHELLGKWRSRFGMAGFTPYPLSPLVNSTIKSLLRNYSDKYRLEERDGALYLGWMHRDL
VASCAWK*
> SEQ ID NO: 126: Arabidopsis thaliana PAT1-At5g48150 coding sequence
> SEQ ID NO: 102: Zea mays PAT1 candidate orthologue-ZM01G25390
> SEQ ID NO: 127: Lea mays PAT1 candidate orthologue-ZM01G25390
coding sequence
> SEQ ID NO: 103: Zea mays PAT1 candidate orthologue-ZM02G37800
> SEQ ID NO: 128: Zea mays PAT1 candidate orthologue-ZM02G37800
coding sequence
> SEQ ID NO: 104: Zea mays PAT1 candidate orthologue-ZM07G25570
> SEQ ID NO: 129: Lea mays PAT1 candidate orthologue-ZM07G25570
coding sequence
> SEQ ID NO: 105: Vitis vinifera PAT1 candidate orthologue-
VV12G09370
> SEQ ID NO: 130: Vitis vinifera PAT1 candidate orthologue-
VV12G09370 coding sequence
> SEQ ID NO: 106: Populus trichocarpa PAT1 candidate orthologue-
PT01G36170
> SEQ ID NO: 131: Populus trichocarpa PAT1 candidate orthologue-
PT01G36170 coding sequence
> SEQ ID NO: 107: Solanum lycopersicum PAT1 candidate orthologue-
SL04G014830
> SEQ ID NO: 132: Solanum lycopersicum PAT1 candidate orthologue-
SL04G014830 coding sequence
> SEQ ID NO: 108: Solanum tuberosum PAT1 candidate orthologue-
ST04G011980
> SEQ ID NO: 133: Solanum tuberosum PAT1 candidate orthologue-
ST04G011980 coding sequence
> SEQ ID NO: 109: Glycine max PAT1 candidate orthologue-GM02G46730
> SEQ ID NO: 134: Glycine max PAT1 candidate orthologue-GM02G46730
coding sequence
> SEQ ID NO: 110: Gossypium raimondii PAT1 candidate orthologue-
GR02G08060
> SEQ ID NO: 135: Gossypium raimondii PAT1 candidate orthologue-
GR02G08060 coding sequence
> SEQ ID NO: 111: Brassica rapa PAT1 candidate orthologue-BR01G18810
> SEQ ID NO: 136: Brassica rapa PAT1 candidate orthologue-BR01G18810
coding sequence
> SEQ ID NO: 112: Hordeum vulgare PAT1 candidate orthologue-
HV50736G00010
> SEQ ID NO: 137: Hordeum vulgare PAT1 candidate orthologue-
HV50736G00010 coding sequence
> SEQ ID NO: 113: Oryza sativa ssp. Indica PAT1 candidate orthologue-
OSINDICA_10G14210
> SEQ ID NO: 138: Oryza sativa ssp. Indica PAT1 candidate orthologue-
OSINDICA_10G14210 coding seuuence
> SEQ ID NO: 114: Oryza sativa ssp. Japonica PAT1 candidate
orthologue-OS10G22430
> SEQ ID NO: 139: Oryza sativa ssp. Japonica PAT1 candidate
orthologue-OS10G224.30 coding sequence
> SEQ ID NO: 115: Setaria italic PAT1 candidate orthologue-
SI009G27720
> SEQ ID NO: 140: Setaria italic PAT1 candidate orthologue-
SI009G27720 coding sequence
> SEQ ID NO: 116: Sorghum bicolor PAT1 candidate orthologue-
SBO2G037650
> SEQ ID NO: 141: Sorghum bicolor PAT1 candidate orthologue-
SB02G037650 coding sequence
> SEQ ID NO: 117: Theobroma cacao PAT1 candidate orthologue-
TC0007G05450
> SEQ ID NO: 142: Theobroma cacao PAT1 candidate orthologue-
TC0007G05450 coding sequence
> SEQ ID NO: 118: Elaeis guineensis PAT1 candidate orthologue-
LOC105032539
> SEQ ID NO: 143: Elaeis guineensis PAT1 candidate orthologue-
LOC105032539 coding sequence
> SEQ ID NO: 119: Elaeis guineensis PAT1 candidate orthologue-
LOC105034289
> SEQ ID NO: 144: Elaeis guineensis PAT1 candidate orthologue-
LOC105034289 coding sequence
> SEQ ID NO: 120: Malus domestica PAT1 candidate orthologue-
LOC103442977
> SEQ ID NO: 145: Malus domestica PAT1 candidate orthologue-
LOC103442977 coding sequence
> SEQ ID NO: 121: Fragaria vesca subsp. Vesca PAT1 candidate
orthologue-LOC101291701
> SEQ ID NO: 146: Fragaria vesca subsp. Vesca PAT1 candidate
orthologue-LOC101291701 coding sequence
> SEQ ID NO: 122: Beta vulgaris subsp. vulgaris PAT1 candidate
orthologue-LOC104891820
> SEQ ID NO: 147: Beta vulgaris subsp. vulgaris PAT1 candidate
orthologue-LOC104891820 coding sequence
> SEQ ID NO: 123: Eucalyptus grandis PAT1 candidate orthologue-
LOC104434750
> SEQ ID NO: 148: Eucalyptus grandis PAT1 candidate orthologue-
LOC104434750
coding sequence
> SEQ ID NO: 124: Citrus sinensis PAT1 candidate orthologue-
LOC102621664
> SEQ ID NO: 149: Citrus sinensis PAT1 candidate orthologue-
LOC102621664 coding sequence
> SEQ ID NO: 125: Musa acuminata subsp. Malaccensis PAT1 candidate
orthologue-LOC103974444
> SEQ ID NO: 150: Musa acuminata subsp. Malaccensis PAT1 candidate
orthologue-LOC103974444 coding sequence

8. The N-Terminal a Fragment of Several A. thaliana ERFs Also Interacts with PAT1 or SCL Transcription Factors in Yeast.

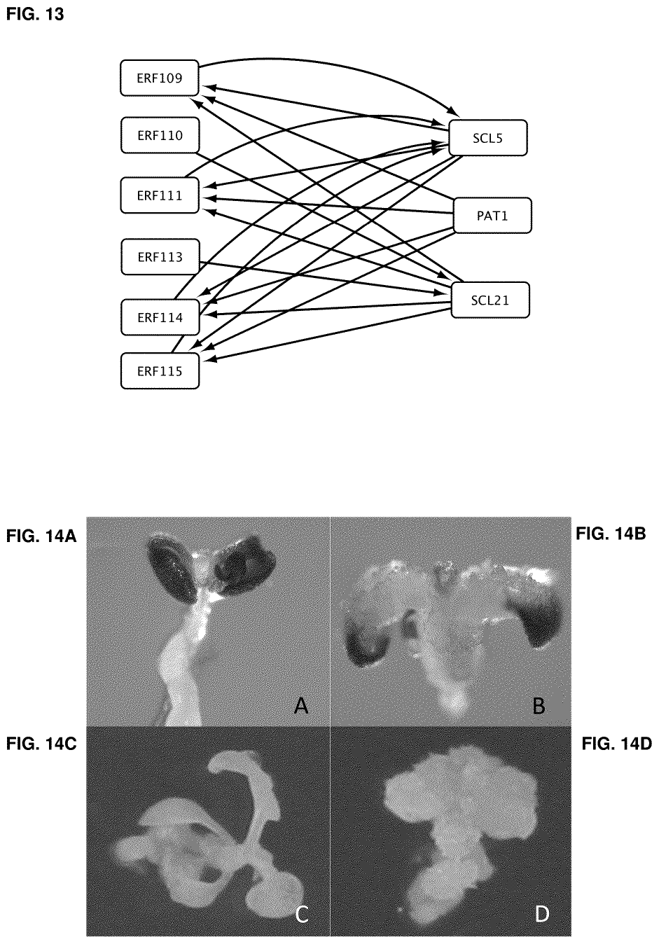

Besides the ERF115-PAT1 and the ERF115-SCL21 interaction that was demonstrated in previous Example 1, the GRAS-domain transcription factors that belong to the PAT1 branch¹⁷ were analysed for interaction with a number of ERFs that contain the SCL-PAT1 interaction motif. As presented in FIG. 13, the A. thaliana ERF transcription factors ERF115 (SEQ ID NO: 1), ERF114 (SEQ ID NO: 160), ERF113 (SEQ ID NO: 159), ERF111 (SEQ ID NO: 156), ERF110 (SEQ ID NO: 155), and ERF109 (SEQ ID NO: 154), which all contain the SCL/PAT1 interaction motif in their A-fragment (see FIG. 6, or bold labelled motif in SEQ ID as provided), showed interaction with PAT1 (SEQ ID NO: 101), SCL5 (SEQ ID NO: 164) and/or SCL21 (SEQ ID NO: 161) in Y2H, whereas no interaction was observed for ERF112 (SEQ ID NO: 57, or SEQ ID NO:58), which lacks the ERF/PAT1 interaction domain.

### 9. PAT1 Overexpression in the Presence of Auxin Triggers Hyperplasia

Although spontaneous callus formation requires the presence of active ERF-PAT1 complex as shown in the present invention, we here also demonstrate for the first time that upon overexpression of PAT1 in seedling grown in medium containing a rather low auxin concentration, increased callus formation resulting in hyperplasia is observed, as compared to wild-type seedlings. Already two weeks post germination, a remarkable difference was observed (FIG. 14b as compared to WT FIG. 14a). Six weeks after germination, the seedlings of the PAT1 overexpression line incubated in low 2,4D containing medium was fully covered by callus tissue (FIG. 14d). This observation suggests that for some crops, or in some transformation protocols, an introduction of PAT1 is sufficient to allow regeneration in a more rapid and more pronounced form.

### 10. Complex Formation Between ERF115 and SCL21 Leads to Spontaneous Callus Formation

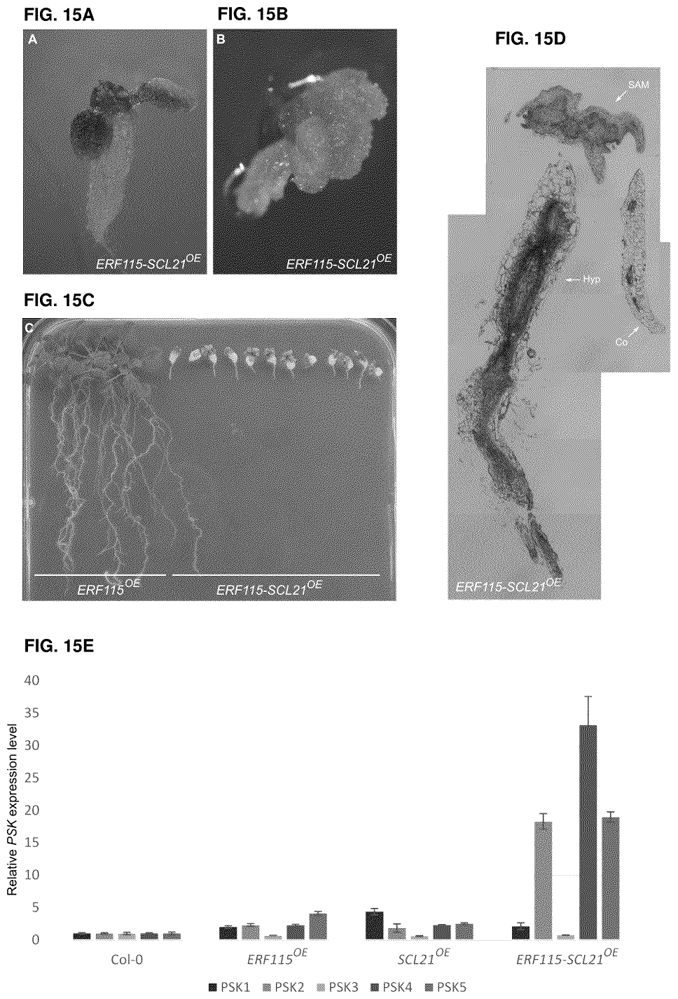

As shown in FIG. 15 a-d, the Arabidopsis seedlings overexpressing ERF115 together with SCL21, for which it was shown that both proteins interact (FIGS. 1 and 5), were also found to induce callus in a spontaneous manner, i.e. without additional hormones such as cytokinin or auxin in the growth medium. Although no biological role in relation to regeneration competence has been identified for the ERF115-SCL21 complex before, the current observation led to the insight that the presence of the interaction motif in the ERF115, and the consequent in vivo interaction with SCL21- or PAT1-related transcription factors is sufficient to provide a bio-tool for increased regeneration of plant cells. Moreover, the expression of the Phytosulfokine peptide precursor genes PSK2, 4 and 5 was also shown to be significantly higher in the co-expressing ERF115-SCL21 overexpression line, indicating that the ERF115 complex activity is triggered, and that the callus induction is not an artefact, but might be linked to responses induced by wounding.

11. Maize ERF115 Orthologues are Induced Upon Wounding, and Shown to Interact with Maize PAT1 Orthologues in Yeast.

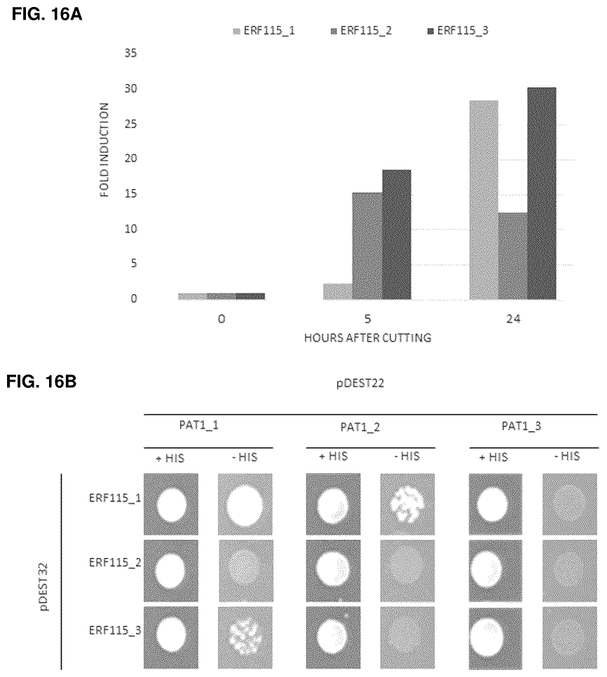

We have shown that the ERF115-PAT1 transcription factor complex appears to play a generic role in tissue regeneration, due to the induction of replenishing cell divisions following ERF115 expression induced by wounding. To identify the orthologues in maize, the expression level was analysed via qRT-PCR in 7 day old B73 maize seedlings upon wounding (i.e. after removal of the root tip). Already few hours after wounding, the expression level of Zea mays ERF115_1 (SEQ ID NO: 2) slightly increased, and more prominently increased more than 10-fold for Zea mays ERF115_2 (SEQ ID NO: 3) and Zea mays ERF115_3 (SEQ ID NO: 4). Twenty four hours after root tip excision, the Zea mays ERF115_2 expression remained around 10-fold higher as compared to before excision, and the Zea mays ERF115_1 expression was induced over 25-fold, as well as the Zea mays ERF115_3 expression (FIG. 16a). This result suggests that the ERF115 potential of cell regeneration induction is retained in maize.

In a Y2H experiment, the interaction of the full length ERF115 maize orthologues with the 3 PAT1 maize orthologues was analysed (FIG. 16b). ERF115_1 showed interaction with PAT1_1 (SEQ ID NO: 102) and PAT1_2 (SEQ ID NO:103), but not with PAT1_3 (SEQ ID NO:104). ERF115_2 did not show interaction with any of the 3 PAT1 maize orthologues. And ERF115_3 showed the clearest growth signal, referring to interaction, with maize PAT1_1, although weaker compared to the ERF115_1/PAT1_1 interaction. These results demonstrate that the protein complex between maize orthologues is also formed in yeast, even for some of the full length maize ERF115 orthologues. In Arabidopsis, the full length ERF115 did not interact with PAT1 in Y2H, but only the A fragment did, indicating that A-fragment interaction is sufficient for activity towards regeneration competence. The functional complex in maize is hence possibly formed by those ERF115_1, ERF115_2, or ERF115_3 A fragments, since these all contain the SCL/PAT1 interaction motif.

### Materials and Methods

### Mutant Lines

The PAT1^{OE} transgenic line was generated by inserting the respective cDNA ORF into the pH7GW2 expression vector under control of the CaMV 35S promoter via Gateway recombination and subsequent Agrobacterium tumefaciens-mediated transformation of Arabidopsis thaliana plants. For GFP reporter constructs, the 2000-bp, 2437-bp, 2163-bp, 3021-bp and 2500-bp promoter regions upstream of the ERF11514, PAT1, SCL21, WOX5 and PLT5 start codons, respectively, were inserted into the pMK7S*NFm14GW plasmid, driving the NLS-GUS/GFP ORF, yielding a nuclear localized GFP/GUS dual reporter. The PERF115:TdTomato line was generated by recombining the respective promoter fragment in front of the NLS-TdTomato ORF into the pB7m24GW,3 plasmid. The pPSK5:PSK5-GFP line was generated by combining the PSK5 genomic ORF in front of the GFP reporter sequence, under control of the 2548-bp PSK5 promoter region into the pK7m34GW plasmid. The erf115, ERF115^{OE}, ERF115^{SRDX}, pat1-2, pCYCD6; 1:GFP, pSCR:YFP-H2B, atm-1, atr-2, and WIND 1^{SRDX} lines were described previously^{14,17,19,23,25,26}. Double mutants or reporter line combinations were generated by crossing. In all experiments, the Col-0 ecotype was used as wild type. Primers used for cloning can be found in Table 1.

### Plant Medium and Growth Conditions.

Plants were grown under a long-day regime (16 h light/8 h darkness) at 21° C. on agar-solidified culture medium (Murashige and Skoog [MS] medium, 10 g/L saccharose, 0.215 g/L 2-(N-morpholino)ethanesulfonic acid [MES], and 1% plant tissue culture agar). The PAT1^{OE} transgenic line was selected using 15 mg/L hygromycin, the pWOX5:NLS-GUS/GFP, pSCL21:NLS-GUS/GFP, pPAT1:NLS-GUS/GFP and pPSK5:PSK5-GFP transgenic lines using 50 mg/L kanamycin, the pERF115:NLS-TdTomato line using 10 mg/L glufosinate-ammonium. For all treatments, 5-day-old seedlings were transferred to culture medium supplemented with 0.6 mg/L bleomycin sulphate (Calbiochem) or 1 mM hydroxyurea (Sigma) for 24 h.

### Callus Induction.

Seedlings were grown in the dark for 1 week in order to obtain etiolated seedlings. Cotyledons and roots were removed with a pair of fine scissors and about 5 mm long hypocotyl segments were placed on callus inducing medium (CIM) (1×MS salts, 1% sucrose, 0.5 g/L MES, 100 mg myo-inositol, 3% Phytagel, 30 mg/L 2,4-D, 1 g/L kinetin) for another 3 weeks.

### Cloning and Chimeric Construct Generation.

For chimeric ERF2 and ERF6 allele generation, the 11-AA conserved motif of Arabidopsis thaliana ERF115 was fused N-terminally to the full-length ORFs (SEQ ID NO: 165 for ERF2, and SEQ ID NO: 166 for ERF6; also see FIGS. 6c and d) using primer adaptor ligation. Basically, the motif-encoding sequence was incorporated 5′ upstream of the ORFs' forward gene-specific primer sequences; as reverse primer, a plasmid-specific primer in which the wild-type ORF was inserted was used.

### Bimolecular Fluorescence Complementation and Protein Localization.

The ERF115, SCL21 and PAT1 ORFs were C-terminally tagged with the head- and tail-fragments of the GFP ORF as indicated in the text and inserted into the pK7m34GW plasmid under control of the CaMV 35S constitutive promoter using Gateway cloning. For Nicotiniana benthamiana leaf blade infiltration, equal concentrations (final OD600=0.5) of a 2-day-old liquid grown Agrobacterium tumefaciens culture containing the respective constructs were incubated in infiltration buffer (10 mM MgCl₂, 10 mM MES [pH5,6], 0.1 mM acetosyringone) for 2 h prior to infiltration of 6-week-old plants. Plants were further grown for two days before interaction analysis. As a negative control for the BiFC, single constructs were infiltrated, yielding no detectable GFP signal.

### Confocal Microscopy.

Infiltrated N. benthamiana leaf segments were analyzed by an Axiovert 100M Confocal Laser Scanning Microscope (Zeiss). Arabidopsis material was counterstained using propidium iodide (PI) or SCRI Renaissance 2200 dye (REN). For PI staining, seedlings were incubated in a 10-μM solution for 3 min prior to imaging. For REN staining, seedlings were grown on culture medium supplemented with the SCRI Renaissance 2200 dye in a final dilution of 1/50,000. For treatments, culture medium was supplemented with the REN dye in combination with the respective compound.

For multicolor confocal microscopy, a Zeiss LSM5 Exciter Confocal was used. GFP fluorescence was observed after excitation using a 488-nm laser and detected using the band-pass 505 nm-530 nm emission filter setting, TdTomato fluorescence was observed after excitation using a 543-nm laser and detected using the band-pass 560 nm-615 nm emission filter, PI fluorescence was observed after excitation using a 543-nm laser and detected using the 650 nm long-pass emission filter, REN fluorescence was observed after excitation using a 405-nm laser and detected using the band-pass 420 nm-480 nm emission filter. For cell specific laser ablation, a ten second pulse was given by the UV-laser (405 nm) at full power using a completely open pinhole with a Zeiss LSM710 Confocal.

For time-tracking, excitation using the 488-nm laser at 5% output was used to reduce laser-dependent tissue damage. Images were acquired by 10 min intervals. The number of anticlinal and replenishing type of divisions of each movie were scored independently by two persons and averaged prior to analysis in order to reduce putative discrepancy.

gRT-PCR Analysis.

RNA was isolated from the respective tissues with the RNeasy isolation kit (Qiagen). DNase treatment with the RQ1 RNase-Free DNase (Promega) was performed prior to cDNA synthesis with the iScript cDNA Synthesis Kit (Bio-Rad). Relative expression levels were determined by qRT-PCR with the LightCycler 480 Real-Time SYBR Green PCR System (Roche). The Arabidopsis thaliana ACT, CAK2 and EMB2386 and maize 18S and EFalpha reference genes were used for normalization. In three biological repetitions, total RNA was isolated by means of the RNeasy Plant mini kit (Qiagen). For the root tips, seedlings were sown and grown for 5 days on nylon meshes (Prosep) to facilitate transfer to medium containing 0.6 mg/L bleomycin for 24 h, and subsequent harvesting using a scalpel. Quantitative PCR data were analyzed using the 2^{(-ΔΔCt)} method. Primer sequences can be found in Table 1.

### Tandem Affinity Purification.

Cloning of a GS-TAP-tagged ERF115 and SCL21 fusion under the control of the constitutive cauliflower mosaic virus 35S promoter and transformation of Arabidopsis cell suspension cultures were performed as described previously²⁷. TAP of protein complexes was done using the GS tag²⁸ followed by protein precipitation and separation²⁹. Proteolysis and peptide isolation, acquisition of mass spectra by a 4800 MALDI TOF/TOF Proteomics Analyzer (AB SCIEX), and mass spectrometry-based protein homology identification based on the TAIR genomic data-base, performed as described previously³⁰. Experimental background proteins were subtracted based on 40 TAP experiments on wild-type cultures and cultures expressing TAP-tagged mock proteins GUS, RFP, and GFP²⁹.

### Histochemical Staining Assay.

GUS staining was performed as described previously³¹. Following staining, samples were cleared in a 75%/25% ethanol/acetic acid solution before mounting on slides using lactic acid. Samples were observed under a BX51 microscope (Olympus).

### Root Cutting Assays.

Root cutting experiments were performed as described previously⁷. Root tip regeneration after 72 hours post cut was scored negative when a collapse of the root meristem was observed.

### Yeast Two-Hybrid.

The Y2H assay was performed as described previously^{31,32}.

### Hypocotyl Sections.

Sections were performed as previously described^{33,34}.

### In Silico Analysis of PAT1 and ERF115 Orthologues

Reciprocal blast searches in combination with phylogenetics and protein domain analysis identified three putative ERF115 and three putative PAT1 maize orthologous sequences (as depicted in SEQ ID NOs:2-4 for ERF115 and SEQ ID NOs:102-104 for PAT1, respectively). The presumptive maize ERF115 genes all contain the conserved amino acid motif in the N-terminal domain that has been identified as the PAT1 interaction motif, as revealed by sequence alignment.

### SEQUENCES LISTED AS REFERENCE

In the ERF sequences, the presence of the SCL/PAT1-interaction domain is indicated in bold.

> SEQ ID NO: 151: Arabidopsis thaliana ERF2-AT5G47220
MYGQCNIESDYALLESITRHLLGGGGENELRLNESTPSSCFTESWGGLPLKENDSEDMLVYGLLKDAFH
FDTSSSDLSCLFDFPAVKVEPTENFTAMEEKPKKAIPVTETAVKAKHYRGVRQRPWGKFAAEIRDPAKN
GARVWLGTFETAEDAALAYDIAAFRMRGSRALLNFPLRVNSGEPDPVRITSKRSSSSSSSSSSSTSSSE
NGKLKRRRKAENLTSEVVQVKCEVGDETRVDELLVS*
> SEQ ID NO: 165: Arabidopsis thaliana ERF2-AT5G47220 coding sequence
> SEQ ID NO: 152: Arabidopsis thaliana ERF6-AT4G17490
MATPNEVSALFLIKKYLLDELSPLPTTATTNRWMNDFTSFDQTGFEFSEFETKPEIIDLVTPKPEIFDF
DVKSEIPSESNDSFTFQSNPPRVTVQSNRKPPLKIAPPNRTKWIQFATGNPKPELPVPVVAAEEKRHYR
GVRMRPWGKFAAEIRDPTRRGTRVWLGTFETAIEAARAYDKEAFRLRGSKAILNFPLEVDKWNPRAEDG
RGLYNKRKRDGEEEEVTVVEKVLKTEESYDVSGGENVESGLTAIDDWDLTEFLSMPLLSPLSPHPPFGY
PQLTVV*
> SEQ ID NO: 166: Arabidopsis thaliana ERF6-AT4G17490 coding sequence
> SEQ ID NO: 153: Arabidopsis thaliana ERF108-AT1G43160
MVSMLTNVVSGETEPSASATWTMGHKREREEFSLPPQPLITGSAVTKECESSMSLERPKKYRGVRQRPW
GKWAAEIRDPHKATRVWLGTFETAEAAARAYDAAALRFRGSKAKLNFPENVGTQTIQRNSHFLQNSMQP
SLTYIDQCPTLLSYSRCMEQQQPLVGMLQPTEEENHFFEKPWTEYDQYNYSSFG*
> SEQ ID NO: 167: Arabidopsis thaliana ERF108-AT1G43160 coding sequence
> SEQ ID NO: 154: Arabidopsis thaliana ERF109-AT4G34410
MHYPNNRTEFVGAPAPTRYQKEQLSPEQELSVIVSALQHVISGENETAPCQGFSSDSTVISAGMPRLDS
DTCQVCRIEGCLGCNYFFAPNQRIEKNHQQEEEITSSSNRRRESSPVAKKAEGGGKIRKRKNKKNGYRG
VRQRPWGKFAAEIRDPKRATRVWLGTFETAEDAARAYDRAAIGFRGPRAKLNFPFVDYTSSVSSPVAAD
DIGAKASASASVSATDSVEAEQWNGGGGDCNMEEWMNMMMMMDFGNGDSSDSGNTIADMFQ
> SEQ ID NO: 168: Arabidopsis thaliana ERF109-AT4G34410 coding sequence
> SEQ ID NO: 155: Arabidopsis thaliana ERF110-AT5G50080
MSAMVSALTQVVSARSQTEAEGAHSSSSSAGHKRGWLGIDSAPIPSSFARVDSSHNPIEESMSKAFPEE
AREKKRRYRGVRQRPWGKWAAEIRDPHRAARVWLGTFDTAEAAARAYDEAALRFRGNKAKLNFPEDVRI
LPPPPPLLRSPADTVANKAEEDLINYWSYTKLLQSSGQRSFLERGQEESSNIFEHSPMEQPLPPSSSGP
SSSNFPAPSLPNT*
> SEQ ID NO: 169: Arabidopsis thaliana ERF110-AT5G50080 coding sequence
> SEQ ID NO: 156: Arabidopsis thaliana ERF111-AT5G64750
MCVLKVANQEDNVGKKAESIRDDDHRTLSEIDQWLYLFAAEDDHHRHSFPTQQPPPSSSSSSLISGFSR
EMEMSAIVSALTHVVAGNVPQHQQGGGEGSGEGTSNSSSSSGQKRRREVEEGGAKAVKAANTLTVDQYF
SGGSSTSKVREASSNMSGPGPTYEYTTTATASSETSSFSGDQPRRRYRGVRQRPWGKWAAEIRDPFKAA
RVWLGTFDNAESAARAYDEAALRFRGNKAKLNFPENVKLVRPASTEAQPVHQTAAQRPTQSRNSGSTTT
LLPIRPASNQSVHSQPLMQSYNLSYSEMARQQQQFQQHHQQSLDLYDQMSFPLRFGHTGGSMMQSTSSS
SSHSRPLFSPAAVQPPPESASETGYLQDIQWPSDKTSNNYNNSPSS*
> SEQ ID NO: 170: Arabidopsis thaliana ERF111-AT5G64750 coding sequence
> SEQ ID NO: 157: Arabidopsis thaliana ERF112-AT2G33710
MHSGKRPLSPESMAGNREEKKELCCCSTLSESDVSDFVSELTGQPIPSSIDDQSSSLTLQEKSNSRQRN
YRGVRQRPWGKWAAEIRDPNKAARVWLGTFDTAEEAALAYDKAAFEFRGHKAKLNFPEHIRVNPTQLYP
SPATSHDRIIVTPPSPPPPIAPDILLDQYGHFQSRSSDSSANLSMNMLSSSSSSLNHQGLRPNLEDGEN
VMTTISTEDDRRRQQHASPDRPIK*
> SEQ ID NO: 158: Arabidopsis thaliana ERF112-AT2G33710-alternatively
spliced
MHSGKRPLSPESMAGNREEKKELCCCSTLSESDVSDFVSELTGQPIPSSIDDQSSSLTLQEKSNSRQRN
YRGVRQRPWGKWAAEIRDPNKAARVWLGTFDTAEEAALAYDKAAFEFRGHKAKLNFPEHIRVNPTQLYP
SPATSHDRIIVTPPSPPPPIAPDILLDQYGHFQSRSSDSSANLSMNMLSSSSSSLNHQGLRPNLEDGEN
VKNISIHKRRK*
> SEQ ID NO: 171: Arabidopsis thaliana ERF112-AT2G33710 coding sequence
> SEQ ID NO: 159: Arabidopsis thaliana ERF113-AT5G13330
MVSALSRVIENPTDPPVKQELDKSDQHQPDQDQPRRRHYRGVRQRPWGKWAAEIRDPKKAARVWLGTFE
TAEEAALAYDRAALKFKGTKAKLNFPERVQGPTTTTTISHAPRGVSESMNSPPPRPGPPSTTTTSWPMT
YNQDILQYAQLLTSNNEVDLSYYTSTLFSQPFSTPSSSSSSSQQTQQQQLQQQQQQREEEEKNYGYNYY
NYPRE*
> SEQ ID NO: 172: Arabidopsis thaliana ERF113-AT5G13330 coding sequence
> SEQ ID NO: 160: Arabidopsis thaliana ERF114-AT5G61890
MYGKRPFGGDESEEREEDENLFPVFSARSQHDMRVMVSALTQVIGNQQSKSHDNISSIDDNYPSVYNPQ
DPNQQVAPTHQDQGDLRRRHYRGVRQRPWGKWAAEIRDPKKAARVWLGTFETAESAALAYDEAALKFKG
SKAKLNFPERVQLGSNSTYYSSNQIPQMEPQSIPNYNQYYHDASSGDMLSFNLGGGYGSGTGYSMSHDN
STTTAATTSSSSGGSSRQQEEQDYARFWRFGDSSSSPHSGY*
> SEQ ID NO: 173: Arabidopsis thaliana ERF114-AT5G61890 coding sequence
> SEQ ID NO: 161: Arabidopsis thaliana Scarecrow-Like 21-AT2G04890
> SEQ ID NO: 174: Arabidopsis thaliana Scarecrow-Like 21-AT2G04890
coding sequence
> SEQ ID NO: 162: Arabidopsis thaliana Scarecrow-Like 1-AT1G21450
> SEQ ID NO: 175: Arabidopsis thaliana Scarecrow-Like 1-AT1G21450
coding sequence
> SEQ ID NO: 163: Arabidopsis thaliana Scarecrow-Like 13-AT4G17230
> SEQ ID NO: 176: Arabidopsis thaliana Scarecrow-Like 13-AT4G17230
coding sequence
> SEQ ID NO: 164: Arabidopsis thaliana Scarecrow-Like 5-AT1G50600,
depicted here as shorter annotated protein (as compared to TAIR)
> SEQ ID NO: 177: Arabidopsis thaliana Scarecrow-Like 5-AT1G50600,
depicted here as shorter annotated encoding nucleotide sequence (as
compared to TAIR)
> SEQ ID NO: 178: Zea mays ERF115_1 synthetic CDS used for cloning
> SEQ ID NO: 179: Zea mays ERF115_2 synthetic CDS used for cloning
> SEQ ID NO: 180: Zea mays ERF115_3 synthetic CDS used for cloning
> SEQ ID NO: 181: Zea mays PAT1_1 synthetic CDS used for cloning
> SEQ ID NO: 182: Zea mays PAT1_2 synthetic CDS used for cloning
> SEQ ID NO: 183: Zea mays PAT1_3 synthetic CDS used for cloning

TABLE 1
Primer sequences

| Cloning primers | Sequence | |
|---|---|---|
| ERF115_A_Fragment_FW | ATGGCGAATTCAGGAAATTATGG | SEQ ID NO: 184 |
| ERF115_A_Fragment_REV | TCACTTCCTCAATAGCCCTTG | SEQ ID NO: 185 |
| ERF115_B_Fragment_FW | ATGAGGCACTATAGAGGGGTAAG | SEQ ID NO: 186 |
| ERF115_B_Fragment_REV | TCAAGCTCTCTCAGGGAAATTG | SEQ ID NO: 187 |
| ERF115_C_Fragment_FW | ATGCAACTAGCAAGTAACACTAG | SEQ ID NO: 188 |
| ERF115_C_Fragment_REV | TCAAAAACCAGAATTAGGAGGTG | SEQ ID NO: 189 |
| ERF2_motif_fusion_FW | ATGGTCTCAGCCTTGACTCAAGTCATTGGAAACATGTACGGACAGTGCAATATAG | SEQ ID NO: 190 |
| ERF2_motif_fusion_REV | GGGGACAAGTTTGTACAAAAAAGCAGGCTTG | SEQ ID NO: 191 |
| ERF6_motif_fusion_FW | ATGGTCTCAGCCTTGACTCAAGTCATTGGAAACATGGCTACACCAAACGAAGTATC | SEQ ID NO: 192 |
| ERF6_motif_fusion_REV | GGGGACAAGTTTGTACAAAAAAGCAGGCTTG | SEQ ID NO: 193 |
| PLT5_promoter_FW | TCTTATATCTAGTGTATTTTCACC | SEQ ID NO: 194 |
| PLT5_promoter_REV | CTTTGGGAATAGGTTTTTTTTTTTC | SEQ ID NO: 195 |
| PAT1_promoter_FW | GACCAATAGATGATATACAAGTG | SEQ ID NO: 196 |
| PAT1_promoter_REV | TTCTAGTCACTTCAATGATCTGC | SEQ ID NO: 197 |
| PAT1_ORF_FW | ATGTACAAGCAGCCTAGACAAGAGC | SEQ ID NO: 198 |
| PAT1_ORF_STOP_REV | TCATTTCCAAGCACACGAGGCAACC | SEQ ID NO: 199 |
| PAT1_ORF_withoutSTOP_REV | TTTCCAAGCACACGAGGCAACCAAATC | SEQ ID NO: 200 |
| PSK5_promoterWFW | CCTGAGTTGCAGAGTGGAGA | SEQ ID NO: 201 |
| PSK5_promoterWREV | TCTTTTATATGACTACTTATGATACAATTC | SEQ ID NO: 202 |
| PSK5_ORF_FW | ATGGTTAAGTTCACAACTTTCCTC | SEQ ID NO: 203 |
| PSK5_ORF_withoutSTOP_REV | GGGATTGTGGTTTTGAGTGTAG | SEQ ID NO: 204 |
| SCL21_promoterWFW | TAGGTCACGATTCGTGCC | SEQ ID NO: 205 |
| SCL21_promoterWREV | CACAAATAAGCAAGTAAGAATCTTT | SEQ ID NO: 206 |
| SCL21_ORF_FW | ATGGACAATGTAAGAGGTTCAATAA | SEQ ID NO: 207 |
| SCL21_ORF_STOP_REV | TCACTTCCATGCACAAGATGAG | SEQ ID NO: 208 |
| SCL21_ORF_withoutSTOP_REV | CTTCCATGCACAAGATGAGAC | SEQ ID NO: 209 |
| WOX5_promoterWFW | AGTAGTTAGAGCCACCATACCCAA | SEQ ID NO: 210 |
| WOX5_promoterWREV | GTTCAGATGTAAAGTCCTCAACTG | SEQ ID NO: 211 |
| RT-qPCR | | |
| ACT_FW | GGCTCCTCTTAACCCAAAGGC | SEQ ID NO: 212 |
| ACT_REV | CACACCATCACCAGAATCCAGC | SEQ ID NO: 213 |
| CAK2_FW | ACCACCATTAACGTGCGTCAAC | SEQ ID NO: 214 |
| CAK2_REV | GATCTTGGCGAGAGAATCGGTATC | SEQ ID NO: 215 |
| EMB2386_FW | CTCTCGTTCCAGAGCTCGCAAAA | SEQ ID NO: 216 |
| EMB2386_REV | AAGAACACGCATCCTACGCATCC | SEQ ID NO: 217 |
| PAT1_FW | TTTGCCTTTGTGCTTCATCA | SEQ ID NO: 218 |
| PAT1_REV | CATCGCGGCATAGTAGTTCA | SEQ ID NO: 219 |
| PLT1_FW | ACGAAAACCAATCCAACCAC | SEQ ID NO: 220 |
| PLT1_REV | ATTGGACGCTAGGCATCAAG | SEQ ID NO: 221 |
| PLT2_FW | GAGGTTCCAAAAGTGGCTGA | SEQ ID NO: 222 |
| PLT2_REV | CGTTGGTTTGATGAATGTCG | SEQ ID NO: 223 |
| PLT3_FW | GGACGGGAAGATATGAAGCA | SEQ ID NO: 224 |
| PLT3_REV | AGGGAAATTGGTGGTAGCAG | SEQ ID NO: 225 |
| PLT5_FW | CAGGCACGAATTGGAAGAGT | SEQ ID NO: 226 |
| PLT5_REV | AGGCATTAGTCCACCCACAG | SEQ ID NO: 227 |
| PLT7_FW | GGATATGACAAGGAAGATAGAGCA | SEQ ID NO: 228 |
| PLT7_REV | TGCAATAAACTCTTGCTTGGTC | SEQ ID NO: 229 |
| PSK5_FW | GCATCAGCTCGGCTCAAT | SEQ ID NO: 230 |
| PSK5_REV | GCATTCTTCTTCTCCAACACC | SEQ ID NO: 231 |
| WIND1_FW | CCCGGCTTAACTTCCCTAAC | SEQ ID NO: 232 |
| WIND1_REV | TAGATCTGGCGACGAAACCT | SEQ ID NO: 233 |
| WOX5_FW | GGCTAGGGAGAGGCAGAAAC | SEQ ID NO: 234 |
| WOX5_REV | TCCACCTTGGAGTTGGAGTC | SEQ ID NO: 235 |
| ERF115_1 FW | AAAGACGCGTGATCGAGAGCTG | SEQ ID NO: 236 |
| ERF115_1 REV | ACGTGTTGCTCAATCTTGTCTCAC | SEQ ID NO: 237 |
| ERF115_2 FW | AAGTAGCTGTGCGGAGGAATGG | SEQ ID NO: 238 |
| ERF115_2 REV | GGGTAAGTGGCAGCGACGATTT | SEQ ID NO: 239 |
| ERF115_3 FW | TGATGACACCCGAGTGGTCTTTG | SEQ ID NO: 240 |
| ERF115_3 REV | ACACCGATGGAGTCTTGGAGAAC | SEQ ID NO: 241 |
| 18S FW | ACCTTACCAGCCCTTGACATATG | SEQ ID NO: 242 |
| 18S REV | GACTTGACCAAACATCTCACGAC | SEQ ID NO: 243 |
| EF alpha FW | AGTCCGTTGAGATGCACCATG | SEQ ID NO: 244 |
| EF alpha REV | CACATACCCACGCTTCAGATCC | SEQ ID NO: 245 |

### REFERENCES

1. Sena, G. & Birnbaum, K. D. Built to rebuild: in search of organizing principles in plant regeneration. Curr. Opin. Genet. Dev. 20, 460-465 (2010).
2. Birnbaum, K. D. & Sánchez Alvarado, A. Slicing across kingdoms: regeneration in plants and animals. Cell 132, 697-710 (2008).
3. Stappenbeck, T. S. & Miyoshi, H. The role of stromal stem cells in tissue regeneration and wound repair. Science 324, 1666-9 (2009).
4. Melnyk, C. W. & Meyerowitz, E. M. Plant grafting. Curr. Biol. 25, R183-R188 (2015).
5. Thorpe, T. A. History of plant tissue culture. Mol. Biotechnol. 37, 169-180 (2007).
6. Sugimoto, K., Gordon, S. P. & Meyerowitz, E. M. Regeneration in plants and animals: dedifferentiation, transdifferentiation, or just differentiation? Trends Cell Biol. 21, 212-218 (2011).
7. Ikeuchi, M., Sugimoto, K. & Iwase, A. Plant callus: mechanisms of induction and repression. Plant Cell 25, 3159-3173 (2013).
8. Vogel, G. How does a single somatic cell become a whole plant? Science 309, 86 (2005).
9. Iwase, A. et al. The AP2/ERF transcription factor WIND1 controls cell dedifferentiation in Arabidopsis. Curr Biol. 21, 508-14 (2011).
10. van den Berg, C., Willemsen, V., Hendriks, G., Weisbeek, P. & Scheres, B. Short-range control of cell differentiation in the Arabidopsis root meristem. Nature 390, 287-289 (1997).
11. Sarkar, A. K. et al. Conserved factors regulate signalling in Arabidopsis thaliana shoot and root stem cell organizers. Nature 446, 811-814 (2007).
12. Haecker, A. et al. Expression dynamics of WOX genes mark cell fate decisions during early embryonic patterning in Arabidopsis thaliana. Development 131, 657-668 (2004).
13. Fulcher, N. & Sablowski, R. Hypersensitivity to DNA damage in plant stem cell niches. Proc. Natl. Acad. Sci. USA 106, 20984-20988 (2009).
14. Heyman, J. et al. ERF115 controls root quiescent center cell division and stem cell replenishment. Science 342, 860-863 (2013).
15. Zhang, Y. et al. TOPOISOMERASE1a acts through two distinct mechanisms to regulate stele and columella stem cell maintenance in the Arabidopsis root. Plant Physiol. 171, 483-93 (2016).
16. Heidstra, R., Welch, D. & Scheres B. Mosaic analyses using marked activation and deletion clones dissect Arabidopsis SCARECROW action in asymmetric cell division. Genes Dev. 18, 1964-9 (2004).
17. Torres-Galea, P., Hirtreiter, B. & Bolle, C. Two GRAS proteins, SCARECROW-LIKE21 and PHYTOCHROME A SIGNAL TRANSDUCTION1, function cooperatively in phytochrome A signal transduction. Plant Physiol. 161, 291-304 (2013).
18. Nakano, T., Suzuki, K., Fujimura, T. & Shinshi, H. Genome-wide analysis of the ERF gene family in Arabidopsis and rice. Plant Physiol. 140, 411-432 (2006).
19. Sozzani, R. et al. Spatiotemporal regulation of cell-cycle genes by SHORTROOT links patterning and growth. Nature 466, 128-32 (2010).
20. Koizumi, K., Hayashi, T., Wu, S., Gallagher, K. L. The SHORT-ROOT protein acts as a mobile, dose-dependent signal in patterning the ground tissue. Proc. Natl. Acad. Sci. U.S.A. 109, 13010-5 (2012).
21. Sena, G., Wang, X., Liu, H.-Y., Hofhuis, H. & Birnbaum, K. D. Organ regeneration does not require a functional stem cell niche in plants. Nature 457, 1150-1153 (2009).
22. Efroni, I. et al. Root Regeneration Triggers an Embryo-like Sequence Guided by Hormonal Interactions. Cell 165, 1721-33 (2016).
23. Kareem, A. et al. PLETHORA genes control regeneration by a two-step mechanism. Curr. Biol. 25, 1017-1030 (2015).24.
24. Chera, S. et al. Apoptotic cells provide an unexpected source of Wnt3 signaling to drive hydra head regeneration. Dev. Cell 17, 279-289 (2009).
25. Garcia, V. et al. AtATM is essential for meiosis and the somatic response to DNA damage in plants. Plant Cell. 1, 119-32 (2003)
26. Culligan, K., Tissier, A., & Britt, A. ATR regulates a G2-phase cell-cycle checkpoint in Arabidopsis thaliana. Plant Cell. 5, 1091-104 (2004)
27. Van Leene, J. et al. A tandem affinity purification-based technology platform to study the cell cycle interactome in Arabidopsis thaliana. Mol Cell Proteomics. 6, 1226-38 (2007)
28. Bürckstümmer, T. et al. An efficient tandem affinity purification procedure for interaction proteomics in mammalian cells. Nat Methods. 3, 1013-9 (2006)
29. Van Leene, J., Witters, E., Inzé, D. & De Jaeger, G. Boosting tandem affinity purification of plant protein complexes. Trends Plant Sci. 13, 517-520 (2008)
30. Van Leene, J. et al. Targeted interactomics reveals a complex core cell cycle machinery in Arabidopsis thaliana. Mol Syst Biol. 10, 397 (2010)
31. Lammens, T. et al. Atypical E2F activity restrains APC/CCCS52A2 function obligatory for endocycle onset. Proc Natl Acad Sci. 38, 14721-6 (2008)
32. Heyman, J. et al. Arabidopsis ULTRAVIOLET-B-INSENSITIVE4 maintains cell division activity by temporal inhibition of the anaphase-promoting complex/cyclosome. Plant Cell. 12, 4394-410 (2011)
33. Gietz, D., St Jean, A., Woods, R. A. & Schiestl, R. H. Improved method for high efficiency transformation of intact yeast cells. Nucleic Acids Res. 20, 1425 (1992)
34. Beeckman, T. and Viane R. Embedding Thin Plant specimens for oriented Sectionings. Biotechn. Histochem. 75, 23-26 (1999)
35. De Smet, I. et al. An Easy and Versatile Embedding method for transverse sections. Journal of Microscopy. 213, 76-80 (2004)
36. Sugiyama, M. Organogenesis in vitro. Curr Opin Plant Biol. 2: 61-64 (1999)
37. Grafi G (2004) How cells dedifferentiate: a lesson from plants. Dev Biol. 268:1-6
38. Mehrnia M., Balazadeh S, Zanor M I, Mueller-Roeber B. EBE, an AP2/ERF transcription factor highly expressed in proliferating cells, affects shoot architecture in Arabidopsis. Plant Physiol. 162(2), 842-857 (2013)
39. Boutilier K, Offringa R, Sharma V K, Kieft H, Ouellet T, Zhang L, Hattori J, Liu C M, van Lammeren A A, Miki B L, Custers J B, van Lookeren Campagne M M. Ectopic expression of BABY BOOM triggers a conversion from vegetative to embryonic growth. The Plant Cell. 14, 1737-1749 (2002)
40. Duan et al. Transgenic rice plants harboring an introduced potato proteinase inhibitor II gene are insect resistant. Nat Biotechnol 14:494-498 (1996)
41. McGurl et al. Structure, expression, and antisense inhibition of the systemin precursor gene. Science 225:1570-1573 (1992)
42. De Veylder et al. Herbicide safener-inducible gene expression in Arabidopsis thaliana. Plant Cell Physiol 38:568-577 (1997)
43. Cao et al. Bacillus thuringiensis protein production, signal transduction, and insect control in chemically inducible PR-1a/cryl Ab broccoli plants. Plant Cell Reports 6:554-60 (2006)
44. Ono et al. Transient assay system for the analysis of PR-1a gene promoter in tobacco BY-2 cells. Biosci. Biotechnol. Biochem. 68:803-7 (2004)
45. Mett et al. Copper-controllable gene expression system for whole plants. PNAS 90:4567-4571 (1993)
46. Caddick et al. An ethanol inducible gene switch for plants used to manipulate carbon metabolism. Nature Biotechnol 16:177-80 (1998)
47. Bruce et al. Expression profiling of the maize flavonoid pathway genes controlled by estradiol-inducible transcription factors CRC and P. Plant cell 12:65-79 (2000)
48. Zuo J, Niu Q W, Chua N H. Technical advance: An estrogen receptor-based transactivator XVE mediates highly inducible gene expression in transgenic plants. Plant J. 24(2):265-73 (2000)
49. Sakuma et al. DNA-binding specificity of the ERF/AP2 domain of Arabidopsis DREBs, transcription factors involved in dehydration- and cold-inducible gene expression. Biochem Biophys Res Comm 290:998-1009 (2002)
50. Bolle et al. PAT1, a new member of the GRAS family, is involved in phytochrome A signal transduction. Genes Dev. 14(10):1269-78 (2000)
51. Straub et al. Structure and promoter analysis of an ABA- and stress-regulated barley gene, HVA1 Plant Mol Biol 26:617-630 (1994)

1. A composition comprising:
a first chimeric gene construct and a second chimeric gene construct;
the first chimeric gene construct comprising the following operably linked DNA elements:
a) a plant expressible promoter,
b) a DNA region encoding an Ethylene Response Factor (ERF) comprising an SCL/PAT1 interaction motif and
c) a 3′ end region comprising transcription termination and polyadenylation signals functioning in cells of a plant, and
the second chimeric gene construct comprising the following operably linked DNA elements:
a) a plant expressible promoter,
b) a DNA region encoding a Phytochrome A Signal Transduction 1 (PAT1) branch transcription factor (TF) and
c) a 3′ end region comprising transcription termination and polyadenylation signals functioning in cells of a plant
wherein expression of the first chimeric gene construct and the second chimeric gene construct in a plant leads to the formation of an ERF/PAT1 complex,
wherein the ERF comprising an SCL/PAT1 interaction motif is ERF115 or ERF114, and wherein the PAT1 branch TF is PAT1 or SCL21,
wherein the plant has increased plant cell regeneration potential without additional hormones as compared to an otherwise identical plant lacking the first chimeric gene construct and the second chimeric gene construct.

2. (canceled)

3. (canceled)

4. The composition of claim 1, wherein the DNA region encoding the ERF is selected from the group consisting of SEQ ID NO: 51-100 and 160, and wherein the DNA region encoding the PAT1 branch TF is selected from the group consisting of SEQ ID NO: 126-150 and 161.

5. The composition of claim 1,
wherein the first and second chimeric gene constructs are flanked by recombination sites, and
wherein the composition further comprises a third chimeric gene construct comprising the following operably linked DNA elements:
a) a plant expressible promoter,
b) a DNA region encoding a site-specific recombinase that is capable of recognizing and implementing recombination at the recombination sites and
c) a 3′ end region comprising transcription termination and polyadenylation signals functioning in cells of a plant.

6. The composition of claim 1, wherein the chimeric gene constructs are comprised in set of at least one recombinant vector.

7. A plant, plant cell or plant seed comprising the chimeric gene constructs of claim 1.

8. The plant, plant cell or plant seed according to claim 7, wherein ERF and PAT1 branch TF are co-expressed.

9. (canceled)

10. (canceled)

11. (canceled)

12. A method for producing a transgenic plant, the method comprising introducing into a plant cell the composition of claim 1 and regenerating a plant from the plant cell.

13. The method according to claim 12, further comprising transiently expressing the chimeric gene constructs.

14. The method according to claim 1, further comprising:
expressing the ERF and PAT1 branch TF encoded by the chimeric gene constructs;
forming a callus from the plant cell,
expressing a recombinase, and
excising the chimeric gene constructs with the recombinase.

15. (canceled)

16. The composition of claim 1,
wherein the first chimeric gene construct and the second chimeric gene construct form part of a multicistronic gene region;
wherein the plant expressible promoters of the first chimeric gene construct and the second chimeric gene construct are the same and wherein the plant expressible promoter promotes transcription of the multicistronic gene region; and
wherein the 3′ end regions comprising transcription termination and polyadenylation signals functioning in cells of a plant of the first chimeric gene construct and the second chimeric gene construct are the same.

17. The composition of claim 16, wherein the DNA region encoding the ERF is selected from the group consisting of SEQ ID NOs: 51-100 and 160, and wherein the DNA region encoding the PAT1 branch TF is selected from the group consisting of SEQ ID NOs: 126-150 and 161.

18. The composition of claim 16, wherein the composition is comprised in a recombinant vector.

19. A method for producing a transgenic plant, the method comprising:
introducing into a plant cell the composition of claim 16, and
regenerating a plant from the cell.

20. The method according to claim 19, further comprising transiently expressing the chimeric gene constructs.

21. The method according to claim 20, further comprising:
expressing the ERF and PAT1 branch TF encoded by the chimeric gene constructs;
forming a callus from the plant cell;
expressing a recombinase; and
excising the chimeric gene construct with the recombinase.

22. A plant comprising:
a first chimeric gene construct, a second chimeric gene construct, and an ERF/PAT1 branch TF complex;
the first chimeric gene construct comprising the following operably linked DNA elements:
a) a plant expressible promoter,
b) a DNA region encoding an Ethylene Response Factor (ERF) comprising an SCL/PAT1 interaction motif, and
c) a 3′ end region comprising transcription termination and polyadenylation signals functioning in cells of the plant, and
the second chimeric gene construct comprising the following operably linked DNA elements:
a) a plant expressible promoter,
b) a DNA region encoding Phytochrome A Signal Transduction 1 (PAT1) branch TF, and
c) a 3′ end region comprising transcription termination and polyadenylation signals functioning in cells of the plant
wherein the ERF comprising an SCL/PAT1 interaction motif is ERF115 or ERF114, and wherein the PAT1 branch TF is PAT1 or SCL21, and
wherein the plant has increased plant cell regeneration potential without additional hormones as compared to an otherwise identical plant lacking the first chimeric gene construct and the second chimeric gene construct.
